SEQUENCE LISTING (41 sequences)

SEQ ID NO: 1 - protein, 153 aa, Artificial Sequence
  Synthetic construct
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 2 - protein, 219 aa, Artificial Sequence
  Synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 3 - protein, 263 aa, Artificial Sequence
  Synthetic construct
SNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 308 aa, Artificial Sequence
  Synthetic construct
TTTPAPRPPTPAPTIASQPLSLRPEACRPAAGGAVHTRGLDFACDSNLFVASWIAVMIIFRIGMAVAIFCCFFFPSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 134 aa, Artificial Sequence
  Synthetic construct
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 6 - protein, 27 aa, Artificial Sequence
  Synthetic construct
FWVLVVVGGVLACYSLLVTVAFIIFWV

SEQ ID NO: 7 - protein, 21 aa, Artificial Sequence
  Synthetic construct
IYIWAPLAGTCGVLLLSLVIT

SEQ ID NO: 8 - protein, 22 aa, Artificial Sequence
  Synthetic construct
MALIVLGGVAGLLLFIGLGIFF

SEQ ID NO: 9 - protein, 55 aa, Artificial Sequence
  Synthetic construct
KNRKAKAKPVTRGAGAGGRQRGQNKERPPPVPNPDYEPIRKGQRDLYSGLNQRRI

SEQ ID NO: 10 - protein, 55 aa, Artificial Sequence
  Synthetic construct
KNRKAKAKPVTRGAGAGGRQRGQNKERPPPVPNPDYEPIRKGQRDLYSGLNQSRI

SEQ ID NO: 11 - protein, 45 aa, Artificial Sequence
  Synthetic construct
GHETGRLSGAADTQALLRNDQVYQPLRDRDDAQYSHLGGNWARNK

SEQ ID NO: 12 - protein, 45 aa, Artificial Sequence
  Synthetic construct
GHETGRLSGAADTQAALRNDQVYQPLRDRDDAQYSHLGGNWAANK

SEQ ID NO: 13 - protein, 45 aa, Artificial Sequence
  Synthetic construct
GQDGVRQSRASDKQTLLPNDQLYQPLKDREDDQYSHLQGNQLRRN

SEQ ID NO: 14 - protein, 45 aa, Artificial Sequence
  Synthetic construct
GQDGVRQSRASDKQTALPNDQLYQPLKDREDDQYSHLQGNQLARN

SEQ ID NO: 15 - protein, 41 aa, Artificial Sequence
  Synthetic construct
RSKRSRLLHSDYMNMTPRRPGPTRKHYQPYAPPRDFAAYRS

SEQ ID NO: 16 - protein, 17 aa, Artificial Sequence
  Synthetic construct
MLRLLLALNLFPSIQVT

SEQ ID NO: 17 - protein, 214 aa, Artificial Sequence
  Synthetic construct
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDGSADDAKKDAAKKDDAKKDDAKKDGSFKIPIEELEDRVFVNCNTSITWVEGTVGTLLSDITRLDLGKRILDPRGIYRCNGTDIYKDKESTVQVHYRMCQSCVELDPATVA

SEQ ID NO: 18 - protein, 224 aa, Artificial Sequence
  Synthetic construct
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDGSADDAKKDAAKKDDAKKDDAKKDGSQSIKGNHLVKVYDYQEDGSVLLTCDAEAKNITWFKDGKMIGFLTEDKKKWNLGSNAKDPRGMYQCKGSQNKSKPLQVYYRMCQNCIELNAATIS

SEQ ID NO: 19 - protein, 340 aa, Artificial Sequence
  Synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHYQVSFCLVMVLLFAVDTGLYFSVKTNIRSSTRDWKDHKFKWRKDPQDK

SEQ ID NO: 20 - protein, 336 aa, Artificial Sequence
  Synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLFFPPGYQVSFCLVMVLLFAVDTGLYFSVKTNIRSSTRDWKDHKFKWRKDPQDK

SEQ ID NO: 21 - protein, 335 aa, Artificial Sequence
  Synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGFFPPGYQVSFCLVMVLLFAVDTGLYFSVKTNIRSSTRDWKDHKFKWRKDPQDK

SEQ ID NO: 22 - DNA, 1032 bp, Artificial Sequence
  Synthetic construct
cttggagacaacatgtggttcttgacaactctgctcctttgggttccagttgatgggcaagtggacaccacaaaggcagtgatcactttgcagcctccatgggtcagcgtgttccaagaggaaaccgtaaccttgcattgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcattaccaagtctctttctgcttggtgatggtactcctttttgcagtggacacaggactatatttctctgtgaagacaaacattcgaagctcaacaagagactggaaggaccataaatttaaatggagaaaggaccctcaagacaaa

SEQ ID NO: 23 - DNA, 1020 bp, Artificial Sequence
  Synthetic construct
cttggagacaacatgtggttcttgacaactctgctcctttgggttccagttgatgggcaagtggacaccacaaaggcagtgatcactttgcagcctccatgggtcagcgtgttccaagaggaaaccgtaaccttgcattgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggtttgttctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaa

SEQ ID NO: 24 - DNA, 1005 bp, Artificial Sequence
  Synthetic construct
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaactctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcttctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaa

SEQ ID NO: 25 - protein, 379 aa, Artificial Sequence
  Synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQ

SEQ ID NO: 26 - DNA, 1140 bp, Artificial Sequence
  Synthetic construct
atggactggacctggattctgttcctggtcgcggctgcaacgcgagtccatagcggtatccatgtttttattcttgggtgtttttctgctgggctgcctaagaccgaggccaactgggtaaatgtcatcagtgacctcaagaaaatagaagaccttatacaaagcatgcacattgatgctactctctacactgagtcagatgtacatccctcatgcaaagtgacggccatgaaatgtttcctcctcgaacttcaagtcatatctctggaaagtggcgacgcgtccatccacgacacggtcgaaaacctgataatactcgctaataatagtctctcttcaaatggtaacgtaaccgagtcaggttgcaaagagtgcgaagagttggaagaaaaaaacataaaggagttcctgcaaagtttcgtgcacattgtgcagatgttcattaatacctctagcggcggaggatcaggtggcggtggaagcggaggtggaggctccggtggaggaggtagtggcggaggttctcttcaaataacttgtcctccaccgatgtccgtagaacatgcggatatttgggtaaaatcctatagcttgtacagccgagagcggtatatctgcaacagcggcttcaagcggaaggccggcacaagcagcctgaccgagtgcgtgctgaacaaggccaccaacgtggcccactggaccacccctagcctgaagtgcatcagagatcccgccctggtgcatcagcggcctgcccctccaagcacagtgacaacagctggcgtgaccccccagcctgagagcctgagcccttctggaaaagagcctgccgccagcagccccagcagcaacaatactgccgccaccacagccgccatcgtgcctggatctcagctgatgcccagcaagagccctagcaccggcaccaccgagatcagcagccacgagtctagccacggcaccccatctcagaccaccgccaagaactgggagctgacagccagcgcctctcaccagcctccaggcgtgtaccctcagggccacagcgataccacagtggccatcagcacctccaccgtgctgctgtgtggactgagcgccgtgtcactgctggcctgctacctgaagtccagacagtga

SEQ ID NO: 27 - protein, 375 aa, Artificial Sequence
  Synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYL

SEQ ID NO: 28 - protein, 242 aa, Artificial Sequence
  Synthetic construct
MDWTWILFLVAAATRVHSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIR

SEQ ID NO: 29 - DNA, 726 bp, Artificial Sequence
  Synthetic construct
atggactggacctggattctgttcctggtcgcggctgcaacgcgagtccatagcggtatccatgtttttattcttgggtgtttttctgctgggctgcctaagaccgaggccaactgggtaaatgtcatcagtgacctcaagaaaatagaagaccttatacaaagcatgcacattgatgctactctctacactgagtcagatgtacatccctcatgcaaagtgacggccatgaaatgtttcctcctcgaacttcaagtcatatctctggaaagtggcgacgcgtccatccacgacacggtcgaaaacctgataatactcgctaataatagtctctcttcaaatggtaacgtaaccgagtcaggttgcaaagagtgcgaagagttggaagaaaaaaacataaaggagttcctgcaaagtttcgtgcacattgtgcagatgttcattaatacctctagcggcggaggatcaggtggcggtggaagcggaggtggaggctccggtggaggaggtagtggcggaggttctcttcaaataacttgtcctccaccgatgtccgtagaacatgcggatatttgggtaaaatcctatagcttgtacagccgagagcggtatatctgcaacagcggcttcaagcggaaggccggcacaagcagcctgaccgagtgcgtgctgaacaaggccaccaacgtggcccactggaccacccctagcctgaagtgcatcaga

SEQ ID NO: 30 - protein, 635 aa, Artificial Sequence
  Synthetic construct
MDWTWILFLVAAATRVHSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGGGSGGGGSGGGGSGGGGSGGGSLQESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 31 - protein, 39 aa, Artificial sequence
  Synthetic construct
IEVMYPPPYLDNEKSNGTIIHVKGKHLCPSPLFPGPSKP

SEQ ID NO: 32 - protein, 88 aa, Artificial sequence
  Synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFL

SEQ ID NO: 33 - protein, 89 aa, Artificial sequence
  Synthetic construct
ESKYGPPCPPCPAPEFEGGPSVFLFPPKPKDTLMISRTPEVTCVVVDVSQEDPEVQFNWYVDGVEVHNAKTKPREEQFQSTYRVVSVLT

SEQ ID NO: 34 - protein, 129 aa, Artificial sequence
  Synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 229 aa, Artificial sequence
  Synthetic construct
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 122 aa, Artificial Sequence
  Synthetic construct
QVQLVESGGGLVQPGGSLRLSCAASGFTFSSYWMYWVRQTPGKGLEWVSTINRDGSATWYADSVKGRFTISRDNAKNTGYLQMNSLKPDDTAVYYCVSDPDNYSSDEMVPYWGQGTQVTVSS

SEQ ID NO: 37 - protein, 122 aa, Artificial Sequence
  Synthetic construct
EVQLVESGGGLVQPGGSLRLSCAASGFTFSSYWMYWVRQAPGKGLVWVSTINRDGSATWYADSVKGRFTISRDNAKNTLYLQMNSLRAEDTAVYYCARDPDNYSSDEMVPYWGQGTLVTVSS

SEQ ID NO: 38 - protein, 122 aa, Artificial Sequence
  Synthetic construct
EVQLVESGGGLVQPGGSLRLSCAASGFTFSSYWMYWVRQAPGKGLVWVSTINRDGSATWYADSVKGRFTISRDNAKNTLYLQMNSLRAEDTAVYYCVSDPDNYSSDEMVPYWGQGTLVTVSS

SEQ ID NO: 39 - protein, 122 aa, Artificial Sequence
  Synthetic construct
EVQLVESGGGLVQPGGSLRLSCAASGFTFSSYWMYWVRQTPGKGLVWVSTINRDGSATWYADSVKGRFTISRDNAKNTLYLQMNSLRAEDTAVYYCVSDPDNYSSDEMVPYWGQGTLVTVSS

SEQ ID NO: 40 - protein, 122 aa, Artificial Sequence
  Synthetic construct
EVQLVESGGGLVQPGGSLRLSCAASGFTFSSYWMYWVRQAPGKGLEWVSTINRDGSATWYADSVKGRFTISRDNAKNTLYLQMNSLRAEDTAVYYCVSDPDNYSSDEMVPYWGQGTLVTVSS

SEQ ID NO: 41 - protein, 122 aa, Artificial Sequence
  Synthetic construct
EVQLVESGGGLVQPGGSLRLSCAASGFTFSSYWMYWVRQTPGKGLEWVSTINRDGSATWYADSVKGRFTISRDNAKNTGYLQMNSLRPEDTAVYYCVSDPDNYSSDEMVPYWGQGTLVTVSS

(19) United States
(12) Patent Application Publication
(10) Pub. No.: US 2023/0405121 A1
(43) Pub. Date: Dec. 21, 2023

(54) ENGINEERED IPSC AND IMMUNE EFFECTOR CELLS FOR HETEROGENOUS TUMOR CONTROL

(71) Applicant: Fate Therapeutics, Inc., San Diego, CA (US)

(72) Inventors: Bahram VALAMEHR, San Diego, CA (US); Martin HOSKING, San Diego, CA (US); Tom Tong LEE, San Diego, CA (US); John Charles GOULDING, San Diego, CA (US)

(21) Appl. No.: 18/250,735

(22) PCT Filed: Nov. 4, 2021
(86) PCT No.: PCT/US2021/058138

§ 371 Date: Apr. 26, 2023

Related U.S. Application Data
(60) Provisional application No. 63/109,829, filed on Nov. 4, 2020.
(60) Provisional application No. 63/172,891, filed on Apr. 9, 2021.

(51) Int. Cl. A61K 39/00; C12N 5/0783; C12N 15/86; C07K 16/28; C07K 16/30; C07K 14/54; C07K 14/735; A61K 45/06; A61P 35/00; C07K 14/725

(52) U.S. Cl. CPC A61K 39/464411; C12N 5/0636; C12N 5/0646; C12N 15/86; C07K 16/2827; C07K 16/30; C07K 16/2809; C07K 16/2818; C07K 16/2851; C07K 14/5443; C07K 14/70535; A61K 45/06; A61P 35/00; A61K 39/4613; A61K 39/4611; C07K 16/283; A61K 39/4631; A61K 39/4635; A61K 39/4633; A61K 39/464466; C07K 14/7051; C12N 2506/45; C12N 2740/15043; C07K 2317/622; A61K 2239/27; C07K 2317/31; A61K 2039/572; C07K 2319/33

(57) ABSTRACT

Provided are methods and compositions for obtaining functionally enhanced derivative effector cells obtained from directed differentiation of genomically engineered iPSCs. The iPSC-derived effector cells provided herein have stable and functional genome editing that delivers improved or enhanced therapeutic effects. Also provided are therapeutic compositions and the use thereof comprising the functionally enhanced derivative effector cells alone, or with antibodies or checkpoint inhibitors in combination therapies.

### RELATED APPLICATIONS

This application claims priority to U.S. Provisional Application Ser. No. 63/109,829, filed Nov. 4, 2020, and to U.S. Provisional Application Ser. No. 63/172,891, filed Apr. 9, 2021, the disclosure of each of which is hereby incorporated by reference in their entireties.

### FIELD OF THE INVENTION

The present disclosure is broadly concerned with the field of off-the-shelf immunocellular products. More particularly, the present disclosure is concerned with strategies for developing multifunctional effector cells capable of delivering therapeutically relevant properties in vivo. The cell products developed under the present disclosure address critical limitations of patient-sourced cell therapies.

### REFERENCE TO SEQUENCE LISTING SUBMITTED ELECTRONICALLY

This application incorporates by reference a Computer Readable Form (CRF) of a Sequence Listing in ASCII text format submitted with this application, entitled 184143-632601_SequenceListing_ST25.txt, which was created on Nov. 4, 2021, and is 61,603 bytes in size.

### BACKGROUND OF THE INVENTION

The field of adoptive cell therapy is currently focused on using patient- and donor-sourced cells, which makes it particularly difficult to achieve consistent manufacturing of cancer immunotherapies and to deliver therapies to all patients who may benefit. There is also a need to improve the efficacy and persistence of adoptively transferred lymphocytes to promote favorable patient outcomes. Lymphocytes such as T cells and natural killer (NK) cells are potent anti-tumor effectors that play an important role in innate and adaptive immunity. However, the use of these immune cells for adoptive cell therapies remains challenging and has unmet needs for improvement. Therefore, there are significant opportunities to harness the full potential of T and NK cells, or other immune effector cells in adoptive immunotherapy.

### SUMMARY OF THE INVENTION

There is a need for functionally improved effector cells that address issues ranging from response rate, cell exhaustion, loss of transfused cells (survival and/or persistence), tumor escape through target loss or lineage switch, tumor targeting precision, off-target toxicity, off-tumor effect, to efficacy against solid tumors, i.e., tumor microenvironment and related immune suppression, recruiting, trafficking and infiltration.

It is an object of the present invention to provide methods and compositions to generate derivative non-pluripotent cells differentiated from a single cell derived iPSC (induced pluripotent stem cell) clonal line, which iPSC comprises one or several genetic modifications in its genome. Said one or several genetic modifications include DNA insertion, deletion, and substitution, and which modifications are retained and remain functional in subsequently derived cells after differentiation, expansion, passaging and/or transplantation.

The iPSC derived non-pluripotent cells of the present application include, but not limited to, CD34⁺ cells, hemogenic endothelium cells, HSCs (hematopoietic stem and progenitor cells), hematopoietic multipotent progenitor cells, T cell progenitors, NK cell progenitors, T cells, NKT cells, NK cells, B cells, and immune effector cells having one or more functional features that are not present in a primary NK, T, and/or NKT cell. The iPSC-derived non-pluripotent cells of the present application comprise one or several genetic modifications in their genome through differentiation from an iPSC comprising the same genetic modifications. The engineered clonal iPSC differentiation strategy for obtaining genetically engineered derivative cells requires that the developmental potential of the iPSC in differentiation is not adversely impacted by the engineered modality in the iPSC, and also that the engineered modality functions as intended in the derivative cell. Further, this strategy overcomes the present barrier in engineering primary lymphocytes, such as T cells or NK cells obtained from peripheral blood, as such cells are difficult to engineer, with engineering of such cells often lacking reproducibility and uniformity, resulting in cells exhibiting poor cell persistence with high cell death and low cell expansion. Moreover, this strategy avoids production of a heterogenous effector cell population otherwise obtained using primary cell sources which are heterogenous to start with.

Some aspects of the present invention provide genome-engineered iPSCs obtained using a method comprising (I), (II) or (III), reflecting a strategy of genomic engineering subsequently to, simultaneously with, and prior to the reprogramming process, respectively:
(I): genetically engineering iPSCs by one or both of (i) and (ii), in any order: (i) introducing into iPSCs one or more construct(s) to allow targeted integration at selected site(s); (ii) (a) introducing into iPSCs one or more double stranded break(s) at selected site(s) using one or more endonucleases capable of selected site recognition; and (b) culturing the iPSCs of step (I)(ii)(a) to allow endogenous DNA repair to generate targeted in/dels at the selected site(s), simultaneously or sequentially; thereby obtaining genome-engineered iPSCs capable of differentiation into partially or fully differentiated cells.
(II): genetically engineering reprogramming non-pluripotent cells to obtain the genome-engineered iPSCs comprising: (i) contacting non-pluripotent cells with one or more reprogramming factors, and optionally a small molecule composition comprising a TGFβ receptor/ALK inhibitor, a MEK inhibitor, a GSK3 inhibitor and/or a ROCK inhibitor to initiate reprogramming of the non-pluripotent cells; and (ii) introducing into the reprogramming non-pluripotent cells of step (II)(i) one or both of (a) and (b), in any order: (a) one or more construct(s) to allow targeted integration at selected site(s); (b) one or more double stranded break(s) at a selected site using at least one endonuclease capable of selected site recognition, then the cells of step (II)(ii)(b) are cultured to allow endogenous DNA repair to generate targeted in/dels at the selected site(s); as such the obtained genome-engineered iPSCs comprise at least one functional targeted genomic editing, and said genome-engineered iPSCs are capable of differentiation into partially or fully differentiated cells.
(III): genetically engineering non-pluripotent cells for reprogramming to obtain genome-engineered iPSCs comprising (i) and (ii): (i) introducing into non-pluripotent cells one or both of (a) and (b), in any order: (a) one or more construct(s) to allow targeted integration at selected site(s); (b) one or more double stranded break(s) at a selected site using at least one endonuclease capable of selected site recognition, wherein the cells of step (III)(i)(b) are cultured to allow endogenous DNA repair to generate targeted in/dels at the selected sites; and (ii) contacting the cells of step (III)(i) with one or more reprogramming factors, and optionally a small molecule composition comprising a TGFβ receptor/ALK inhibitor, a MEK inhibitor, a GSK3 inhibitor and/or a ROCK inhibitor, to obtain genome-engineered iPSCs comprising targeted editing at selected sites; thereby obtaining genome-engineered iPSCs comprising at least one functional targeted genomic editing, and said genome-engineered iPSCs are capable of being differentiated into partially differentiated cells or fully-differentiated cells.

In one embodiment of the above method, the at least one targeted genomic editing at one or more selected sites comprises insertion of one or more exogenous polynucleotides encoding safety switch proteins, targeting modalities, receptors, signaling molecules, transcription factors, pharmaceutically active proteins and peptides, drug target candidates, or proteins promoting engraftment, trafficking, homing, viability, self-renewal, persistence, and/or survival of the genome-engineered iPSCs or derivative cells therefrom. In some embodiments, the exogenous polynucleotides for insertion are operatively linked to (1) one or more exogenous promoters comprising CMV, EF1α, PGK, CAG, UBC, or other constitutive, inducible, temporal-, tissue-, or cell type-specific promoters; or (2) one or more endogenous promoters comprised in the selected sites comprising AAVS1, CCR5, ROSA26, collagen, HTRP, H11, beta-2 microglobulin, CD38, GAPDH, TCR or RUNX1, or other locus meeting the criteria of a genome safe harbor. In some embodiments, the genome-engineered iPSCs generated using the above method comprise one or more different exogenous polynucleotides encoding protein comprising caspase, thymidine kinase, cytosine deaminase, modified EGFR, or B-cell CD20, wherein when the genome-engineered iPSCs comprise two or more suicide genes, the suicide genes are integrated in different safe harbor locus comprising AAVS1, CCR5, ROSA26, collagen, HTRP, H11, beta-2 microglobulin, CD38, GAPDH, TCR or RUNX1. In one embodiment, the exogenous polynucleotide encodes a partial or full length peptide of IL2, IL4, IL6, IL7, IL9, IL10, IL11, IL12, IL15, IL18, IL21, and/or respective receptors thereof. In some embodiments, the partial or full peptide of IL2, IL4, IL6, IL7, IL9, IL10, IL11, IL12, IL15, IL18, IL21, and/or respective receptors thereof encoded by the exogenous polynucleotide is in the form of a fusion protein.

In some other embodiments, the genome-engineered iPSCs generated using the method provided herein comprise in/dels at one or more endogenous genes associated with targeting modality, receptors, signaling molecules, transcription factors, drug target candidates, immune response regulation and modulation, or proteins suppressing engraftment, trafficking, homing, viability, self-renewal, persistence, and/or survival of the iPSCs or derivative cells therefrom. In some embodiments, the endogenous gene for disruption comprises at least one of CD38, B2M, TAP1, TAP2, Tapasin, NLRC5, PD1, LAG3, TIM3, RFXANK, CIITA, RFX5, RFXAP, RAG1, and any gene in the chromosome 6p21 region.

In yet some other embodiments, the genome-engineered iPSCs generated using the method provided herein comprise a caspase encoding exogenous polynucleotide at AAVS1 locus, and a thymidine kinase encoding exogenous polynucleotide at H11 locus.

In still some other embodiments, approach (I), (II) and/or (III) further comprises: contacting the genome-engineered iPSCs with a small molecule composition comprising a MEK inhibitor, a GSK3 inhibitor and a ROCK inhibitor, to maintain the pluripotency of the genomic-engineered iPSCs. In one embodiment, the obtained genome engineered iPSCs comprising at least one targeted genomic editing are functional, are differentiation potent, and are capable of differentiating into non-pluripotent cells comprising the same functional genomic editing.

Accordingly, in one aspect, the present invention provides a cell or a population thereof, wherein the cell comprises a polynucleotide encoding a CAR targeting a B7H3 tumor antigen, wherein the CAR comprises a binding domain comprising: (i) an amino acid sequence that is of at least about 99%, 98%, 96%, 95%, 90%, 85%, or 80% identity to SEQ ID NO: 36, 37, 38, 39, 40, or 41; (ii) an amino acid sequence represented by a variant of SEQ ID NO: 36, and wherein the variant has one or more mutations at positions comprising 1, 40, 46, 79, 87, 88, 89, 97, 98, and 117 of SEQ ID NO: 36; (iii) an amino acid sequence represented by a variant of SEQ ID NO: 36, wherein the variant has one or more substitutions comprising Q1E, T40A, E46V, G79L, K87R, P88A, D89E, V97A, S98R, and Q117L according to SEQ ID NO:36; or (iv) an amino acid sequence represented by any of SEQ ID NOs: 36, 37, 38, 39, 40, and 41; and wherein the cell is an eukaryotic cell, an animal cell, a human cell, an immune cell, an induced pluripotent cell (iPSC), or a derivative cell differentiated therefrom.

In various embodiments, the cell further comprises one or more polynucleotides encoding an engager, and optionally a CFR (chimeric fusion receptor), wherein the engager has a tumor antigen targeting specificity that is not B7H3. In some embodiments, (i) the engager comprises a first binding domain having a different tumor targeting specificity from the CAR, and wherein the first binding domain is specific to any one of ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CD5, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb□ B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NY-ESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; (ii) the engager comprises a second binding domain having specificity that is different from the specificity of the first binding domain and is to an extracellular portion of CD3, CD28, CD5, CD16, CD64, CD32, CD33, CD89, NKG2C, NKG2D, or any functional variants thereof, or the second binding domain of the engager is specific to an ectodomain of the CFR; or (iii) the engager comprises a cytokine or a variant thereof between the first and the second binding domains, wherein the cytokine comprises at least one of IL2, IL4, IL6, IL7, IL9, IL10, IL11, IL12, IL15, IL18, and IL21.

In some embodiments of the cell or population thereof, (i) the CFR comprises an ectodomain fused to a transmembrane domain, which is operatively connected to an endodomain, and wherein the ectodomain, transmembrane domain and the endodomain do not comprise any endoplasmic reticulum (ER) retention signals or endocytosis signals; (ii) the CFR comprises an ectodomain that comprises a full or partial length of an extracellular portion of a signaling protein comprising at least one of CD3ε, CD3γ, CD3δ, CD28, CD5, CD16, CD64, CD32, CD33, CD89, NKG2C, NKG2D, any functional variants, and a combination or a chimera thereof; (iii) the CFR comprises an ectodomain that initiates signal transduction upon binding to a selected agonist; (iv) the CFR comprises an endodomain that comprises a cytotoxicity domain comprising at least a full length or a portion of CD3ζ, 2B4, DAP10, DAP12, DNAM1, CD137 (4-1B), IL21, IL7, IL12, IL15, NKp30, NKp44, NKp46, NKG2C, or NKG2D polypeptide; and optionally wherein the endodomain further comprises one or more of: (a) a co-stimulatory domain comprising a full length or a portion of CD2, CD27, CD28, CD40L, 4-1BB, OX40, ICOS, PD-1, LAG-3, 2B4, BTLA, DAP10, DAP12, CTLA-4, or NKG2D polypeptide, or any combination thereof, (b) a co-stimulatory domain comprising a full length or a portion of CD28, 4-1BB, CD27, CD40L, ICOS, CD2, or any combination thereof, (c) a persistency signaling domain comprising a full length or a portion of an endodomain of a cytokine receptor comprising IL7R, IL15R, IL18R, IL12R, IL23R, or combinations thereof; and/or (d) a full or a partial intracellular portion of a receptor tyrosine kinase (RTK), a tumor necrosis factor receptor (TNFR), an EGFR or a FAS receptor; or (iv) the CFR is co-expressed with the engager or the CAR in separate constructs or in a bicistronic expression cassette.

In some embodiments of the cell or population thereof, the cell further comprises one or more of the following edits: CD38 knockout; HLA-I deficiency and/or HLA-II deficiency; introduction of HLA-G or non-cleavable HLA-G; an exogenous CD16 or a variant thereof, a signaling complex comprising a partial or full peptide of a cell surface expressed exogenous cytokine and/or a receptor thereof; at least one of the genotypes listed in Table 1; disruption of at least one of B2M, TAP1, TAP2, Tapasin, NLRC5, CIITA, RFXANK, RFX5, RFXAP, TCR α or β constant region, NKG2A, NKG2D, CD56, CIS, CBL-B, SOCS2, PD1, CTLA4, LAG3, TIM3, and TIGIT; and/or introduction of at least one of HLA-E, 4-1BBL, CD3, CD4, CD8, CD16, CD47, CD113, CD131, CD137, CD80, PDL1, A_{2A}R, antigen-specific TCR, Fc receptor, an engager, and surface triggering receptor for coupling with bi- or multi-specific or universal engagers, in comparison to its native counterpart cell. In some embodiments, the cell or population thereof has therapeutic properties comprising one or more of: (i) increased cytotoxicity; (ii) improved persistency and/or survival; (iii) enhanced ability in migrating, and/or activating or recruiting bystander immune cells, to tumor sites; (iv) improved tumor penetration; (v) enhanced ability to reduce tumor immunosuppression; (vi) improved ability in rescuing tumor antigen escape; (vii) controlled apoptosis; (viii) enhanced or acquired ADCC; and (ix) ability to avoid fratricide, in comparison to its counterpart primary cell obtained from peripheral blood, umbilical cord blood, or any other donor tissues.

In some other embodiments of said cell or population thereof, the cell further comprises an exogenous CD16 or a variant thereof. In some embodiments, the exogenous CD16 is a high affinity non-cleavable CD16 (hnCD16) or a variant thereof. In some embodiments, the hnCD16 or a variant thereof comprises: F176V and S197P in an ectodomain domain of CD16; or a full or partial ectodomain originated from CD64; a non-CD16 (non-native) transmembrane domain; a non-CD16 intracellular domain; a non-CD16 signaling domain; and/or a stimulatory domain; or transmembrane, signaling, and stimulatory domains that are originated from a same or different non-CD16 polypeptide. In some other embodiments of said cell or population thereof, the cell further comprises surface expressed exogenous cytokine or receptor thereof, wherein the surface expressed exogenous cytokine or receptor thereof comprises: (a) at least one of IL2, IL4, IL6, IL7, IL9, IL10, IL11, IL12, IL15, IL18, IL21, and its respective receptor(s); or (b) comprises at least one of: (i) co-expression of IL15 and IL15Rα by using a self-cleaving peptide; (ii) a fusion protein of IL15 and IL15Rα; (iii) an IL15/IL15Rα fusion protein with intracellular domain of IL15Rα truncated or eliminated; (iv) a fusion protein of IL15 and membrane bound Sushi domain of IL15Rα; (v) a fusion protein of IL15 and IL15Rβ; (vi) a fusion protein of IL15 and common receptor γC, wherein the common receptor γC is native or modified; and a homodimer of IL15Rβ; wherein any one of (i)-(vii) is optionally co-expressed with a CAR in separate constructs or in a bi-cistronic construct; or (c) comprises at least one of: (i) a fusion protein of IL7 and IL7Rα; (ii) a fusion protein of IL7 and common receptor γC, wherein the common receptor γC is native or modified; and (iii) a homodimer of IL7Rβ, wherein any one of (c)(i)-(iii) is optionally co-expressed with a CAR in separate constructs or in a bi-cistronic expression cassette; and optionally, (d) is transiently expressed.

In those embodiments of the cell or population thereof where the cell comprises introduction of a checkpoint inhibitor, the checkpoint inhibitor may be an antagonist to one or more checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, and inhibitory KIR.

In various embodiments of the cell or population thereof, the cell comprises: (i) one or more exogenous polynucleotides integrated in a safe harbor locus or a selected gene locus; or (ii) more than two exogenous polynucleotides integrated in different safe harbor loci or two or more selected gene loci. In various embodiments, the safe harbor locus comprises at least one of AAVS1, CCR5, ROSA26, collagen, HTRP, H11, GAPDH, or RUNX1; and wherein the selected gene locus is one of B2M, TAP1, TAP2, Tapasin, NLRC5, CIITA, RFXANK, RFX5, RFXAP, TCR, NKG2A, NKG2D, CD38, CD25, CD69, CD44, CD58, CD54, CD56, CIS, CBL-B, SOCS2, PD1, CTLA4, LAG3, TIM3, or TIGIT; and/or wherein the integration of the exogenous polynucleotides knocks out expression of the gene in the locus. In particular embodiments, the TCR locus is a constant region of TCR alpha and/or TCR beta (TRAC and/or TRBC).

In various embodiments of the cell or population thereof, the iPSC is a clonal iPSC, a single cell dissociated iPSC, an iPSC cell line cell, or an iPSC master cell bank (MCB) cell; or wherein the derivative cell comprises a derivative CD34⁺ cell, a derivative hematopoietic stem and progenitor cell, a derivative hematopoietic multipotent progenitor cell, a derivative T cell progenitor, a derivative NK cell progenitor, a derivative T lineage cell, a derivative NKT lineage cell, a derivative NK lineage cell, a derivative B lineage cell, or a derivative effector cell having one or more functional features that are not present in a counterpart primary T, NK, NKT, and/or B cell.

In another aspect, the application provides a composition comprising a cell or population thereof as described herein. In yet another aspect, the application provides a composition for therapeutic use comprising a cell or population thereof as described herein, and one or more therapeutic agents. In various embodiments, the one or more therapeutic agents comprise a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD). In those embodiments where the therapeutic agent is a checkpoint inhibitor, the checkpoint inhibitor may comprise: (a) one or more antagonists to checkpoint molecules comprising PD-1, PDL-1, TIM-3, TIGIT, LAG-3, CTLA-4, 2B4, 4-1BB, 4-1BBL, A2AR, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, or inhibitory KIR; (b) one or more of atezolizumab, avelumab, durvalumab, ipilimumab, IPH4102, IPH43, IPH33, lirimumab, monalizumab, nivolumab, pembrolizumab, and their derivatives or functional equivalents; (c) at least one of atezolizumab, nivolumab, and pembrolizumab; or (ii) the therapeutic agents comprise one or more of venetoclax, azacitidine, and pomalidomide. In those embodiments where the therapeutic agent is an antibody, the antibody may comprise: (a) anti-CD20, anti-HER2, anti-CD52, anti-EGFR, anti-CD123, anti-GD2, anti-PDL1, and/or anti-CD38 antibody; (b) one or more of rituximab, veltuzumab, ofatumumab, ublituximab, ocaratuzumab, obinutuzumab, trastuzumab, pertuzumab, alemtuzumab, cetuximab, dinutuximab, avelumab, daratumumab, isatuximab, MOR202, 7G3, CSL362, elotuzumab, and their humanized or Fc modified variants or fragments and their functional equivalents and biosimilars; or (c) daratumumab, and wherein the derivative hematopoietic cells comprise derivative NK cells or derivative T cells comprising a CD38 knockout, and optionally an expression of exogenous CD16 or a variant thereof. Thus, the application provides for therapeutic use of the compositions described herein by introducing the composition to a subject suitable for adoptive cell therapy, wherein the subject has an autoimmune disorder; a hematological malignancy; a solid tumor; cancer, or a virus infection.

In yet another aspect, the application provides a composition comprising a cell or a population thereof, wherein the cell comprises one or more polynucleotides encoding a chimeric antigen receptor (CAR), an engager, and optionally a CFR (chimeric fusion receptor), wherein the CFR is optionally for engager coupling, and wherein the cell is an eukaryotic cell, an animal cell, a human cell, an immune cell, an induced pluripotent cell (iPSC), or a derivative cell differentiated therefrom. In various embodiments of the composition, (i) the engager has a different tumor targeting specificity from the CAR; or (ii) the engager is co-expressed with the CAR or the CFR. In various embodiments of the composition, the CAR is: (i) T cell specific or NK cell specific; (ii) a bi-specific antigen binding CAR; (iii) a switchable CAR; (iv) a dimerized CAR; (v) a split CAR; (vi) a multi-chain CAR; (vii) an inducible CAR; (viii) co-expressed with another CAR; (ix) co-expressed with a partial or full peptide of a cell surface expressed exogenous cytokine or a receptor thereof, optionally in separate constructs or in a bi-cistronic construct; (x) co-expressed with a checkpoint inhibitor, optionally in separate constructs or in a bi-cistronic construct; and/or (xi) specific to any one of ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CD5, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NY-ESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; and wherein the CAR of any one of (i) to (xi) is optionally inserted at TRAC or TRBC locus, and/or is driven by an endogenous promoter of TCR, and/or the TCR is knocked out by the CAR insertion.

In various embodiments of the composition, the CAR comprises: (i) an antigen recognition region specific to B7H3; (ii) a binding domain comprising an amino acid sequence that is of at least about 99%, 98%, 96%, 95%, 90%, 85%, or 80% identity to SEQ ID NO: 36, 37, 38, 39, 40, or 41; (iii) a binding domain comprising an amino acid sequence represented by a variant of SEQ ID NO: 36, wherein the variant has one or more mutations at positions comprising 1, 40, 46, 79, 87, 88, 89, 97, 98, and 117 of SEQ ID NO: 36; (iv) a binding domain comprising an amino acid sequence represented by a variant of SEQ ID NO: 36, wherein the variant has one or more substitutions comprising Q1E, T40A, E46V, G79L, K87R, P88A, D89E, V97A, S98R, and Q117L according to SEQ ID NO:36; or (v) a binding domain comprising an amino acid sequence represented by any of SEQ ID NOs: 36, 37, 38, 39, 40, and 41.

In various embodiments of the composition, (i) the engager comprises a first binding domain having a different tumor targeting specificity from the CAR, and wherein the first binding domain is specific to any one of ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CD5, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NY-ESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; or (ii) the engager comprises a first binding domain having a different tumor targeting specificity from the CAR, and wherein the CAR comprises a binding domain specific to any one of B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, or ROR1; or (iii) the engager comprises a second binding domain having specificity that is different from the specificity of the first binding domain and is to an extracellular portion of CD3, CD28, CD5, CD16, CD64, CD32, CD33, CD89, NKG2C, NKG2D, or any functional variants thereof, or (iv) the engager comprises a cytokine or a variant thereof between the first and the second binding domains, wherein the cytokine comprises at least one of IL2, IL4, IL6, IL7, IL9, IL10, IL11, IL12, IL15, IL18, and IL21.

In various embodiments of the composition, the cell of the composition further comprises one or more of: (i) CD38 knockout; (ii) HLA-I deficiency and/or HLA-II deficiency; (iii) introduction of HLA-G or non-cleavable HLA-G, or knockout of one or both of CD58 and CD54; (iv) an exogenous CD16 or a variant thereof, (v) a chimeric fusion receptor (CFR); (vi) an inactivation CAR; (vii) a signaling complex comprising a partial or full peptide of a cell surface expressed exogenous cytokine and/or a receptor thereof, (viii) at least one of the genotypes listed in Table 1; (ix) disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or (x) introduction of at least one of HLA-E, 4-1BBL, CD3, CD4, CD8, CD16, CD47, CD113, CD131, CD137, CD80, PDL1, A_{2A}R, antigen-specific TCR, Fc receptor, an antibody or functional variant or fragment thereof, a checkpoint inhibitor, and surface triggering receptor for coupling with an agonist, in comparison to its counterpart primary cell. In various embodiments of the composition, the cell has therapeutic properties comprising one or more of: (i) increased cytotoxicity; (ii) improved persistency and/or survival; (iii) enhanced ability in migrating, and/or activating or recruiting bystander immune cells, to tumor sites; (iv) improved tumor penetration; (v) enhanced ability to reduce tumor immunosuppression; (vi) improved ability in rescuing tumor antigen escape; (vii) controlled apoptosis; (viii) enhanced or acquired ADCC; and (ix) ability to avoid fratricide, in comparison to its counterpart primary cell obtained from peripheral blood, umbilical cord blood, or any other donor tissues.

In those embodiments where the cell of the composition comprises an exogenous CD16 or variant thereof, the CD16 or a variant thereof may comprise at least one of: (a) a high affinity non-cleavable CD16 (hnCD16); (b) F176V and S197P in ectodomain domain of CD16; (c) a full or partial ectodomain originated from CD64; (d) a non-native (or non-CD16) transmembrane domain; (e) a non-native (or non-CD16) intracellular domain; (f) a non-native (or non-CD16) signaling domain; (g) a non-native stimulatory domain; and (h) transmembrane, signaling, and stimulatory domains that are not originated from CD16, and are originated from a same or different polypeptide.

In those embodiments where the cell of the composition comprises a CFR, the CFR may comprise an ectodomain fused to a transmembrane domain, which is operatively connected to an endodomain, and wherein the ectodomain, transmembrane domain and the endodomain do not comprise any endoplasmic reticulum (ER) retention signals or endocytosis signals. In some embodiments, (i) the ectodomain of the CFR comprises a full or partial length of an extracellular portion of a signaling protein comprising at least one of CD3ε, CD3γ, CD3δ, CD28, CD5, CD16, CD64, CD32, CD33, CD89, NKG2C, NKG2D, any functional variants, and a combination or a chimera thereof, (ii) the ectodomain of the CFR initiates signal transduction upon binding to a selected agonist; (iii) the endodomain of the CFR comprises a cytotoxicity domain comprising at least a full length or a portion of CD3ζ, 2B4, DAP10, DAP12, DNAM1, CD137 (4-1B), IL21, IL7, IL12, IL15, NKp30, NKp44, NKp46, NKG2C, or NKG2D polypeptide; and optionally wherein the endodomain further comprises one or more of: (a) a co-stimulatory domain comprising a full length or a portion of CD2, CD27, CD28, CD40L, 4-1BB, OX40, ICOS, PD-1, LAG-3, 2B4, BTLA, DAP10, DAP12, CTLA-4, or NKG2D polypeptide, or any combination thereof, (b) a co-stimulatory domain comprising a full length or a portion of CD28, 4-1BB, CD27, CD40L, ICOS, CD2, or combinations thereof, (c) a persistency signaling domain comprising a full length or a portion of an endodomain of a cytokine receptor comprising TL7R, IL15R, IL18R, IL12R, IL23R, or combinations thereof, and/or (d) a full or a partial intracellular portion of a receptor tyrosine kinase (RTK), a tumor necrosis factor receptor (TNFR), an EGFR or a FAS receptor; or (iv) the CFR is co-expressed with the engager in separate constructs or in a bicistronic expression cassette, and optionally the engager has binding specificity to the ectodomain of the CFR.

In some embodiments, the selected agonist is (i) an antibody or a functional variant or fragment thereof; (ii) an agonistic ligand; or (iii) an engager; and the selected ligand may: (a) be encoded by the polynucleotide comprised in the cell of the composition or is comprised in the composition; (b) comprise at least a first binding domain that is specific to at least one tumor antigen comprising B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, or ROR1; and optionally, (c) comprise a second binding domain that is specific to a cell surface protein of: (1) the cell of the composition; or (2) a bystander effector cell; wherein the cell surface protein comprises an extracellular portion of CD3, CD28, CD5, CD16, CD64, CD32, CD33, CD89, NKG2C, NKG2D, or any functional variants thereof.

In those embodiments where the cell of the composition comprises an inactivation CAR, the inactivation CAR may: (i) target an upregulated surface protein in activated recipient immune cells; or (ii) comprise at least one of a CD38-CAR, a CD25-CAR, a CD69-CAR, a CD44-CAR, a 4-1BB-CAR, an OX40-CAR, and a CD40L-CAR.

In those embodiments where the cell of the composition comprises a cell surface expressed exogenous cytokine or receptor thereof, the cell surface expressed exogenous cytokine or receptor thereof may: (a) comprise at least one of IL2, IL4, IL6, IL7, IL9, IL10, IL11, IL12, IL15, IL18, IL21, and its respective receptor(s); or (b) comprise at least one of: (i) co-expression of IL15 and IL15Rα by using a self-cleaving peptide; (ii) a fusion protein of IL15 and IL15Rα; (iii) an IL15/IL15Rα fusion protein with intracellular domain of IL15Rα truncated or eliminated; (iv) a fusion protein of IL15 and membrane bound Sushi domain of IL15Rα; (v) a fusion protein of IL15 and IL15Rβ; (vi) a fusion protein of IL15 and common receptor γC, wherein the common receptor γC is native or modified; and (vii) a homodimer of IL15Rβ, wherein any one of (b)(i)-(vii) is optionally co-expressed with a CAR in separate constructs or in a bi-cistronic construct; or (c) comprise at least one of: (i) a fusion protein of IL7 and IL7Rα; (ii) a fusion protein of IL7 and common receptor γC, wherein the common receptor γC is native or modified; and (iii) a homodimer of IL7Rβ, wherein any one of (c)(i)-(iii) is optionally co-expressed with a CAR in separate constructs or in a bi-cistronic construct; and optionally, (d) be transiently expressed.

In those embodiments where the cell of the composition comprises a checkpoint inhibitor, the checkpoint inhibitor may be an antagonist to one or more checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, and inhibitory KIR.

In some embodiments, the cell of the composition may comprise: (i) one or more exogenous polynucleotides integrated in a safe harbor locus or a selected gene locus; or (ii) more than two exogenous polynucleotides integrated in different safe harbor loci or two or more selected gene loci. In some embodiments, the safe harbor locus comprises at least one of AAVS1, CCR5, ROSA26, collagen, HTRP, H11, GAPDH, or RUNX1; and wherein the selected gene locus is one of B2M, TAP1, TAP2, Tapasin, NLRC5, CIITA, RFXANK, RFX5, RFXAP, TCR, NKG2A, NKG2D, CD38, CD25, CD69, CD44, CD58, CD54, CD56, CIS, CBL-B, SOCS2, PD1, CTLA4, LAG3, TIM3, or TIGIT; and/or wherein the integration of the exogenous polynucleotides knocks out expression of the gene in the locus. In particular embodiments, the TCR locus may be a constant region of TCR alpha and/or TCR beta (TRAC and/or TRBC).

In various embodiments of the composition, the derivative cell comprises a derivative CD34⁺ cell, a derivative hematopoietic stem and progenitor cell, a derivative hematopoietic multipotent progenitor cell, a derivative T cell progenitor, a derivative NK cell progenitor, a derivative T lineage cell, a derivative NKT lineage cell, a derivative NK lineage cell, a derivative B lineage cell, or a derivative effector cell having one or more functional features that are not present in a counterpart primary T, NK, NKT, and/or B cell

In some embodiments, the composition further comprises one or more therapeutic agents. In some embodiments, the one or more therapeutic agents comprise a peptide, a cytokine, a checkpoint inhibitor, an antibody or functional variant or fragment thereof, a mitogen, a growth factor, a small RNA, a dsRNA (double stranded RNA), mononuclear blood cells, feeder cells, feeder cell components or replacement factors thereof, a vector comprising one or more polynucleic acids of interest, a chemotherapeutic agent or a radioactive moiety, or an immunomodulatory drug (IMiD). In some embodiments, the one or more therapeutic agents comprise one or more of venetoclax, azacitidine, and pomalidomide. In some embodiments where the therapeutic agent comprises a checkpoint inhibitor, the checkpoint inhibitor comprises: (i) one or more antagonist checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, or inhibitory KIR; (ii) one or more of atezolizumab, avelumab, durvalumab, ipilimumab, IPH4102, IPH43, IPH33, lirimumab, monalizumab, nivolumab, pembrolizumab, and their derivatives or functional equivalents; or (iii) at least one of atezolizumab, nivolumab, and pembrolizumab. In those embodiments where the therapeutic agent comprises an antibody, or functional variant or fragment thereof, the antibody, or functional variant or fragment thereof may comprise: (a) anti-CD20, anti-CD22, anti-HER2, anti-CD52, anti-EGFR, anti-CD123, anti-GD2, anti-PDL1, and/or anti-CD38 antibody; (b) one or more of rituximab, veltuzumab, ofatumumab, ublituximab, ocaratuzumab, obinutuzumab, ibritumomab, ocrelizumab, inotuzumab, moxetumomab, epratuzumab, trastuzumab, pertuzumab, alemtuzumab, cetuximab, dinutuximab, avelumab, daratumumab, isatuximab, MOR202, 7G3, CSL362, elotuzumab, and their humanized or Fc modified variants or fragments and their functional equivalents and biosimilars; or (c) daratumumab, and wherein the derivative effector cell comprises a CD38 knockout, and optionally expresses CD16 or a variant thereof.

In another aspect, the invention provides for therapeutic use of the composition provided herein by introducing the composition to a subject suitable for adoptive cell therapy, wherein the subject has an autoimmune disorder, a hematological malignancy, a solid tumor, cancer, or a viral infection.

In yet another aspect, the invention provides a method of manufacturing a derivative effector cell comprising a first polynucleotide encoding a chimeric antigen receptor (CAR) and one or more additional polynucleotides encoding one or both of an engager and a CFR, wherein the engager has a different tumor targeting specificity from the CAR, wherein the method comprises: differentiating a genetically engineered iPSC, wherein the iPSC comprises the first and the one or more additional polynucleotides and optionally one or more genomic edits comprising: (i) CD38 knockout; (ii) HLA-I deficiency and/or HLA-II deficiency; (iii) introduction of HLA-G or non-cleavable HLA-G, or knockout of one or both of CD58 and CD54; (iv) an exogenous CD16 or a variant thereof, (v) a chimeric fusion receptor (CFR); (vi) an inactivation CAR; (vii) a signaling complex comprising a partial or full peptide of a cell surface expressed exogenous cytokine and/or a receptor thereof, (viii) at least one of the genotypes listed in Table 1; (ix) disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or (x) introduction of at least one of HLA-E, 4-1BBL, CD3, CD4, CD8, CD16, CD47, CD113, CD131, CD137, CD80, PDL1, A_{2A}R, antigen-specific TCR, Fc receptor, an antibody or functional variant or fragment thereof, a checkpoint inhibitor, and surface triggering receptor for coupling with an agonist, in comparison to its counterpart primary cell.

In some embodiments of the method, (i) the engager comprises a first binding domain having a different tumor targeting specificity from the CAR, and wherein the binding domain is specific to any one of ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CD5, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NY-ESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; or (ii) the engager comprises a first binding domain having a different tumor targeting specificity from the CAR, and wherein the binding domain is specific to any one of B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, or ROR1; or (iii) the engager comprises a second binding domain having specificity that is different from the specificity of the first binding domain and is to an extracellular portion of CD3, CD28, CD5, CD16, CD64, CD32, CD33, CD89, NKG2C, NKG2D, or any functional variants thereof, or (iv) the engager comprises a cytokine or a variant thereof between the first and the second binding domains, wherein the cytokine comprises at least one of IL2, IL4, IL6, IL7, IL9, IL10, IL11, IL12, IL15, IL18, and IL21. In some embodiments of the method, the derivative effector cell comprises a derivative CD34⁺ cell, a derivative hematopoietic stem and progenitor cell, a derivative hematopoietic multipotent progenitor cell, a derivative T cell progenitor, a derivative NK cell progenitor, a derivative T lineage cell, a derivative NKT lineage cell, a derivative NK lineage cell, a derivative B lineage cell; or a derivative effector cell having one or more functional features that are not present in a counterpart primary T, NK, NKT, and/or B cell.

In some embodiments of the method, the CAR may be: (i) T cell specific or NK cell specific; (ii) a bi-specific antigen binding CAR; (iii) a switchable CAR; (iv) a dimerized CAR; (v) a split CAR; (vi) a multi-chain CAR; (vii) an inducible CAR; (viii) co-expressed with another CAR; (ix) co-expressed with a partial or full peptide of a cell surface expressed exogenous cytokine and/or a receptor thereof, optionally in separate constructs or in a bi-cistronic construct; (x) co-expressed with a checkpoint inhibitor, optionally in separate constructs or in a bi-cistronic construct; and/or (xi) specific to any one of ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CD5, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NY-ESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; wherein the CAR of any one of (i) to (xi) is optionally inserted at a TCR locus, and/or is driven by an endogenous promoter of TCR, and/or the TCR is knocked out by the CAR insertion. In various embodiments of the method, the CAR comprises an antigen recognition region specific to B7H3.

In those embodiments of the method where the iPSC comprises a genomic edit for an exogenous CD16 or a variant thereof, the CD16 or a variant thereof may comprise at least one of: (a) a high affinity non-cleavable CD16 (hnCD16); (b) F176V and S197P in ectodomain domain of CD16; (c) a full or partial ectodomain originated from CD64; (d) a non-native (or non-CD16) transmembrane domain; (e) a non-native (or non-CD16) intracellular domain; (f) a non-native (or non-CD16) signaling domain; (g) a non-native stimulatory domain; and (h) transmembrane, signaling, and stimulatory domains that are not originated from CD16, and are originated from a same or different polypeptide.

In those embodiments of the method where the iPSC comprises a genomic edit for a CFR, the CFR may comprise an ectodomain fused to a transmembrane domain, which is operatively connected to an endodomain, and wherein the ectodomain, transmembrane domain and the endodomain do not comprise any endoplasmic reticulum (ER) retention signals or endocytosis signals. In some embodiments, (i) the ectodomain of the CFR comprises a full or partial length of an extracellular portion of a signaling protein comprising at least one of CD3ε, CD3γ, CD3δ, CD28, CD5, CD16, CD64, CD32, CD33, CD89, NKG2C, NKG2D, any functional variants, and a combination or a chimera thereof, (ii) the ectodomain of the CFR initiates signal transduction upon binding to a selected agonist; or (iii) the endodomain of the CFR comprises a cytotoxicity domain comprising at least a full length or a portion of CD3ζ, 2B4, DAP10, DAP12, DNAM1, CD137 (4-1BB), IL21, IL7, IL12, IL15, NKp30, NKp44, NKp46, NKG2C, or NKG2D polypeptide; and optionally wherein the endodomain further comprises one or more of: (a) a co-stimulatory domain comprising a full length or a portion of CD2, CD27, CD28, CD40L, 4-1BB, OX40, ICOS, PD-1, LAG-3, 2B4, BTLA, DAP10, DAP12, CTLA-4, or NKG2D polypeptide, or any combination thereof, (b) a co-stimulatory domain comprising a full length or a portion of CD28, 4-1BB, CD27, CD40L, ICOS, CD2, or combinations thereof, (c) a persistency signaling domain comprising a full length or a portion of an endodomain of a cytokine receptor comprising IL7R, IL15R, IL18R, IL12R, IL23R, or combinations thereof; and/or (d) a full or a partial intracellular portion of a receptor tyrosine kinase (RTK), a tumor necrosis factor receptor (TNFR), an EGFR or a FAS receptor. In some embodiments, the selected agonist may be (i) an antibody or a functional variant or fragment thereof, or (ii) an engager; and may: (a) be encoded by the polynucleotide comprised in the cell of the composition or is comprised in the composition; (b) comprise at least a first binding domain that is specific to at least one tumor antigen comprising B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, or ROR1; and optionally, (c) comprise a second binding domain that is specific to a cell surface protein of: (1) the cell of the composition; or (2) a bystander effector cell; wherein the cell surface protein comprises an extracellular portion of CD3, CD28, CD5, CD16, CD64, CD32, CD33, CD89, NKG2C, NKG2D, or any functional variants thereof.

In those embodiments of the method where the iPSC comprises a genomic edit for an inactivation CAR, the inactivation CAR may: (i) target an upregulated surface protein in activated recipient immune cells; or (ii) comprise at least one of a CD38-CAR, a CD25-CAR, a CD69-CAR, a CD44-CAR, a 4-1BB-CAR, an OX40-CAR, and a CD40L-CAR. In those embodiments of the method where the iPSC comprises a genomic edit for introduced or increased expression of a checkpoint inhibitor, the checkpoint inhibitor may be an antagonist to one or more checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, and inhibitory KIR.

In some embodiments of the method, the iPSC may comprise: (i) one or more exogenous polynucleotides integrated in a safe harbor locus or a selected gene locus; or (ii) more than two exogenous polynucleotides integrated in different safe harbor loci or two or more selected gene loci. In some embodiments, the safe harbor locus comprises at least one of AAVS1, CCR5, ROSA26, collagen, HTRP, H11, GAPDH, or RUNX1; and wherein the selected gene locus is one of B2M, TAP1, TAP2, Tapasin, NLRC5, CIITA, RFXANK, RFX5, RFXAP, TCR, NKG2A, NKG2D, CD38, CD25, CD69, CD44, CD58, CD54, CD56, CIS, CBL-B, SOCS2, PD1, CTLA4, LAG3, TIM3, or TIGIT; and/or wherein the integration of the exogenous polynucleotides knocks out expression of the gene in the locus. In particular embodiments, the TCR locus may be a constant region of TCR alpha and/or TCR beta (TRAC and/or TRBC).

In those embodiments of the method where the iPSC comprises a genomic edit for a signaling complex comprising a partial or full peptide of a cell surface expressed exogenous cytokine or receptor thereof, the cell surface expressed exogenous cytokine or receptor thereof may: (a) comprise at least one of IL2, IL4, IL6, IL7, IL9, IL10, IL11, IL12, IL15, IL18, IL21, and its respective receptor(s); or (b) comprise at least one of: (i) co-expression of IL15 and IL15Rα by using a self-cleaving peptide; (ii) a fusion protein of IL15 and IL15Rα; (iii) an IL15/IL15Rα fusion protein with intracellular domain of IL15Rα truncated or eliminated; (iv) a fusion protein of IL15 and membrane bound Sushi domain of IL15Rα; (v) a fusion protein of IL15 and IL15Rβ; (vi) a fusion protein of IL15 and common receptor γC, wherein the common receptor γC is native or modified; and (vii) a homodimer of IL15Rβ, wherein any one of (b)(i)-(vii) is optionally co-expressed with the CAR in separate constructs or in a bi-cistronic construct; or (c) comprise at least one of: (i) a fusion protein of IL7 and IL7Rα; (ii) a fusion protein of IL7 and common receptor γC, wherein the common receptor γC is native or modified; and (iii) a homodimer of IL7Rβ, wherein any one of (c)(i)-(iii) is optionally co-expressed with the CAR in separate constructs or in a bi-cistronic construct; and optionally, (d) be transiently expressed.

In some embodiments of the method, the method may further comprise genomically engineering a clonal iPSC to knock in polynucleotides encoding the CAR and one or both of the engager and the CFR; and optionally: (i) to knock out CD38, (ii) to knock out B2M and/or CIITA, (iii) to knock out one or both of CD58 and CD54, and/or (iv) to introduce expression of HLA-G or non-cleavable HLA-G, a high affinity non-cleavable CD16 or a variant thereof, and/or a partial or full peptide of a cell surface expressed exogenous cytokine and/or a receptor thereof. In some embodiments, the genomic engineering comprises targeted editing. In some embodiments, the targeted editing comprises deletion, insertion, or in/del, and wherein the targeted editing is carried out by CRISPR, ZFN, TALEN, homing nuclease, homology recombination, or any other functional variation of these methods.

In yet another aspect, the invention provides a method of improving tumor cell control and clearance comprising administering to a subject in need thereof the composition provided herein, wherein the tumor is a solid tumor or the tumor is heterogenous. In some embodiments, the cells of the composition express an antibody or functional variant or fragment thereof, or an engager. In other embodiments, the composition comprises an engager. In some embodiments, (i) the engager comprises a first binding domain having a different tumor targeting specificity from the CAR, and wherein the binding domain is specific to any one of ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CD5, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NY-ESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; or (ii) the engager comprises a first binding domain having a different tumor targeting specificity from the CAR, and wherein the binding domain is specific to any one of B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, or ROR1; or (iii) the engager comprises a second binding domain having specificity that is different from the specificity of the first binding domain and is to an extracellular portion of CD3, CD28, CD5, CD16, CD64, CD32, CD33, CD89, NKG2C, NKG2D, or any functional variants thereof; or (iv) the engager comprises a cytokine or a variant thereof between the first and the second binding domains, wherein the cytokine comprises at least one of IL2, IL4, IL6, IL7, IL9, IL10, IL11, IL12, IL15, IL18, and IL21. In some embodiments, the cells of the composition are iPSC-derived effector cells further comprising one or more of: (i) a CD38 knockout; (ii) an exogenous CD16 or a variant thereof; (iii) HLA-I and/or HLA-II deficiency; (iv) introduction of HLA-G or non-cleavable HLA-G, or knockout of one or both of CD58 and CD54; (v) introduction of a CFR; (vi) a signaling complex comprising a partial or full peptide of a cell surface expressed exogenous cytokine or a receptor thereof; (vii) an inactivation CAR; (viii) disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; and/or (ix) introduction of at least one of HLA-E, 4-1BBL, CD3, CD4, CD8, CD16, CD47, CD113, CD131, CD137, CD80, PDL1, A_{2A}R, antigen-specific TCR, Fc receptor, an antibody or functional variant or fragment thereof, a checkpoint inhibitor, and surface triggering receptor for coupling with an agonist. In some embodiments, administration of the cells of the composition results in one or more of: (i) increased cytotoxicity; (ii) improved persistency and/or survival; (iii) enhanced ability in migrating, and/or activating or recruiting bystander immune cells, to tumor sites; (iv) improved tumor penetration; (v) enhanced ability to reduce tumor immunosuppression; (vi) improved ability in rescuing tumor antigen escape; (vii) controlled apoptosis; (viii) enhanced or acquired ADCC; and (ix) ability to avoid fratricide, in comparison to administration of their counterpart primary cells.

Another aspect of the present application provides a chimeric antigen receptor (CAR) comprising a binding domain that comprises: (i) an amino acid sequence that is of at least about 99%, 98%, 96%, 95%, 90%, 85%, or 80% identity to SEQ ID NO: 36, 37, 38, 39, 40, or 41; (ii) an amino acid sequence represented by a variant of SEQ ID NO: 36, and wherein the variant has one or more mutations at positions comprising 1, 40, 46, 79, 87, 88, 89, 97, 98, and 117 of SEQ ID NO: 36; (iii) an amino acid sequence represented by a variant of SEQ ID NO: 36, wherein the variant has one or more substitutions comprising Q1E, T40A, E46V, G79L, K87R, P88A, D89E, V97A, S98R, and Q117L according to SEQ ID NO:36; or (iv) an amino acid sequence represented by any of SEQ ID NOs: 36, 37, 38, 39, 40, and 41. In various embodiments of the CAR, the CAR further comprises a hinge peptide that comprises no more than 80 amino acids, or comprises between 80 to 180 amino acids. In various embodiments, the CAR has at least one of the following characteristics: (i) being T cell specific; (ii) being NK cell specific; (iii) binding to tumor cell surface B7H3; (iv) reducing tumor cell surface shedding of B7H3 antigen; or (v) increasing tumor cell surface B7H3 density. In another embodiment of the CAR, when the CAR is expressed in an effector cell, said effector cell has one or more of the following characteristics: (i) enhancing effector cell activation and killing function compared to a corresponding effector cell lacking the chimeric receptor; and (ii) capable of in vivo tumor progression control, tumor cell burden reduction, tumor clearance, and/or improving rate of survival of a subject carrying the tumor compared to a corresponding cell lacking the chimeric receptor.

Yet another aspect of the present application provides a method of manufacturing a derivative cells comprising a polynucleotide encoding the CAR described herein, wherein the method comprises differentiating an iPSC to obtain the derivative cells, wherein the polynucleotide encoding the CAR is introduced into the iPSC before differentiation or is introduced to the derivative cells after iPSC differentiation.

Various objects and advantages of the compositions and methods as provided herein will become apparent from the following description taken in conjunction with the accompanying drawings wherein are set forth, by way of illustration and example, certain embodiments of this invention.

### BRIEF DESCRIPTION OF THE DRAWINGS

FIGS. 1A-1E demonstrate that T cell engagers improve CAR-T cell efficacy in a range of tumor models. FIG. 1A shows profiling of Antigen and EpCAM expression in SKOV3, MDA-MB-231, Jimt1, and K562 tumor cell lines. FIG. 1B shows a dose-dependent increase in cytolysis by CD8⁺ T cells co-cultured with SKOV3 tumor cells in the presence of EpCAM BiTEs. FIG. 1C shows specific cytolysis results of SKOV3 (Antigen^{High}), JIMT1 (Antigen⁺), and MDA-MB-231 (Antigen^{Low}) tumor cells co-cultured with Antigen-specific CAR transduced T cells with and without EpCAM BiTE. FIG. 1D shows the IFNγ production of the CAR-T cell with or without the BiTE via intracellular cytokine staining and FIG. 1E shows the comparison of the effector cell IFNγ production with or without the presence of BiTE using ELISA (P<0.001).

FIGS. 2A-2C show that T cell engager enhances tumor growth control in 3D tumor models. FIG. 2A shows improved tumor penetration of CAR-T cells in the presence of BiTEs using SKOV3 tumor spheroids co-cultured with the CAR-T cells. FIG. 2B shows increased tumor clearance using SKOV3 tumor spheroids co-cultured with effector cells at indicated ET ratios in the presence of BiTE (**** P<0.0001, ** P<0.01). FIG. 2C shows increased tumor clearance using MDA-MB-231 tumor spheroids co-cultured with effector cells at indicated ET ratios in the presence of BiTE (**** P<0.0001, ** P<0.01).

FIGS. 3A and 3B show multi-antigen targeting enhances control of heterogenous tumor cultures. FIG. 3A shows tumor cell cytolysis of the standard, or individual, cultures of two tumor types with Antigen-specific CAR-T cells with or without BiTE, as monitored via Incucyte assay. FIG. 3B shows tumor cell cytolysis of the mixed tumor cell culture of two tumor types with Antigen-specific CAR-T with or without BiTE.

FIGS. 4A and 4B shows that B7H3 TriKE increases NK cell function (FIG. 4A) and proliferation (FIG. 4B) compared to exogenous IL-15 culturing condition.

FIG. 5 shows that engineered iNK cells penetrate tumor spheroids and target B7H3 expressing prostate cancer cells more effectively with the presence of the B7H3 TriKE.

FIG. 6 shows that a combination of engineered iNK cells and the B7H3 TriKE synergize to target and eliminate ovarian cancer cells.

FIGS. 7A and 7B show that engineered iPSC-derived CAR-iT cells secreting BiTEs activate allogeneic bystander CD8⁺ T cells and control tumor clearance in concert. FIG. 7A illustrates iPSC derived effector cells expressing CAR and EpCAM BiTEs. FIG. 7B shows SKOV3 spheroid clearance by primary CD8⁺ T cells with or without addition of a BiTE, and CAR-iT cells expressing a BiTE with or without bystander T cells.

FIG. 8 illustrates exemplary CAR constructs to generate B7H3-CAR iPSC and effector cells, and the cell surface camB7H3-CAR expression is shown in effector cells compared to control cells without B7H3-CAR expression.

FIGS. 9A and 9B show that the B7H3-CAR effector cells demonstrate effective functional response (FIG. 9A) and durable anti-tumor cytotoxicity (FIG. 9B) across multiple solid tumor lines compared to control cells.

FIG. 10 shows that B7H3-CAR T cells activation upon antigen-specific stimulation in a broad panel of tumor cell lines.

FIG. 11 shows evaluation of three exemplary B7H3-CAR motifs to determine the optimal configuration for B7H3 targeting using a cytoxicity assay against PC3 prostate cancer cells.

FIG. 12 shows that CAR-T cells expressing the three exemplary CAR motifs are reactive against a broad array of tumor cell lines.

FIG. 13 shows phenotype profiling of engineered iPSC cells transduced with a camB7H3-CAR.

FIGS. 14A-14C show pheonotype and function profiling of B7H3-CAR⁺ iNK cells. FIG. 14 A shows the homogeneous iPSC-derived iNK cell population and surface camB7H3-CAR expression. FIG. 14B demonstrate antigen specific cytokine release and degranulation by B7H3-CAR⁺ iNK under antigen specific stimulation in vitro. FIG. 14C shows that B7H3-CAR⁺ iNK cells respond to cell surface B7H3 in an antigen dose dependent manner.

FIG. 15 illustrates iPSC-derived CAR-T effector cell recognition and targeting of heterogenous tumor cells having CAR antigen expressed at different levels, and the intratumoral production by the iPSC derived effector cell of T cell engagers (BiTEs) specific to secondary tumor associated antigen(s) that differ from that of the CAR, and BiTE-dependent recognition of secondary tumor associated antigen(s) leading to HLA-independent targeting of heterogenous tumor cells by bystander T cells, or by iPSC-derived effector cells comprising a surface triggering receptor, such as a CFR (Chimeric Fusion Receptor), for coupling with the BiTE.

### DETAILED DESCRIPTION OF THE INVENTION

Genomic modification of iPSCs (induced pluripotent stem cells) includes polynucleotide insertion, deletion and substitution. Exogenous gene expression in genome-engineered iPSCs often encounters problems such as gene silencing or reduced gene expression after prolonged clonal expansion of the original genome-engineered iPSCs, after cell differentiation, and in dedifferentiated cell types from the cells derived from the genome-engineered iPSCs. On the other hand, direct engineering of primary immune cells such as T or NK cells is challenging, and presents a hurdle to the preparation and delivery of engineered immune cells for adoptive cell therapy. The present invention provides an efficient, reliable, and targeted approach for stably integrating one or more exogenous genes, including suicide genes and other functional modalities, which provide improved therapeutic properties relating to engraftment, trafficking, homing, migration, cytotoxicity, viability, maintenance, expansion, longevity, self-renewal, persistence, and/or survival, into iPSC derivative cells, including but not limited to HSCs (hematopoietic stem and progenitor cell), T cell progenitor cells, NK cell progenitor cells, T lineage cells, NKT lineage cells, NK lineage cells, and immune effector cells having one or more functional features that are not present in primary NK, T, and/or NKT cells.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein, the articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell population or culture media, refer to a composition that is free of a specified substance or its source thereof, such as, 95% free, 96% free, 97% free, 98% free, 99% free of the specified substance or its source thereof, or is undetectable as measured by conventional means. The term “free of” or “essentially free of” a certain ingredient or substance in a composition also means that no such ingredient or substance is (1) included in the composition at any concentration, or (2) included in the composition functionally inert, but at a low concentration. Similar meaning can be applied to the term “absence of,” where referring to the absence of a particular substance or its source thereof of a composition.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours, but including up to 48 or 72 hours or longer, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism.

As used herein, the terms “reprogramming” or “dedifferentiation” or “increasing cell potency” or “increasing developmental potency” refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “differentiation” is the process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the term “induced pluripotent stem cells” or “iPSCs”, means that the stem cells are produced in vitro, using reprogramming factor and/or small molecule chemical driven methods, from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature.

As used herein, the term “embryonic stem cell” refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extra-embryonic membranes or the placenta, i.e., are not totipotent.

As used herein, the term “multipotent stem cell” refers to a cell that has the developmental potential to differentiate into cells of one or more germ layers (ectoderm, mesoderm and endoderm), but not all three. Thus, a multipotent cell can also be termed a “partially differentiated cell.” Multipotent cells are well known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to a state of a cell with a degree of developmental potential that is less than totipotent and pluripotent.

Pluripotency can be determined, in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

Two types of pluripotency have previously been described: the “primed” or “metastable” state of pluripotency akin to the epiblast stem cells (EpiSC) of the late blastocyst, and the “Naïve” or “Ground” state of pluripotency akin to the inner cell mass of the early/preimplantation blastocyst. While both pluripotent states exhibit the characteristics as described above, the naïve or ground state further exhibits: (i) pre-inactivation or reactivation of the X-chromosome in female cells; (ii) improved clonality and survival during single-cell culturing; (iii) global reduction in DNA methylation; (iv) reduction of H3K27me3 repressive chromatin mark deposition on developmental regulatory gene promoters; and (v) reduced expression of differentiation markers relative to primed state pluripotent cells. Standard methodologies of cellular reprogramming in which exogenous pluripotency genes are introduced to a somatic cell, expressed, and then either silenced or removed from the resulting pluripotent cells are generally seen to have characteristics of the primed-state of pluripotency. Under standard pluripotent cell culture conditions such cells remain in the primed state unless the exogenous transgene expression is maintained, wherein characteristics of the ground-state are observed.

As used herein, the term “pluripotent stem cell morphology” refers to the classical morphological features of an embryonic stem cell. Normal embryonic stem cell morphology is characterized by being round and small in shape, with a high nucleus-to-cytoplasm ratio, the notable presence of nucleoli, and typical inter-cell spacing.

As used herein, the term “subject” refers to any animal, preferably a human patient, livestock, or other domesticated animal.

A “pluripotency factor,” or “reprogramming factor,” refers to an agent capable of increasing the developmental potency of a cell, either alone or in combination with other agents. Pluripotency factors include, without limitation, polynucleotides, polypeptides, and small molecules capable of increasing the developmental potency of a cell. Exemplary pluripotency factors include, for example, transcription factors and small molecule reprogramming agents.

“Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

“Cultivate” or “maintain,” refers to the sustaining, propagating (growing) and/or differentiating of cells outside of tissue or the body, for example in a sterile plastic (or coated plastic) cell culture dish or flask. “Cultivation” or “maintaining,” may utilize a culture medium as a source of nutrients, hormones and/or other factors helpful to propagate and/or sustain the cells.

As used herein, the term “mesoderm” refers to one of the three germinal layers that appears during early embryogenesis and which gives rise to various specialized cell types including blood cells of the circulatory system, muscles, the heart, the dermis, skeleton, and other supportive and connective tissues.

As used herein, the term “definitive hemogenic endothelium” (HE) or “pluripotent stem cell-derived definitive hemogenic endothelium” (iHE) refers to a subset of endothelial cells that give rise to hematopoietic stem and progenitor cells in a process called endothelial-to-hematopoietic transition. The development of hematopoietic cells in the embryo proceeds sequentially from lateral plate mesoderm through the hemangioblast to the definitive hemogenic endothelium and hematopoietic progenitors.

The term “hematopoietic stem and progenitor cells,” “hematopoietic stem cells,” “hematopoietic progenitor cells,” or “hematopoietic precursor cells” refers to cells which are committed to a hematopoietic lineage but are capable of further hematopoietic differentiation and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). The term “definitive hematopoietic stem cell” as used herein, refers to CD34⁺ hematopoietic cells capable of giving rise to both mature myeloid and lymphoid cell types including T lineage cells, NK lineage cells and B lineage cells. Hematopoietic cells also include various subsets of primitive hematopoietic cells that give rise to primitive erythrocytes, megakarocytes and macrophages.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells in an MHC class I-restricted manner. A T cell can be any T cell, such as a cultured T cell, e.g., a primary T cell, or a T cell from a cultured T cell line, e.g., Jurkat, SupT1, etc., or a T cell obtained from a mammal. The T cell can be CD3⁺ cells. The T cell can be any type of T cell and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), peripheral blood mononuclear cells (PBMCs), peripheral blood leukocytes (PBLs), tumor infiltrating lymphocytes (TILs), memory T cells, naïve T cells, regulator T cells, gamma delta T cells (δδ T cells), and the like. Additional types of helper T cells include cells such as Th3 (Treg), Th17, Th9, or Tfh cells. Additional types of memory T cells include cells such as central memory T cells (Tcm cells), effector memory T cells (Tem cells and TEMRA cells). The T cell can also refer to a genetically engineered T cell, such as a T cell modified to express a T cell receptor (TCR) or a chimeric antigen receptor (CAR). A T cell, or a T cell like effector cell can also be differentiated from a stem cell or progenitor cell (“a derived T cell” or “a derived T cell like effector cell”, or collectively, “a derivative T lineage cell”). A T cell like derivative effector cell may have a T cell lineage in some respects, but at the same time has one or more functional features that are not present in a primary T cell. In this application, a T cell, a T cell like effector cell, a derived T cell, a derived T cell like effector cell, or a derivative T lineage cell, are collectively termed as “a T lineage cell”.

“CD4⁺ T cells” refers to a subset of T cells that express CD4 on their surface and are associated with cell-mediated immune response. They are characterized by the secretion profiles following stimulation, which may include secretion of cytokines such as IFN-gamma, TNF-alpha, IL2, IL4 and IL10. “CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8⁺ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refer to a subset of peripheral blood lymphocytes defined by the expression of CD56 or CD16 and the absence of the T cell receptor (CD3). As used herein, the terms “adaptive NK cell” and “memory NK cell” are interchangeable and refer to a subset of NK cells that are phenotypically CD3⁻ and CD56⁺, expressing at least one of NKG2C and CD57, and optionally, CD16, but lack expression of one or more of the following: PLZF, SYK, FceRγ, and EAT-2. In some embodiments, isolated subpopulations of CD56⁺ NK cells comprise expression of CD16, NKG2C, CD57, NKG2D, NCR ligands, NKp30, NKp40, NKp46, activating and inhibitory KIRs, NKG2A and/or DNAM-1. CD56⁺ can be dim or bright expression. An NK cell, or an NK cell like effector cell may be differentiated from a stem cell or progenitor cell (“a derived NK cell” or “a derived NK cell like effector cell”, or collectively, “a derivative NK lineage cell”). An NK cell like derivative effector cell may have an NK cell lineage in some respects, but at the same time has one or more functional features that are not present in a primary NK cell. In this application, an NK cell, an NK cell like effector cell, a derived NK cell, a derived NK cell like effector cell, or a derivative NK lineage cell, are collectively termed as “an NK lineage cell”.

As used herein, the term “NKT cells” or “natural killer T cells” refers to CD1d-restricted T cells, which express a T cell receptor (TCR). Unlike conventional T cells that detect peptide antigens presented by conventional major histocompatibility (MHC) molecules, NKT cells recognize lipid antigens presented by CD1d, a non-classical MHC molecule. Two types of NKT cells are recognized. Invariant or type I NKT cells express a very limited TCR repertoire—a canonical α-chain (Vα24-Jα18 in humans) associated with a limited spectrum of β chains (Vβ11 in humans). The second population of NKT cells, called non-classical or non-invariant type II NKT cells, display a more heterogeneous TCR αβ usage. Type I NKT cells are considered suitable for immunotherapy. Adaptive or invariant (type I) NKT cells can be identified with the expression of at least one or more of the following markers, TCR Vα24-Jα18, Vb11, CD1d, CD3, CD4, CD8, aGalCer, CD161 and CD56.

The term “effector cell” generally is applied to certain cells in the immune system that carry out a specific activity in response to stimulation and/or activation, or to cells that effect a specific function upon activation. As used herein, the term “effector cell” includes, and in some contexts is interchangeable with, immune cells, “differentiated immune cells,” and primary or differentiated cells that are edited and/or modulated to carry out a specific activity in response to stimulation and/or activation. Non-limiting examples of effector cells include primary-sourced or iPSC-derived T cells, NK cells, NKT cells, B cells, macrophages, and neutrophils.

As used herein, the term “isolated” or the like refers to a cell, or a population of cells, which has been separated from its original environment, i.e., the environment of the isolated cells is substantially free of at least one component as found in the environment in which the “un-isolated” reference cells exist. The term includes a cell that is removed from some or all components as it is found in its natural environment, for example, isolated from a tissue or biopsy sample. The term also includes a cell that is removed from at least one, some or all components as the cell is found in non-naturally occurring environments, for example, isolated form a cell culture or cell suspension. Therefore, an isolated cell is partly or completely separated from at least one component, including other substances, cells or cell populations, as it is found in nature or as it is grown, stored or subsisted in non-naturally occurring environments. Specific examples of isolated cells include partially pure cell compositions, substantially pure cell compositions and cells cultured in a medium that is non-naturally occurring. Isolated cells may be obtained from separating the desired cells, or populations thereof, from other substances or cells in the environment, or from removing one or more other cell populations or subpopulations from the environment.

As used herein, the term “purify” or the like refers to increasing purity. For example, the purity can be increased to at least 50%, 60%, 70%, 80%, 90%, 95%, 99%, or 100%.

As used herein, the term “encoding” refers to the inherent property of specific sequences of nucleotides in a polynucleotide, such as a gene, a cDNA, or a mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” may further comprise replacement of the endogenous sequence or a nucleotide that is deleted with the one or more inserted nucleotides.

As used herein, the term “exogenous” is intended to mean that the referenced molecule or the referenced activity is introduced into, or non-native to, the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell. Similarly, the term when used in reference to expression of an encoding nucleic acid refers to expression of an encoding nucleic acid contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e., a polypeptide found in nature) or fragment thereof; a variant polypeptide (i.e., a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

As used herein, the term “polynucleotide” refers to a polymeric form of nucleotides of any length, either deoxyribonucleotides or ribonucleotides or analogs thereof. The sequence of a polynucleotide is composed of four nucleotide bases: adenine (A); cytosine (C); guanine (G); thymine (T); and uracil (U) for thymine when the polynucleotide is RNA. A polynucleotide can include a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. Polynucleotide also refers to both double- and single-stranded molecules.

As used herein, the term “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “subunit” as used herein refers to each separate polypeptide chain of a protein coinplex, where each separate polypeptide chain can form a stable folded structure by itself. Many protein molecules are composed of more than one subunit, where the amino acid sequences can either be identical for each subunit, or similar, or completely different. For example, CD3 complex is composed of CD3α, CD3ε, CD3δ, CD3γ, and CD3ζ subunits, which form the CD3ε/CD3γ, CD3ε/CD3δ, and CD3ζ/CD3ζ dimers. Within a single subunit, contiguous portions of the polypeptide chain frequently fold into compact, local, semi-independent units that are called “domains”. Many protein domains may further comprise independent “structural subunits”, also called subdomains, contributing to a common function of the domain. As such, the term “subdomain” as used herein refers to a protein domain inside of a larger domain, for example, a binding domain within an ectodomain of a cell surface receptor; or a stimulatory domain or a signaling domain of an endodomain of a cell surface receptor.

“Operably-linked” or “operatively linked,” interchangeable with “operably connected” or “operatively connected,” refers to the association of nucleic acid sequences on a single nucleic acid fragment (or amino acids in a polypeptide with multiple domains) so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation. As a further example, a receptor-binding domain can be operatively connected to an intracellular signaling domain, such that binding of the receptor to a ligand transduces a signal responsive to said binding.

“Fusion proteins” or “chimeric proteins”, as used herein, are proteins created through genetic engineering to join two or more partial or whole polynucleotide coding sequences encoding separate proteins, and the expression of these joined polynucleotides results in a single peptide or multiple polypeptides with functional properties derived from each of the original proteins or fragments thereof. Between two neighboring polypeptides of different sources in the fusion protein, a linker (or spacer) peptide can be added. The chimeric fusion receptors (CFRs) described herein are fusion, or chimeric, proteins.

As used herein, the term “genetic imprint” refers to genetic or epigenetic information that contributes to preferential therapeutic attributes in a source cell or an iPSC, and is retainable in the source cell derived iPSCs, and/or the iPSC-derived hematopoietic lineage cells. As used herein, “a source cell” is a non-pluripotent cell that may be used for generating iPSCs through reprogramming, and the source cell derived iPSCs may be further differentiated to specific cell types including any hematopoietic lineage cells. The source cell derived iPSCs, and differentiated cells therefrom are sometimes collectively called “derived” or “derivative” cells depending on the context. For example, derivative effector cells, or derivative NK lineage cells or derivative T lineage cells, as used throughout this application are cells differentiated from an iPSC, as compared to their counterpart primary cells obtained from natural/native sources such as peripheral blood, umbilical cord blood, or other donor tissues. As used herein, the genetic imprint(s) conferring a preferential therapeutic attribute is incorporated into the iPSCs either through reprogramming a selected source cell that is donor-, disease-, or treatment response-specific, or through introducing genetically modified modalities to iPSC using genomic editing. In the aspect of a source cell obtained from a specifically selected donor, disease or treatment context, the genetic imprint contributing to preferential therapeutic attributes may include any context specific genetic or epigenetic modifications which manifest a retainable phenotype, i.e., a preferential therapeutic attribute, that is passed on to iPSC-derived cells of the selected source cell, irrespective of the underlying molecular events being identified or not. Donor-, disease-, or treatment response-specific source cells may comprise genetic imprints that are retainable in iPSCs and derived hematopoietic lineage cells, which genetic imprints include but are not limited to, prearranged monospecific TCR, for example, from a viral specific T cell or invariant natural killer T (iNKT) cell; trackable and desirable genetic polymorphisms, for example, homozygous for a point mutation that encodes for the high-affinity CD16 receptor in selected donors; and predetermined HLA requirements, i.e., selected HLA-matched donor cells exhibiting a haplotype with increased population. As used herein, preferential therapeutic attributes include improved engraftment, trafficking, homing, viability, self-renewal, persistence, immune response regulation and modulation, survival, and cytotoxicity of a derived cell. A preferential therapeutic attribute may also relate to antigen targeting receptor expression; HLA presentation or lack thereof; resistance to tumor microenvironment; induction of bystander immune cells and immune modulations; improved on-target specificity with reduced off-tumor effect; resistance to treatment such as chemotherapy. When derivative cells having one or more therapeutic attributes are obtained from differentiating an iPSC that has genetic imprint(s) conferring a preferential therapeutic attribute incorporated thereto, such derivative cells are also called “synthetic cells”. In general, a synthetic cell possesses one or more non-native cell functions when compared to its closest counterpart primary cell, whether the synthetic cell is differentiated from engineered pluripotent cells or obtained by engineering a primary cell from natural/native sources, such as peripheral blood, umbilical cord blood, or other donor tissues.

The term “enhanced therapeutic property” as used herein, refers to a therapeutic property of a cell that is enhanced as compared to a typical immune cell of the same general cell type. For example, an NK cell with an “enhanced therapeutic property” will possess an enhanced, improved, and/or augmented therapeutic property as compared to a typical, unmodified, and/or naturally occurring NK cell. Therapeutic properties of an immune cell may include, but are not limited to, cell engraftment, trafficking, homing, viability, self-renewal, persistence, immune response regulation and modulation, survival, and cytotoxicity. Therapeutic properties of an immune cell are also manifested by antigen targeting receptor expression; HLA presentation or lack thereof, resistance to tumor microenvironment; induction of bystander immune cells and immune modulations; improved on-target specificity with reduced off-tumor effect; resistance to treatment such as chemotherapy.

As used herein, the term “engager” refers to a molecule, e.g., a fusion polypeptide, which is capable of forming a link between an immune cell (e.g., a T cell, a NK cell, a NKT cell, a B cell, a macrophage, or a neutrophil), and a tumor cell; and activating the immune cell. Examples of engagers include, but are not limited to, bi-specific T cell engagers (BiTEs), bi-specific killer cell engagers (BiKEs), tri-specific killer cell engagers, or multi-specific killer cell engagers, and universal engagers compatible with multiple immune cell types.

As used herein, the term “surface triggering receptor” refers to a receptor capable of triggering or initiating an immune response, e.g., a cytotoxic response. Surface triggering receptors may be engineered, and may be expressed on effector cells, e.g., a T cell, a NK cell, a NKT cell, a B cell, a macrophage, a neutrophil. In some embodiments, the surface triggering receptor facilitates bi- or multi-specific antibody engagement between the effector cells and specific target cell (e.g., a tumor cell) independent of the effector cell's natural receptors and cell types. Using this approach, one may generate iPSCs comprising a universal surface triggering receptor, and then differentiate such iPSCs into populations of various effector cell types that express the universal surface triggering receptor. By “universal”, it is meant that the surface triggering receptor can be expressed in, and activate, any effector cells irrespective of the cell type, and all effector cells expressing the universal receptor can be coupled or linked to the engagers recognizable by the surface triggering receptor, regardless of the engager's tumor binding specificities. In some embodiments, engagers having the same tumor targeting specificity are used to couple with the universal surface triggering receptor. In some embodiments, engagers having different tumor targeting specificity are used to couple with the universal surface triggering receptor. As such, one or multiple effector cell types can be engaged to kill one specific type of tumor cells in some case, and to kill two or more types of tumors in some other cases. A surface triggering receptor generally comprises a co-stimulatory domain for effector cell activation and an epitope that is specific to the epitope binding region of an engager. A bi-specific engager is specific to the epitope of a surface triggering receptor on one end, and is specific to a tumor antigen on the other end.

As used herein, the term “safety switch protein” refers to an engineered protein designed to prevent potential toxicity or otherwise adverse effects of a cell therapy. In some instances, the safety switch protein expression is conditionally controlled to address safety concerns for transplanted engineered cells that have permanently incorporated the gene encoding the safety switch protein into its genome. This conditional regulation could be variable and might include control through a small molecule-mediated post-translational activation and tissue-specific and/or temporal transcriptional regulation. The safety switch could mediate induction of apoptosis, inhibition of protein synthesis, DNA replication, growth arrest, transcriptional and post-transcriptional genetic regulation and/or antibody-mediated depletion. In some instance, the safety switch protein is activated by an exogenous molecule, e.g., a prodrug, that when activated, triggers apoptosis and/or cell death of a therapeutic cell. Examples of safety switch proteins, include, but are not limited to suicide genes such as caspase 9 (or caspase 3 or 7), thymidine kinase, cytosine deaminase, B-cell CD20, modified EGFR, and any combination thereof. In this strategy, a prodrug that is administered in the event of an adverse event is activated by the suicide-gene product and kills the transduced cell.

As used herein, the term “pharmaceutically active proteins or peptides” refers to proteins or peptides that are capable of achieving a biological and/or pharmaceutical effect on an organism. A pharmaceutically active protein has healing curative or palliative properties against a disease and may be administered to ameliorate relieve, alleviate, reverse or lessen the severity of a disease. A pharmaceutically active protein also has prophylactic properties and is used to prevent the onset of a disease or to lessen the severity of such disease or pathological condition when it does emerge. Pharmaceutically active proteins include an entire protein or peptide or pharmaceutically active fragments thereof. It also includes pharmaceutically active analogs of the protein or peptide or analogs of fragments of the protein or peptide. The term pharmaceutically active protein also refers to a plurality of proteins or peptides that act cooperatively or synergistically to provide a therapeutic benefit. Examples of pharmaceutically active proteins or peptides include, but are not limited to, receptors, binding proteins, transcription and translation factors, tumor growth suppressing proteins, antibodies or fragments thereof, growth factors, and/or cytokines.

As used herein, the term “signaling molecule” refers to any molecule that modulates, participates in, inhibits, activates, reduces, or increases, the cellular signal transduction. Signal transduction refers to the transmission of a molecular signal in the form of chemical modification by recruitment of protein complexes along a pathway that ultimately triggers a biochemical event in the cell. Signal transduction pathways are well known in the art, and include, but are not limited to, G protein coupled receptor signaling, tyrosine kinase receptor signaling, integrin signaling, toll gate signaling, ligand-gated ion channel signaling, ERK/MAPK signaling pathway, Wnt signaling pathway, cAMP-dependent pathway, and IP3/DAG signaling pathway.

As used herein, the term “targeting modality” refers to a molecule, e.g., a polypeptide, that is genetically incorporated into a cell to promote antigen and/or epitope specificity that includes but is not limited to i) antigen specificity as it relates to a unique chimeric antigen receptor (CAR) or T cell receptor (TCR), ii) engager specificity as it relates to monoclonal antibodies or bi-specific engagers, iii) targeting of transformed cells, iv) targeting of cancer stem cells, and v) other targeting strategies in the absence of a specific antigen or surface molecule.

As used herein, the term “specific” or “specificity” can be used to refer to the ability of a molecule, e.g., a receptor or an engager, to selectively bind to a target molecule, in contrast to non-specific or non-selective binding.

The term “adoptive cell therapy” as used herein refers to a cell-based immunotherapy that, as used herein, relates to the transfusion of autologous or allogenic lymphocytes, identified as T or B cells, genetically modified or not, that have been expanded ex vivo prior to said transfusion.

A “therapeutically sufficient amount”, as used herein, includes within its meaning a non-toxic but sufficient and/or effective amount of the particular therapeutic and/or pharmaceutical composition to which it is referring to provide a desired therapeutic effect. The exact amount required will vary from subject to subject depending on factors such as the patient's general health, the patient's age and the stage and severity of the condition. In particular embodiments, a therapeutically sufficient amount is sufficient and/or effective to ameliorate, reduce, and/or improve at least one symptom associated with a disease or condition of the subject being treated.

Differentiation of pluripotent stem cells requires a change in the culture system, such as changing the stimuli agents in the culture medium or the physical state of the cells. The most conventional strategy utilizes the formation of embryoid bodies (EBs) as a common and critical intermediate to initiate the lineage-specific differentiation. “Embryoid bodies” are three-dimensional clusters that have been shown to mimic embryo development as they give rise to numerous lineages within their three-dimensional area. Through the differentiation process, typically a few hours to days, simple EBs (for example, aggregated pluripotent stem cells elicited to differentiate) continue maturation and develop into a cystic EB at which time, typically days to a few weeks, they are further processed to continue differentiation. EB formation is initiated by bringing pluripotent stem cells into close proximity with one another in three-dimensional multilayered clusters of cells, typically this is achieved by one of several methods including allowing pluripotent cells to sediment in liquid droplets, sedimenting cells into “U” bottomed well-plates or by mechanical agitation. To promote EB development, the pluripotent stem cell aggregates require further differentiation cues, as aggregates maintained in pluripotent culture maintenance medium do not form proper EBs. As such, the pluripotent stem cell aggregates need to be transferred to differentiation medium that provides eliciting cues towards the lineage of choice. EB-based culture of pluripotent stem cells typically results in generation of differentiated cell populations (ectoderm, mesoderm and endoderm germ layers) with modest proliferation within the EB cell cluster. Although proven to facilitate cell differentiation, EBs, however, give rise to heterogeneous cells in variable differentiation state because of the inconsistent exposure of the cells in the three-dimensional structure to differentiation cues from the environment. In addition, EBs are laborious to create and maintain. Moreover, cell differentiation through EB formation is accompanied with modest cell expansion, which also contributes to low differentiation efficiency.

In comparison, “aggregate formation,” as distinct from “EB formation,” can be used to expand the populations of pluripotent stem cell derived cells. For example, during aggregate-based pluripotent stem cell expansion, culture media are selected to maintain proliferation and pluripotency. Cell proliferation generally increases the size of the aggregates forming larger aggregates, these aggregates can be routinely mechanically or enzymatically dissociated into smaller aggregates to maintain cell proliferation within the culture and increase numbers of cells. As distinct from EB culture, cells cultured within aggregates in maintenance culture maintain markers of pluripotency. The pluripotent stem cell aggregates require further differentiation cues to induce differentiation.

As used herein, “monolayer differentiation” is a term referring to a differentiation method distinct from differentiation through three-dimensional multilayered clusters of cells, i.e., “EB formation.” Monolayer differentiation, among other advantages disclosed herein, avoids the need for EB formation for differentiation initiation. Because monolayer culturing does not mimic embryo development such as EB formation, differentiation towards specific lineages is deemed as minimal as compared to all three germ layer differentiation in EB.

As used herein, a “dissociated cell” or “single dissociated cell” refers to a cell that has been substantially separated or purified away from other cells or from a surface (e.g., a culture plate surface). For example, cells can be dissociated from an animal or tissue by mechanical or enzymatic methods. Alternatively, cells that aggregate in vitro can be dissociated from each other, such as by dissociation into a suspension of clusters, single cells or a mixture of single cells and clusters, enzymatically or mechanically. In yet another alternative embodiment, adherent cells are dissociated from a culture plate or other surface. Dissociation thus can involve breaking cell interactions with extracellular matrix (ECM) and substrates (e.g., culture surfaces), or breaking the ECM between cells.

As used herein, a “master cell bank” or “MCB” refers to a clonal master engineered iPSC line, which is a clonal population of iPSCs that have been engineered to comprise one or more therapeutic attributes, have been characterized, tested, qualified, and expanded, and have been shown to reliably serve as the starting cellular material for the production of cell-based therapeutics through directed differentiation in manufacturing settings. In various embodiments, an MCB is maintained, stored, and/or cryopreserved in multiple vessels to prevent genetic variation and/or potential contamination by reducing and/or eliminating the total number of times the iPS cell line is passaged, thawed or handled during the manufacturing processes.

As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type. The feeder cells are optionally from a different species as the cells they are supporting. For example, certain types of human cells, including stem cells, can be supported by primary cultures of mouse embryonic fibroblasts, or immortalized mouse embryonic fibroblasts. In another example, peripheral blood derived cells or transformed leukemia cells support the expansion and maturation of natural killer cells. The feeder cells may typically be inactivated when being co-cultured with other cells by irradiation or treatment with an antagonistic mitotic agent such as mitomycin to prevent them from outgrowing the cells they are supporting. Feeder cells may include endothelial cells, stromal cells (for example, epithelial cells or fibroblasts), and leukemic cells. Without limiting the foregoing, one specific feeder cell type may be a human feeder, such as a human skin fibroblast. Another feeder cell type may be mouse embryonic fibroblasts (MEF). In general, various feeder cells can be used in part to maintain pluripotency, direct differentiation towards a certain lineage, enhance proliferation capacity and promote maturation to a specialized cell type, such as an effector cell.

As used herein, a “feeder-free” (FF) environment refers to an environment such as a culture condition, cell culture or culture media which is essentially free of feeder or stromal cells, and/or which has not been pre-conditioned by the cultivation of feeder cells. “Pre-conditioned” medium refers to a medium harvested after feeder cells have been cultivated within the medium for a period of time, such as for at least one day. Pre-conditioned medium contains many mediator substances, including growth factors and cytokines secreted by the feeder cells cultivated in the medium. In some embodiments, a feeder-free environment is free of both feeder or stromal cells and is also not pre-conditioned by the cultivation of feeder cells.

“Functional” as used in the context of genomic editing or modification of iPSC, and derived non-pluripotent cells differentiated therefrom, or genomic editing or modification of non-pluripotent cells and derived iPSCs reprogrammed therefrom, refers to (1) at the gene level successful knocked-in, knocked-out, knocked-down gene expression, transgenic or controlled gene expression such as inducible or temporal expression at a desired cell development stage, which is achieved through direct genomic editing or modification, or through “passing-on” via differentiation from or reprogramming of a starting cell that is initially genomically engineered; or (2) at the cell level-successful removal, addition, or alteration of a cell function/characteristics via (i) gene expression modification obtained in said cell through direct genomic editing, (ii) gene expression modification maintained in said cell through “passing-on” via differentiation from or reprogramming of a starting cell that is initially genomically engineered; (iii) down-stream gene regulation in said cell as a result of gene expression modification that only appears in an earlier development stage of said cell, or only appears in the starting cell that gives rise to said cell via differentiation or reprogramming; or (iv) enhanced or newly attained cellular function or attribute displayed within the mature cellular product, initially derived from the genomic editing or modification conducted at the iPSC, progenitor or dedifferentiated cellular origin.

“HLA deficient”, including HLA class I deficient, or HLA class II deficient, or both, refers to cells that either lack, or no longer maintain, or have a reduced level of surface expression of a complete MHIC complex comprising an HLA class I protein heterodimer and/or an HLA class II heterodimer, such that the diminished or reduced level is less than the level naturally detectable by other cells or by synthetic methods.

“Modified HLA deficient iPSC,” as used herein, refers to an HLA deficient iPSC that is further modified by introducing genes expressing proteins related but not limited to improved differentiation potential, antigen targeting, antigen presentation, antibody recognition, persistence, immune evasion, resistance to suppression, proliferation, co-stimulation, cytokine stimulation, cytokine production (autocrine or paracrine), chemotaxis, and cellular cytotoxicity, such as non-classical HLA class I proteins (e.g., HLA-E and HLA-G), chimeric antigen receptor (CAR), T cell receptor (TCR), CD16 Fc Receptor, BCL11b, NOTCH, RUNX1, IL15, 4-1BB, DAP10, DAP12, CD24, CD3ζ, 4-1BBL, CD47, CD113, and PDL1. The cells that are “modified HLA deficient” also include cells other than iPSCs.

The term “ligand” refers to a substance that forms a complex with a target molecule to produce a signal by binding to a site on the target. The ligand may be a natural or artificial substance capable of specific binding to the target. The ligand may be in the form of a protein, a peptide, an antibody, an antibody complex, a conjugate, a nucleic acid, a lipid, a polysaccharide, a monosaccharide, a small molecule, a nanoparticle, an ion, a neurotransmitter, or any other molecular entity capable of specific binding to a target. The target to which the ligand binds, may be a protein, a nucleic acid, an antigen, a receptor, a protein complex, or a cell. A ligand that binds to and alters the function of the target and triggers a signaling response is called “agonistic” or “an agonist”. A ligand that binds to a target and blocks or reduced a signaling response is “antagonistic” or “an antagonist.”

The term “antibody” is used herein in the broadest sense and refers generally to an immune-response generating molecule that contains at least one binding site that specifically binds to a target, wherein the target may be an antigen, or a receptor that is capable of interacting with certain antibodies. For example, an NK cell can be activated by the binding of an antibody or the Fc region of an antibody to its Fc-gamma receptors (FcγR), thereby triggering the ADCC (antibody-dependent cellular cytotoxicity) mediated effector cell activation. A specific piece or portion of an antigen or receptor, or a target in general, to which an antibody binds is known as an epitope or an antigenic determinant. The term “antibody” includes, but is not limited to, native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. An antibody may be a murine antibody, a human antibody, a humanized antibody, a camel IgG, a single variable new antigen receptor (VNAR), a shark heavy-chain antibody (Ig-NAR), a chimeric antibody, a recombinant antibody, a single-domain antibody (dAb), an anti-idiotype antibody, a bi-specific-, multi-specific- or multimeric-antibody, or antibody fragment thereof. Anti-idiotype antibodies are specific for binding to an idiotope of another antibody, wherein the idiotope is an antigenic determinant of an antibody. A bi-specific antibody may be a BiTE (bi-specific T cell engager) or a BiKE (bi-specific killer cell engager), and a multi-specific antibody may be a TriKE (tri-specific Killer cell engager). Non-limiting examples of antibody fragments include Fab, Fab′, F(ab′)2, F(ab′)3, Fv, Fabc, pFc, Fd, single chain fragment variable (scFv), tandem scFv (scFv)2, single chain Fab (scFab), disulfide stabilized Fv (dsFv), minibody, diabody, triabody, tetrabody, single-domain antigen binding fragments (sdAb), camelid heavy-chain IgG and Nanobody® fragments, recombinant heavy-chain-only antibody (VHH), and other antibody fragments that maintain the binding specificity of the antibody.

“Fc receptors,” abbreviated FcR, are classified based on the type of antibody that they recognize. For example, those that bind the most common class of antibody, IgG, are called Fc-gamma receptors (FcγR), those that bind IgA are called Fc-alpha receptors (FcαR) and those that bind IgE are called Fc-epsilon receptors (FcεR). The classes of FcRs are also distinguished by the cells that express them (macrophages, granulocytes, natural killer cells, T and B cells) and the signaling properties of each receptor. Fc-gamma receptors (FcγR) include several members, FcγRI (CD64), FcγRIIA (CD32), FcγRIIB (CD32), FcγRIIIA (CD16a), and FcγRIIIB (CD16b), which differ in their antibody affinities due to their different molecular structures.

“Chimeric Receptor” is a general term used to describe an engineered, artificial, or a hybrid receptor protein molecule that is made to comprise two or more portions of amino acid sequences that are originated from at least two different proteins. The chimeric receptor proteins have been engineered to give a cell the ability to initiate signal transduction and carry out downstream function upon binding of an agonistic ligand to the receptor. Exemplary “chimeric receptors” include, but are not limited to, chimeric antigen receptors (CARs), chimeric fusion receptors (CFRs), chimeric Fc receptors (CFcRs), as well as fusions of two or more receptors.

“Chimeric Fc Receptor,” abbreviated as “CFcR”, is a term used to describe engineered Fc receptors having their native transmembrane and/or intracellular signaling domains modified, or replaced, with non-native transmembrane and/or intracellular signaling domains. In some embodiments of the chimeric Fc receptor, in addition to having one of, or both, transmembrane and signaling domains being non-native, one or more stimulatory domains can be introduced to the intracellular portion of the engineered Fc receptor to enhance cell activation, expansion and function upon triggering of the receptor. Unlike a chimeric antigen receptor (CAR) which contains an antigen binding domain to target antigen, the chimeric Fc receptor binds to an Fc fragment, or the Fc region of an antibody, or the Fc region comprised in an engager or a binding molecule and activating the cell function with or without bringing the targeted cell close in vicinity. For example, a Fcγ receptor can be engineered to comprise selected transmembrane, stimulatory, and/or signaling domains in the intracellular region that respond to the binding of IgG at the extracellular domain, thereby generating a CFcR. In one example, a CFcR is produced by engineering CD16, a Fc receptor, by replacing its transmembrane domain and/or intracellular domain. To further improve the binding affinity of the CD16-based CFcR, the extracellular domain of CD64 or the high-affinity variants of CD16 (F176V, for example) can be incorporated. In some embodiments of the CFcR where high affinity CD16 extracellular domain is involved, the proteolytic cleavage site comprising a serine at position 197 is eliminated or is replaced such that the extracellular domain of the receptor is non-cleavable, i.e., not subject to shedding, thereby obtaining a hnCD16-based CFcR.

CD16, an FcγR receptor, has been identified to have two isoforms, Fc receptors FcγRIIIa (CD16a) and FcγRIIIb (CD16b). CD16a is a transmembrane protein expressed by NK cells, which binds monomeric IgG attached to target cells to activate NK cells and facilitate antibody-dependent cell-mediated cytotoxicity (ADCC). “High affinity CD16,” “non-cleavable CD16,” or “high affinity non-cleavable CD16” (abbreviated as hnCD16), as used herein, refers to a natural or non-natural variant of CD16. The wildtype CD16 has low affinity and is subject to ectodomain shedding, a proteolytic cleavage process that regulates the cell's surface density of various cell surface molecules on leukocytes upon NK cell activation. F176V and F158V are exemplary CD16 polymorphic variants having high affinity. A CD16 variant having the cleavage site (position 195-198) in the membrane-proximal region (position 189-212) altered or eliminated is not subject to shedding. The cleavage site and the membrane-proximal region are described in detail in International Publication No. WO 2015/148926, the complete disclosure of which is incorporated herein by reference. The CD16 S197P variant is an engineered non-cleavable version of CD16. A CD16 variant comprising both F158V and S197P has high affinity and is non-cleavable. Another exemplary high affinity and non-cleavable CD16 (hnCD16) variant is an engineered CD16 comprising an ectodomain originated from one or more of the 3 exons of the CD64 ectodomain.

I. Cells and Compositions Useful for Adoptive Cell Therapies with Enhanced Properties

Provided herein is a strategy to systematically engineer the regulatory circuitry of a clonal iPSC without impacting the differentiation potency of the iPSC and cell development biology of the iPSC and its derivative cells, while enhancing the therapeutic properties of the derivative cells differentiated from the iPSC. The iPSC-derived cells are functionally improved and suitable for adoptive cell therapies following a combination of selective modalities being introduced to the cells at the level of iPSC through genomic engineering. It was previously unclear whether altered iPSCs comprising one or more provided genetic edits still have the capacity to enter cell development, and/or to mature and generate functional differentiated cells while retaining modulated activities and/or properties. Unanticipated failures during directed cell differentiation from iPSCs have been attributed to aspects including, but not limited to, development stage specific gene expression or lack thereof, requirements for HLA complex presentation, protein shedding of introduced surface expressing modalities, and the need for reconfiguration of differentiation protocols enabling phenotypic and/or functional changes in the cell. The present application shows that the one or more selected genomic modifications as provided herein do not negatively impact iPSC differentiation potency, and the functional effector cells derived from the engineered iPSC have enhanced and/or acquired therapeutic properties attributable to the individual or combined genomic modifications retained in the effector cells following the iPSC differentiation. Further, all genomic modifications and combinations thereof as may be described in the context of iPSC and iPSC-derived effector cells are applicable to primary sourced cells, including primary immune cells such as T, NK, or immunoregulatory cells, whether cultured or expanded, the modification of which results in engineered immune cells useful for adoptive cell therapy.

Further, while CAR-T cells have been shown to be effective and potent in treating several hematologic malignancies, engineered T cell therapies have had limited success in addressing solid tumors. Unlike liquid tumors where uniformly-expressed antigens are accessible and can be effectively targeted, tumor access and antigen heterogeneity are significant barriers to the successful development of CAR-T cells in solid tumors. As demonstrated herein, the combination of a bi-specific T cell engager (BiTE) with a CAR-T cell, including an iPSC-derived CAR-effector cell, enhances anti-tumor activity against heterogenous solid tumors.

1. Chimeric Antigen Receptor (CAR) expression

Applicable to the genetically engineered iPSC and derivative effector cell thereof may be any CAR design known in the art. CAR is a fusion protein generally including an ectodomain that comprises a target binding region (for example, an antigen recognition domain), a transmembrane domain, and an endodomain. In some embodiments, the ectodomain can further include a signal peptide or leader sequence and/or a spacer. In some embodiments, the endodomain can further comprise a signaling peptide that activates the effector cell expressing the CAR. In some embodiments, the antigen recognition domain can specifically bind an antigen. In some embodiments, the antigen recognition domain can specifically bind an antigen associated with a disease or pathogen. In some embodiments, the disease-associated antigen is a tumor antigen, wherein the tumor may be a liquid or a solid tumor. In some embodiments, the CAR is suitable to activate either T or NK lineage cells expressing the CAR. In some embodiments, the CAR is NK cell specific for comprising NK-specific signaling components. In certain embodiments, the T cells are derived from CAR expressing iPSCs, and the derivative T lineage cells may comprise T helper cells, cytotoxic T cells, memory T cells, regulatory T cells, natural killer T cells, αβ T cells, γδ T cells, or a combination thereof. In certain embodiments, the NK cells are derived from CAR expressing iPSCs.

In certain embodiments, the antigen recognition domain comprises a murine antibody, a human antibody, a humanized antibody, a camel Ig, a single variable new antigen receptor (VNAR), shark heavy-chain antibody (Ig-NAR), a chimeric antibody, a recombinant antibody, a single-domain antibody (dAb), an anti-idiotype antibody, a bispecific-, multi-specific- or multimeric-antibody, or antibody fragment thereof. Anti-idiotype antibodies are specific for binding to an idiotope of another antibody, wherein the idiotope is an antigenic determinant of an antibody. A bi-specific antibody may be a BiTE (bi-specific T cell engager) or a BiKE (bi-specific killer cell engager), and a multi-specific antibody may be a TriKE (tri-specific Killer cell engager). Non-limiting examples of antibody fragments include Fab, Fab′, F(ab′)2, F(ab′)3, Fv, Fabc, pFc, Fd, single chain fragment variable (scFv), tandem scFv (scFv)2, single chain Fab (scFab), disulfide stabilized Fv (dsFv), minibody, diabody, triabody, tetrabody, single-domain antigen binding fragments (sdAb), camelid heavy-chain IgG and Nanobody® fragments, recombinant heavy-chain-only antibody (VHH), and other antibody fragments that maintain the binding specificity of the antibody. Non-limiting examples of antigens that may be targeted by a CAR include ADGRE2, B7H3, carbonic anhydrase IX (CAIX), CCR1, CCR4, carcinoembryonic antigen (CEA), CD3, CD5, CD7, CD8, CD10, CD19, CD20, CD22, CD30, CD33, CD34, CD38, CD41, CD44, CD44V6, CD49f, CD52, CD56, CD70, CD74, CD99, CD123, CD133, CD138, CD269 (BCMA), CD5, CLEC12A, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A1 (MAGE-A1), MICA/B, MR1, Mucin 1 (Muc-1), Mucin 16 (Muc-16), Mesothelin (MSLN), NKCSI, NKG2D ligands, c-Met, cancer-testis antigen NY-ESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and various pathogen antigen known in the art. Non-limiting examples of pathogens include viruses, bacteria, fungi, parasites and protozoa capable of causing diseases.

B7H3 (CD276) belongs to the B7 family of immune checkpoint inhibitors and is expressed on immune cells (such as antigen-presenting cells or macrophages) and tumor cells, and has inhibitory roles on T cells, contributing to tumor cell immune evasion. Recent studies have shown that B7H3 is a crucial player in tumor growth and metastasis beyond the immune regulatory roles. Further, B7H3 expression has been correlated with poor prognosis in ovarian, RCC, NSCLC, pancreatic cancer, prostate cancer and colon cancer. As such, the present specification provides a genetically engineered iPSC and its derivative cell, wherein the cell comprises a polynucleotide encoding a CAR targeting a B7H3 tumor antigen.

In various embodiments of the CAR targeting a B7H3 tumor antigen, the CAR comprises a recombinant heavy-chain-only antibody (VHH) that specifically binds to B7H3. In one embodiment, the CAR comprises a binding domain comprising an amino acid sequence that is of at least about 99%, about 98%, about 96%, about 95%, about 90%, about 85%, or at least about 80% identity to SEQ ID NO: 36. In another embodiment, the CAR comprises a binding domain comprising an amino acid sequence that is of at least about 99%, about 98%, about 96%, about 95%, about 90%, about 85%, or at least about 80% identity to SEQ ID NO: 37. In another embodiment, the CAR comprises a binding domain comprising an amino acid sequence that is of at least about 99%, about 98%, about 96%, about 95%, about 90%, about 85%, or at least about 80% identity to SEQ ID NO: 38. In another embodiment, the CAR comprises a binding domain comprising an amino acid sequence that is of at least about 99%, about 98%, about 96%, about 95%, about 90%, about 85%, or at least about 80% identity to SEQ ID NO: 39. In another embodiment, the CAR comprises a binding domain comprising an amino acid sequence that is of at least about 99%, about 98%, about 96%, about 95%, about 90%, about 85%, or at least about 80% identity to SEQ ID NO: 40. In another embodiment, the CAR comprises a binding domain comprising an amino acid sequence that is of at least about 99%, about 98%, about 96%, about 95%, about 90%, about 85%, or at least about 80% identity to SEQ ID NO: 41.

In certain embodiments, the CAR comprises a binding domain comprising a variant of SEQ ID NO: 36, and wherein the variant has one or more mutations at positions comprising 1, 40, 46, 79, 87, 88, 89, 97, 98, and 117 of SEQ ID NO: 36. In other embodiments, the CAR comprises an amino acid sequence represented by a variant of SEQ ID NO: 36, wherein the variant has one or more substitutions comprising Q1E, T40A, E46V, G79L, K87R, P88A, D89E, V97A, S98R, and Q117L according to SEQ ID NO: 36. In other embodiments, the CAR comprises an amino acid sequence represented by any of SEQ ID NOs: 36, 37, 38, 39, 40, and 41.

SEQ ID NO: 36
QVQLVESGGGLVQPGGSLRLSCAASGFTFSSYWMYWVRQT
PGKGLEWVSTINRDGSATWYADSVKGRFTISRDNAKNTGY
LQMNSLKPDDTAVYYCVSDPDNYSSDEMVPYWGQGTQVTV
SS
(122 a. a. VHH camelid B7H3)
SEQ ID NO: 37
EVQLVESGGGLVQPGGSLRLSCAASGFTFSSYWMYWVRQA
PGKGLVWVSTINRDGSATWYADSVKGRFTISRDNAKNTLY
LQMNSLRAEDTAVYYCARDPDNYSSDEMVPYWGQGTLVTV
SS
(122 a.a. VHH1)
SEQ ID NO: 38
EVQLVESGGGLVQPGGSLRLSCAASGFTESSYWMYWVRQA
PGKGLVWVSTINRDGSATWYADSVKGRFTISRDNAKNTLY
LQMNSLRAEDTAVYYCVSDPDNYSSDEMVPYWGQGTLVTV
SS
(122 a.a. VHH2)
SEQ ID NO: 39
EVQLVESGGGLVQPGGSLRLSCAASGFTFSSYWMYWVRQT
PGKGLVWVSTINRDGSATWYADSVKGRFTISRDNAKNTLY
LQMNSLRAEDTAVYYCVSDPDNYSSDEMVPYWGQGTLVTV
SS
(122 a.a. VHH3)
SEQ ID NO: 40
EVQLVESGGGLVQPGGSLRLSCAASGFTFSSYWMYWVRQA
PGKGLEWVSTINRDGSATWYADSVKGRFTISRDNAKNTLY
LQMNSLRAEDTAVYYCVSDPDNYSSDEMVPYWGQGTLVTV
SS
(122 a.a. VHH4)
SEQ ID NO: 41
EVQLVESGGGLVQPGGSLRLSCAASGFTFSSYWMYWVRQT
PGKGLEWVSTINRDGSATWYADSVKGRFTISRDNAKNTGY
LQMNSLRPEDTAVYYCVSDPDNYSSDEMVPYWGQGTLVTV
SS
(122 AA. VHH5)

In some embodiments, there is a spacer/hinge between the antigen recognition region and the transmembrane domain of the CAR, although in some other embodiments such spacer/hinge is not required. Exemplary spacers that may be included in a CAR are commonly known in the art, including, but not limited to, IgG4 spacers, CD28 spacers, CD8 spacers, or combinations of more than one spacer. The length of the spacers may also vary, from about 15 amino acids to about 300 amino acids or more. In this application, for ease of description, a spacer less than around 80 amino acids, for example 10-80 amino acids, is considered short; a spacer of about 80-180 amino acids is considered medium; and a spacer more than 180 amino acids is considered long. Non-limiting exemplary spacer peptides include those represented by an amino acid sequence of at least about 80%, about 85%, about 90%, about 95%, about 96%, about 97%, about 98%, or about 99% identity to any of SEQ ID NOs: 31-35.

SEQ ID NO: 31
IEVMYPPPYLDNEKSNGTIIHVKGKHLCPSPLFPGPSKP
(39 a.a.)
SEQ ID NO: 32
ESKYGPPCPPCPGGGSSGGGSGGQPREPQVYTLPPSQEEM
TKNQVSLTCLVKGFYPSDIAVEWESNGQPENNYKTTPPVL
DSDGSFFL
(88 a.a.)
SEQ ID NO: 33
ESKYGPPCPPCPAPEFEGGPSVELFPPKPKDTLMISRTPE
VTCVVVDVSQEDPEVQENWYVDGVEVHNAKTKPREEQFQS
TYRVVSVLT
(89 a.a.)
SEQ ID NO: 34
ESKYGPPCPPCPGGGSSGGGSGGQPREPQVYTLPPSQEEM
TKNQVSLTCLVKGFYPSDIAVEWESNGQPENNYKTTPPVL
DSDGSFFLYSRLTVDKSRWQEGNVFSCSVMHEALHNHYTQ
KSLSLSLGK
(129 a.a.)
SEQ ID NO: 35
ESKYGPPCPPCPAPEFEGGPSVFLFPPKPKDTLMISRTPE
VTCVVVDVSQEDPEVQFNWYVDGVEVHNAKTKPREEQFQS
TYRVVSVLTVLHQDWLNGKEYKCKVSNKGLPSSIEKTISK
AKGQPREPQVYTLPPSQEEMTKNQVSLTCLVKGFYPSDIA
VEWESNGQPENNYKTTPPVLDSDGSFFLYSRLTVDKSRWQ
EGNVESCSVMHEALHNHYTQKSLSLSLGK
(229 a.a.)

In some embodiments, the transmembrane domain of a CAR comprises a full length or at least a portion of the native or modified transmembrane region of 2B4, 4-1BB, BTLA, CD2, CD3δ, CD3δ, CD3γ, CD3ζ, CD4, CD8, CD8a, CD8b, CD16, CD27, CD28, CD28H, CD40, CD84, CD166, CS1, CTLA-4, DNAM1, DAP1, DAP12, FcεRIγ, ICOS, ICAM-1, IL7, IL12, IL15, KIR2DL4, KIR2DS1, KIR2DS2, LAG3, PD1, NKp30, NKp44, NKp46, NKG2C, NKG2D, OX40, or T cell receptor polypeptide.

In some embodiments, the signaling peptide of the endodomain (or intracellular domain) comprises a full length or at least a portion of a polypeptide of 2B4, CD2, CD3ζ, CD3ζ1XX, CD8, CD28, CD28H, CD137 (4-1B), CS1, DAP10, DAP12, DNAM1, FcεRIγ, IL2Ry, TL7R, IL21R, IL2Rβ (IL15Rβ), IL21, IL7, IL12, IL15, IL21, KIR2DS2, NKp30, NKp44, NKp46, NKG2C, or NKG2D. In one embodiment, the signaling peptide of a CAR comprises an amino acid sequence that has at least about 85%, about 90%, about 95%, about 96%, about 97%, about 98%, or about 99% identity to at least one ITAM (immunoreceptor tyrosine-based activation motif) of CD3ζ.

In certain embodiments, the endodomain further comprises at least one co-stimulatory signaling region. The co-stimulatory signaling region can comprise a full length or at least a portion of a polypeptide of CD27, CD28, 4-1BB, OX40, ICOS, PD-1, LAG-3, 2B4, BTLA, DAP10, DAP12, CTLA-4, or NKG2D, or any combination thereof.

In one embodiment, the CAR applicable to the cells provided herein comprises a co-stimulatory domain derived from CD28, and a signaling domain comprising the native or modified ITAM1 of CD3ζ, represented by an amino acid sequence having at least about 85%, about 90%, about 95%, about 96%, about 97%, about 98%, or about 99% identity to SEQ ID NO: 1. In a further embodiment, the CAR comprising a co-stimulatory domain derived from CD28, and a native or modified ITAM1 of CD3ζ also comprises a hinge domain and trans-membrane domain derived from CD28, wherein an scFv may be connected to the trans-membrane domain through the hinge, and the CAR comprises an amino acid sequence of at least about 85%, about 90%, about 95%, about 96%, about 97%, about 98%, or about 99% identity to SEQ ID NO: 2. In some embodiments, the sequence identity is at least 80%. In some embodiments, the sequence identity is at least 90%. In some embodiments, the sequence identity is at least 95%. In some embodiments, the sequence identity is 100%.

SEQ ID NO: 1
RSKRSRLLHSDYMNMTPRRPGPTRKHYQPYAPPRDFAAYRSR
VKESRSADAPAYQQGQNQLYNELNLGRREEYDVLDKRRGRDP
EMGGKPRRKNPQEGLFNELQKDKMAEAFSEIGMKGERRRGKG
HDGLFQGLSTATKDTFDALHMQALPPR
(153 a.a. CD28 co-stim + CD3ζITAM)
SEQ ID NO: 2
IEVMYPPPYLDNEKSNGTIIHVKGKHLCPSPLFPGPSKPFWV
LVVVGGVLACYSLLVTVAFIIFWVRSKRSRLLHSDYMNMTPR
RPGPTRKHYQPYAPPRDFAAYRSRVKESRSADAPAYQQGQNQ
LYNELNLGRREEYDVLDKRRGRDPEMGGKPRRKNPQEGLENE
LQKDKMAEAFSEIGMKGERRRGKGHDGLFQGLSTATKDTFDA
LHMQALPPR
(219 a.a. CD28 hinge + CD28 TM +
CD28 co-stim + CD3ζITAM)

In another embodiment, the CAR applicable to the cells provided herein comprises a transmembrane domain derived from NKG2D, a co-stimulatory domain derived from 2B4, and a signaling domain comprising the native or modified CD3ζ, represented by an amino acid sequence of at least about 85%, about 90%, about 95%, about 96%, about 97%, about 98%, or about 99% identity to SEQ ID NO: 3. Said CAR comprising a transmembrane domain derived from NKG2D, a co-stimulatory domain derived from 2B4, and a signaling domain comprising the native or modified CD3ζ may further comprise a CD8 hinge, wherein the amino acid sequence of such a structure is of at least about 85%, about 90%, about 95%, about 96%, about 97%, about 98%, or about 99% identity to SEQ ID NO: 4. In some embodiments, the sequence identity is at least 80%. In some embodiments, the sequence identity is at least 90%. In some embodiments, the sequence identity is at least 95%. In some embodiments, the sequence identity is 100%.

SEQ ID NO: 3
SNLFVASWIAVMIIFRIGMAVAIFCCFFFPSWRRKRKEKQ
SETSPKEFLTIYEDVKDLKTRRNHEQEQTEPGGGSTIYSM
IQSQSSAPTSQEPAYTLYSLIQPSRKSGSRKRNHSPSFNS
TIYEVIGKSQPKAQNPARLSRKELENFDVY SRVKESRSA
DAPAYKQGQNQLYNELNLGRREEYDVLDKRRGRDPEMGGK
PRRKNPQEGLYNELQKDKMAEAYSEIGMKGERRRGKGHDG
LYQGLSTATKDTYDALHMQALPPR
(263 a.a NKG2D TM + 2B4 + CD3ζ)
SEQ ID NO: 4
TTTPAPRPPTPAPTIASQPLSLRPEACRPAAGGAVHTRGL
DFACDSNLEVASWIAVMIIFRIGMAVAIFCCFFFPSWRRK
RKEKQSETSPKEFLTIYEDVKDLKTRRNHEQEQTFPGGGS
TIYSMIQSQSSAPTSQEPAYTLYSLIQPSRKSGSRKRNHS
PSENSTIYEVIGKSQPKAQNPARLSRKELENFDVYSRVKE
SRSADAPAYKQGQNQLYNELNLGRREEYDVLDKRRGRDPE
MGGKPRRKNPQEGLYNELQKDKMAEAYSEIGMKGERRRGK
GHDGLYQGLSTATKDTYDALHMQALPPR
(308 a.a CD8 hinge + NKG2D TM + 2B4 + CD3ζ)

Non-limiting CAR strategies further include heterodimeric, conditionally activated CAR through dimerization of a pair of intracellular domains (see for example, U.S. Pat. No. 9,587,020); split CAR, where homologous recombination of antigen binding, hinge, and endo-domains to generate a CAR (see for example, U.S. Pub. No. 2017/0183407); multi-chain CAR that allows non-covalent link between two transmembrane domains connected to an antigen binding domain and a signaling domain, respectively (see for example, U.S. Pub. No. 2014/0134142); CARs having bispecific antigen binding domain (see for example, U.S. Pat. No. 9,447,194), or having a pair of antigen binding domains recognizing same or different antigens or epitopes (see for example, U.S. Pat. No. 8,409,577), or a tandem CAR (see for example, Hegde et al., J Clin Invest. 2016; 126(8):3036-3052); inducible CAR (see for example, U.S. Pub. Nos. 2016/0046700, 2016/0058857, and 2017/0166877); switchable CAR (see for example, U.S. Pub. No. 2014/0219975); and any other designs known in the art.

In a further embodiment, the iPSC and its derivative effector cells comprising a CAR have the CAR inserted in a TCR constant region (TRAC or TRBC), leading to TCR knockout, and optionally placing CAR expression under the control of the endogenous TCR promoter. The disruption of the constant region of TCR alpha or TCR beta (TRAC or TRBC) produces a TCR^{reg} cell. In addition, the expression of TCR is also negative in a NK lineage effector cell that differentiated from iPSC. TCR^{reg} cells do not require HLA matching, have reduced alloreactivity, and are able to prevent GvHD (Graft versus Host Disease) when used in allogeneic adoptive cell therapies. Additional insertion sites of a CAR include, but are not limited to, AAVS1, CCR5, ROSA26, collagen, HTRP, H11, GAPDH, RUNX1, B2M, TAP1, TAP2, tapasin, NLRC5, CIITA, RFXANK, RFX5, RFXAP, NKG2A, NKG2D, CD25, CD38, CD44, CD58, CD54, CD56, CD69, CD71, CIS, CBL-B, SOCS2, PD1, CTLA4, LAG3, TIM3, and TIGIT. In another embodiment, the iPSC and its derivative NK cells comprise a CAR, where the CAR is inserted in the NKG2A locus or NKG2D locus, leading to NKG2A or NKG2D knock out, thereby placing CAR expression under the control of the endogenous NKG2A or NKG2D promoter. Alpha-beta T cell receptors (TCRαβ) are antigen specific receptors essential to the immune response and are present on the cell surface of αβ T lymphocytes. Binding of TCRαβ to peptide-major histocompatibility complex (pMHC) initiates TCR-CD3 intracellular activation, recruitment of numerous signaling molecules, and branching and integrating signaling pathways, leading to mobilization of transcription factors that are critical for gene expression and T cell growth and function acquisition.

As such, in addition to genetically engineered immune cells comprising the functional modality as provided herein, aspects of the present invention provide derivative cells obtained from differentiating genomically engineered iPSCs, wherein both the iPSCs and the derivative cells comprise polynucleotides encoding one or more CARs and optionally, an engager having a different tumor targeting specificity from the CAR, along with additional modified modalities. As shown in the present specification, effector cells comprising a CAR targeting the B7H3 tumor antigen provide enhanced targeting and destruction of B7H3-expressing cancer cells, facilitating infiltration of immune cells to tumor site(s) and enhancing/extending anti-cancer responses, while activating the immune cells expressing the B7H3-CAR, including, but not limited to, primary T cells, NK cells, iPSC-derived T lineage cells, and iPSC-derived NK lineage cells to carry out targeted tumor cell killing. Additionally provided in this application is a master cell bank comprising single cell sorted and expanded clonal engineered iPSCs having at least a CAR targeting the B7H3 tumor antigen and optionally any one of the genotypes listed in Table 1, wherein the cell bank provides a platform for additional iPSC engineering and a renewable source for manufacturing off-the-shelf, engineered, homogeneous cell therapy products.

2. Engagers

In some embodiments, the iPSC, and its derivative effector cells comprising a CAR may additionally comprise introduced expression of an engager having a different tumor targeting specificity from the CAR. An engager is specific to at least one tumor antigen and is optionally also specific to at least one surface triggering receptor of an immune effector cell. Examples of engagers include, but are not limited to, bi-specific T cell engagers (BiTEs), bi-specific killer cell engagers (BiKEs), tri-specific killer cell engagers (TriKEs), multi-specific killer cell engagers, or universal engagers compatible with multiple immune cell types. Bi- or multi-specific engagers are fusion proteins consisting of two or more ligand binding domains. In some embodiments, the ligand binding domain comprises a single-chain variable fragment (scFv), or other functional variants, of different antibodies or fragments thereof, with the scFv binding to an effector cell surface molecule or surface triggering receptor, or a tumor cell via a tumor specific surface molecule. Such bi-specific or multi-specific engagers are capable of directing an effector cell (e.g., a T cell, a NK cell, an NKT cell, a B cell, a macrophage, and/or a neutrophil) to a tumor cell and activating the immune effector cell, and have shown great potential to maximize the benefits of CAR-T cell therapy.

In some embodiments, an engager is added to a composition comprising effector cells before, during or after the transfusion of the cell to a subject in need thereof. In some embodiments, an engager is engineered to be comprised in an effector cell, such that the effector cell expresses and/or secretes the enagager. In some embodiments, the engager expressed by an engineered iPSC derivative effector cell engages a bystander immune cell that comprises a surface molecule recognized and bound by the engager. In some embodiments, the engager expressed by an engineered iPSC derivative CAR effector cell binds to the derivative effector cell and activates the derivative effector cell upon binding to a tumor antigen different from the CAR antigen. In some embodiments, the engager expressed by an engineered iPSC derivative CAR effector cell binds to the derivative effector cell through an endogenous surface molecule of the effector cell. In some embodiments, the engager expressed by an engineered iPSC derivative CAR effector cell binds to the derivative effector cell through an exogenous surface triggering receptor of the effector cell. In some embodiments, the exogenous surface triggering receptor of the effector cell expressing the engager comprises a CFR (chimeric fusion receptor), as further provided herein.

In some embodiments, the surface triggering receptor facilitates bi- or multi-specific antibody engagement between the effector cells and a specific target cell (e.g., a tumor cell), independent of the effector cell's natural receptors and cell types. In some other embodiments, one or more exogenous surface triggering receptors may be introduced to the effector cells using the methods and compositions provided herein, i.e., through engineering of an iPSC, optionally generating a master cell bank comprising single cell sorted and expanded clonal engineered iPSCs, and then directing the differentiation of the iPSC to T, NK or any other effector cells comprising the same genotype as the source iPSC.

Using this approach, one may also generate iPSCs comprising a universal surface triggering receptor, and then differentiate such iPSCs into populations of various effector cell types that express the universal surface triggering receptor. In some embodiments, engagers having the same tumor targeting specificity are used to couple with different universal surface triggering receptors. In some embodiments, engagers having different tumor targeting specificity are used to couple with the same universal surface triggering receptor. As such, one or multiple effector cell types can be engaged to kill one specific type of tumor cell in some cases, and to kill two or more types of tumor cells in other cases. A surface triggering receptor generally comprises a co-stimulatory domain for effector cell activation and an anti-epitope that is specific to the epitope of an engager, or vice versa, the surface triggering receptor comprises an epitope that is recognizable or specific to the anti-epitope of the engager. For example, a bi-specific engager is specific to the epitope of a surface triggering receptor on one end and is specific to a tumor antigen on the other end.

Exemplary effector cell surface molecules, or surface triggering receptors, that can be used for bi- or multi-specific engager recognition, or coupling, or binding, include, but are not limited to, CD3, CD28, CD5, CD16, CD64, CD32, CD33, CD89, NKG2C, NKG2D, or any functional variants or chimeric receptor forms thereof as disclosed herein, including but not limited to CFRs as further detailed in this application. In some embodiments, the CD16 expressed on the surface of effector cells for engager recognition is a hnCD16, comprising the CD16 (containing F176V and optionally S197P) or CD64 extracellular domain, and native or non-native transmembrane, stimulatory and/or signaling domains as described herein. In some embodiments, the CD16 expressed on the surface of effector cells for engager recognition is a CD16-based chimeric Fc receptor (CFcR). In some embodiments, the CD16-based CFcR comprises a transmembrane domain of NKG2D, a stimulatory domain of 2B4, and a signaling domain of CD3ζ; wherein the extracellular domain of the CD16 is derived from a full length or partial sequence of the extracellular domain of CD64 or CD16; and optionally wherein the extracellular domain of CD16 comprises F176V and optionally S197P.

Exemplary tumor cell surface molecules for bi- or multi-specific engager recognition include, but are not limited to, B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, and ROR1. In one embodiment, the bi-specific engager is a bi-specific antibody specific to CD3 and CD19 (CD3×CD19 or CD3-CD19); and in another embodiment, the bi-specific antibody is CD3-CD33. In another embodiment, the bi-specific antibody is CD3-EpCAM. For engaging CD16 on an effector cell, the bi-specific antibody comprises CD16-CD30 or CD64-CD30. In another embodiment, the bi-specific antibody comprises CD16-BCMA or CD64-BCMA. In yet another embodiment, the bi-specific antibody further comprises a linker between the effector cell and tumor cell antigen binding domains, for example, a modified IL15 may be used as a linker for effector NK cells to facilitate effector cell expansion/autonomy (called TriKE, or Tri-specific Killer Engager, in some publications). In one embodiment, the TriKE is CD16-IL15-EpCAM or CD64-IL15-EpCAM. In one embodiment, the TriKE is CD16-IL15-B7H3, or CD64-IL15-B7H3. In another embodiment, the TriKE is CD16-IL15-CD33 or CD64-IL15-CD33. In yet another embodiment, the TriKE is NKG2C-IL15-CD33. The IL15 in the TriKE may also originate from other cytokines including, but not limited to, IL2, IL4, IL6, IL7, IL9, IL10, IL11, IL12, IL18, and IL21.

In some embodiments, the engager comprises a first binding domain specific to any one of ADGRE2, B7H3, carbonic anhydrase IX (CAIX), CCR1, CCR4, carcinoembryonic antigen (CEA), CD3, CD5, CD7, CD8, CD10, CD19, CD20, CD22, CD30, CD33, CD34, CD38, CD41, CD44, CD44V6, CD49f, CD52, CD56, CD70, CD74, CD99, CD123, CD133, CD138, CD269 (BCMA), CD5, CLEC12A, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A1 (MAGE-A1), MICA/B, MR1, Mucin 1 (Muc-1), Mucin 16 (Muc-16), Mesothelin (MSLN), NKCSI, NKG2D ligands, c-Met, cancer-testis antigen NY-ESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and various pathogen antigen.

In some embodiments, the engager comprises a second binding domain having specificity that is different from the specificity of the first binding domain. In various embodiments, the second binding domain is to an extracellular portion of CD3, CD28, CD5, CD16, CD64, CD32, CD33, CD89, NKG2C, NKG2D, or any functional variants thereof. In some embodiments, the engager comprises a cytokine or a variant thereof between the first and the second binding domains, wherein the cytokine comprises at least one of IL2, IL4, IL6, IL7, IL9, IL10, IL11, IL12, IL15, IL18, and IL21, as further discussed below.

In some embodiments, the surface triggering receptor for bi- or multi-specific engager could be endogenous to the effector cells, sometimes depending on the cell types. In some other embodiments, one or more exogenous surface triggering receptors could be introduced to the effector cells using the methods and compositions provided herein, i.e., through additional engineering of an iPSC comprising a genotype listed in Table 1, then directing the differentiation of the iPSC to effector cells comprising the same genotype and the surface triggering receptor as the source iPSC.

In some embodiments, as an alternative to the genomically engineered effector cells expressing an engager as provided herein, the engager may be used as a therapeutic agent together with effector cells in a combinational therapy composition targeting one or more antigens associated with a condition, a disease, or an indication (as described above). In some embodiments, the engager is a bi-specific T cell engager (BiTE). In some embodiments, the engager is a bi-specific killer cell engager (BiKE). In some embodiments, the engager is a tri-specific killer cell engager (TriKE). In some embodiments, the engager is a multi-specific killer cell engager. In some embodiments, the engager is a universal engager compatible with multiple immune cell types. In some embodiments, the engager in the combinational therapy composition activates bystander immune cells for tumor killing in a recipient of the composition. In some embodiments, the engager in the combinational therapy composition activates the effector cells comprised in the combinational therapy composition. In some embodiments of a combinational therapy composition useful for treating liquid or solid tumors, the composition comprises iPSC-derived effector cells comprising at least a CAR, as provided herein. In some embodiments, the iPSC-derived effector cells comprise hematopoietic lineage cells comprising a genotype listed in Table 1. In some embodiments, the iPSC-derived effector cells comprise NK lineage cells comprising a genotype listed in Table 1. In some embodiments, the iPSC-derived effector cells comprise T lineage cells comprising a genotype listed in Table 1.

3. Cell Surface CFR (Chimeric Fusion Receptor)

Implementation of a CFR enables an effector cell to initiate an appropriate signal transduction cascade through CFR binding with a selected agonist for enhanced therapeutic properties of the effector cell expressing the CFR. Such enhanced effector cell therapeutic properties include, but are not limited to, increased activation and cytoxicity, acquired dual targeting capability, prolonged persistency, improved trafficking and tumor penetration, enhanced ability in priming, activating or recruiting bystander immune cells to tumor sites, enhanced ability to resist immunosuppression, improved ability in rescuing tumor antigen escape, and/or controlled cell signaling feedback, metabolism and apoptosis.

Accordingly, in various aspects, the iPSCs and derivative cells comprising a CAR and optionally an engager, may further comprise a CFR that generally comprises an ectodomain fused to a transmembrane domain, which is operatively connected to an endodomain, and the CFR does not have ER (endoplasmic reticulum) retention signals or endocytosis signals in any of the ecto-, transmembrane- or endo-domains. The ectodomain of the CFR is for initiating signal transduction upon binding to an engager; the transmembrane domain is for membrane anchoring of the CFR; and the endodomain comprises at least one signaling domain that regulates (i.e., activates or deactivates) a signaling pathway of choice for enhancing cell therapeutic properties including, but not limited to, tumor killing, persistence, mobility, differentiation, TME counteracting, and/or controlled apoptosis. The elimination of ER retention signals from the CFR permits CFR cell surface presentation by itself when expressed, and the elimination of endocytosis signals from the CFR reduces CFR internalization and surface downregulation. It is important to either select domain components that have neither ER retention nor endocytosis signals, or remove ER retention or endocytosis signals from selected components of the CFR using molecular engineering tools. In addition, the domains of the CFRs as provided herein are modular, meaning for a given endodomain of a CFR, the ectodomain of the CFR is switchable depending on the binding specificity of a selected agonist, such as an antibody, a BiTE, a TriKE, or any other type of engager, to be used with said CFR; and for a given ectodomain and a specificity matching agonist, the endodomain is switchable depending on the desired signaling pathway to be activated.

In some embodiments, the ectodomain of a CFR described herein comprises a full or partial length of the extracellular portion of a protein that is involved in cell-cell signaling or interactions. In some embodiments, the ectododomain of the CFR comprises a full or partial length of the extracellular portion of CD3ε, CD3γ, CD3δ, CD28, CD5, CD16, CD64, CD32, CD33, CD89, NKG2C, NKG2D, or any functional variants, or combinations and chimera thereof. In some embodiments, the ectodomain of the CFR is recognized by at least an agonist, for example, an antibody or an engager (e.g., BiTE, BiKE or TriKE), that comprises a binding domain specific to an epitope comprised in the ectodomain of the CFR. In some embodiments, the antibody or engager to be used as an agonist with a CFR-expressing cell binds to at least one extracellular epitope of the CFR, wherein the CFR comprises a full or partial length of the extracellular portion of CD3ε, CD3γ, CD3δ, CD28, CD5, CD16, CD64, CD32, CD33, CD89, NKG2C, NKG2D, or any functional variants or combined/chimeric forms thereof. In some embodiments, the engager recognizes at least one tumor antigen comprising B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, or ROR1. In particular embodiments, both ER retention and endocytosis signals are absent, or are removed or eliminated, from the CFR ectodomain using genetic engineering methods.

In some embodiments, the ectodomain of the CFR comprises a full or partial length of the extracellular portion of CD3ε, CD3γ, CD36 or any functional variants or combined/chimeric forms thereof, to utilize a CD3-based agonist. Non-limiting exemplary CD3-based agonists, including but not limited to antibodies or engagers, comprise CD3×CD19, CD3×CD20, CD3×CD33, CD3×EpCAM, CD3×B7H3, blinatumomab, catumaxomab, ertumaxomab, R06958688, AFM11, MT110/AMG 110, MT111/AMG211/MEDI-565, AMG330, MT112/BAY2010112, MOR209/ES414, MGD006/S80880, MGD007, and/or FBTA05. In some embodiments, the ectodomain of the CFR comprises a full or partial length of the extracellular portion of NKG2C, or any functional variants thereof, to utilize an NKG2C-based agonist. Non-limiting exemplary NKG2C-based agonists, including but not limited to antibodies or engagers, comprise NKG2C-IL15-CD33, NKG2C-IL15-CD19, and/or NKG2C-IL15-CD20. In some other embodiments, the ectodomain of the CFR comprises a full or partial length of the extracellular portion of CD28 or any functional variants thereof, to utilize a CD28-based agonist. Non-limiting exemplary CD28-based agonists, including but not limited to antibodies or engagers, comprise at least one of 15E8, CD28.2, CD28.6, YTH913.12, 37.51, 9D7 (TGN1412), 5.11A1, ANC28.1/5D10, and/or 37407.

In some embodiments, the ectodomain of the CFR comprises a full or partial length of the extracellular portion of CD16, CD64, or any functional variants or combined/chimeric forms thereof, to utilize a CD16- or CD64-based agonist. Non-limiting exemplary CD16- or CD64-based agonists, including but not limited to antibodies or engagers, comprise IgG antibodies, or CD16- or CD64-based engagers. When the Fc portion of an IgG antibody binds the CD16- or CD64-based CFRs, it activates antibody dependent cell mediated cytotoxicity (ADCC) in the CFR-expressing cells along with other enhanced therapeutic properties that are imparted by the signaling domains comprised in the endodomains of the CFR. Non-limiting exemplary CD16- or CD64-based agonists, including but not limited to antibodies or engagers, comprise at least one of CD16×CD30, CD64×CD30, CD16×BCMA, CD64×BCMA, CD16-IL-B7H3, CD64-IL-B7H3, CD16-IL-EpCAM or CD64-IL-EpCAM, CD16-IL-CD33 or CD64-IL-CD33, wherein “IL” comprised in a TriKE comprises all or a portion of at least one cytokine comprising IL2, IL4, IL6, IL7, IL9, IL10, IL11, IL12, IL15, IL18, IL21, or any functional variants or combined/chimeric forms thereof.

In general, a transmembrane domain is a three-dimensional protein structure which is thermodynamically stable in a membrane such as the phospholipid bilayer of a biological membrane (e.g., a membrane of a cell or cell vesicle). Thus, in some embodiments, the transmembrane domain of a CFR of the present invention comprises a single alpha helix, a stable complex of several transmembrane alpha helices, a transmembrane beta barrel, a beta-helix of gramicidin A, or any combination thereof. In various embodiments, the transmembrane domain of the CFR comprises all or a portion of a “transmembrane protein” or “membrane protein” that is within the membrane. As used herein, a “transmembrane protein” or “membrane protein” is a protein located at and/or within a membrane. Examples of transmembrane proteins that are suitable for providing a transmembrane domain comprised in a CFR of the invention include, but are not limited to, a receptor, a ligand, an immunoglobulin, a glycophorin, or a combination thereof. In some embodiments, the transmembrane domain comprised in the CFR comprises all or a portion of a transmembrane domain of 2B4, 4-1BB, BTLA, CD2, CD3δ, CD3ε, CD3γ, CD3ζ, CD4, CD8, CD8a, CD8b, CD16, CD27, CD28, CD28H, CD40, CD84, CD166, CS1, CTLA-4, DNAM1, DAP10, DAP12, FcεRIγ, ICOS, ICAM-1, L7, IL12, IL15, KIR2DL4, KIR2DS1, KIR2DS2, LAG3, PD1, NKp30, NK p44, NKp46, NKG2¹C, NKG2D, OX40, T cell receptor polypeptide (such as TCRα and/or TCRβ), a nicotinic acetylcholine receptor, a GABA receptor, or any combination thereof.

In some embodiments, the transmembrane domain comprises all or a portion of a transmembrane domain of IgG, IgA, IgM, IgE, IgD, or any combination thereof. In some embodiments, the transmembrane domain comprises all or a portion of a transmembrane domain of glycophorin A, glycophorin D, or any combination thereof. In particular embodiments of the CFR transmembrane domain, both ER retention and endocytosis signals are absent or are removed using genetic engineering. In various embodiments, both ER retention and endocytosis signals are absent or are removed or eliminated from the CFR transmembrane domain using genetic engineering methods. In some embodiments, the transmembrane domain comprises all or a portion of a transmembrane domain of CD28, CD8, or CD4.

In some embodiments, the endodomain of a CFR comprises at least one signaling domain that activates an intracellular signaling pathway of choice. In various embodiments of the CFR endodomain, both ER retention and endocytosis signals are absent or are removed or eliminated therefrom using genetic engineering methods. In some embodiments, the endodomain comprises at least a cytoxicity domain. In some other embodiments, the endodomain may optionally comprise, in addition to a cytoxicity domain, one or more of a co-stimulatory domain, a persistency signaling domain, a death-inducing signaling domain, a tumor cell control signaling domain, or any combinations thereof. In some embodiments, the signaling peptide of the endodomain (or intracellular domain) comprises a full length or at least a portion of a polypeptide of 2B4, CD2, CD3ζ, CD3ζ1XX, CD8, CD28, CD28H, CD137 (4-1B), CS1, DAP10, DAP12, DNAM1, FcεRIγ, IL2Rγ, IL7R, IL21R, IL2Rβ (IL15Rβ), IL21, IL7, IL12, IL15, IL21, KIR2DS2, NKp30, NKp44, NKp46, NKG2C, or NKG2D. In some embodiments, the cytoxicity domain of the CFR comprises at least a full length or a portion of a polypeptide of CD3ζ, 2B4, DAP10, DAP12, DNAM1, CD137 (4-1B), IL21, IL7, IL12, IL15, NKp30, NKp44, NKp46, NKG2C, or NKG2D. In one embodiment, the cytoxicity domain of a CFR comprises an amino acid sequence that has at least about 85%, about 90%, about 95%, about 96%, about 97%, about 98%, or about 99% identity to at least one ITAM (immunoreceptor tyrosine-based activation motif) of CD3ζ. In one embodiment, the cytoxicity domain of the CFR comprises a modified CD3ζ.

In some embodiments, the CFR comprises an endodomain that comprises a co-stimulatory domain in addition to a cytotoxicity signaling domain. Co-stimulatory domains suitable for use in the CFR include, but are not limited to, a full length or at least a portion of a polypeptide of CD2, CD27, CD28, CD40L, 4-1BB, OX40, ICOS, PD-1, LAG-3, 2B4, BTLA, DAP10, DAP12, CTLA-4, or NKG2D, or any combination thereof. In some embodiments, the co-stimulatory domain of the CFR comprises a full length or at least a portion of a polypeptide of CD28, 4-1B, CD27, CD40L, ICOS, CD2, or combinations thereof. In some embodiments, the CFR comprises an endodomain comprising a co-stimulatory domain of CD28 and a cytoxicity domain of CD3ζ (also referred to as “28ζ”). In some embodiments, the -CD28-CD3ζ portion of an endodomain of the CFR is represented by an amino acid sequence having at least about 85%, about 90%, about 95%, about 96%, about 97%, about 98%, or about 99% identity to SEQ ID NO: 1.

In some embodiments, the CFR comprises an endodomain that comprises a persistency signaling domain in addition to a cytotoxicity signaling domain and/or a co-stimulatory domain. Persistency signaling domains suitable for use in the CFR include, but are not limited to, all or a part of an endodomain of a cytokine receptor such as, IL7R, IL12R, IL15R, IL18R, IL23R, or combinations thereof. In additional embodiments, the endodomain of the CFR may comprise a full or a partial intracellular portion of a receptor tyrosine kinase (RTK) such as EGFR to provide tumor cell control, or a tumor necrosis factor receptor (TNFR) such as FAS, to provide controlled cell death ability.

In various embodiments, exemplary CFRs comprise at least one extracellular portion of a CD3 subunit-CD3ε, CD3δ, or CD3γ, or CD28, represented, for example, by an amino acid sequence of at least about 90%, about 95%, about 96%, about 97%, about 98%, or about 99% identity to SEQ ID NO: 5; a transmembrane domain of CD28, CD8, or CD4, represented respectively by an amino acid sequence of at least about 90%, about 95%, about 96%, about 97%, about 98%, or about 99% identity to SEQ ID NO: 6, SEQ ID NO: 7, and SEQ ID NO: 8; and an endodomain of CD3ε, CD3γ, CD3δ, or CD28, with ER retention motifs and/or endocytosis motifs in ecto-, transmembrane-, and/or endo-domains eliminated. For example, the introduction of an R183S mutation to the CD3F wildtype endodomain sequence (SEQ ID NO: 9) eliminates an ER retention motif, resulting in a CD3F endodomain variant represented by an amino acid sequence of at least about 90%, about 95%, about 96%, about 97%, about 98%, or about 99% identity to SEQ ID NO: 10. The introduction of L142A and R169A mutations to the CD36 wildtype endodomain sequence (SEQ ID NO: 11) eliminates an endocytosis motif and an ER retension motif from the WT sequence, resulting in a CD36 endodomain variant represented by an amino acid sequence of at least about 90%, about 95%, about 96%, about 97%, about 98%, or about 99% identity to SEQ ID NO: 12. Further, the introduction of L131A and R158A mutations to the CD37 wildtype endodomain sequence (SEQ ID NO: 13) eliminates ER retension motifs from the WT sequence, resulting in a CD37 endodomain variant represented by an amino acid sequence of at least about 90%, about 95%, about 96%, about 97%, about 98%, or about 99% identity to SEQ ID NO: 14. The CD28 wildtype endodomain does not have either ER retension or endocytosis motifs, and is represented by an amino acid sequence of at least about 90%, about 95%, about 96%, about 97%, about 98%, or about 99% identity to SEQ ID NO: 15. In various embodiments, a CFR as provided herein further comprises a signal peptide at the N-terminal of the CFR ectodomain. Non-limiting exemplary signal peptides include those represented by an amino acid sequence of at least about 90%, about 95%, about 96%, about 97%, about 98%, or about 99% identity to SEQ ID NO: 16.

SEQ ID NO: 5
NKILVKQSPMLVAYDNAVNLSCKYSYNLESREFRA
SLHKGLDSAVEVCVVYGNYSQQLQVYSKTGENCDG
KLGNESVTFYLQNLYVNQTDIYFCKIEVMYPPPYL
DNEKSNGTIIHVKGKHLCPSPLFPGPSKP
(ecto-CD28)
SEQ ID NO: 6
FWVLVVVGGVLACYSLLVTVAFIIFWV
(TM-CD28)
SEQ ID NO: 7
IYIWAPLAGTCGVLLLSLVIT
(TM-CD8)
SEQ ID NO: 8
MALIVLGGVAGLLLFIGLGIFF
(TM-CD4)
SEQ ID NO: 9
KNRKAKAKPVTRGAGAGGRQRGQNKERPPPVPNPDYE
PIRKGQRDLYSGLNQRRI
(endo-CD3ϵ WT)
SEQ ID NO: 10
KNRKAKAKPVTRGAGAGGRQRGQNKERPPPVPNPDYE
PIRKGQRDLYSGLNQSRI
(endo-CD3_{ϵmut})
SEQ ID NO: 11
GHETGRLSGAADTQALLRNDQVYQPLRDRDDAQYSHLGGNWARNK
(endo-CD3δ WT)
SEQ ID NO: 12
GHETGRLSGAADTQAALRNDQVYQPLRDRDDAQYSHLGGNWAANK
(endo-CD3_{δmut})
SEQ ID NO: 13
GQDGVRQSRASDKQTLLPNDQLYQPLKDREDDQYSHLQGNQLRRN
(endo-CD3γ WT)
SEQ ID NO: 14
GQDGVRQSRASDKQTALPNDQLYQPLKDREDDQYSHLQGNQLARN
(endo-CD3_{γmut})
SEQ ID NO: 15
RSKRSRLLHSDYMNMTPRRPGPTRKHYQPYAPPRDFAAYRS
(endo-CD28)
SEQ ID NO: 16
MLRLLLALNLEPSIQVT

In some exemplary embodiments, the CFR comprises an ectodomain of one CD3 subunit, in some other embodiments the CFR comprises a single chain heterodimeric ectodomain that comprises the ectodomain of CD3F linked with that of CD36 or CD37 (SEQ ID NO: 17 or SEQ ID NO: 18, respectively). The linker type, length, and sequence in the single chain heterodimeric ectodomain may vary.

SEQ ID NO: 17
DGNEEMGGITQTPYKVSISGTTVILTCPQYPGSEILWQHNDKNIGGDE
DDKNIGSDEDHLSLKEFSELEQSGYYVCYPRGSKPEDANFYLYLRARV
CENCMEMDGSADDAKKDAAKKDDAKKDDAKKDGSEKIPIEELEDRVFV
NCNTSITWVEGTVGTLLSDITRLDLGKRILDPRGIYRCNGTDIYKDKE
STVQVHYRMCQSCVELDPATVA
(3ϵ-linker-3δ; linker sequence and
length may vary)
SEQ ID NO: 18
DGNEEMGGITQTPYKVSISGTTVILTCPQYPGSEILWQHNDKNIGGDE
DDKNIGSDEDHLSLKEFSELEQSGYYVCYPRGSKPEDANFYLYLRARV
CENCMEMDGSADDAKKDAAKKDDAKKDDAKKDGSQSIKGNHLVKVYDY
QEDGSVLLTCDAEAKNITWFKDGKMIGFLTEDKKKWNLGSNAKDPRGM
YQCKGSQNKSKPLQVYYRMCQNCIELNAATIS
(3ϵ-linker-3γ; linker sequence and
length may vary)

The cell surface expressed CFR (including CD3-based CFR, also called a cs-CD3 in some contexts) in various constructions as described herein can function as a cell surface triggering receptor for binding with molecules having selected binding specificity, which molecules include antibodies, engagers, and/or CARs. The cell surface expressed CFR of an effector cell may also function with an engager expressed by said effector cell. The cells comprising polynucleotides encoding a CAR, optionally an engager, and/or one or more CFRs of the present invention may be any type of cells, including human cells and non-human cells, pluripotent cells or non-pluripotent cells, immune cells or immune regulatory cells, APC (antigen presenting cells) or feeder cells, cells from primary sources (e.g., PMBC), or from cultured or engineered cells (e.g., cell lines, cells, and/or derivative cells differentiated from iPSCs). In some embodiments, the cells comprising polynucleotides encoding a CAR, optionally an engager, and/or one or more CFRs comprise primary or derivative CD34⁺ cells, hematopoietic stem and progenitor cells, hematopoietic multipotent progenitor cells, T cell progenitors, NK cell progenitors, T lineage cells, NKT lineage cells, NK lineage cells, or B lineage cells. In some embodiments, the derivative cells comprising polynucleotides encoding a CAR, optionally an engager, and/or one or more CFRs are effector cells obtained from differentiating an iPSC comprising polynucleotides encoding the CAR, optionally an engager, and/or the one or more CFRs. In some embodiments, the derivative effector cells comprising polynucleotides encoding a CAR, optionally an engager, and/or one or more CFRs are obtained from engineering the derivative effector cells to incorporate a CAR, optionally an engager, and/or the one or more CFRs after generating the derivative effector cells from an iPSC. In iPSCs and derivative cells therefrom comprising a CAR, optionally an engager, TCR^{neg}, and/or one or more CFRs, the one or more CFRs may be may be expressed with the CAR and optionally with the engager in separate constructs, or may be co-expressed in a bi-cistronic construct comprising the one or more CFRs, the CAR and optionally the engager.

As provided further, the cell or population thereof, comprising polynucleotides encoding a CAR, optionally an engager, TCR^{neg}, and/or one or more CFRs may further comprise one or more additional engineered modalities described herein, and/or as shown in Table 1. Further provided in this application is a master cell bank comprising single cell sorted and expanded clonal engineered iPSCs having at least one phenotype as provided herein, wherein the cell bank provides a platform for additional iPSC engineering and a renewable source for manufacturing off-the-shelf, engineered, homogeneous effector cells, which are well-defined and uniform in composition, and can be mass produced at significant scale in a cost-effective manner.

4. CD16 Knock-In

CD16 has been identified as two isoforms, Fc receptors FcγRIIIa (CD16a; NM_000569.6) and FcγRIIIb (CD16b; NM_000570.4). CD16a is a transmembrane protein expressed by NK cells, which binds monomeric IgG attached to target cells to activate NK cells and facilitate antibody-dependent cell-mediated cytotoxicity (ADCC). CD16b is exclusively expressed by human neutrophils. “High affinity CD16,” “non-cleavable CD16,” or “high affinity non-cleavable CD16” (abbreviated as hnCD16), as used herein, refers to various CD16 variants. The wildtype CD16 has low affinity and is subject to down regulation including ectodomain shedding, a proteolytic cleavage process that regulates the cells surface density of various cell surface molecules on leukocytes upon NK cell activation. F176V (also called F158V in some publications) is an exemplary CD16 polymorphic allele/variant having high affinity; whereas S197P variant is an example of genetically engineered non-cleavable version of CD16. An engineered CD16 variant comprising both F176V and S197P has high affinity and is non-cleavable, which was described in greater detail in WO2015/148926, the complete disclosure of which is incorporated herein by reference. In addition, a chimeric CD16 receptor with the ectodomain of CD16 essentially replaced with at least a portion of CD64 ectodomain can also achieve the desired high affinity and non-cleavable features of a CD16 receptor capable of carrying out ADCC. In some embodiments, the replacement ectodomain of a chimeric CD16 comprises one or more of EC1, EC2, and EC3 exons of CD64 (UniPRotKB_P12314 or its isoform or polymorphic variant).

As such, various embodiments of an exogenous CD16 introduced to a cell include functional CD16 variants and chimeric receptors thereof. In some embodiments, the functional CD16 variant is a high-affinity non-cleavable CD16 receptor (hnCD16). An hnCD16, in some embodiments, comprises both F176V and S197P; and in some embodiments, comprises F176V and with the cleavage region eliminated. In some other embodiments, an hnCD16 comprises a sequence having an identity of at least 50%, 55%, 60%, 65%, 70%, 75%, 80%, 85%, 90%, 95%, 99%, 100%, or any percentage in-between, when compared to any of the exemplary sequences, SEQ ID NOs: 19, 20 and 21, each comprising at least a portion of the CD64 ectodomain. In some embodiments, the sequence identity is at least 80%. In some embodiments, the sequence identity is at least 90%. In some embodiments, the sequence identity is at least 95%. In some embodiments, the sequence identity is 100%. SEQ ID NOs: 19, 20 and 21 are encoded respectively by, for example, SEQ ID NOs: 22-24. As used herein and throughout the application, the percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm recognized in the art.

SEQ ID NO: 19:
MWFLTTLLLWVPVDGQVDTTKAVITLQPPWVSVFQEETVTLHCEVLHLPGSSSTQWELNGTATQTSTPSYR
ITSASVNDSGEYRCQRGLSGRSDPIQLEIHRGWLLLQVSSRVFTEGEPLALRCHAWKDKLVYNVLYYRNGK
AFKFFHWNSNLTILKTNISHNGTYHCSGMGKHRYTSAGISVTVKELFPAPVLNASVTSPLLEGNLVTLSCE
TKLLLQRPGLQLYFSFYMGSKTLRGRNTSSEYQILTARREDSGLYWCEAATEDGNVLKRSPELELQVLGLQ
LPTPVWFHYQVSFCLVMVLLFAVDTGLYFSVKTNIRSSTRDWKDHKEKWRKDPQDK
(340 a.a. CD64 domain-based construction; CD16TM; CD16ICD)
SEQ ID NO: 20
MWFLTTLLLWVPVDGQVDTTKAVITLQPPWVSVFQEETVTLHCEVLHLPGSSSTQWELNGTATQTSTPSYR
ITSASVNDSGEYRCQRGLSGRSDPIQLEIHRGWLLLQVSSRVFTEGEPLALRCHAWKDKLVYNVLYYRNGK
AFKFFHWNSNLTILKTNISHNGTYHCSGMGKHRYTSAGISVTVKELFPAPVLNASVTSPLLEGNLVTLSCE
TKLLLQRPGLQLYFSFYMGSKTLRGRNTSSEYQILTARREDSGLYWCEAATEDGNVLKRSPELELQVLGLF
FPPGYQVSFCLVMVLLFAVDTGLYFSVKTNIRSSTRDWKDHKFKWRKDPQDK
(336 a.a. CD64 exon-based construction; CD16TM;
CD16ICD)
SEQ ID NO: 21
MWFLTTLLLWVPVDGQVDTTKAVITLQPPWVSVFQEETVTLHCEVLHLPGSSSTQWELNGTATQTSTPSYR
ITSASVNDSGEYRCQRGLSGRSDPIQLEIHRGWLLLQVSSRVFTEGEPLALRCHAWKDKLVYNVLYYRNGK
AFKFFHWNSNLTILKTNISHNGTYHCSGMGKHRYTSAGISVTVKELFPAPVLNASVTSPLLEGNLVTLSCE
TKLLLQRPGLQLYFSFYMGSKTLRGRNTSSEYQILTARREDSGLYWCEAATEDGNVLKRSPELELQVLGFF
PPGYQVSFCLVMVLLFAVDTGLYFSVKTNIRSSTRDWKDHKFKWRKDPQDK
(335 a.a. CD64 exon-based construction; CD16TM;
CD16ICD)
SEQ ID NO: 22
cttggagaca acatgtggtt cttgacaact ctgctccttt gggttccagt tgatgggcaa
gtggacacca caaaggcagt gatcactttg cagcctccat gggtcagcgt gttccaagag
gaaaccgtaa ccttgcattg tgaggtgctc catctgcctg ggagcagctc tacacagtgg
tttctcaatg gcacagccac tcagacctcg acccccagct acagaatcac ctctgccagt
gtcaatgaca gtggtgaata caggtgccag agaggtctct cagggcgaag tgaccccata
cagctggaaa tccacagagg ctggctacta ctgcaggtct ccagcagagt cttcacggaa
ggagaacctc tggccttgag gtgtcatgcg tggaaggata agctggtgta caatgtgctt
tactatcgaa atggcaaagc ctttaagttt ttccactgga attctaacct caccattctg
aaaaccaaca taagtcacaa tggcacctac cattgctcag gcatgggaaa gcatcgctac
acatcagcag gaatatctgt cactgtgaaa gagctatttc cagctccagt gctgaatgca
tctgtgacat ccccactcct ggaggggaat ctggtcaccc tgagctgtga aacaaagttg
ctcttgcaga ggcctggttt gcagctttac ttctccttct acatgggcag caagaccctg
cgaggcagga acacatcctc tgaataccaa atactaactg ctagaagaga agactctggg
ttatactggt gcgaggctgc cacagaggat ggaaatgtcc ttaagcgcag ccctgagttg
gagcttcaag tgcttggcct ccagttacca actcctgtct ggtttcatta ccaagtctct
ttctgcttgg tgatggtact cctttttgca gtggacacag gactatattt ctctgtgaag
acaaacattc gaagctcaac aagagactgg aaggaccata aatttaaatg gagaaaggac
cctcaagaca aa
SEQ ID NO: 23
cttggagaca acatgtggtt cttgacaact ctgctccttt gggttccagt tgatgggcaa
gtggacacca caaaggcagt gatcactttg cagcctccat gggtcagcgt gttccaagag
gaaaccgtaa ccttgcattg tgaggtgctc catctgcctg ggagcagctc tacacagtgg
tttctcaatg gcacagccac tcagacctcg acccccagct acagaatcac ctctgccagt
gtcaatgaca gtggtgaata caggtgccag agaggtctct cagggcgaag tgaccccata
cagctggaaa tccacagagg ctggctacta ctgcaggtct ccagcagagt cttcacggaa
ggagaacctc tggccttgag gtgtcatgcg tggaaggata agctggtgta caatgtgctt
tactatcgaa atggcaaagc ctttaagttt ttccactgga attctaacct caccattctg
aaaaccaaca taagtcacaa tggcacctac cattgctcag gcatgggaaa gcatcgctac
acatcagcag gaatatctgt cactgtgaaa gagctatttc cagctccagt gctgaatgca
tctgtgacat ccccactcct ggaggggaat ctggtcaccc tgagctgtga aacaaagttg
ctcttgcaga ggcctggttt gcagctttac ttctccttct acatgggcag caagaccctg
cgaggcagga acacatcctc tgaataccaa atactaactg ctagaagaga agactctggg
ttatactggt gcgaggctgc cacagaggat ggaaatgtcc ttaagcgcag ccctgagttg
gagcttcaag tgcttggttt gttctttcca cctgggtacc aagtctcttt ctgcttggtg
atggtactcc tttttgcagt ggacacagga ctatatttct ctgtgaagac aaacattcga
agctcaacaa gagactggaa ggaccataaa tttaaatgga gaaaggaccc tcaagacaaa
SEQ ID NO: 24
atgtggttct tgacaactct gctcctttgg gttccagttg atgggcaagt ggacaccaca
aaggcagtga tcactttgca gcctccatgg gtcagcgtgt tccaagagga aaccgtaacc
ttgcactgtg aggtgctcca tctgcctggg agcagctcta cacagtggtt tctcaatggc
acagccactc agacctcgac ccccagctac agaatcacct ctgccagtgt caatgacagt
ggtgaataca ggtgccagag aggtctctca gggcgaagtg accccataca gctggaaatc
cacagaggct ggctactact gcaggtctcc agcagagtct tcacggaagg agaacctctg
gccttgaggt gtcatgcgtg gaaggataag ctggtgtaca atgtgcttta ctatcgaaat
ggcaaagcct ttaagttttt ccactggaac tctaacctca ccattctgaa aaccaacata
agtcacaatg gcacctacca ttgctcaggc atgggaaagc atcgctacac atcagcagga
atatctgtca ctgtgaaaga gctatttcca gctccagtgc tgaatgcatc tgtgacatcc
ccactcctgg aggggaatct ggtcaccctg agctgtgaaa caaagttgct cttgcagagg
cctggtttgc agctttactt ctccttctac atgggcagca agaccctgcg aggcaggaac
acatcctctg aataccaaat actaactgct agaagagaag actctgggtt atactggtgc
gaggctgcca cagaggatgg aaatgtcctt aagcgcagcc ctgagttgga gcttcaagtg
cttggcttct ttccacctgg gtaccaagtc tctttctgct tggtgatggt actccttttt
gcagtggaca caggactata ttttctgtg aagacaaaca ttcgaagctc aacaagagac
tggaaggacc ataaatttaa atggagaaag gaccctcaag acaaa

Accordingly, provided herein are effector cells or iPSCs genetically engineered to comprise, among other editing as contemplated and described herein, an exogenous CD16 or a variant thereof, wherein the effector cells are cells from primary sources or derived from iPSC differentiation, or wherein the genetically engineered iPSCs are capable of differentiating into derived effector cells comprising the exogenous CD16 or variant thereof introduced to the iPSCs. In some embodiments, the exogenous CD16 is a high-affinity non-cleavable CD16 receptor (hnCD16). In some embodiments the exogenous CD16 comprises at least a portion of the CD64 ectodomain. In some embodiments the exogenous CD16 is in a form of CD16-based chimeric Fc receptor (CFcR) that comprises a transmembrane domain, a stimulatory domain and/or a signaling domain that is not derived from CD16.

In some embodiments, the primary-sourced or derived effector cells comprising the exogenous CD16 or a variant thereof are NK lineage cells. In some embodiments, the primary-sourced or derived effector cells comprising the exogenous CD16 or a variant thereof are T lineage cells. In some embodiments, the exogenous CD16 comprises hnCD16. In some embodiments, the hnCD16 comprises a full or a partial length extracellular domain of CD64. The exogenous CD16 or functional variants thereof comprised in iPSC or effector cells has high affinity in binding to a ligand that triggers downstreaming signaling upon the binding. The ligand binding to the exogenous CD16 or functional variants include not only ADCC antibodies or fragments thereof, but also bi-, tri-, or multi-specific engagers or binders that recognize the CD16 or CD64 extracellular binding domains of said exogenous CD16. As such, at least one of the aspects of the present application provides a derivative effector cell or a cell population thereof preloaded with one or more pre-selected ADCC antibodies through an exogenous CD16 expressed on the effector cell, in an amount sufficient for therapeutic use in a treatment of a condition, a disease, or an infection as further detailed in this application, wherein said exogenous CD16 comprises an extracellular binding domain of CD64, or of a CD16 having F176V and S197P.

In some other embodiments, an exogenous CD16 comprises a CD16-, or variants thereof, based CFcR. A chimeric Fc receptor (CFcR) is produced to comprise a non-native transmembrane domain, a non-native stimulatory domain and/or a non-native signaling domain by modifying or replacing the native CD16 transmembrane- and/or the intracellular-domain. The term “non-native” used herein means that the transmembrane, stimulatory or signaling domain are derived from a different receptor other than the receptor which provides the extracellular domain. In some embodiments, the CFcR based on CD16 or variants thereof does not have a transmembrane, stimulatory or signaling domain that is derived from CD16. In some embodiments, the exogenous CD16-based CFcR comprises a non-native transmembrane domain derived from CD3δ, CD3ε, CD3γ, CD3ζ, CD4, CD8, CD8a, CD8b, CD27, CD28, CD40, CD84, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or T cell receptor polypeptide. In some embodiments, the exogenous CD16-based CFcR comprises a non-native stimulatory/inhibitory domain derived from CD27, CD28, 4-1BB, OX40, ICOS, PD-1, LAG-3, 2B4, BTLA, DAP10, DAP12, CTLA-4, or NKG2D polypeptide. In some embodiments, the exogenous CD16-based CFcR comprises a non-native signaling domain derived from CD3ζ, 2B4, DAP10, DAP12, DNAM1, CD137 (4-1BB), IL21, IL7, IL12, IL15, NKp30, NKp44, NKp46, NKG2C, or NKG2D polypeptide. In one embodiment of the CD16-based CFcR, the provided chimeric Fc receptor comprises a transmembrane domain and a signaling domain both derived from one of 1L7, IL12, IL15, NKp30, NKp44, NKp46, NKG2C, or NKG2D polypeptide. One particular exemplary embodiment of the CD16-based chimeric Fc receptor comprises a transmembrane domain of NKG2D, a stimulatory domain of 2B4, and a signaling domain of CD3ζ; wherein the extracellular domain of the CFcR is derived from a full length or partial sequence of the extracellular domain of CD64 or CD16, and wherein the extracellular domain of CD16 comprises F176V and S197P. Another exemplary embodiment of the CD16-based chimeric Fc receptor comprises a transmembrane domain and a signaling domain of CD3ζ; wherein the extracellular domain of the CFcR is derived from a full length or partial sequence of the extracellular domain of CD64 or CD16, and wherein the extracellular domain of CD16 comprises F176V and S197P.

The various embodiments of CD16-based chimeric Fc receptor as described above are capable of binding, with high affinity, to the Fc region of an antibody or fragment thereof, or to a bi-, tri-, or multi-specific engager or binder. Upon binding, the stimulatory and/or signaling domains of the chimeric receptor enable the activation and cytokine secretion of the effector cells, and the killing of the tumor cells targeted by the antibody, or said bi-, tri-, or multi-specific engager or binder having a tumor antigen binding component as well as the Fc region. Without being limited by theory, through the non-native transmembrane, stimulatory and/or signaling domains, or through an engager binding to the ectodomain, of the CD16-based chimeric Fc receptor, the CFcR could contribute to effector cells' killing ability while increasing the effector cells' proliferation and/or expansion potential. The antibody and the engager can bring tumor cells expressing the antigen and the effector cells expressing the CFcR into a close proximity, which also contributes to the enhanced killing of the tumor cells. Exemplary tumor antigens for bi-, tri-, multi-specific engagers or binders include, but are not limited to, B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, and ROR1. Some non-limiting exemplary bi-, tri-, multi-specific engagers or binders suitable for engaging effector cells expressing the CD16-based CFcR in attacking tumor cells include CD16 (or CD64)-CD30, CD16 (or CD64)-BCMA, CD16 (or CD64)-IL15-EpCAM, and CD16 (or CD64)-IL15-CD33.

Unlike the endogenous CD16 expressed by primary NK cells which gets cleaved from the cellular surface following NK cell activation, the various non-cleavable versions of CD16 in derivative NK cells avoid CD16 shedding and maintain constant expression. In derivative NK cells, non-cleavable CD16 increases expression of TNFα and CD107a, indicative of improved cell functionality. Non-cleavable CD16 also enhances the antibody-dependent cell-mediated cytotoxicity (ADCC), and the engagement of bi-, tri-, or multi-specific engagers. ADCC is a mechanism of NK cell mediated lysis through the binding of CD16 to antibody-coated target cells. The additional high affinity characteristics of the introduced hnCD16 in a derived NK cell also enables in vitro loading of an ADCC antibody to the NK cell through hnCD16 before administering the cell to a subject in need of a cell therapy. As provided herein, the hnCD16 may comprise F176V and S197P in some embodiments, or may comprise a full or partial length ectodomain originated from CD64, as exemplified by SEQ ID NOs: 19, 20 or 21, or may further comprise at least one of non-native transmembrane domain, stimulatory domain and signaling domain. As disclosed, the present application also provides a derivative NK cell or a cell population thereof, preloaded with one or more pre-selected ADCC antibodies in an amount sufficient for therapeutic use in a treatment of a condition, a disease, or an infection as further detailed in this application.

Unlike primary NK cells, mature T cells from a primary source (i.e., natural/native sources such as peripheral blood, umbilical cord blood, or other donor tissues) do not express CD16. It was unexpected that iPSC comprising an expressed exogenous non-cleavable CD16 did not impair the T cell developmental biology and was able to differentiate into functional derivative T lineage cells that not only express the exogenous CD16, but also are capable of carrying out function through an acquired ADCC mechanism. This acquired ADCC in the derivative T lineage cell can additionally be used as an approach for dual targeting and/or to rescue antigen escape often occurred with CAR-T cell therapy, where the tumor relapses with reduced or lost CAR-T targeted antigen expression or expression of a mutated antigen to avoid recognition by the CAR. When said derivative T lineage cell comprises acquired ADCC through exogenous CD16, including functional variants and CD16-based CFcR, expression, and when an antibody or an engager targets a different tumor antigen from the one targeted by the CAR, the antibody or an engager can be used to rescue CAR-T antigen escape and reduce or prevent relapse or recurrence of the targeted tumor often seen in CAR-T treatment. Such a strategy to reduce and/or prevent antigen escape while achieving dual targeting is equally applicable to NK cells expressing one or more CARs.

As such, embodiments of the present invention provide a derivative T lineage cell comprising an exogenous CD16 or variant thereof in addition to a CAR, and optionally an engager, as provided herein. In some embodiments, the CD16 comprised in the derivative T lineage cell is an hnCD16 that comprises the CD16 ectodomain comprising F176V and S197P. In some other embodiments, the hnCD16 comprised in the derivative T lineage cell comprises a full or partial length ectodomain originated from CD64, as exemplified by SEQ ID NOs: 19, 20 or 21; or may further comprise at least one of non-native transmembrane domain, stimulatory domain and signaling domain. As explained herein, such derivative T lineage cells have an acquired mechanism to target tumors with a monoclonal antibody meditated by ADCC to enhance the therapeutic effect of the antibody. As disclosed, the present application also provides a derivative T lineage cell or a population thereof, preloaded with one or more pre-selected ADCC antibodies in an amount sufficient for therapeutic use in a treatment of a condition, a disease, or an infection as further detailed below.

As provided further, the cell or population thereof, comprising polynucleotides encoding a CAR, optionally an engager, TCR^{neg}, and/or one or more CFRs, and an exogenous CD16 or a variant thereof, may further comprise one or more additional engineered modalities described herein, and/or as shown in Table 1. Additionally provided in this application is a master cell bank comprising single cell sorted and expanded clonal engineered iPSCs having at least one phenotype as provided herein, wherein the cell bank provides a platform for additional iPSC engineering and a renewable source for manufacturing off-the-shelf, engineered, homogeneous cell therapy products, including but not limited to derivative NK and T cells, which are well-defined and uniform in composition, and can be mass produced at significant scale in a cost-effective manner.

5. CD38 Knockout

The cell surface molecule CD38 is highly upregulated in multiple hematologic malignancies derived from both lymphoid and myeloid lineages, including multiple myeloma and a CD20 negative B-cell malignancy, which makes it an attractive target for antibody therapeutics to deplete cancer cells. Antibody mediated cancer cell depletion is usually attributable to a combination of direct cell apoptosis induction and activation of immune effector mechanisms such as ADCC (antibody-dependent cell-mediated cytotoxicity). In addition to ADCC, the immune effector mechanisms in concert with the therapeutic antibody may also include antibody-dependent cell-mediated phagocytosis (ADCP) and/or complement-dependent cytotoxicity (CDC).

Other than being highly expressed on malignant cells, CD38 is also expressed on plasma cells, as well as on NK cells and activated T and B cells. During hematopoiesis, CD38 is expressed on CD34⁺ stem cells and lineage-committed progenitors of lymphoid, erythroid, and myeloid, and during the final stages of maturation which continues through the plasma cell stage. As a type II transmembrane glycoprotein, CD38 carries out cell functions as both a receptor and a multifunctional enzyme involved in the production of nucleotide-metabolites. As an enzyme, CD38 catalyzes the synthesis and hydrolysis of the reaction from NAD⁺ to ADP-ribose, thereby producing secondary messengers CADPR and NAADP which stimulate release of calcium from the endoplasmic reticulum and lysosomes, critical for the process of cell adhesion, which process is calcium dependent. As a receptor, CD38 recognizes CD31 and regulates cytokine release and cytotoxicity in activated NK cells. CD38 is also reported to associate with cell surface proteins in lipid rafts, to regulate cytoplasmic Ca²⁺ flux, and to mediate signal transduction in lymphoid and myeloid cells.

In malignancy treatment, systemic use of CD38 antigen binding receptor transduced T cells have been shown to lyse the CD38⁺ fractions of CD34⁺ hematopoietic progenitor cells, monocytes, NK cells, T cells and B cells, leading to incomplete treatment responses and reduced or eliminated efficacy because of the impaired recipient immune effector cell function. In addition, in multiple myeloma patients treated with daratumumab, a CD38 specific antibody, NK cell reduction in both bone marrow and peripheral blood was observed, although other immune cell types, such as T cells and B cells, were unaffected despite their CD38 expression (Casneuf et al., Blood Advances. 2017; 1(23):2105-2114). Without being limited by theories, the present application provides a strategy to leverage the full potential of CD38 targeted cancer treatment by overcoming CD38 specific antibody and/or CD38 antigen binding domain induced effector cell depletion or reduction through fratricide. In addition, since CD38 is upregulated on activated lymphocytes such as T or B cells, by suppressing activation of these recipient lymphocytes using a CD38 specific antibody such as daratumumab in the recipient of allogeneic effector cells, the allorejection against these effector cells would be reduced and/or prevented, thereby increasing effector cell survival and persistency.

As such, the present application also provides a strategy to enhance effector cell persistency and/or survival through reducing or preventing allorejection by using a CD38 specific antibody, a secreted CD38 specific engager or a CD38-CAR (chimeric antigen receptor) against activation of recipient T and B cells, i.e., lymphodepletion of activated T and B cells, often prior to adoptive cell transferring. Specifically, the strategies as provided include generating an iPSC line comprising a CD38 knockout, a master cell bank comprising single cell sorted and expanded clonal CD38 negative iPSCs, and obtaining CD38 negative (CD38^{neg}) derivative effector cells through directed differentiation of the engineered iPSC line, wherein the derivative effector cells are protected against fratricide and allorejection among other advantages when CD38 targeted therapeutic moieties are employed with the effector cells. In addition, anti-CD38 monoclonal antibody therapy significantly depletes a patient's activated immune system without adversely affecting the patient's hematopoietic stem cell compartment. A CD38 negative derivative cell has the ability to resist CD38 antibody mediated depletion, and may be effectively administered in combination with anti-CD38 antibody or CD38-CAR without the use of toxic conditioning agents and thus reduce and/or replace chemotherapy-based lymphodepletion.

In one embodiment as provided herein, the CD38 knockout in an iPSC line is a bi-allelic knockout. As disclosed herein, the provided CD38 negative iPSC line further comprises at least a CAR, optionally an engager and/or one or more of TCR^{neg}, CFR, exogenous CD16 or a variant thereof, and may further comprise one or more additional engineered modalities described herein, and as shown in Table 1; and said iPSC is capable of directed differentiation to produce functional derivative effector cells, including, but not limited to, mesodermal cells with definitive hemogenic endothelium (HE) potential, definitive HE, CD34⁺ hematopoietic cells, hematopoietic stem and progenitor cells, hematopoietic multipotent progenitors (MPP), T cell progenitors, NK cell progenitors, common myeloid progenitor cells, common lymphoid progenitor cells, erythrocytes, myeloid cells, neutrophil progenitors, T cells, NKT cells, NK cells, B cells, neutrophils, dendritic cells, macrophages, and derivative immune effector cells having one or more functional features not present in primary NK, T and/or NKT cells. In some embodiments, when an anti-CD38 antibody is used to induce ADCC or an anti-CD38 CAR is used for targeted cell killing, the CD38^{neg} iPSC and/or derivative effector cells thereof are not eliminated by the anti-CD38 antibody, the anti-CD38 CAR, or recipient activated T or B cells, thereby increasing the iPSC and its effector cell persistence and/or survival in the presence of, and/or after exposure to, such therapeutic moieties. In some embodiments, the effector cell has increased persistence and/or survival in vivo in the presence of, and/or after exposure to, such therapeutic moieties.

6. HLA-I- and HLA-H-Deficiency

Multiple HLA class I and class II proteins must be matched for histocompatibility in allogeneic recipients to avoid allogeneic rejection problems. Provided herein is an iPSC cell line and its derivative cells differentiated therefrom with eliminated or substantially reduced expression of one or both of HLA class I and HLA class II proteins. HLA class I deficiency can be achieved by functional deletion of any region of the HLA class I locus (chromosome 6p21), or deletion or disruption of HLA class I associated genes including, but not limited to, beta-2 microglobulin (B2M) gene, TAP1 gene, TAP2 gene and Tapasin. For example, the B2M gene encodes a common subunit essential for cell surface expression of all HLA class I heterodimers. B2M negative cells are HLA-I deficient. HLA class II deficiency can be achieved by functional deletion or disruption of HLA class II associated genes including, but not limited to, RFXANK, CIITA, RFX5 and RFXAP. CIITA is a transcriptional coactivator, functioning through activation of the transcription factor RFX5 required for class II protein expression. CIITA negative cells are HLA-II deficient. Provided herein is an iPSC line and its derivative cells having HLA-I deficiency and/or HLA-II deficiency, for example lacking both B2M and CIITA expression, wherein the obtained derivative effector cells enable allogeneic cell therapies by eliminating the need for MHC (major histocompatibility complex) matching, and avoiding recognition and killing by host (allogeneic) T cells.

For some cell types, a lack of HLA class I expression leads to lysis by NK cells. To overcome this “missing self” response, HLA-G may be optionally knocked in to avoid NK cell recognition and killing of the HLA-I deficient effector cells derived from an engineered iPSC. In one embodiment, the HLA-I deficient iPSC and its derivative cells further comprise HLA-G knock-in. In some embodiments, the provided HLA-I deficient iPSC and its derivative cells further comprise one or both of CD58 knockout and CD54 knockout. CD58 (or LFA-3) and CD54 (or ICAM-1) are adhesion proteins initiating signal-dependent cell interactions, and facilitating cell, including immune cell, migration. It was shown that CD58 knockout has a higher efficiency in reducing allogeneic NK cell activation than CD54 knockout; while double knockout of both of CD58 and CD54 has the most enhanced reduction of NK cell activation. In some observations, the CD58 and CD54 double knockout is even more effective than HLA-G overexpression for HLA-I deficient cells in overcoming the “missing-self” effect.

As provided herein, in some embodiments, the HLA-I and HLA-II deficient iPSC and its derivative cells have an exogenous polynucleotide encoding HLA-G. In some embodiments, the HLA-I and HLA-II deficient iPSC and its derivative cells are CD58 negative. In some other embodiments, the HLA-I and HLA-II deficient iPSC and its derivative cells are CD54 negative. In yet some other embodiments, the HLA-I and HLA-II deficient iPSC and its derivative cells are CD58 negative and CD54 negative.

In some embodiments, the engineering for HLA-I and/or HLA-II deficiency may be bypassed, or kept intact, by expressing an inactivation CAR targeting an upregulated surface protein in activated recipient immune cells to avoid allorejection. In some embodiments, the upregulated surface protein in the activated recipient immune cells includes, but is not limited to, CD38, CD25, CD69, CD44, 4-1BB, OX40, or CD40L. When the cell expresses such an inactivation CAR, it is preferable that the cell does not express, or has knockout of, the same surface protein targeted by CAR. In some embodiments, the inactivation CAR comprises at least one of a CD38-CAR, a CD25-CAR, a CD69-CAR, a CD44-CAR, a 4-1BB-CAR, an OX40-CAR, and a CD40L-CAR.

Further, in some embodiments of the iPSC and its derivative cells comprising polynucleotides encoding a CAR, optionally an engager, and/or one or more CFRs, TCR^{neg}, exogenous CD16 or a variant thereof, and CD38 knockout, said cells are HLA-I and HLA-II deficient and have an exogenous polynucleotide encoding HLA-G. In some embodiments of the iPSC and its derivative cells comprising polynucleotides encoding a CAR, optionally an engager, and/or one or more CFRs, TCR^{neg}, exogenous CD16 or a variant thereof, and CD38 knockout, the cells are HLA-I and HLA-II deficient and are CD58 negative. In some embodiments of the iPSC and its derivative cells comprising polynucleotides encoding a CAR, optionally an engager, and/or one or more CFRs, TCR^{neg}, exogenous CD16 or a variant thereof, and CD38 knockout, the cells are HLA-I and HLA-II deficient and are CD54 negative. In yet some other embodiments of the iPSC and its derivative cells comprising polynucleotides encoding a CAR, optionally an engager, and/or one or more CFRs, TCR^{neg}, exogenous CD16 or a variant thereof, and CD38 knockout, the cells are HLA-I and HLA-II deficient, and are both CD58 negative and CD54 negative.

Additionally provided in this application is a master cell bank comprising single cell sorted and expanded clonal engineered iPSCs having at least one phenotype as provided herein, including but not limited to, HLA-I and/or HLA-II deficiency, wherein the cell bank provides a platform for additional iPSC engineering and a renewable source for manufacturing off-the-shelf, engineered, homogeneous cell therapy products, including but not limited to derivative NK and T cells, which are well-defined and uniform in composition, and can be mass produced at significant scale in a cost-effective manner.

6. Exogenously Introduced Cytokine and/or Cytokine Signaling

By avoiding systemic high-dose administration of clinically relevant cytokines, the risk of dose-limiting toxicities due to such a practice is reduced while cytokine mediated cell autonomy is being established. To achieve lymphocyte autonomy without the need to additionally administer soluble cytokines, a signaling complex comprising a partial or full length peptide of one or more of IL2, IL4, IL6, IL7, IL9, IL10, IL11, IL12, IL15, IL18, IL21, and/or their respective receptor may be introduced to the cell to enable cytokine signaling with or without the expression of the cytokine itself to achieve lymphocyte autonomy without administered soluble cytokines thereby maintaining or improving cell growth, proliferation, expansion, persistency and/or effector function with reduced risk of cytokine toxicities. In some embodiments, the introduced cytokine and/or its respective native or modified receptor for cytokine signaling (signaling complex) is expressed on the cell surface. In some embodiments, the cytokine signaling is constitutively activated. In some embodiments, the activation of the cytokine signaling is inducible. In some embodiments, the activation of the cytokine signaling is transient and/or temporal.

Various construct designs for introducing a protein complex for signaling of cytokines including, but not limited to, IL2, IL4, IL6, IL7, IL9, IL10, IL11, IL12, IL15, IL18 and IL21, into the cell are provided herein. In embodiments where the signaling complex is for IL15, the transmembrane (TM) domain can be native to the IL15 receptor or may be modified or replaced with transmembrane domain of any other membrane bound proteins. In some embodiments, IL15 and IL15Rα are co-expressed by using a self-cleaving peptide, mimicking trans-presentation of IL15, without eliminating cis-presentation of IL15. In other embodiments, IL15Rα is fused to IL15 at the C-terminus through a linker, mimicking trans-presentation without eliminating cis-presentation of IL15 as well as ensuring that IL15 is membrane-bound. In other embodiments, IL15Rα with truncated intracellular domain is fused to IL15 at the C-terminus through a linker, mimicking trans-presentation of IL15, maintaining IL15 membrane-bound, and additionally eliminating cis-presentation and/or any other potential signal transduction pathways mediated by a normal IL15R through its intracellular domain.

Such a truncated construct comprises an amino acid sequence of at least 75%, 80%, 85%, 90%, 95% or 99% identity to SEQ ID NO: 25, which may be encoded by an exemplary nucleic acid sequence represented by SEQ ID NO: 26. In one embodiment of the truncated IL15/IL15Rα, the construct does not comprise the last 4 amino acid residues “KSRQ” of SEQ ID NO: 25, and comprises an amino acid sequence of at least 75%, 80%, 85%, 90%, 95% or 99% identity to SEQ ID NO: 27. In some embodiments, the sequence identity is at least 80%. In some embodiments, the sequence identity is at least 90%. In some embodiments, the sequence identity is at least 95%. In some embodiments, the sequence identity is 100%.

SEQ ID NO: 25
MDWTWILFLVAAATRVHSGIHVFILGCFSAGLPKTEANWVNVISDLKKIEDLIQSMHIDATLYTE
SDVHPSCKVTAMKCFLLELQVISLESGDASIHDTVENLIILANNSLSSNGNVTESGCKECEELEE
KNIKEFLQSFVHIVQMFINTSSGGGSGGGGSGGGGSGGGGSGGGSLQITCPPPMSVEHADIWVKS
YSLYSRERYICNSGFKRKAGTSSLTECVLNKATNVAHWTTPSLKCIRDPALVHQRPAPPSTVTTA
GVTPQPESLSPSGKEPAASSPSSNNTAATTAAIVPGSQLMPSKSPSTGTTEISSHESSHGTPSQT
TAKNWELTASASHQPPGVYPQGHSDTTVAISTSTVLLCGLSAVSLLACYLKSRQ
(379 a.a .; signal and linker peptides are underlined)
SEQ ID NO: 26
ATGGACTGGACCTGGATTCTGTTCCTGGTCGCGGCTGCAACGCGAGTCCATAGCGGTATCCATGT
TTTTATTCTTGGGTGTTTTTCTGCTGGGCTGCCTAAGACCGAGGCCAACTGGGTAAATGTCATCA
GTGACCTCAAGAAAATAGAAGACCTTATACAAAGCATGCACATTGATGCTACTCTCTACACTGAG
TCAGATGTACATCCCTCATGCAAAGTGACGGCCATGAAATGTTTCCTCCTCGAACTTCAAGTCAT
ATCTCTGGAAAGTGGCGACGCGTCCATCCACGACACGGTCGAAAACCTGATAATACTCGCTAATA
ATAGTCTCTCTTCAAATGGTAACGTAACCGAGTCAGGTTGCAAAGAGTGCGAAGAGTTGGAAGAA
AAAAACATAAAGGAGTTCCTGCAAAGTTTCGTGCACATTGTGCAGATGTTCATTAATACCTCTAG
CGGCGGAGGATCAGGTGGCGGTGGAAGCGGAGGTGGAGGCTCCGGTGGAGGAGGTAGTGGCGGAG
GTTCTCTTCAAATAACTTGTCCTCCACCGATGTCCGTAGAACATGCGGATATTTGGGTAAAATCC
TATAGCTTGTACAGCCGAGAGCGGTATATCTGCAACAGCGGCTTCAAGCGGAAGGCCGGCACAAG
CAGCCTGACCGAGTGCGTGCTGAACAAGGCCACCAACGTGGCCCACTGGACCACCCCTAGCCTGA
AGTGCATCAGAGATCCCGCCCTGGTGCATCAGCGGCCTGCCCCTCCAAGCACAGTGACAACAGCT
GGCGTGACCCCCCAGCCTGAGAGCCTGAGCCCTTCTGGAAAAGAGCCTGCCGCCAGCAGCCCCAG
CAGCAACAATACTGCCGCCACCACAGCCGCCATCGTGCCTGGATCTCAGCTGATGCCCAGCAAGA
GCCCTAGCACCGGCACCACCGAGATCAGCAGCCACGAGTCTAGCCACGGCACCCCATCTCAGACC
ACCGCCAAGAACTGGGAGCTGACAGCCAGCGCCTCTCACCAGCCTCCAGGCGTGTACCCTCAGGG
CCACAGCGATACCACAGTGGCCATCAGCACCTCCACCGTGCTGCTGTGTGGACTGAGCGCCGTGT
CACTGCTGGCCTGCTACCTGAAGTCCAGACAGTGA
(1140 n.a.)
SEQ ID NO: 27
MDWTWILFLVAAATRVHSGIHVFILGCFSAGLPKTEANWVNVISDLKKIEDLIQSMHIDATLYTE
SDVHPSCKVTAMKCFLLELQVISLESGDASIHDTVENLIILANNSLSSNGNVTESGCKECEELEE
KNIKEFLQSFVHIVQMFINTSSGGGSGGGGSGGGGSGGGGSGGGSLQITCPPPMSVEHADIWVKS
YSLYSRERYICNSGFKRKAGTSSLTECVLNKATNVAHWTTPSLKCIRDPALVHQRPAPPSTVTTA
GVTPQPESLSPSGKEPAASSPSSNNTAATTAAIVPGSQLMPSKSPSTGTTEISSHESSHGTPSQT
TAKNWELTASASHQPPGVYPQGHSDTTVAISTSTVLLCGLSAVSLLACYL
(375 a.a .; signal and linker peptides are underlined)

In yet other embodiments, the cytoplasmic domain of IL15Rα can be omitted without negatively impacting the autonomous feature of the effector cell equipped with IL15. In other embodiments, essentially the entire IL15Rα is removed except for the Sushi domain fused with IL15 at one end and a transmembrane domain on the other (mb-Sushi), optionally with a linker between the Sushi domain and the trans-membrane domain. The fused IL15/mb-Sushi is expressed at the cell surface through the transmembrane domain of any membrane bound protein. Thus, unnecessary signaling through IL15Rα, including cis-presentation, is eliminated when only the desirable trans-presentation of IL15 is retained. In some embodiments, the component comprising IL15 fused with the Sushi domain comprises an amino acid sequence of at least 75%, 80%, 85%, 90%, 95% or 99% identity to SEQ ID NO: 28, which may be encoded by an exemplary nucleic acid sequence represented by SEQ ID NO: 29. In some embodiments, the sequence identity is at least 80%. In some embodiments, the sequence identity is at least 90%. In some embodiments, the sequence identity is at least 95%. In some embodiments, the sequence identity is 100%.

SEQ ID NO: 28
MDWTWILFLVAAATRVHSGIHVFILGCFSAGLPKTEANWVNVISDLKKIEDLIQSMHIDATLYTE
SDVHPSCKVTAMKCFLLELQVISLESGDASIHDTVENLIILANNSLSSNGNVTESGCKECEELEE
KNIKEFLQSFVHIVQMFINTSSGGGSGGGGSGGGGSGGGGSGGGSLQITCPPPMSVEHADIWVKS
YSLYSRERYICNSGFKRKAGTSSLTECVLNKATNVAHWTTPSLKCIR
(242 a.a .; signal and linker peptides are underlined)
SEQ ID NO: 29
ATGGACTGGACCTGGATTCTGTTCCTGGTCGCGGCTGCAACGCGAGTCCATAGCGGTATCCATGT
TTTTATTCTTGGGTGTTTTTCTGCTGGGCTGCCTAAGACCGAGGCCAACTGGGTAAATGTCATCA
GTGACCTCAAGAAAATAGAAGACCTTATACAAAGCATGCACATTGATGCTACTCTCTACACTGAG
TCAGATGTACATCCCTCATGCAAAGTGACGGCCATGAAATGTTTCCTCCTCGAACTTCAAGTCAT
ATCTCTGGAAAGTGGCGACGCGTCCATCCACGACACGGTCGAAAACCTGATAATACTCGCTAATA
ATAGTCTCTCTTCAAATGGTAACGTAACCGAGTCAGGTTGCAAAGAGTGCGAAGAGTTGGAAGAA
AAAAACATAAAGGAGTTCCTGCAAAGTTTCGTGCACATTGTGCAGATGTTCATTAATACCTCTAG
CGGCGGAGGATCAGGTGGCGGTGGAAGCGGAGGTGGAGGCTCCGGTGGAGGAGGTAGTGGCGGAG
GTTCTCTTCAAATAACTTGTCCTCCACCGATGTCCGTAGAACATGCGGATATTTGGGTAAAATCC
TATAGCTTGTACAGCCGAGAGCGGTATATCTGCAACAGCGGCTTCAAGCGGAAGGCCGGCACAAG
CAGCCTGACCGAGTGCGTGCTGAACAAGGCCACCAACGTGGCCCACTGGACCACCCCTAGCCTGA
AGTGCATCAGA
(726 n.a.)

One having ordinary skill in the art would appreciate that the signal peptide and the linker sequences above are illustrative and in no way limit their variations suitable for use as a signal peptide or linker. There are many suitable signal peptide or linker sequences known and available to those in the art. The ordinary skilled person in the art understands that the signal peptide and/or linker sequences may be substituted for another sequence without altering the activity of the functional peptide led by the signal peptide or linked by the linker.

In other embodiments, a native or modified IL15Rβ is fused to IL15 at the C-terminus through a linker, enabling constitutive signaling and maintaining IL15 membrane-bound and trans-representation. In other embodiments, a native or modified common receptor γC is fused to IL15 at the C-terminus through a linker for constitutive signaling and membrane bound trans-presentation of the cytokine. The common receptor γC is also called the common gamma chain or CD132, which is also known as IL2 receptor subunit gamma or IL2RG. γC is a cytokine receptor sub-unit that is common to the receptor complexes for many interleukin receptors, including, but not limited to, IL2, IL4, IL7, IL9, TL15 and IL21 receptor. In other embodiments, engineered IL15Rβ that forms a homodimer in the absence of IL15 is useful for producing constitutive signaling of the cytokine.

In embodiments where the signaling complex is for IL7, the transmembrane (TM) domain of any of a variety of designs can be native to the cytokine receptor or may be modified or replaced with a transmembrane domain of any other membrane bound proteins. In some embodiments, a native (or wildtype) or modified IL7R may be fused to IL7 at the C-terminus through a linker, enabling constitutive signaling and maintaining membrane-bound IL7. In some embodiments, such a construct comprises an amino acid sequence of at least 75%, 80%, 85%, 90%, 95% or 99% identity to SEQ ID NO: 30, with transmembrane domain, signal peptide and linker being flexible and varying in length and/or sequences. In some embodiments, the sequence identity is at least 80%. In some embodiments, the sequence identity is at least 90%. In some embodiments, the sequence identity is at least 95%. In some embodiments, the sequence identity is 100%.

SEQ ID NO: 30
MDWTWILFLVAAATRVHSDCDIEGKDGKQYESVLMVSIDQLLDSMKEIGSNCLNNEFNFFKRHIC
DANKEGMFLFRAARKLRQFLKMNSTGDFDLHLLKVSEGTTILLNCTGQVKGRKPAALGEAQPTKS
LEENKSLKEQKKLNDLCFLKRLLQEIKTCWNKILMGTKEHSGGGSGGGGSGGGGSGGGGSGGGSL
QESGYAQNGDLEDAELDDYSFSCYSQLEVNGSQHSLTCAFEDPDVNITNLEFEICGALVEVKCLN
FRKLQEIYFIETKKELLIGKSNICVKVGEKSLTCKKIDLTTIVKPEAPFDLSVVYREGANDFVVT
FNTSHLQKKYVKVLMHDVAYRQEKDENKWTHVNLSSTKLTLLQRKLQPAAMYEIKVRSIPDHYFK
GFWSEWSPSYYFRTPEINNSSGEMDPILLTISILSFFSVALLVILACVLWKKRIKPIVWPSLPDH
KKTLEHLCKKPRKNLNVSFNPESFLDCQIHRVDDIQARDEVEGFLQDTFPQQLEESEKQRLGGDV
QSPNCPSEDVVITPESFGRDSSLTCLAGNVSACDAPILSSSRSLDCRESGKNGPHVYQDLLLSLG
TTNSTLPPPFSLQSGILTLNPVAQGQPILTSLGSNQEEAYVTMSSFYQNQ
(Signal peptide-IL7-linker-IL7R; transmembrane domain (TM),
signal peptide and linker can vary in length and sequences)

One having ordinary skill in the art would appreciate that the signal peptide and the linker sequences above are illustrative and in no way limit their variations suitable for use as a signal peptide or linker. There are many suitable signal peptide or linker sequences known and available to those in the art. The ordinary skilled in the art understands that the signal peptide and/or linker sequences may be substituted for another sequence without altering the activity of the functional peptide led by the signal peptide or linked by the linker.

In one embodiment, a native or modified common receptor γC is fused to IL7 at the C-terminus through a linker for constitutive and membrane-bound cytokine signaling complex. The common receptor γC is also called the common gamma chain or CD132, which is also known as IL2 receptor subunit gamma or IL2RG. γC is a cytokine receptor sub-unit that is common to the receptor complexes for many interleukin receptors, including, but not limited to, IL2, IL4, IL7, IL9, and IL21 receptor. In another embodiment, engineered IL7R that forms a homodimer in the absence of IL7 is useful for producing constitutive signaling of the cytokine as well.

As such, in various embodiments, the cytokines IL15 or IL7 and/or their receptors, may be introduced to iPSC using one or more of the construct designs described above, and to its derivative cells upon iPSC differentiation. In addition to an induced pluripotent cell (iPSC), a clonal iPSC, a clonal iPS cell line, or iPSC-derived cells comprising at least one engineered modality as disclosed herein are provided. Also provided is a master cell bank comprising single cell sorted and expanded clonal engineered iPSCs having at least a signaling complex comprising a partial or full peptide of a cell surface expressed exogenous cytokine and/or a receptor thereof, as described in this section, wherein the cell bank provides a platform for additional iPSC engineering and a renewable source for manufacturing off-the-shelf, engineered, homogeneous cell therapy products, which are well-defined and uniform in composition, and can be mass produced at a significant scale in a cost-effective manner.

In iPSCs and derivative cells therefrom comprising both CAR and exogenous cytokine and/or cytokine receptor signaling (signaling complex or “IL”), the CAR and IL may be expressed in separate constructs, or may be co-expressed in a bi-cistronic construct comprising both CAR and IL. In some further embodiments, the signaling complex can be linked to either the 5′ or the 3′ end of a CAR expression construct through a self-cleaving 2A coding sequence. As such, an IL signaling complex (e.g., IL7 signaling complex) and CAR may be in a single open reading frame (ORF). In one embodiment, the signaling complex is comprised in CAR-2A-IL or IL-2A-CAR construct. When CAR-2A-IL or IL-2A-CAR is expressed, the self-cleaving 2A peptide allows the expressed CAR and IL to dissociate, and the dissociated IL can then be presented at the cell surface, with the transmembrane domain anchored in the cell membrane. The CAR-2A-IL or IL-2A-CAR bi-cistronic design allows for coordinated CAR and IL signaling complex expression both in timing and quantity, and under the same control mechanism that may be chosen to incorporate, for example, an inducible promoter or promoter with temporal or spatial specificity for the expression of the single ORF. Self-cleaving peptides are found in members of the Picornaviridae virus family, including aphthoviruses such as foot-and-mouth disease virus (FMDV), equine rhinitis A virus (ERAV), Thosea asigna virus (TaV) and porcine tescho virus-1 (PTV-I) (Donnelly, M L, et al, J. Gen. Virol, 82, 1027-101 (2001); Ryan, M D, et al., J. Gen. Virol., 72, 2727-2732 (2001)), and cardioviruses such as Theilovirus (e.g., Theiler's murine encephalomyelitis) and encephalomyocarditis viruses. The 2A peptides derived from FMDV, ERAV, PTV-I, and TaV are sometimes also referred to as “F2A”, “E2A”, “P2A”, and “T2A”, respectively.

The bi-cistronic CAR-2A-IL or IL-2A-CAR embodiment as disclosed herein is also contemplated for expression of any other cytokine or cytokine signaling complex provided herein, for example, IL2, IL4, IL6, IL9, IL10, IL11, IL12, IL18, and IL21. In some embodiments, the bi-cistronic CAR-2A-IL or IL-2A-CAR is for expression of one or more of IL2, IL4, IL7, IL9, IL15 and IL21.

In iPSCs and derivative cells therefrom comprising both CAR, optionally an engager, and a signaling complex comprising a partial or full peptide of a cell surface expressed exogenous cytokine and/or cytokine receptor thereof, the iPSCs and derivative cells may further comprise one or more of CFR, TCR^{neg}, exogenous CD16 or a variant thereof, CD38 negative, HLA-I and/or HLA-II deficiency, and/or HLA-G.

In some embodiments, the iPSC, and its derivative effector cells comprising any one of the genotypes in Table 1 may additionally comprise disruption of at least one of TAP1, TAP2, Tapasin, NLRC5, PD1, LAG3, TIM3, RFXANK, RFX5, RFXAP, RAG1, and any gene in the chromosome 6p21 region; or introduction of at least one of HLA-E, 4-1BBL, CD4, CD8, CD47, CD113, CD131, CD137, CD80, PDL1, A_{2A}R, TCR, Fc receptor, an antibody, and surface triggering receptor for coupling with bi-, multi-specific or universal engagers.

7. Genetically Engineered iPSC Line and iPSC-Derived Cells Provided Herein

In light of the above, the present application provides an iPSC, an iPS cell line cell, or a population thereof, and a derivative effector cell obtained from differentiating the iPSC, wherein each cell comprises at least a polynucleotide encoding a CAR and optionally a polynucleotide encoding an engager having a different tumor targeting specificity from the CAR, wherein the cell is an eukaryotic cell, an animal cell, a human cell, an induced pluripotent cell (iPSC), an iPSC derived effector cell, an immune cell, or a feeder cell. Also provided is a master cell bank comprising single cell sorted and expanded clonal engineered iPSCs having a phenotype as described herein, wherein the cell bank provides a renewable source for manufacturing off-the-shelf, engineered, homogeneous cell therapy products, which are well-defined and uniform in composition, and can be mass produced at a significant scale in a cost-effective manner. In some embodiments, the iPSC-derived cells are hematopoietic cells including, but not limited to, mesodermal cells with definitive hemogenic endothelium (HE) potential, definitive HE, CD34⁺ hematopoietic cells, hematopoietic stem and progenitor cells, hematopoietic multipotent progenitors (MPP), T cell progenitors, NK cell progenitors, myeloid cells, neutrophil progenitors, and/or sharing features with T cells, NKT cells, NK cells, B cells, neutrophils, dendritic cells, and macrophages. In some embodiments, the iPSC-derived hematopoietic cells comprise immune effector cells expressing at least a CAR and optionally an engager having a different tumor targeting specificity from the CAR. Further provided herein is an iPSC, an iPS cell line cell, or a clonal population thereof, and a derivative functional cell obtained from differentiating the iPSC, wherein each cell comprises a polynucleotide encoding a CAR and optionally, a polynucleotide encoding an engager having a different tumor targeting specificity from the CAR, and one or more of CD38 knockout; HLA-I deficiency and/or HLA-II deficiency; introduction of HLA-G or non-cleavable HLA-G, or knockout of one or both of CD58 and CD54; an exogenous CD16 or a variant thereof, a CFR; a signaling complex comprising a cytokine and/or its receptor or variants thereof; and, wherein the iPSC is capable of directed differentiation to produce functional derivative hematopoietic cells. In some embodiments, the functional derivative hematopoietic cells are immune effector cells. In some embodiments, the functional derivative immune effector cells share features with NK and/or T cells. In some embodiments, the functional derivative immune effector cells sharing features with NK and/or T cells are not NK cells or T cells.

In some embodiments of the iPSC, the iPS cell line cell, or the clonal population thereof, and the derivative functional cell obtained from differentiating the iPSC, wherein each cell comprises at least a polynucleotide encoding a CAR and optionally, a polynucleotide encoding an engager having a different tumor targeting specificity from the CAR, the cell is TCR^{neg}. As used herein, TCR^{neg} is also referred to as TCR negative, TCR^{-/-}, “TCR null”, or TCR knockout, which comprises cells without endogenous TCR expression either by nature (for example, NK cell or iPSC derived NK cell), by gene expression regulation, or by genomic editing of an iPSC cell (for example, iPSC, iPSC reprogrammed from T cell (TiPSC)) or a T cell to knock out an endogenous TCR or one or more subunits thereof, or by obtaining TCR negative derivative cells differentiated from iPSC having TCR knocked out. As such, the TCR that is knocked out in a cell as disclosed is an endogenous TCR complex. Disrupting expression of the constant region of either TCRα or TCRβ in a cell is one of many methods of knocking out the endogenous TCR complex of the cell. TCR^{neg} cells are not able to present a CD3 complex at the cell surface despite expressing all CD3 subunits in the TCR^{neg} cells, which adversely affects cell functions that require cell surface CD3 recognition, binding and/or signaling. Thus, in some embodiments of the TCR^{neg} cells comprising a polynucleotide encoding a CAR, optionally, a polynucleotide encoding an engager having a different tumor targeting specificity from the CAR, and a CFR, the CFR is CD3-based. In some embodiments, the TCR^{neg} cells which comprise a polynucleotide encoding a CAR and optionally, a polynucleotide encoding an engager having a different tumor targeting specificity from the CAR, also comprise a cell surface CD3 complex, or one or more subunits or subdomains thereof (cs-CD3) when expressed.

In some embodiments, the cell comprising a CAR and optionally an engager, also comprises a CAR inserted in a constant region of a TCR. In some embodiments, the cell comprising a CAR and optionally an engager, is TCR^{neg} and comprises a CAR inserted in a constant region of a TCR and the expression of the CAR is driven by an endogenous TCR promoter. In some embodiments, the cell comprising a CAR and optionally an engager, also comprises an exogenous cytokine signaling of IL2, IL4, IL7, IL9, IL15, IL21, or any combinations thereof. In some embodiments, the exogenous cytokine signaling is cell membrane bound. In some embodiments, the exogenous cytokine signaling comprises an introduced partial or full peptide of a cytokine and/or its respective receptor or mutated or truncated variants thereof. In some embodiments, the cytokine signaling is constitutively activated. In some embodiments, the activation of the cytokine signaling is inducible. In some embodiments, the activation of the cytokine signaling is transient and/or temporal. In some embodiments, the transient/temporal expression of a cell surface cytokine signaling is through a retrovirus, Sendai virus, an adenovirus, an episome, mini-circle, or RNAs including mRNA. In some embodiments, the exogenous cell surface cytokine signaling enables IL2 signaling. In some embodiments, the exogenous cell surface cytokine signaling enables IL4 signaling. In some embodiments, the exogenous cell surface cytokine signaling enables IL7 signaling. In some embodiments, the exogenous cell surface cytokine signaling enables IL9 signaling. In some embodiments, the exogenous cell surface cytokine signaling enables TL15 signaling. In some embodiments, the exogenous cell surface cytokine signaling enables IL21 signaling. In some embodiments, the cell comprising a CAR and optionally an engager, further comprises an exogenous CD16 or functional variants or chimeric receptors thereof. In some embodiments, the exogenous CD16 comprises an ectodomain comprising F176V and S197P. In some embodiments, the exogenous CD16 comprises a full or a partial length of an ectodomain of CD64. In some other embodiments, the exogenous CD16 comprises a chimeric Fc receptor. The exogenous CD16 enables cell killing through ADCC, thereby providing a dual targeting mechanism to an effector cell expressing, for example, a CAR.

In some embodiments, the cell comprising a CAR and optionally an engager, further comprises a CD38 knockout. The cell surface molecule CD38 is highly upregulated in multiple hematologic malignancies derived from both lymphoid and myeloid lineages, including multiple myeloma and a CD20 negative B-cell malignancy, which makes it an attractive target for antibody therapeutics to deplete cancer cells. Other than being highly expressed on malignant cells, CD38 is also expressed on plasma cells as well as on NK cells, and activated T and B cells. In some embodiments, effector cells that are CD38^{-/-} can avoid CD38 induced fractricide. In some embodiments, when an anti-CD38 antibody, a CD38 binding CAR, or a CD3 engager comprising anti-CD38 scFV is used to induce the ADCC and/or tumor cell targeting, the CD38^{-/-} iPSC and/or its derivative effector cells can target the CD38 expressing (tumor) cells without causing effector cell elimination, i.e., reduction or depletion of CD38 expressing effector cells, thereby increasing the iPSC and its effector cell persistence and/or survival.

In some embodiments of the cell comprising a polynucleotide encoding a CAR and optionally, a polynucleotide encoding an engager, the cell further comprises HLA-I and/or HLA-II deficiency (e.g., a B2M knockout and/or a CIITA knockout), and optionally, a polynucleotide encoding HLA-G or HLA-E. In some embodiments of the cell comprising a polynucleotide encoding a CAR and optionally, a polynucleotide encoding an engager, the cell further comprises HLA-I and/or HLA-II deficiency (e.g., a B2M knockout and/or a CIITA knockout), and optionally, one or both of CD58 and CD54 knockout.

In view of the above, provided herein is an iPSC comprising a polynucleotide encoding a CAR and optionally, a polynucleotide encoding an engager, and further optionally one, two, three, or more, or all of: TCR^{neg}, an exogenous CD16 or a variant thereof, a CFR, a signaling complex comprising a cell surface expressed exogenous IL, CD38 knockout, and B2M/CIITA knockout; wherein when B2M is knocked out, a polynucleotide encoding HLA-G, or alternatively, one or both of CD58 and CD54 knockout, is optionally introduced, and wherein the iPSC is capable of directed differentiation to produce functional derivative hematopoietic cells.

As such, the present application provides iPSCs and functional derivative hematopoietic cells thereof, which comprise any one of the following genotypes in Table 1. Also provided herein is a master cell bank comprising single cell sorted and expanded clonal engineered iPSCs comprising any one of the following genotypes in Table 1, i.e., having a CAR and one or both of an engager (“Eg” in Table 1) and a CFR, and optionally, one or more of an exogenous CD16 or a variant thereof, TCR^{neg}, a signaling complex comprising a cell surface expressed exogenous IL, CD38 knockout, and HLA-I and/or HLA-II deficiency, without adversely impacting the differentiation potential of the iPSC and function of the derived effector cells. Said cell bank provides a platform for additional iPSC engineering, and a renewable source for manufacturing off-the-shelf, engineered, homogeneous cell therapy products. Depending on the insertion site of any one of the exogenous polynucleotides, the engineered effector cell may be negative in endogenous TCR expression. Furthermore, if the engineered effector cell is of an NK cell lineage, the cell is also TCR negative.

“IL”, as provided in Table 1 stands for one of IL2, IL4, IL6, IL7, IL9, IL10, IL11, IL12, IL15, IL18, and IL21, depending on which specific cytokine/receptor or combination expression is selected; and when IL7 is selected, IL stands for IL7, including IL7Ra and IL7Rβ. Likewise, when IL15 is selected, IL stands for IL15, including IL15Rα and IL15Rβ. In some embodiments, the cell surface expressed exogenous cytokine and/or a receptor thereof comprises at least one of co-expression of IL15 and IL15Rα by using a self-cleaving peptide, a fusion protein of IL15 and IL15Rα, an IL15/IL15Rα fusion protein with intracellular domain of IL15Rα truncated or eliminated, a fusion protein of IL15 and IL15Rβ, a fusion protein of IL15 and common receptor γC, wherein the common receptor γC is native or modified, and a homodimer of IL15Rβ. In some embodiments, the IL15/IL15Rα fusion protein comprises an amino acid sequence of at least 75%, 80%, 85%, 90%, 95% or 99% identity to SEQ ID NOs: 25, 27 or 28. In some embodiments, the truncated IL15/IL15Rα fusion protein lacking an intracellular domain comprises an amino acid sequence of SEQ ID NO: 25. In some embodiments, the truncated IL15/IL15Rα fusion protein lacking an intracellular domain comprises an amino acid sequence of SEQ ID NO: 27. In some embodiments, the truncated IL15/IL15Rα fusion protein lacking an intracellular domain comprises an amino acid sequence of SEQ ID NO: 28.

In some embodiments, the cell surface expressed exogenous cytokine and/or a receptor thereof comprises at least one of co-expression of IL7 and IL7Rα by using a self-cleaving peptide, a fusion protein of IL7 and IL7Rα, an IL7/IL7Rα fusion protein with intracellular domain of IL7Rα truncated or eliminated, a fusion protein of IL7 and IL7Rβ, a fusion protein of IL7 and common receptor γC, wherein the common receptor γC is native or modified, and a homodimer of IL7Rβ. In some embodiments, the IL7/IL7Rα fusion protein comprises an amino acid sequence of at least 75%, 80%, 85%, 90%, 95% or 99% identity to SEQ ID NO: 30. In some embodiments, the IL7/IL7Rα fusion protein comprises an amino acid sequence of SEQ ID NO: 30.

Further, when iPSCs and functional derivative hematopoietic cells thereof have a genotype comprising both CAR and IL, the CAR and IL may optionally be comprised in a bi-cistronic expression cassette comprising a 2A sequence. As comparison, in some other embodiments, CAR and IL are in separate expression cassettes comprised in iPSCs and functional derivative hematopoietic cells thereof.

TABLE 1
Applicable Genotypes of the Cells Provided:

| | Engager | | | | | | |
|---|---|---|---|---|---|---|---|
| | and/or | | | | | HLA-G or (CD58^{-/-} | |
| CAR | CFR | CD16 | IL | CD38^{-/-} | B2M^{-/-}CIITA^{-/-} | w/or w/o CD54^{-/-}) | Genotype |
| ✓ | | | | | | | 1. camB7H3 CAR |
| ✓ | ✓ | | | | | | 2. CAR Eg |
| | | | | | | | 3. CAR CFR |
| | | | | | | | 4. CAR Eg CFR |
| ✓ | ✓ | ✓ | | | | | 5. CAR Eg CD16 |
| | | | | | | | 6. CAR CFR CD16 |
| | | | | | | | 7. CAR Eg CFR CD16 |
| ✓ | ✓ | | ✓ | | | | 8. CAR Eg IL |
| | | | | | | | 9. CAR CFR IL |
| | | | | | | | 10. CAR Eg CFR IL |
| ✓ | ✓ | | | ✓ | | | 11. CAR Eg CD38^{-/-} |
| | | | | | | | 12. CAR CFR CD38^{-/-} |
| | | | | | | | 13. CAR Eg CFR CD38^{-/-} |
| ✓ | ✓ | | | | ✓ | | 14. CAR Eg B2M^{-/-}CIITA^{-/-} |
| | | | | | | | 15. CAR CFR B2M^{-/-}CIITA^{-/-} |
| | | | | | | | 16. CAR Eg CFR B2M^{-/-}CIITA^{-/-} |
| ✓ | ✓ | | | | ✓ | ✓ | 17. CAR Eg B2M^{-/-}CIITA^{-/-}CD58^{-/-} |
| | | | | | | | 18. CAR CFR B2M^{-/-}CIITA^{-/-}CD58^{-/-} |
| | | | | | | | 19. CAR Eg CFR B2M^{-/-}CIITA^{-/-}CD58^{-/-} |
| | | | | | | | 20. CAR Eg B2M^{-/-}CIITA^{-/-}CD54^{-/-} |
| | | | | | | | 21. CAR CFR B2M^{-/-}CIITA^{-/-}CD54^{-/-} |
| | | | | | | | 22. CAR Eg CFR B2M^{-/-}CIITA^{-/-}CD54^{-/-} |
| | | | | | | | 23. CAR Eg B2M^{-/-}CIITA^{-/-}CD58^{-/-}CD54^{-/-} |
| | | | | | | | 24. CAR CFR B2M^{-/-}CIITA^{-/-}CD58^{-/-}CD54^{-/-} |
| | | | | | | | 25. CAR Eg CFR B2M^{-/-}CIITA^{-/-}CD58^{-/-}CD54^{-/-} |
| | | | | | | | 26. CAR Eg B2M^{-/-}CIITA^{-/-}HLA-G |
| | | | | | | | 27. CAR CFR B2M^{-/-}CIITA^{-/-}HLA-G |
| | | | | | | | 28. CAR Eg CFR B2M^{-/-}CIITA^{-/-}HLA-G |
| ✓ | ✓ | ✓ | ✓ | | | | 29. CAR Eg CD16 IL |
| | | | | | | | 30. CAR CFR CD16 IL |
| | | | | | | | 31. CAR Eg CFR CD16 IL |
| ✓ | ✓ | ✓ | | ✓ | | | 32. CAR Eg CD16 CD38^{-/-} |
| | | | | | | | 33. CAR CFR CD16 CD38^{-/-} |
| | | | | | | | 34. CAR Eg CFR CD16 CD38^{-/-} |
| ✓ | ✓ | ✓ | | | ✓ | | 35. CAR Eg CD16 B2M^{-/-}CIITA^{-/-} |
| | | | | | | | 36. CAR CFR CD16 B2M^{-/-}CIITA^{-/-} |
| | | | | | | | 37. CAR Eg CFR CD16 B2M^{-/-}CIITA^{-/-} |
| ✓ | ✓ | ✓ | | | ✓ | ✓ | 38. CAR Eg CD16 B2M^{-/-}CIITA^{-/-}CD58^{-/-} |
| | | | | | | | 39. CAR CFR CD16 B2M^{-/-}CIITA^{-/-}CD58^{-/-} |
| | | | | | | | 40. CAR Eg CFR CD16 B2M^{-/-}CIITA^{-/-}CD58^{-/-} |
| | | | | | | | 41. CAR Eg CD16 B2M^{-/-}CIITA^{-/-}CD54^{-/-} |
| | | | | | | | 42. CAR CFR CD16 B2M^{-/-}CIITA^{-/-}CD54^{-/-} |
| | | | | | | | 43. CAR Eg CFR CD16 B2M^{-/-}CIITA^{-/-}CD54^{-/-} |
| | | | | | | | 44. CAR Eg CD16 B2M^{-/-}CIITA^{-/-}CD58^{-/-}CD54^{-/-} |
| | | | | | | | 45. CAR CFR CD16 B2M^{-/-}CIITA^{-/-}CD58^{-/-}CD54^{-/-} |
| | | | | | | | 46. CAR Eg CFR CD16 B2M^{-/-}CIITA^{-/-}CD58^{-/-}CD54^{-/-} |
| | | | | | | | 47. CAR Eg CD16 B2M^{-/-}CIITA^{-/-}HLA-G |
| | | | | | | | 48. CAR CFR CD16 B2M^{-/-}CIITA^{-/-}HLA-G |
| | | | | | | | 49. CAR Eg CFR CD16 B2M^{-/-}CIITA^{-/-}HLA-G |
| ✓ | ✓ | | ✓ | ✓ | | | 50. CAR Eg IL CD38^{-/-} |
| | | | | | | | 51. CAR CFR IL CD38^{-/-} |
| | | | | | | | 52. CAR Eg CFR IL CD38^{-/-} |
| ✓ | ✓ | | ✓ | | ✓ | | 53. CAR Eg IL CD38^{-/-}B2M/-CIITA^{-/-} |
| | | | | | | | 54. CAR CFR IL CD38^{-/-}B2M^{-/-}CIITA^{-/-} |
| | | | | | | | 55. CAR Eg CFR IL CD38^{-/-}B2M^{-/-}CIITA^{-/-} |
| ✓ | ✓ | | ✓ | ✓ | | ✓ | 56. CAR Eg IL CD38^{-/-}B2M^{-/-}CIITA^{-/-}CD58^{-/-} |
| | | | | | | | 57. CAR CFR IL CD38^{-/-}B2M^{-/-}CIITA^{-/-}CD58^{-/-} |
| | | | | | | | 58. CAR Eg CFR IL CD38^{-/-}B2M^{-/-}CIITA^{-/-}CD58^{-/-} |
| | | | | | | | 59. CAR Eg IL CD38^{-/-}B2M^{-/-}CIITA^{-/-}CD54^{-/-} |
| | | | | | | | 60. CAR CFR IL CD38^{-/-}B2M^{-/-}CIITA^{-/-}CD54^{-/-} |
| | | | | | | | 61. CAR Eg CFR IL CD38^{-/-}B2M^{-/-}CIITA^{-/-}CD54^{-/-} |
| | | | | | | | 62. CAR Eg IL CD38^{-/-}B2M-/-CIITA^{-/-}CD58^{-/-}CD54^{-/-} |
| | | | | | | | 63. CAR CFR IL CD38^{-/-}B2M^{-/-}CIITA^{-/-}CD58^{-/-}CD54^{-/-} |
| | | | | | | | 64. CAR Eg CFR IL CD38^{-/-}B2M^{-/-}CIITA^{-/-}CD58^{-/-}CD54^{-/-} |
| | | | | | | | 65. CAR Eg IL CD38^{-/-}B2M^{-/-}CIITA^{-/-}HLA-G |
| | | | | | | | 66. CAR CFR IL CD38^{-/-}B2M^{-/-}CIITA^{-/-}HLA-G |
| | | | | | | | 67. CAR Eg CFR IL CD38^{-/-}B2M^{-/-}CIITA^{-/-}HLA-G |
| ✓ | ✓ | | | ✓ | ✓ | | 68. CAR Eg CD38^{-/-}B2M^{-/-}CIITA^{-/-} |
| | | | | | | | 69. CAR CFR CD38^{-/-}B2M^{-/-}CIITA^{-/-} |
| | | | | | | | 70. CAR Eg CFR CD38^{-/-}B2M^{-/-}CIITA^{-/-} |
| ✓ | ✓ | | | ✓ | ✓ | ✓ | 71. CAR Eg CD38^{-/-}B2M^{-/-}CIITA^{-/-}CD58^{-/-} |
| | | | | | | | 72. CAR CFR CD38^{-/-}B2M^{-/-}CIITA^{-/-}CD58^{-/-} |
| | | | | | | | 73. CAR Eg CFR CD38^{-/-}B2M^{-/-}CIITA^{-/-}CD58^{-/-} |
| | | | | | | | 74. CAR Eg CD38^{-/-}B2M^{-/-}CIITA^{-/-}CD54^{-/-} |
| | | | | | | | 75. CAR CFR CD38^{-/-}B2M^{-/-}CIITA^{-/-}CD54^{-/-} |
| | | | | | | | 76. CAR Eg CFR CD38^{-/-}B2M^{-/-}CIITA^{-/-}CD54^{-/-} |
| | | | | | | | 77. CAR Eg CD38^{-/-}B2M^{-/-}CIITA^{-/-}CD58^{-/-}CD54^{-/-} |
| | | | | | | | 78. CAR CFR CD38^{-/-}B2M^{-/-}CIITA^{-/-}CD58^{-/-}CD54^{-/-} |
| | | | | | | | 79. CAR Eg CFR CD38^{-/-}B2M^{-/-}CIITA^{-/-}CD58^{-/-}CD54^{-/-} |
| | | | | | | | 80. CAR Eg CD38^{-/-}B2M^{-/-}CIITA^{-/-}HLA-G |
| | | | | | | | 81. CAR CFR CD38^{-/-}B2M^{-/-}CIITA^{-/-}HLA-G |
| | | | | | | | 82. CAR Eg CFR CD38^{-/-}B2M^{-/-}CIITA^{-/-}HLA-G |
| ✓ | ✓ | ✓ | ✓ | ✓ | | | 83. CAR Eg CD16 IL CD38^{-/-} |
| | | | | | | | 84. CAR CFR CD16 IL CD38^{-/-} |
| | | | | | | | 85. CAR Eg CFR CD16 IL CD38^{-/-} |
| ✓ | ✓ | ✓ | ✓ | | ✓ | | 86. CAR Eg CD16 IL B2M^{-/-}CIITA^{-/-} |
| | | | | | | | 87. CAR CFR CD16 IL B2M^{-/-}CIITA^{-/-} |
| | | | | | | | 88. CAR Eg CFR CD16 IL B2M^{-/-}CIITA^{-/-} |
| ✓ | ✓ | ✓ | ✓ | | ✓ | ✓ | 89. CAR Eg CD16 IL B2M^{-/-}CIITA^{-/-}CD58^{-/-} |
| | | | | | | | 90. CAR CFR CD16 IL B2M^{-/-}CIITA^{-/-}CD58^{-/-} |
| | | | | | | | 91. CAR Eg CFR CD16 IL B2M^{-/-}CIITA^{-/-}CD58^{-/-} |
| | | | | | | | 92. CAR Eg CD16 IL B2M^{-/-}CIITA^{-/-}CD54^{-/-} |
| | | | | | | | 93. CAR CFR CD16 IL B2M^{-/-}CIITA^{-/-}CD54^{-/-} |
| | | | | | | | 94. CAR Eg CFR CD16 IL B2M^{-/-}CIITA^{-/-}CD54^{-/-} |
| | | | | | | | 95. CAR Eg CD16 IL B2M^{-/-}CIITA^{-/-}CD58^{-/-}CD54^{-/-} |
| | | | | | | | 96. CAR CFR CD16 IL B2M^{-/-}CIITA^{-/-}CD58^{-/-}CD54^{-/-} |
| | | | | | | | 97. CAR Eg CFR CD16 IL B2M^{-/-}CIITA^{-/-}CD58^{-/-}CD54^{-/-} |
| | | | | | | | 98. CAR Eg CD16 IL B2M^{-/-}CIITA^{-/-}HLA-G |
| | | | | | | | 99. CAR CFR CD16 IL B2M^{-/-}CIITA^{-/-}HLA-G |
| | | | | | | | 100. CAR Eg CFR CD16 IL B2M^{-/-}CIITA^{-/-}HLA-G |
| ✓ | ✓ | | ✓ | ✓ | ✓ | | 101. CAR Eg IL CD38^{-/-}B2M^{-/-}CIITA^{-/-} |
| | | | | | | | 102. CAR CFR IL CD38^{-/-}B2M^{-/-}CIITA^{-/-} |
| | | | | | | | 103. CAR Eg CFR IL B2M^{-/-}CIITA^{-/-} |
| ✓ | ✓ | | ✓ | ✓ | ✓ | ✓ | 104. CAR Eg IL CD38^{-/-}B2M^{-/-}CIITA^{-/-}CD58^{-/-} |
| | | | | | | | 105. CAR CFR IL CD38^{-/-}B2M^{-/-}CIITA^{-/-}CD58^{-/-} |
| | | | | | | | 106. CAR Eg CFR IL B2M^{-/-}CIITA^{-/-}CD58^{-/-} |
| | | | | | | | 107. CAR Eg IL CD38^{-/-}B2M^{-/-}CIITA^{-/-}CD54^{-/-} |
| | | | | | | | 108. CAR CFR IL CD38^{-/-}B2M^{-/-}CIITA^{-/-}CD54^{-/-} |
| | | | | | | | 109. CAR Eg CFR IL B2M^{-/-}CIITA^{-/-}CD54^{-/-} |
| | | | | | | | 110. CAR Eg IL CD38^{-/-}B2M^{-/-}CIITA^{-/-}CD58^{-/-}CD54^{-/-} |
| | | | | | | | 111. CAR CFR IL CD38^{-/-}B2M^{-/-}CIITA^{-/-}CD58^{-/-}CD54^{-/-} |
| | | | | | | | 112. CAR Eg CFR IL B2M^{-/-}CIITA^{-/-}CD58^{-/-}CD54^{-/-} |
| | | | | | | | 113. CAR Eg IL CD38^{-/-}B2M^{-/-}CIITA^{-/-}HLA-G |
| | | | | | | | 114. CAR CFR IL CD38^{-/-}B2M^{-/-}CIITA^{-/-}HLA-G |
| | | | | | | | 115. CAR Eg CFR IL B2M^{-/-}CIITA^{-/-}HLA-G |
| ✓ | ✓ | ✓ | ✓ | ✓ | ✓ | | 116. CAR Eg CD16 IL CD38^{-/-}B2M^{-/-}CIITA^{-/-} |
| | | | | | | | 117. CAR CFR CD16 IL CD38^{-/-}B2M^{-/-}CIITA^{-/-} |
| | | | | | | | 118. CAR Eg CFR CD16 IL CD38^{-/-}B2M^{-/-}CIITA^{-/-} |
| ✓ | ✓ | ✓ | ✓ | ✓ | ✓ | ✓ | 119. CAR Eg CD16 IL CD38^{-/-}B2M^{-/-}CIITA^{-/-}CD58^{-/-} |
| | | | | | | | 120. CAR CFR CD16 CD38^{-/-}B2M^{-/-}CIITA^{-/-}CD58^{-/-} |
| | | | | | | | 121. CAR Eg CFR CD16 CD38^{-/-}B2M^{-/-}CIITA^{-/-}CD58^{-/-} |
| | | | | | | | 122. CAR Eg CD16 CD38^{-/-}B2M^{-/-}CIITA^{-/-}CD54^{-/-} |
| | | | | | | | 123. CAR CFR CD16 CD38^{-/-}B2M^{-/-}CIITA^{-/-}CD54^{-/-} |
| | | | | | | | 124. CAR Eg CFR CD16 CD38^{-/-}B2M^{-/-}CIITA^{-/-}CD54^{-/-} |
| | | | | | | | 125. CAR Eg CD16 CD38^{-/-}B2M^{-/-}CIITA^{-/-}CD58^{-/-}CD54^{-/-} |
| | | | | | | | 126. CAR CFR CD16 CD38^{-/-}B2M /-CIITA^{-/-}CD58^{-/-}CD54^{-/-} |
| | | | | | | | 127. CAR Eg CFR CD16 CD38^{-/-}B2M^{-/-}CIITA^{-/-}CD58^{-/-}CD54^{-/-} |
| | | | | | | | 128. CAR Eg CD16 CD38^{-/-}B2M^{-/-}CIITA^{-/-}HLA-G |
| | | | | | | | 129. CAR CFR CD16 CD38^{-/-}B2M^{-/-}CIITA^{-/-}HLA-G |
| | | | | | | | 130. CAR Eg CFR CD16 CD38^{-/-}B2M^{-/-}CIITA^{-/-}HLA-G |

8. Antibodies for Immunotherapy

In some embodiments, in addition to the genomically engineered effector cells as provided herein, additional therapeutic agents comprising an antibody, or an antibody fragment thereof, that targets an antigen associated with a condition, a disease, or an indication may be used with these effector cells in a combinational therapy, as compared to being expressed by the genomically engineered effector cells. In some embodiments, the antibody is a monoclonal antibody. In some embodiments, the antibody is a humanized antibody, a humanized monoclonal antibody, or a chimeric antibody. In some embodiments, the antibody, or antibody fragment, specifically binds to a viral antigen. In other embodiments, the antibody, or antibody fragment, specifically binds to a tumor antigen. In some embodiments, the tumor or viral specific antigen activates the administered iPSC-derived effector cells to enhance their killing ability. In some embodiments, the antibodies suitable for combinational treatment as an additional therapeutic agent to the administered iPSC-derived effector cells include, but are not limited to, anti-CD20 (rituximab, veltuzumab, ofatumumab, ublituximab, ocaratuzumab, obinutuzumab, ibritumomab, ocrelizumab), anti-CD22 (inotuzumab, moxetumomab, epratuzumab), anti-HER2 (trastuzumab, pertuzumab), anti-CD52 (alemtuzumab), anti-EGFR (cetuximab), anti-GD2 (dinutuximab), anti-PDL1 (avelumab), anti-CD38 (daratumumab, isatuximab, MOR202), anti-CD123 (7G3, CSL362), anti-SLAMF7 (elotuzumab), and their humanized or Fc modified variants or fragments or their functional equivalents and biosimilars.

In some embodiments, the antibodies suitable for combinational treatment as an additional therapeutic agent to the administered iPSC-derived effector cells further include bi-specific or multi-specific antibodies that target more than one antigen or epitope on a target cell or recruit effector cells (e.g., T cells, NK cells, or macrophage cells) toward target cells while targeting the target cells. Such bi-specific or multi-specific antibodies function as engagers capable of directing an effector cell, whether a bystander immune cell (e.g., a T cell, a NK cell, an NKT cell, a B cell, a macrophage, and/or a neutrophil in the recipient of the therapy) or the engineered effector cell in the therapeutic composition, to a tumor cell and activating the immune effector cell upon binding of the tumor antigen, and have shown great potential to maximize the benefits of antibody therapy. An engager is specific to at least one tumor antigen and is specific to at least one surface triggering receptor of an immune effector cell, which could provide a multi-targeting approach for the engineered cell disclosed herein to address tumor antigen evasion and tumor heterogeneity. Examples of engagers include, but are not limited to, bi-specific T cell engagers (BiTEs), bi-specific killer cell engagers (BiKEs), tri-specific killer cell engagers (TriKEs), or multi-specific killer cell engagers, or universal engagers compatible with multiple immune cell types.

In some embodiments, the iPSC-derived effector cells comprise hematopoietic lineage cells comprising a genotype listed in Table 1. In some embodiments, the iPSC-derived effector cells comprise a genotype listed in Table 1. In some embodiments of a combination useful for treating liquid or solid tumors, the combination comprises iPSC-derived effector cells comprising at least a CAR and optionally a CFR, as provided herein, optionally where the CFR is for engager coupling. In some other embodiments of a combination useful for treating liquid or solid tumors, the combination comprises a preselected monoclonal antibody and iPSC-derived effector cells comprising at least a CAR, and optionally one or more of a CFR and an exogenous CD16 or variant thereof. In some embodiments of a combination useful for treating liquid or solid tumors, the combination comprises a monoclonal antibody and iPSC-derived effector cells comprising at least a CAR, and optionally one or more of TCR^{neg}; an exogenous CD16 or a variant thereof, a CFR; an additional cytokine signaling complex comprising a cytokine and/or its receptor or variants thereof, and CD38 knockout. In various embodiments, the exogenous CD16 is hnCD16. Without being limited by the theory, hnCD16 provides enhanced ADCC of the monoclonal antibody, whereas the CAR not only targets a specific tumor antigen but also prevents tumor antigen escape using a dual targeting strategy in combination with an monoclonal antibody targeting a different tumor antigen.

In some further embodiments, the iPSC-derived NK cells comprised in the combination with daratumumab comprise a CAR, and optionally one or more of exogenous CD16 or a variant thereof, IL7 or IL15, and the CAR targets at least one of B7H3, MICA/B, CD19, BCMA, CD20, CD22, CD123, HER2, CD52, EGFR, GD2, MSLN, VEGF-R2, PSMA and PDL1; wherein the IL7 or IL15 signaling complex is co- or separately expressed with the CAR.

9. Checkpoint Inhibitors

Checkpoints are cell molecules, often cell surface molecules, capable of suppressing or downregulating immune responses when not inhibited. It is now clear that tumors co-opt certain immune-checkpoint pathways as a major mechanism of immune resistance, particularly against T cells that are specific for tumor antigens. Checkpoint inhibitors (CIs) are antagonists capable of reducing checkpoint gene expression or gene products, or decreasing activity of checkpoint molecules, thereby block inhibitory checkpoints, restoring immune system function. The development of checkpoint inhibitors targeting PD1/PDL1 or CTLA4 has transformed the oncology landscape, with these agents providing long term remissions in multiple indications. However, many tumor subtypes are resistant to checkpoint blockade therapy, and relapse remains a significant concern. Thus, one aspect of the present application provides a therapeutic approach to overcome CI resistance by including genomically engineered functional iPSC-derived cells as provided herein in a combination therapy with CI. In one embodiment of the combination therapy, the iPSC-derived cells are NK cells. In another embodiment of the combination therapy, the iPSC-derived cells are T cells. In addition to exhibiting direct antitumor capacity, the derivative NK cells provided herein have been shown to resist PDL1-PD1 mediated inhibition, and to have the ability to enhance T cell migration, to recruit T cells to the tumor microenvironment, and to augment T cell activation at the tumor site. Therefore, the tumor infiltration of T cells facilitated by the functionally potent genomically engineered derivative NK cells indicate that said NK cells are capable of synergizing with T cell targeted immunotherapies, including the checkpoint inhibitors, to relieve local immunosuppression and to reduce tumor burden.

In one embodiment, the iPSC-derived effector cell for checkpoint inhibitor combination therapy comprises a CAR, and optionally one, two, three, four, five or more of: engager expression, exogenous CD16 expression, CFR expression, HLA-I and/or HLA-II deficiency, CD38 knockout, and an exogenous cell surface cytokine and/or receptor expression; wherein when B2M is knocked out, a polynucleotide encoding HLA-G or knockout of one or both of CD58 and CD54 is optionally included. In some embodiments, the derivative NK cell comprises any one of the genotypes listed in Table 1. In some embodiments, the above derivative effector cell additionally comprises deletion, disruption, or reduced expression of at least one of TAP1, TAP2, Tapasin, NLRC5, PD1, LAG3, TIM3, RFXANK, RFX5, RFXAP, RAG1, and any gene in the chromosome 6p21 region; or introduction of at least one of HLA-E, 4-1BBL, CD3, CD4, CD8, CD47, CD113, CD131, CD137, CD80, PDL1, A_{2A}R, CAR, Fc receptor, and surface triggering receptor for coupling with bi-, multi-specific or universal engagers.

In various embodiments, the derivative effector cell is obtained from differentiating an iPSC clonal line comprising one, two, three, four, five or more of: CAR expression, engager expression, exogenous CD16 expression, HLA-I and/or HLA-II deficiency, CD38 knockout, and exogenous cell surface cytokine expression; wherein when B2M is knocked out, a polynucleotide encoding HLA-G or knockout of one or both of CD58 and CD54 is optionally introduced. In some embodiments, the above-described iPSC clonal line further comprises deletion, disruption, or reduced expression of at least one of TAP1, TAP2, Tapasin, NLRC5, PD1, LAG3, TIM3, RFXANK, RFX5, RFXAP, RAG1, and any gene in the chromosome 6p21 region; or introduction of at least one of HLA-E, 4-1BBL, CD3, CD4, CD8, CD47, CD113, CD131, CD137, CD80, PDL1, A_{2A}R, CAR, Fc receptor, and surface triggering receptor for coupling with bi-, multi-specific or universal engagers.

Suitable checkpoint inhibitors for combination therapy with the derivative effector cells as provided herein include, but are not limited to, antagonists of PD-1 (Pdcdl, CD279), PDL-1 (CD274), TIM-3 (Havcr2), TIGIT (WUCAM and Vstm3), LAG-3 (Lag3, CD223), CTLA-4 (Ctla4, CD152), 2B4 (CD244), 4-1BB (CD137), 4-1BBL (CD137L), A_{2A}R, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, and inhibitory KIR (for example, 2DL1, 2DL2, 2DL3, 3DL1, and 3DL2).

In some embodiments, the antagonist inhibiting any of the above checkpoint molecules is an antibody. In some embodiments, the checkpoint inhibitory antibodies may be murine antibodies, human antibodies, humanized antibodies, a camel Ig, single variable new antigen receptors (VNAR), shark heavy-chain antibodies (Ig-NAR), chimeric antibodies, recombinant antibodies, single-domain antibodies (dAb), anti-idiotype antibodies, bispecific-, multi-specific- or multimeric-antibodies, or antibody fragments thereof. Non-limiting examples of antibody fragments include Fab, Fab′, F(ab′)2, F(ab′)3, Fv, Fabc, pFc, Fd, single chain antigen binding fragments (scFv), tandem scFv (scFv)2, disulfide stabilized Fv (dsFv), minibody, diabody, triabody, tetrabody, single-domain antigen binding fragments (sdAb), camelid heavy-chain IgG and Nanobody® fragments, recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of atezolizumab (anti-PDL1 mAb), avelumab (anti-PDL1 mAb), durvalumab (anti-PDL1 mAb), tremelimumab (anti-CTLA4 mAb), ipilimumab (anti-CTLA4 mAb), IPH4102 (anti-KIR), IPH43 (anti-MICA), IPH33 (anti-TLR3), lirimumab (anti-KIR), monalizumab (anti-NKG2A), nivolumab (anti-PD1 mAb), pembrolizumab (anti-PD1 mAb), and any derivatives, functional equivalents, or biosimilars thereof.

In some embodiments, the antagonist inhibiting any of the above checkpoint molecules is microRNA-based, as many miRNAs are found as regulators that control the expression of immune checkpoints (Dragomir et al., Cancer Biol Med. 2018, 15(2):103-115). In some embodiments, the checkpoint antagonistic miRNAs include, but are not limited to, miR-28, miR-15/16, miR-138, miR-342, miR-20b, miR-21, miR-130b, miR-34a, miR-197, miR-200c, miR-200, miR-17-5p, miR-570, miR-424, miR-155, miR-574-3p, miR-513, and miR-29c.

Some embodiments of the combination therapy with the provided iPSC-derived effector cells comprise at least one checkpoint inhibitor to target at least one checkpoint molecule; wherein the iPSC-derived cells have a genotype listed in Table 1. Some other embodiments of the combination therapy with the provided derivative effector cells comprise two, three or more checkpoint inhibitors such that two, three, or more checkpoint molecules are targeted. In some embodiments of the combination therapy comprising at least one checkpoint inhibitor and the iPSC-derived cells having a genotype listed in Table 1, said checkpoint inhibitor is an antibody, or a humanized or Fc modified variant or fragment, or a functional equivalent or biosimilar thereof, and said checkpoint inhibitor is produced by the iPSC-derived cells by expressing an exogenous polynucleotide sequence encoding said antibody, or a fragment or variant thereof. In some embodiments, the exogenous polynucleotide sequence encoding the antibody, or a fragment or a variant thereof that inhibits a checkpoint is co-expressed with a CAR, either in separate constructs or in a bi-cistronic construct comprising both the CAR and the sequence encoding the antibody, or the fragment thereof. In some further embodiments, the sequence encoding the antibody or the fragment thereof can be linked to either the 5′ or the 3′ end of a CAR expression construct through a self-cleaving 2A coding sequence, illustrated as, for example, CAR-2A-CI or CI-2A-CAR. As such, the coding sequences of the checkpoint inhibitor and the CAR may be in a single open reading frame (ORF). When the checkpoint inhibitor is delivered, expressed and secreted as a payload by the derivative effector cells capable of infiltrating the tumor microenvironment (TME), it counteracts the inhibitory checkpoint molecule upon engaging the TME, allowing activation of the effector cells by activating modalities such as CAR or activating receptors. In some embodiments, the checkpoint inhibitor co-expressed with CAR inhibits at least one of the checkpoint molecules: PD-1, PDL-1, TIM-3, TIGIT, LAG-3, CTLA-4, 2B4, 4-1BB, 4-1BBL, A_{2A}R, BATE, BTLA, CD39 (Entpdl), CD47, CD73 (NT5E), CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, and inhibitory KIR. In some embodiments, the checkpoint inhibitor co-expressed with CAR in a derivative cell having a genotype listed in Table 1 is selected from a group comprising atezolizumab, avelumab, durvalumab, tremelimumab, ipilimumab, IPH4102, IPH43, IPH33, lirimumab, monalizumab, nivolumab, pembrolizumab, and their humanized, or Fc modified variants, fragments and their functional equivalents or biosimilars. In some embodiments, the checkpoint inhibitor co-expressed with CAR is atezolizumab, or its humanized, or Fc modified variants, fragments or their functional equivalents or biosimilars. In some other embodiments, the checkpoint inhibitor co-expressed with CAR is nivolumab, or its humanized, or Fc modified variants, fragments or their functional equivalents or biosimilars. In some other embodiments, the checkpoint inhibitor co-expressed with CAR is pembrolizumab, or its humanized, or Fc modified variants, fragments or their functional equivalents or biosimilars.

In some other embodiments of the combination therapy comprising the iPSC-derived cells provided herein and at least one antibody inhibiting a checkpoint molecule, said antibody is not produced by, or in, the iPSC-derived cells and is additionally administered before, with, or after the administering of the iPSC-derived cells having a genotype listed in Table 1. In some embodiments, the administering of one, two, three or more checkpoint inhibitors in a combination therapy with the provided derivative effector cells are simultaneous or sequential. In one embodiment of the combination treatment comprising derived NK cells or T cells having a genotype listed in Table 1, the checkpoint inhibitor included in the treatment is one or more of atezolizumab, avelumab, durvalumab, tremelimumab, ipilimumab, IPH4102, IPH43, IPH33, lirimumab, monalizumiab, nivolumab, pembrolizumab, and their humanized or Fc modified variants, fragments and their functional equivalents or biosimilars. In some embodiments of the combination treatment comprising derived NK cells or T cells having a genotype listed in Table 1, the checkpoint inhibitor included in the treatment is atezolizumab, or its humanized or Fc modified variant, fragment and its functional equivalent or biosimilar. In some embodiments of the combination treatment comprising derived NK cells or T cells having a genotype listed in Table 1, the checkpoint inhibitor included in the treatment is nivolumab, or its humanized or Fc modified variant, fragment or its functional equivalent or biosimilar. In some embodiments of the combination treatment comprising derived NK cells or T cells having a genotype listed in Table 1, the checkpoint inhibitor included in the treatment is pembrolizumab, or its humanized or Fc modified variant, fragment or its functional equivalent or biosimilar.

II. Methods for Targeted Genome Editing at Selected Locus in iPSCs

Genome editing, or genomic editing, or genetic editing, as used interchangeably herein, is a type of genetic engineering in which DNA is inserted, deleted, and/or replaced in the genome of a targeted cell. Targeted genome editing (interchangeable with “targeted genomic editing” or “targeted genetic editing”) enables insertion, deletion, and/or substitution at pre-selected sites in the genome. When an endogenous sequence is deleted at the insertion site during targeted editing, an endogenous gene comprising the affected sequence may be knocked-out or knocked-down due to the sequence deletion. Therefore, targeted editing may also be used to disrupt endogenous gene expression with precision. Similarly used herein is the term “targeted integration,” referring to a process involving insertion of one or more exogenous sequences, with or without deletion of an endogenous sequence at the insertion site. In comparison, randomly integrated genes are subject to position effects and silencing, making their expression unreliable and unpredictable. For example, centromeres and sub-telomeric regions are particularly prone to transgene silencing. Reciprocally, newly integrated genes may affect the surrounding endogenous genes and chromatin, potentially altering cell behavior or favoring cellular transformation. Therefore, inserting exogenous DNA in a pre-selected locus such as a safe harbor locus, or genomic safe harbor (GSH) is important for safety, efficiency, copy number control, and for reliable gene response control.

Targeted editing can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted editing could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.” In some situations, the targeted integration site is intended to be within a coding region of a selected gene, and thus the targeted integration could disrupt the gene expression, resulting in simultaneous knock-in and knockout (KI/KO) in one single editing step.

Inserting one or more transgenes at a selected position in a gene locus of interest (GOI) to knock out the gene at the same time can be achieved. Gene loci suitable for simultaneous knock-in and knockout (KI/KO) include, but are not limited to, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, and TIGIT. With respective site-specific targeting homology arms for position-selective insertion, it allows the transgene(s) to express either under an endogenous promoter at the site or under an exogenous promoter comprised in the construct. When two or more transgenes are to be inserted at a selected location (e.g., in a CD38 locus), a linker sequence, for example, a 2A linker or IRES, is placed between any two transgenes. The 2A linker encodes a self-cleaving peptide derived from FMDV, ERAV, PTV-I, or TaV (referred to as “F2A”, “E2A”, “P2A”, and “T2A”, respectively), allowing for separate proteins to be produced from a single translation. In some embodiments, insulators are included in the construct to reduce the risk of transgene and/or exogenous promoter silencing. The exogenous promoter may be CAG, or other constitutive, inducible, temporal-, tissue-, or cell type-specific promoters including, but not limited to CMV, EF1α, PGK, and UBC.

Available endonucleases capable of introducing specific and targeted DSBs include, but are not limited to, zinc-finger nucleases (ZFN), transcription activator-like effector nucleases (TALEN), RNA-guided CRISPR (Clustered Regular Interspaced Short Palindromic Repeats) systems. Additionally, DICE (dual integrase cassette exchange) system utilizing phiC31 and Bxb1 integrases is also a promising tool for targeted integration.

ZFNs are targeted nucleases comprising a nuclease fused to a zinc finger DNA binding domain. By a “zinc finger DNA binding domain” or “ZFBD” it is meant a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but are not limited to, C₂H₂ zinc fingers, C₃H zinc fingers, and C₄ zinc fingers. A “designed” zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also International Pub. Nos. WO98/53058; WO98/53059; WO98/53060; WO02/016536 and WO03/016496. A “selected” zinc finger domain is a domain not found in nature whose production results primarily from an empirical process such as phage display, interaction trap or hybrid selection. ZFNs are described in greater detail in U.S. Pat. Nos. 7,888,121 and 7,972,854, the complete disclosures of which are incorporated herein by reference. The most recognized example of a ZFN in the art is a fusion of the FokI nuclease with a zinc finger DNA binding domain.

A TALEN is a targeted nuclease comprising a nuclease fused to a TAL effector DNA binding domain. By “transcription activator-like effector DNA binding domain”, “TAL effector DNA binding domain”, or “TALE DNA binding domain” it is meant the polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Pub. No. 2011/0145940, which is herein incorporated by reference. The most recognized example of a TALEN in the art is a fusion polypeptide of the FokI nuclease to a TAL effector DNA binding domain.

Another example of a targeted nuclease that finds use in the subject methods is a targeted Spo11 nuclease, a polypeptide comprising a Spo11 polypeptide having nuclease activity fused to a DNA binding domain, e.g., a zinc finger DNA binding domain, a TAL effector DNA binding domain, etc. that has specificity for a DNA sequence of interest.

Additional examples of targeted nucleases suitable for the present invention include, but are not limited to, Bxb1, phiC31, R4, PhiBT1, and WP/SPBc/TP901-1, whether used individually or in combination.

Other non-limiting examples of targeted nucleases include naturally occurring and recombinant nucleases; CRISPR related nucleases from families including cas, cpf, cse, csy, csn, csd, cst, csh, csa, csm, and cmr; restriction endonucleases; meganucleases; homing endonucleases, and the like.

Using Cas9 as an example, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction. When using the CRISPR/Cpf system, it requires a Cpf endonuclease (Cpf1, MAD7 and many more known in the art) and (2) the gNA, which often does not need tracrRNA, to guide Cpf endonuclease to target selected sequences.

DICE-mediated insertion uses a pair of recombinases, for example, phiC31 and Bxb1, to provide unidirectional integration of an exogenous DNA that is tightly restricted to each enzymes' own small attB and attP recognition sites. Because these target att sites are not naturally present in mammalian genomes, therefore they must be first introduced into the genome at the desired integration site. See, for example, U.S. Pub. No. 2015/0140665, the disclosure of which is incorporated herein by reference.

One aspect of the present invention provides a construct comprising one or more exogenous polynucleotides for targeted genome integration. In one embodiment, the construct further comprises a pair of homologous arms specific to a desired integration site, and the method of targeted integration comprises introducing the construct to cells to enable site specific homologous recombination by the cell host enzymatic machinery. In another embodiment, the method of targeted integration in a cell comprises introducing a construct comprising one or more exogenous polynucleotides to the cell and introducing a ZFN expression cassette comprising a DNA-binding domain specific to a desired integration site to the cell to enable a ZFN-mediated insertion. In yet another embodiment, the method of targeted integration in a cell comprises introducing a construct comprising one or more exogenous polynucleotides to the cell and introducing a TALEN expression cassette comprising a DNA-binding domain specific to a desired integration site to the cell to enable a TALEN-mediated insertion. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration.

Promising sites for targeted integration include, but are not limited to, safe harbor loci, or genomic safe harbor (GSH), which are intragenic or extragenic regions of the human genome that, theoretically, are able to accommodate predictable expression of newly integrated DNA without adverse effects on the host cell or organism. A useful safe harbor must permit sufficient transgene expression to yield desired levels of the vector-encoded protein or non-coding RNA. A safe harbor also must not predispose cells to malignant transformation nor alter cellular functions. For an integration site to be a potential safe harbor locus, it ideally needs to meet criteria including, but not limited to: absence of disruption of regulatory elements or genes, as judged by sequence annotation; is an intergenic region in a gene dense area, or a location at the convergence between two genes transcribed in opposite directions; keep distance to minimize the possibility of long-range interactions between vector-encoded transcriptional activators and the promoters of adjacent genes, particularly cancer-related and microRNA genes; and has apparently ubiquitous transcriptional activity, as reflected by broad spatial and temporal expressed sequence tag (EST) expression patterns, indicating ubiquitous transcriptional activity. This latter feature is especially important in stem cells, where during differentiation, chromatin remodeling typically leads to silencing of some loci and potential activation of others. Within the region suitable for exogenous insertion, a precise locus chosen for insertion should be devoid of repetitive elements and conserved sequences and to which primers for amplification of homology arms could easily be designed.

Suitable sites for human genome editing, or specifically, targeted integration, include, but are not limited to, the adeno-associated virus site 1 (AAVS1), the chemokine (CC motif) receptor 5 (CCR5) gene locus and the human orthologue of the mouse ROSA26 locus. Additionally, the human orthologue of the mouse H11 locus may also be a suitable site for insertion using the composition and method of targeted integration disclosed herein. Further, collagen and HTRP gene loci may also be used as safe harbor for targeted integration. However, validation of each selected site has been shown to be necessary especially in stem cells for specific integration events, and optimization of insertion strategy including promoter election, exogenous gene sequence and arrangement, and construct design is often needed.

For targeted in/dels, the editing site is often comprised in an endogenous gene whose expression and/or function is intended to be disrupted. In one embodiment, the endogenous gene comprising a targeted in/del is associated with immune response regulation and modulation. In some other embodiments, the endogenous gene comprising a targeted in/del is associated with targeting modality, receptors, signaling molecules, transcription factors, drug target candidates, immune response regulation and modulation, or proteins suppressing engraftment, trafficking, homing, viability, self-renewal, persistence, and/or survival of stem cells and/or progenitor cells, and the derived cells therefrom.

As such, another aspect of the present invention provides a method of targeted integration in a selected locus including genome safe harbor or a preselected locus known or proven to be safe and well-regulated for continuous or temporal gene expression such as the B2M, TAP1, TAP2, Tapasin, TRAC, or CD38 locus as provided herein. In one embodiment, the genome safe harbor for the method of targeted integration comprises one or more desired integration sites comprising AAVS1, CCR5, ROSA26, collagen, HTRP, H11, beta-2 microglobulin, CD38, GAPDH, TCR or RUNX1, or other loci meeting the criteria of a genome safe harbor. In some embodiments, the targeted integration is in one or more gene loci where the knock-down or knockout of the gene as a result of the integration is desired, wherein such gene loci include, but are not limited to, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, and TIGIT.

In one embodiment, the method of targeted integration in a cell comprises introducing a construct comprising one or more exogenous polynucleotides to the cell, and introducing a construct comprising a pair of homologous arm specific to a desired integration site and one or more exogenous sequence, to enable site specific homologous recombination by the cell host enzymatic machinery, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, or TIGIT.

In another embodiment, the method of targeted integration in a cell comprises introducing a construct comprising one or more exogenous polynucleotides to the cell, and introducing a ZFN expression cassette comprising a DNA-binding domain specific to a desired integration site to the cell to enable a ZFN-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, or TIGIT. In yet another embodiment, the method of targeted integration in a cell comprises introducing a construct comprising one or more exogenous polynucleotides to the cell, and introducing a TALEN expression cassette comprising a DNA-binding domain specific to a desired integration site to the cell to enable a TALEN-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, or TIGIT. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, or TIGIT. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration, wherein the desired integration site comprises AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, or TIGIT.

Further, as provided herein, the above method for targeted integration in a safe harbor is used to insert any polynucleotide of interest, for example, polynucleotides encoding safety switch proteins, targeting modality, receptors, signaling molecules, transcription factors, pharmaceutically active proteins and peptides, drug target candidates, and proteins promoting engraftment, trafficking, homing, viability, self-renewal, persistence, and/or survival of stem cells and/or progenitor cells. In some other embodiments, the construct comprising one or more exogenous polynucleotides further comprises one or more marker genes. In one embodiment, the exogenous polynucleotide in a construct of the invention is a suicide gene encoding a safety switch protein. Suitable suicide gene systems for induced cell death include, but are not limited to Caspase 9 (or caspase 3 or 7) and AP1903; thymidine kinase (TK) and ganciclovir (GCV); cytosine deaminase (CD) and 5-fluorocytosine (5-FC). Additionally, some suicide gene systems are cell type specific, for example, the genetic modification of T lymphocytes with the B-cell molecule CD20 allows their elimination upon administration of mAb Rituximab. Further, modified EGFR containing epitope recognized by cetuximab can be used to deplete genetically engineered cells when the cells are exposed to cetuximab. As such, one aspect of the invention provides a method of targeted integration of one or more suicide genes encoding safety switch proteins selected from caspase 9 (caspase 3 or 7), thymidine kinase, cytosine deaminase, modified EGFR, and B-cell CD20.

In some embodiments, one or more exogenous polynucleotides integrated by the method herein are driven by operatively linked exogenous promoters comprised in the construct for targeted integration. The promoters may be inducible, or constitutive, and may be temporal-, tissue- or cell type-specific. Suitable constitutive promoters for methods of the invention include, but are not limited to, cytomegalovirus (CMV), elongation factor 1α (EF1α), phosphoglycerate kinase (PGK), hybrid CMV enhancer/chicken β-actin (CAG) and ubiquitin C (UBC) promoters. In one embodiment, the exogenous promoter is CAG.

The exogenous polynucleotides integrated by the method provided herein may be driven by endogenous promoters in the host genome, at the integration site. In one embodiment, the method of the invention is used for targeted integration of one or more exogenous polynucleotides at the AAVS1 locus in the genome of a cell. In one embodiment, at least one integrated polynucleotide is driven by the endogenous AAVS1 promoter. In another embodiment, the method of the invention is used for targeted integration at the ROSA26 locus in the genome of a cell. In one embodiment, at least one integrated polynucleotide is driven by the endogenous ROSA26 promoter. In still another embodiment, the method of the invention is used for targeted integration at the H11 locus in the genome of a cell. In one embodiment, at least one integrated polynucleotide is driven by the endogenous H11 promoter. In another embodiment, the method of the invention is used for targeted integration at collagen locus in the genome of a cell. In one embodiment, at least one integrated polynucleotide is driven by the endogenous collagen promoter. In still another embodiment, the method of the invention is used for targeted integration at HTRP locus in the genome of a cell. In one embodiment, at least one integrated polynucleotide is driven by the endogenous HTRP promoter. Theoretically, only correct insertions at the desired location would enable gene expression of an exogenous gene driven by an endogenous promoter.

In some embodiments, the one or more exogenous polynucleotides comprised in the construct for the methods of targeted integration are driven by one promoter. In some embodiments, the construct comprises one or more linker sequences between two adjacent polynucleotides driven by the same promoter to provide greater physical separation between the moieties and maximize the accessibility to enzymatic machinery. The linker peptide of the linker sequences may consist of amino acids selected to make the physical separation between the moieties (exogenous polynucleotides, and/or the protein or peptide encoded therefrom) more flexible or more rigid depending on the relevant function. The linker sequence may be cleavable by a protease or cleavable chemically to yield separate moieties. Examples of enzymatic cleavage sites in the linker include sites for cleavage by a proteolytic enzyme, such as enterokinase, Factor Xa, trypsin, collagenase, and thrombin. In some embodiments, the protease is one which is produced naturally by the host or it is exogenously introduced. Alternatively, the cleavage site in the linker may be a site capable of being cleaved upon exposure to a selected chemical or condition, e.g., cyanogen bromide, hydroxylamine, or low pH. The optional linker sequence may serve a purpose other than the provision of a cleavage site. The linker sequence should allow effective positioning of the moiety with respect to another adjacent moiety for the moieties to function properly. The linker may also be a simple amino acid sequence of a sufficient length to prevent any steric hindrance between the moieties. In addition, the linker sequence may provide for post-translational modification including, but not limited to, e.g., phosphorylation sites, biotinylation sites, sulfation sites, γ-carboxylation sites, and the like. In some embodiments, the linker sequence is flexible so as not hold the biologically active peptide in a single undesired conformation. The linker may be predominantly comprised of amino acids with small side chains, such as glycine, alanine, and serine, to provide for flexibility. In some embodiments about 80 or 90 percent or greater of the linker sequence comprises glycine, alanine, or serine residues, particularly glycine and serine residues. In several embodiments, a G4S linker peptide separates the end-processing and endonuclease domains of the fusion protein. In other embodiments, a 2A linker sequence allows for two separate proteins to be produced from a single translation. Suitable linker sequences can be readily identified empirically. Additionally, suitable size and sequences of linker sequences also can be determined by conventional computer modeling techniques. In one embodiment, the linker sequence encodes a self-cleaving peptide. In one embodiment, the self-cleaving peptide is 2A. In some other embodiments, the linker sequence provides an Internal Ribosome Entry Sequence (IRES). In some embodiments, any two consecutive linker sequences are different.

The method of introducing into cells a construct comprising exogenous polynucleotides for targeted integration can be achieved using a method of gene transfer to cells known per se. In one embodiment, the construct comprises backbones of viral vectors such as adenovirus vectors, adeno-associated virus vectors, retrovirus vectors, lentivirus vectors, or Sendai virus vectors. In some embodiments, the plasmid vectors are used for delivering and/or expressing the exogenous polynucleotides to target cells (e.g., pAl-11, pXTI, pRc/CMV, pRc/RSV, pcDNAI/Neo) and the like. In some other embodiments, the episomal vector is used to deliver the exogenous polynucleotide to target cells. In some embodiments, recombinant adeno-associated viruses (rAAVs) can be used for genetic engineering to introduce insertions, deletions or substitutions through homologous recombinations. Unlike lentiviruses, rAAVs do not integrate into the host genome. In addition, episomal rAAV vectors mediate homology-directed gene targeting at much higher rates compared to transfection of conventional targeting plasmids. In some embodiments, an AAV6 or AAV2 vector is used to introduce insertions, deletions or substitutions in a target site in the genome of iPSCs. In some embodiments, the genomically modified iPSCs and its derivative cells obtained using the methods and compositions herein comprise at least one genotype listed in Table 1.

III. Method of Obtaining and Maintaining Genome-Engineered iPSCs

The present invention provides a method of obtaining and maintaining genome-engineered iPSCs comprising one or more targeted editing at one or more desired sites, wherein the targeted editing remains intact and functional in expanded genome-engineered iPSCs or the iPSCs derived non-pluripotent cells at the respective selected editing site. The targeted editing introduces into the genome of the iPSC, and derivative cells therefrom, insertions, deletions, and/or substitutions, i.e., targeted integration and/or in/dels at selected sites. In comparison to direct engineering of patient-sourced, peripheral blood originated primary effector cells, the many benefits of obtaining genomically engineered iPSC-derived effector cells through editing and differentiating iPSC as provided herein include, but are not limited to: unlimited source for engineered effector cells; no need for repeated manipulation of the effector cells especially when multiple engineered modalities are involved; the obtained effector cells are rejuvenated for having elongated telomere and experiencing less exhaustion; the effector cell population is homogeneous in terms of editing site, copy number, and void of allelic variation, random mutations and expression variegation, largely due to the enabled clonal selection in engineered iPSCs as provided herein.

In particular embodiments, the genome-engineered iPSCs comprising one or more targeted editing at one or more selected sites are maintained, passaged and expanded as single cells for an extended period in the cell culture medium shown in Table 2 as Fate Maintenance Medium (FMM), wherein the iPSCs retain the targeted editing and functional modification at the selected site(s). The components of the medium may be present in the medium in amounts within an optimal range shown in Table 2. The iPSCs cultured in FMM have been shown to continue to maintain their undifferentiated, and ground or naïve, profile; genomic stability without the need for culture cleaning or selection; and are readily to give rise to all three somatic lineages, in vitro differentiation via embryoid bodies or monolayer (without formation of embryoid bodies); and in vivo differentiation by teratoma formation. See, for example, International Pub. No. WO2015/134652, the disclosure of which is incorporated herein by reference.

TABLE 2
Exemplary media for iPSC reprogramming and maintenance

| Conventional | | |
|---|---|---|
| hESC Medium | Fate Reprogramming | Fate Maintenance |
| (Conv.) | Medium (FRM) | Medium (FMM) |
| DMEM/F12 | DMEM/F12 | DMEM/F12 |
| Knockout Serum | Knockout Serum | Knockout Serum |
| Replacement (20%) | Replacement (20%) | Replacement (20%) |
| | N2 | |
| | B27 | |
| Glutamine | Glutamine | Glutamine (1x) |
| Non-Essential | Non-Essential | Non-Essential |
| Amino Acids (1x) | Amino Acids (1x) | Amino Acids (1x) |
| β-mercaptoethanol | β-mercaptoethanol | β-mercaptoethanol |
| (100 μM) | (100 μM) | (100 μM) |
| bFGF (0.2-50 ng/mL) | bFGF (2-500 ng/mL) | bFGF (2-500 ng/mL) |
| | LIF (0.2-50 ng/mL) | LIF (0.2-50 ng/mL) |
| | Thiazovivin | Thiazovivin |
| | (0.1-25 μM) | (0.1-25 μM) |
| | PD0325901 | PD0325901 |
| | (0.005-2 μM) | (0.005-2 μM) |
| | CHIR99021 | CHIR99021 |
| | (0.02-5 μM) | (0.02-5 μM) |
| | SB431542 | |
| | (0.04-10 μM) | |

| | |
|---|---|
| In combination with | Feeder-free, in combination with Matrigel ™ or |
| MEF feeder cells | Vitronectin |

In some embodiments, the genome-engineered iPSCs comprising one or more targeted integration and/or in/dels are maintained, passaged and expanded in a medium comprising a MEK inhibitor, a GSK3 inhibitor, and a ROCK inhibitor, and free of, or essentially free of, TGFβ receptor/ALK5 inhibitors, wherein the iPSCs retain the intact and functional targeted editing at the selected sites.

Another aspect of the invention provides a method of generating genome-engineered iPSCs through targeted editing of iPSCs; or through first generating genome-engineered non-pluripotent cells by targeted editing, and then reprogramming the selected/isolated genome-engineered non-pluripotent cells to obtain iPSCs comprising the same targeted editing as the non-pluripotent cells. A further aspect of the invention provides genome-engineering non-pluripotent cells which are concurrently undergoing reprogramming by introducing targeted integration and/or targeted in/dels to the cells, wherein the contacted non-pluripotent cells are under sufficient conditions for reprogramming, and wherein the conditions for reprogramming comprise contacting non-pluripotent cells with one or more reprogramming factors and small molecules. In various embodiments of the method for concurrent genome-engineering and reprogramming, the targeted integration and/or targeted in/dels may be introduced to the non-pluripotent cells prior to, or essentially concomitantly with, initiating reprogramming by contacting the non-pluripotent cells with one or more reprogramming factors and optionally small molecules.

In some embodiments, to concurrently genome-engineer and reprogram non-pluripotent cells, the targeted integration and/or in/dels may also be introduced to the non-pluripotent cells after the multi-day process of reprogramming is initiated by contacting the non-pluripotent cells with one or more reprogramming factors and small molecules, and wherein the vectors carrying the constructs are introduced before the reprogramming cells present stable expression of one or more endogenous pluripotent genes including but not limited to SSEA4, Tra181 and CD30.

In some embodiments, the reprogramming is initiated by contacting the non-pluripotent cells with at least one reprogramming factor, and optionally a combination of a TGFβ receptor/ALK inhibitor, a MEK inhibitor, a GSK3 inhibitor and a ROCK inhibitor (FRM; Table 2). In some embodiments, the genome-engineered iPSCs through any methods above are further maintained and expanded using a mixture of comprising a combination of a MEK inhibitor, a GSK3 inhibitor and a ROCK inhibitor (FMM; Table 2).

In some embodiments of the method of generating genome-engineered iPSCs, the method comprises: genomic engineering an iPSC by introducing one or more targeted integration and/or in/dels into iPSCs to obtain genome-engineered iPSCs having at least one genotype listed in Table 1. Alternatively, the method of generating genome-engineered iPSCs comprises: (a) introducing one or more targeted editing into non-pluripotent cells to obtain genome-engineered non-pluripotent cells comprising targeted integration and/or in/dels at selected sites, and (b) contacting the genome-engineered non-pluripotent cells with one or more reprogramming factors, and optionally a small molecule composition comprising a TGFβ receptor/ALK inhibitor, a MEK inhibitor, a GSK3 inhibitor and/or a ROCK inhibitor, to obtain genome-engineered iPSCs comprising targeted integration and/or in/dels at selected sites. Alternatively, the method of generating genome-engineered iPSCs comprises: (a) contacting non-pluripotent cells with one or more reprogramming factors, and optionally a small molecule composition comprising a TGFβ receptor/ALK inhibitor, a MEK inhibitor, a GSK3 inhibitor and/or a ROCK inhibitor to initiate the reprogramming of the non-pluripotent cells; (b) introducing one or more targeted integration and/or in/dels into the reprogramming non-pluripotent cells for genome-engineering; and (c) obtaining genome-engineered iPSCs comprising targeted integration and/or in/dels at selected sites. Any of the above methods may further comprise single cell sorting genome-engineered iPSCs to obtain a clonal iPSC. Through clonal expansion of this genome-engineered iPSC, a master cell bank is generated to comprise single cell sorted and expanded clonal engineered iPSCs having at least one phenotype as provided herein in Table 1. The master cell bank is subsequently cryopreserved, providing a platform for additional iPSC engineering and a renewable source for manufacturing off-the-shelf, engineered, homogeneous cell therapy products, which are well-defined and uniform in composition, and can be mass produced at significant scale in a cost-effective manner.

The reprogramming factors are selected from the group consisting of OCT4, SOX2, NANOG, KLF4, LIN28, C-MYC, ECAT1, UTF1, ESRRB, SV40LT, HESRG, CDH1, TDGF1, DPPA4, DNMT3B, ZIC3, L1TD1, and any combinations thereof as disclosed in International Pub. Nos. WO2015/134652 and WO2017/066634, the disclosures of which are incorporated herein by reference. The one or more reprogramming factors may be in the form of a polypeptide. The reprogramming factors may also be in the form of polynucleotides, and thus are introduced to the non-pluripotent cells by vectors such as, a retrovirus, a Sendai virus, an adenovirus, an episome, a plasmid, and a mini-circle. In particular embodiments, the one or more polynucleotides encoding at least one reprogramming factor are introduced by a lentiviral vector. In some embodiments, the one or more polynucleotides introduced by an episomal vector. In various other embodiments, the one or more polynucleotides are introduced by a Sendai viral vector. In some embodiments, the one or more polynucleotides introduced by a combination of plasmids. See, for example, International Pub. No. WO2019/075057, the disclosure of which is incorporated herein by reference.

In some embodiments, the non-pluripotent cells are transferred with multiple constructs comprising different exogenous polynucleotides and/or different promoters by multiple vectors for targeted integration at the same or different selected sites. These exogenous polynucleotides may comprise a suicide gene, or a gene encoding targeting modality, receptors, signaling molecules, transcription factors, pharmaceutically active proteins and peptides, drug target candidates, or a gene encoding a protein promoting engraftment, trafficking, homing, viability, self-renewal, persistence, and/or survival of the iPSCs or derivative cells therefrom. In some embodiments, the exogenous polynucleotides encode RNA, including but not limited to siRNA, shRNA, miRNA and antisense nucleic acids. These exogenous polynucleotides may be driven by one or more promoters selected form the group consisting of constitutive promoters, inducible promoters, temporal-specific promoters, and tissue or cell type specific promoters. Accordingly, the polynucleotides are expressible when under conditions that activate the promoter, for example, in the presence of an inducing agent or in a particular differentiated cell type. In some embodiments, the polynucleotides are expressed in iPSCs and/or in cells differentiated from the iPSCs. In one embodiment, one or more suicide gene is driven by a constitutive promoter, for example Caspase-9 driven by CAG. These constructs comprising different exogenous polynucleotides and/or different promoters can be transferred to non-pluripotent cells either simultaneously or consecutively. The non-pluripotent cells subjecting to targeted integration of multiple constructs can simultaneously contact the one or more reprogramming factors to initiate the reprogramming concurrently with the genomic engineering, thereby obtaining genome-engineered iPSCs comprising multiple targeted integration in the same pool of cells. As such, this robust method enables a concurrent reprogramming and engineering strategy to derive a clonal genomically engineered hiPSC with multiple modalities integrated to one or more selected target sites. In some embodiments, the genomically modified iPSCs and their derivative cells obtained using the methods and composition herein comprise at least one genotype listed in Table 1.

IV. A Method of Obtaining Genetically-Engineered Effector Cells by Differentiating Genome-Engineered iPSC

A further aspect of the present invention provides a method of in vivo differentiation of genome-engineered iPSC by teratoma formation, wherein the differentiated cells derived in vivo from the genome-engineered iPSCs retain the intact and functional targeted editing including targeted integration and/or in/dels at the desired site(s). In some embodiments, the differentiated cells derived in vivo from the genome-engineered iPSCs via teratoma comprise one or more inducible suicide genes integrated at one or more desired site comprising AAVS1, CCR5, ROSA26, collagen, HTRP H11, beta-2 microglobulin, CD38, GAPDH, TCR or RUNX1, or other loci meeting the criteria of a genome safe harbor. In some other embodiments, the differentiated cells derived in vivo from the genome-engineered iPSCs via teratoma comprise polynucleotides encoding targeting modality, or encoding proteins promoting trafficking, homing, viability, self-renewal, persistence, and/or survival of stem cells and/or progenitor cells. In some embodiments, the differentiated cells derived in vivo from the genome-engineered iPSCs via teratoma comprising one or more inducible suicide genes further comprises one or more in/dels in endogenous genes associated with immune response regulation and mediation. In some embodiments, the in/del is comprised in one or more endogenous checkpoint genes. In some embodiments, the in/del is comprised in one or more endogenous T cell receptor genes. In some embodiments, the in/del is comprised in one or more endogenous MHC class I suppressor genes. In some embodiments, the in/del is comprised in one or more endogenous genes associated with the major histocompatibility complex. In some embodiments, the in/del is comprised in one or more endogenous genes including, but not limited to, AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, or TIGIT. In one embodiment, the genome-engineered iPSC comprising one or more exogenous polynucleotides at selected site(s) further comprises a targeted editing in B2M (beta-2-microglobulin) encoding gene.

In particular embodiments, the genome-engineered iPSCs comprising one or more genetic modifications as provided herein are used to derive hematopoietic cell lineages or any other specific cell types in vitro, wherein the derived non-pluripotent cells retain the functional genetic modifications including targeted editing at the selected site(s). In some embodiments, the genome-engineered iPSCs used to derive hematopoietic cell lineages or any other specific cell types in vitro are master cell bank cells that are cryopreserved and thawed right before their usage. In one embodiment, the genome-engineered iPSC-derived cells include, but are not limited to, mesodermal cells with definitive hemogenic endothelium (HE) potential, definitive HE, CD34⁺ hematopoietic cells, hematopoietic stem and progenitor cells, hematopoietic multipotent progenitors (MPP), T cell progenitors, NK cell progenitors, myeloid cells, neutrophil progenitors, T cells, NKT cells, NK cells, B cells, neutrophils, dendritic cells, and macrophages, wherein these cells derived from the genome-engineered iPSCs retain the functional genetic modifications including targeted editing at the desired site(s).

Applicable differentiation methods and compositions for obtaining iPSC-derived hematopoietic cell lineages include those depicted in, for example, International Pub. No. WO2017/078807, the disclosure of which is incorporated herein by reference. As provided, the methods and compositions for generating hematopoietic cell lineages are through definitive hemogenic endothelium (HE) derived from pluripotent stem cells, including iPSCs, under serum-free, feeder-free, and/or stromal-free conditions and in a scalable and monolayer culturing platform without the need of EB formation. Cells that may be differentiated according to the provided methods range from pluripotent stem cells, to progenitor cells that are committed to particular terminally differentiated cells and transdifferentiated cells, and to cells of various lineages directly transitioned to hematopoietic fate without going through a pluripotent intermediate. Similarly, the cells that are produced by differentiating stem cells range from multipotent stem or progenitor cells, to terminally differentiated cells, and to all intervening hematopoietic cell lineages.

The methods for differentiating and expanding cells of the hematopoietic lineage from pluripotent stem cells in monolayer culturing comprise contacting the pluripotent stem cells with a BMP pathway activator, and optionally, bFGF. As provided, the pluripotent stem cell-derived mesodermal cells are obtained and expanded without forming embryoid bodies from pluripotent stem cells. The mesodermal cells are then subjected to contact with a BMP pathway activator, bFGF, and a WNT pathway activator to obtain expanded mesodermal cells having definitive hemogenic endothelium (HE) potential without forming embryoid bodies from the pluripotent stem cells. By subsequent contact with bFGF, and optionally, a ROCK inhibitor, and/or a WNT pathway activator, the mesodermal cells having definitive HE potential are differentiated to definitive HE cells, which are also expanded during differentiation.

The methods provided herein for obtaining cells of the hematopoietic lineage are superior to EB-mediated pluripotent stem cell differentiation, because EB formation leads to modest to minimal cell expansion, does not allow monolayer culturing which is important for many applications requiring homogeneous expansion, and homogeneous differentiation of the cells in a population, and is laborious and low efficiency.

The provided monolayer differentiation platform facilitates differentiation towards definitive hemogenic endothelium resulting in the derivation of hematopoietic stem cells and differentiated progeny such as T, B, NKT and NK cells. The monolayer differentiation strategy combines enhanced differentiation efficiency with large-scale expansion enables the delivery of therapeutically relevant number of pluripotent stem cell-derived hematopoietic cells for various therapeutic applications. Further, the monolayer culturing using the methods provided herein leads to functional hematopoietic lineage cells that enable full range of in vitro differentiation, ex vivo modulation, and in vivo long term hematopoietic self-renewal, reconstitution and engraftment. As provided, the iPSC-derived hematopoietic lineage cells include, but are not limited to, definitive hemogenic endothelium, hematopoietic multipotent progenitor cells, hematopoietic stem and progenitor cells, T cell progenitors, NK cell progenitors, T cells, NK cells, NKT cells, B cells, macrophages, and neutrophils.

The method for directing differentiation of pluripotent stem cells into cells of a definitive hematopoietic lineage, wherein the method comprises: (i) contacting pluripotent stem cells with a composition comprising a BMP activator, and optionally bFGF, to initiate differentiation and expansion of mesodermal cells from the pluripotent stem cells; (ii) contacting the mesodermal cells with a composition comprising a BMP activator, bFGF, and a GSK3 inhibitor, wherein the composition is optionally free of TGFβ receptor/ALK inhibitor, to initiate differentiation and expansion of mesodermal cells having definitive HE potential from the mesodermal cells; (iii) contacting the mesodermal cells having definitive HE potential with a composition comprising a ROCK inhibitor; one or more growth factors and cytokines selected from the group consisting of bFGF, VEGF, SCF, IGF, EPO, IL6, and IL11; and optionally, a Wnt pathway activator, wherein the composition is optionally free of TGFβ receptor/ALK inhibitor, to initiate differentiation and expansion of definitive hemogenic endothelium from pluripotent stem cell-derived mesodermal cells having definitive hemogenic endothelium potential.

In some embodiments, the method further comprises contacting pluripotent stem cells with a composition comprising a MEK inhibitor, a GSK3 inhibitor, and a ROCK inhibitor, wherein the composition is free of TGFβ receptor/ALK inhibitors, to seed and expand the pluripotent stem cells. In some embodiments, the pluripotent stem cells are iPSCs, or naïve iPSCs, or iPSCs comprising one or more genetic imprints; and the one or more genetic imprints comprised in the iPSC are retained in the hematopoietic cells differentiated therefrom. In some embodiments of the method for directing differentiation of pluripotent stem cells into cells of a hematopoietic lineage, the differentiation of the pluripotent stem cells into cells of hematopoietic lineage is void of generation of embryoid bodies and is in a monolayer culturing form.

In some embodiments of the above method, the obtained pluripotent stem cell-derived definitive hemogenic endothelium cells are CD34⁺. In some embodiments, the obtained definitive hemogenic endothelium cells are CD34⁺CD43⁻. In some embodiments, the definitive hemogenic endothelium cells are CD34⁺CD43⁻CXCR4⁻CD73⁻. In some embodiments, the definitive hemogenic endothelium cells are CD34⁺CXCR4⁻CD73. In some embodiments, the definitive hemogenic endothelium cells are CD34⁺CD43⁻CD93. In some embodiments, the definitive hemogenic endothelium cells are CD34⁺CD93⁻.

In some embodiments of the above method, the method further comprises (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, and IL7; and optionally a BMP activator; to initiate the differentiation of the definitive hemogenic endothelium to pre-T cell progenitors; and optionally, (ii) contacting the pre-T cell progenitors with a composition comprising one or more growth factors and cytokines selected from the group consisting of SCF, Flt3L, and IL7, but free of one or more of VEGF, bFGF, TPO, BMP activators and ROCK inhibitors, to initiate the differentiation of the pre-T cell progenitors to T cell progenitors or T cells. In some embodiments of the method, the pluripotent stem cell-derived T cell progenitors are CD34⁺CD45⁺CD7⁺. In some embodiments of the method, the pluripotent stem cell-derived T cell progenitors are CD45⁺CD7⁺.

In yet some embodiments of the above method for directing differentiation of pluripotent stem cells into cells of a hematopoietic lineage, the method further comprises: (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, IL3, IL7, and IL15; and optionally, a BMP activator, to initiate differentiation of the definitive hemogenic endothelium to pre-NK cell progenitor; and optionally, (ii) contacting pluripotent stem cell-derived pre-NK cell progenitors with a composition comprising one or more growth factors and cytokines selected from the group consisting of SCF, Flt3L, IL3, IL7, and IL15, wherein the medium is free of one or more of VEGF, bFGF, TPO, BMP activators and ROCK inhibitors, to initiate differentiation of the pre-NK cell progenitors to NK cell progenitors or NK cells. In some embodiments of the above method, step (ii) comprises contacting the pluripotent stem cell-derived pre-NK cell progenitors. In some embodiments, the pluripotent stem cell-derived NK progenitors are CD3⁻ CD45⁺CD56⁺CD7⁺. In some embodiments, the pluripotent stem cell-derived NK cells are CD3-CD45⁺CD56⁺, and optionally further defined by NKp46⁺, CD57⁺ and CD16⁺.

Therefore, using the above differentiation methods, one may obtain one or more population of iPSC-derived hematopoietic cells that are: (i) CD34⁺ HE cells (iCD34), using one or more culture medium selected from iMPP-A, iTC-A2, iTC-B2, iNK-A2, and iNK-B2; (ii) definitive hemogenic endothelium (iHE), using one or more culture medium selected from iMPP-A, iTC-A2, iTC-B2, iNK-A2, and iNK-B2; (iii) definitive HSCs, using one or more culture medium selected from iMPP-A, iTC-A2, iTC-B2, iNK-A2, and iNK-B2; (iv) multipotent progenitor cells (iMPP), using iMPP-A; (v) T cell progenitors (ipro-T), using one or more culture medium selected from iTC-A2, and iTC-B2; (vi) T cells (iTC), using iTC-B2; (vii) NK cell progenitors (ipro-NK), using one or more culture medium selected from iNK-A2, and iNK-B2; and/or (viii) NK cells (iNK), and iNK-B2. In some embodiments, the medium:
a. iCD34-C comprises a ROCK inhibitor, one or more growth factors and cytokines selected from the group consisting of bFGF, VEGF, SCF, IL6, IL11, IGF, and EPO, and optionally, a Wnt pathway activator; and is free of TGFβ receptor/ALK inhibitor;
b. iMPP-A comprises a BMP activator, a ROCK inhibitor, and one or more growth factors and cytokines selected from the group consisting of TPO, IL3, GMCSF, EPO, bFGF, VEGF, SCF, IL6, Flt3L and IL11;
c. iTC-A2 comprises a ROCK inhibitor; one or more growth factors and cytokines selected from the group consisting of SCF, Flt3L, TPO, and IL7; and optionally, a BMP activator;
d. iTC-B2 comprises one or more growth factors and cytokines selected from the group consisting of SCF, Flt3L, and IL7;
e. iNK-A2 comprises a ROCK inhibitor, and one or more growth factors and cytokines selected from the group consisting of SCF, Flt3L, TPO, IL3, IL7, and IL15; and optionally, a BMP activator, and
f. iNK-B2 comprises one or more growth factors and cytokines selected from the group consisting of SCF, Flt3L, IL7 and IL15.

In some embodiments, the genome-engineered iPSC-derived cells obtained from the above methods comprise one or more inducible suicide genes integrated at one or more desired integration sites comprising AAVS1, CCR5, ROSA26, collagen, HTRP, H11, GAPDH, RUNX1, B2M, TAP1, TAP2, tapasin, NLRC5, CIITA, RFXANK, RFX5, RFXAP, TCR α or β constant region, NKG2A, NKG2D, CD25, CD38, CD44, CD58, CD54, CD56, CD69, CD71, CIS, CBL-B, SOCS2, PD1, CTLA4, LAG3, TIM3, or TIGIT, or other loci meeting the criteria of a genome safe harbor. In some other embodiments, the genome-engineered iPSC-derived cells comprise polynucleotides encoding safety switch proteins, targeting modality, receptors, signaling molecules, transcription factors, pharmaceutically active proteins and peptides, drug target candidates, or proteins promoting trafficking, homing, viability, self-renewal, persistence, and/or survival of stem cells and/or progenitor cells. In some embodiments, the genome-engineered iPSC-derived cells comprising one or more suicide genes further comprise one or more in/del comprised in one or more endogenous genes associated with immune response regulation and mediation, including, but not limited to, check point genes, endogenous T cell receptor genes, and MHC class I suppressor genes. In one embodiment, the genome-engineered iPSC-derived cells comprising one or more suicide genes further comprise an in/del in B2M gene, wherein the B2M is knocked out.

Additionally, applicable dedifferentiation methods and compositions for obtaining genomic-engineered hematopoietic cells of a first fate to genomic-engineered hematopoietic cells of a second fate include those depicted in, for example, International Pub. No. WO2011/159726, the disclosure of which is incorporated herein by reference. The method and composition provided therein allows partially reprogramming a starting non-pluripotent cell to a non-pluripotent intermediate cell by limiting the expression of endogenous Nanog gene during reprogramming; and subjecting the non-pluripotent intermediate cell to conditions for differentiating the intermediate cell into a desired cell type. In some embodiments, the genomically modified iPSCs and its derivative cells obtained using the methods and composition herein comprise at least one genotype listed in Table 1.

V. Therapeutic Use of Derivative Immune Cells with Functional Modalities Differentiated from Genetically Engineered iPSCs

The present invention provides, in some embodiments, a composition comprising an isolated population or subpopulation of functionally enhanced derivative immune cells that have been differentiated from genomically engineered iPSCs using the methods and compositions as disclosed. In some embodiments, the iPSCs of the composition comprise one or more targeted genetic edits as disclosed, which are retainable in the iPSC-derived immune cells, wherein the genetically engineered iPSCs and derivative cells therefrom are suitable for cell based adoptive therapies. In one embodiment, the isolated population or subpopulation of genetically engineered effector cells of the composition comprise iPSC-derived CD34⁺ cells. In one embodiment, the isolated population or subpopulation of genetically engineered effector cells of the composition comprises iPSC-derived HSC cells. In one embodiment, the isolated population or subpopulation of genetically engineered effector cells of the composition comprises iPSC-derived proT or T cells. In one embodiment, the isolated population or subpopulation of genetically engineered effector cells of the composition comprises iPSC-derived proNK or NK cells. In one embodiment, the isolated population or subpopulation of genetically engineered effector cells of the composition comprises iPSC-derived immune regulatory cells or myeloid derived suppressor cells (MDSCs). In some embodiments, the iPSC-derived genetically engineered effector cells of the composition are further modulated ex vivo for improved therapeutic potential. In one embodiment of the composition, an isolated population or subpopulation of genetically engineered effector cells that have been derived from iPSC comprises an increased number or ratio of naïve T cells, stem cell memory T cells, and/or central memory T cells. In one embodiment of the composition, the isolated population or subpopulation of genetically engineered immune cells that have been derived from iPSC comprises an increased number or ratio of type I NKT cells. In another embodiment of the composition, the isolated population or subpopulation of genetically engineered immune cells that have been derived from iPSC comprises an increased number or ratio of adaptive NK cells. In some embodiments of the composition, the isolated population or subpopulation of genetically engineered CD34⁺ cells, HSC cells, T cells, NK cells, or myeloid derived suppressor cells derived from iPSC are allogeneic. In some other embodiments of the composition, the isolated population or subpopulation of genetically engineered CD34⁺ cells, HSC cells, T cells, NK cells, or MDSC derived from iPSC are autologous.

In some embodiments of the composition, the iPSC for differentiation comprises genetic imprints selected to convey desirable therapeutic attributes in effector cells, provided that cell development biology during differentiation is not disrupted, and provided that the genetic imprints are retained and functional in the differentiated hematopoietic cells derived from said iPSC.

In some embodiments of the composition, the genetic imprints of the pluripotent stem cells comprise (i) one or more genetically modified modalities obtained through genomic insertion, deletion or substitution in the genome of the pluripotent cells during or after reprogramming a non-pluripotent cell to iPSC; or (ii) one or more retainable therapeutic attributes of a source specific immune cell that is donor-, disease-, or treatment response-specific, and wherein the pluripotent cells are reprogrammed from the source specific immune cell, wherein the iPSC retain the source therapeutic attributes, which are also comprised in the iPSC-derived hematopoietic lineage cells.

In some embodiments of the composition, the genetically modified modalities comprise one or more of: safety switch proteins, targeting modalities, receptors, signaling molecules, transcription factors, pharmaceutically active proteins and peptides, drug target candidates; or proteins promoting engraftment, trafficking, homing, viability, self-renewal, persistence, immune response regulation and modulation, and/or survival of the iPSCs or derivative cells therefrom. In some embodiments of the composition, the genetically modified iPSC and the derivative cells therefrom comprise a genotype listed in Table 1. In some other embodiments of the composition, the genetically modified iPSC and the derivative cells therefrom comprising a genotype listed in Table 1 further comprise additional genetically modified modalities comprising (1) deletion, disruption, or reduced expression of one or more of TAP1, TAP2, Tapasin, NLRC5, PD1, LAG3, TIM3, RFXANK, CIITA, RFX5, or RFXAP, RAG1, and any gene in the chromosome 6p21 region; and (2) introduction of at least one of HLA-E, 4-1BBL, CD3, CD4, CD8, CD47, CD113, CD131, CD137, CD80, PDL1, A_{2A}R, CAR, Fc receptor, or surface triggering receptors for coupling with bi- or multi-specific or universal engagers.

In still some other embodiments of the composition, the hematopoietic lineage cells comprise the therapeutic attributes of the source specific immune cell relating to a combination of at least two of the following: (i) one or more antigen targeting receptor expression; (ii) modified HLA; (iii) resistance to tumor microenvironment; (iv) recruitment of bystander immune cells and immune modulations; (iv) improved on-target specificity with reduced off-tumor effect; and (v) improved homing, persistence, cytotoxicity, or antigen escape rescue.

In some embodiments of the composition, the iPSC-derived hematopoietic cells comprising a genotype listed in Table 1, and said cells express at least a CAR and one or both of an engager and a CFR, and optionally express at least one cytokine and/or its receptor comprising IL2, IL4, IL6, IL7, IL9, IL10, IL11, IL12, IL15, IL18, or IL21, or any modified protein thereof. In some embodiments of the composition, the cells express at least one cytokine and/or its receptor comprising IL2, IL4, IL7, IL9, IL15, and IL21. In some embodiments of the composition, the cells express at least one cytokine and/or its receptor comprising IL7 or IL15. In some embodiments of the composition, the engineered expression of the cytokine(s) and the CAR(s) is NK lineage cell specific. In some other embodiments of the composition, the engineered expression of the cytokine(s) and the CAR(s) is T lineage cell specific. In some embodiments of the composition, the iPSC-derived hematopoietic effector cells are antigen specific. In some embodiments of the composition, the antigen specific derivative effector cells target a liquid tumor. In some embodiments of the composition, the antigen specific derivative effector cells target a solid tumor. In some embodiments of the composition, the antigen specific iPSC-derived hematopoietic effector cells are capable of rescuing tumor antigen escape.

A variety of diseases may be ameliorated by introducing the effector cells and/or compositions of the invention to a subject suitable for adoptive cell therapy. In some embodiments, the iPSC-derived hematopoietic cells or compositions as provided is for allogeneic adoptive cell therapies. Additionally, the present invention provides, in some embodiments, therapeutic use of the above immune cells and/or therapeutic compositions and/or combination therapies by introducing the cells or composition to a subject suitable for adoptive cell therapy, wherein the subject has an autoimmune disorder; a hematological malignancy; a solid tumor; or an infection associated with HIV, RSV, EBV, CMV, adenovirus, or BK polyomavirus. Examples of hematological malignancies include, but are not limited to, acute and chronic leukemias (acute myelogenous leukemia (AML), acute lymphoblastic leukemia (ALL), chronic myelogenous leukemia (CML), lymphomas, non-Hodgkin lymphoma (NHL), Hodgkin's disease, multiple myeloma, and myelodysplastic syndromes. Examples of solid cancers include, but are not limited to, cancer of the brain, prostate, breast, lung, colon, uterus, skin, liver, bone, pancreas, ovary, testes, bladder, kidney, head, neck, stomach, cervix, rectum, larynx, and esophagus. Examples of various autoimmune disorders include, but are not limited to, alopecia areata, autoimmune hemolytic anemia, autoimmune hepatitis, dermatomyositis, diabetes (type 1), some forms of juvenile idiopathic arthritis, glomerulonephritis, Graves' disease, Guillain-Barre syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus, erythematosus, some forms of thyroiditis, some forms of uveitis, vitiligo, granulomatosis with polyangiitis (Wegener's). Examples of viral infections include, but are not limited to, HIV- (human immunodeficiency virus), HSV- (herpes simplex virus), KSHV- (Kaposi's sarcoma-associated herpesvirus), RSV- (Respiratory Syncytial Virus), EBV- (Epstein-Barr virus), CMV- (cytomegalovirus), VZV (Varicella zoster virus), adenovirus-, a lentivirus-, a BK polyomavirus-associated disorders.

The treatment using the derived hematopoietic lineage cells of embodiments disclosed herein, or the compositions provided herein, could be carried out upon symptom, or for relapse prevention. The terms “treating,” “treatment,” and the like are used herein to generally mean obtaining a desired pharmacologic and/or physiologic effect. The effect may be prophylactic in terms of completely or partially preventing a disease and/or may be therapeutic in terms of a partial or complete cure for a disease and/or adverse effect attributable to the disease. “Treatment” as used herein covers any intervention of a disease in a subject and includes: preventing the disease from occurring in a subject which may be predisposed to the disease but has not yet been diagnosed as having it; inhibiting the disease, i.e., arresting its development; or relieving the disease, i.e., causing regression of the disease. The therapeutic agent(s) and/or compositions may be administered before, during or after the onset of a disease or an injury. The treatment of ongoing disease, where the treatment stabilizes or reduces the undesirable clinical symptoms of the patient, is also of particular interest. In particular embodiments, the subject in need of a treatment has a disease, a condition, and/or an injury that can be contained, ameliorated, and/or improved in at least one associated symptom by a cell therapy. Certain embodiments contemplate that a subject in need of cell therapy, includes, but is not limited to, a candidate for bone marrow or stem cell transplantation, a subject who has received chemotherapy or irradiation therapy, a subject who has or is at risk of having a hyperproliferative disorder or a cancer, e.g., a hyperproliferative disorder or a cancer of hematopoietic system, a subject having or at risk of developing a tumor, e.g., a solid tumor, a subject who has or is at risk of having a viral infection or a disease associated with a viral infection.

When evaluating responsiveness to the treatment comprising the derived hematopoietic lineage cells of embodiments disclosed herein, the response can be measured by criteria comprising at least one of: clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST (Response Evaluation Criteria In Solid Tumors) criteria.

The therapeutic composition comprising iPSC-derived hematopoietic lineage cells as disclosed can be administered in a subject before, during, and/or after other treatments. As such the method of a combinational therapy can involve the administration or preparation of iPSC-derived immune cells before, during, and/or after the use of one or more additional therapeutic agents. As provided above, the one or more additional therapeutic agents comprise a peptide, a cytokine, an engager, a checkpoint inhibitor, an engager, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD). The administration of the iPSC-derived immune cells can be separated in time from the administration of an additional therapeutic agent by hours, days, or even weeks. Additionally, or alternatively, the administration can be combined with other biologically active agents or modalities such as, but not limited to, an antineoplastic agent, a non-drug therapy, such as, surgery.

In some embodiments of a combinational cell therapy, the therapeutic combination comprises the iPSC-derived hematopoietic lineage cells provided herein and an additional therapeutic agent that is an engager, where the engager targets an antigen associated with a condition, a disease, or an indication (as described above). In some embodiments, the engager has a different tumor targeting specificity from the CAR of the engineered iPSC-derived hematopoietic lineage cells. In some embodiments, the engager is a bi-specific T cell engager (BiTE). In some embodiments, the engager is a bi-specific killer cell engager (BiKE). In some embodiments, the engager is a tri-specific killer cell engager (TriKE). In some embodiments, the engager is a multi-specific killer cell engager. In some embodiments, the engager is a universal engager compatible with multiple immune cell types.

In some embodiments of a combinational cell therapy, the therapeutic combination comprises the iPSC-derived hematopoietic lineage cells provided herein and an additional therapeutic agent that is an antibody, or an antibody fragment. In some embodiments, the antibody is a monoclonal antibody. In some embodiments, the antibody may be a humanized antibody, a humanized monoclonal antibody, or a chimeric antibody. In some embodiments, the antibody, or antibody fragment, specifically binds to a viral antigen. In other embodiments, the antibody, or antibody fragment, specifically binds to a tumor antigen. In some embodiments, the tumor or viral specific antigen activates the administered iPSC-derived hematopoietic lineage cells to enhance their killing ability. In some embodiments, the antibodies suitable for combinational treatment as an additional therapeutic agent to the administered iPSC-derived hematopoietic lineage cells include, but are not limited to, anti-CD20 (e.g., rituximab, veltuzumab, ofatumumab, ublituximab, ocaratuzumab, obinutuzumab, ibritumomab, ocrelizumab), anti-CD22 (inotuzumab, moxetumomab, epratuzumab), anti-HER2 (e.g., trastuzumab, pertuzumab), anti-CD52 (e.g., alemtuzumab), anti-EGFR (e.g., cetuximab), anti-GD2 (e.g., dinutuximab), anti-PDL1 (e.g., avelumab), anti-CD38 (e.g., daratumumab, isatuximab, MOR202), anti-CD123 (e.g., 7G3, CSL362), anti-SLAMF7 (elotuzumab), and their humanized or Fc modified variants or fragments or their functional equivalents or biosimilars. The present invention provides therapeutic compositions comprising the iPSC-derived hematopoietic lineage cells having a genotype listed in Table 1 and provided herein and an additional therapeutic agent that is an antibody, or an antibody fragment, as described above.

In some embodiments, the additional therapeutic agent comprises one or more checkpoint inhibitors. Checkpoints are referred to cell molecules, often cell surface molecules, capable of suppressing or downregulating immune responses when not inhibited. Checkpoint inhibitors are antagonists capable of reducing checkpoint gene expression or gene products, or deceasing activity of checkpoint molecules. Suitable checkpoint inhibitors for combination therapy with the derivative effector cells are provided above.

Some embodiments of the combination therapy comprising the provided derivative effector cells further comprise at least one inhibitor targeting a checkpoint molecule. Some other embodiments of the combination therapy with the provided derivative effector cells comprise two, three or more inhibitors such that two, three, or more checkpoint molecules are targeted. In some embodiments, the effector cells for combination therapy as described herein are derivative NK lineage cells as provided. In some embodiments, the effector cells for combination therapy as described herein are derivative T lineage cells. In some embodiments, the derivative NK or T lineage cells for combination therapies are functionally enhanced as provided herein. In some embodiments, the two, three or more checkpoint inhibitors may be administered in a combination therapy with, before, or after the administering of the derivative effector cells. In some embodiments, the two or more checkpoint inhibitors are administered at the same time, or one at a time (sequential). The present invention provides therapeutic compositions comprising effector cells, including, the iPSC-derived effector cells, having a genotype listed in Table 1 and one or more checkpoint inhibitors, as described above.

In some embodiments, the antagonist inhibiting any of the above checkpoint molecules is an antibody. In some embodiments, the checkpoint inhibitory antibodies may be murine antibodies, human antibodies, humanized antibodies, a camel Ig, single variable new antigen receptors (VNAR), shark heavy-chain antibodies (Ig-NAR), chimeric antibodies, recombinant antibodies, single-domain antibodies (dAb), anti-idiotype antibodies, bispecific-, multi-specific- or multimeric-antibodies, or antibody fragments thereof. Non-limiting examples of antibody fragments include Fab, Fab′, F(ab′)2, F(ab′)3, Fv, Fabc, pFc, Fd, single chain antigen binding fragments (scFv), tandem scFv (scFv)2, disulfide stabilized Fv (dsFv), minibody, diabody, triabody, tetrabody, single-domain antigen binding fragments (sdAb), camelid heavy-chain IgG and Nanobody® fragments, recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of atezolizumab, avelumab, durvalumab, ipilimumab, IPH4102, IPH43, IPH33, lirimumab, monalizumab, nivolumab, pembrolizumab, and their derivatives or functional equivalents.

The combination therapies comprising the derivative effector cells and one or more check inhibitors are applicable to treatment of liquid and solid cancers, including but not limited to cutaneous T-cell lymphoma, non-Hodgkin lymphoma (NHL), Mycosis fungoides, Pagetoid reticulosis, Sezary syndrome, Granulomatous slack skin, Lymphomatoid papulosis, Pityriasis lichenoides chronica, Pityriasis lichenoides et varioliformis acuta, CD30⁺ cutaneous T-cell lymphoma, Secondary cutaneous CD30⁺ large cell lymphoma, non-mycosis fungoides CD30 cutaneous large T-cell lymphoma, Pleomorphic T-cell lymphoma, Lennert lymphoma, subcutaneous T-cell lymphoma, angiocentric lymphoma, blastic NK-cell lymphoma, B-cell Lymphomas, hodgkins lymphoma (HL), Head and neck tumor; Squamous cell carcinoma, rhabdomyocarcoma, Lewis lung carcinoma (LLC), non-small cell lung cancer, esophageal squamous cell carcinoma, esophageal adenocarcinoma, renal cell carcinoma (RCC), colorectal cancer (CRC), acute myeloid leukemia (AML), breast cancer, gastric cancer, prostatic small cell neuroendocrine carcinoma (SCNC), liver cancer, glioblastoma, liver cancer, oral squamous cell carcinoma, pancreatic cancer, thyroid papillary cancer, intrahepatic cholangiocellular carcinoma, hepatocellular carcinoma, bone cancer, metastasis, and nasopharyngeal carcinoma.

In some embodiments, other than the derivative effector cells as provided herein, a combination for therapeutic use comprises one or more additional therapeutic agents comprising a chemotherapeutic agent or a radioactive moiety. Chemotherapeutic agent refers to cytotoxic antineoplastic agents, that is, chemical agents which preferentially kill neoplastic cells or disrupt the cell cycle of rapidly-proliferating cells, or which are found to eradicate stem cancer cells, and which are used therapeutically to prevent or reduce the growth of neoplastic cells. Chemotherapeutic agents are also sometimes referred to as antineoplastic or cytotoxic drugs or agents, and are well known in the art.

In some embodiments, the chemotherapeutic agent comprises an anthracycline, an alkylating agent, an alkyl sulfonate, an aziridine, an ethylenimine, a methylmelamine, a nitrogen mustard, a nitrosourea, an antibiotic, an antimetabolite, a folic acid analog, a purine analog, a pyrimidine analog, an enzyme, a podophyllotoxin, a platinum-containing agent, an interferon, and an interleukin. Exemplary chemotherapeutic agents include, but are not limited to, alkylating agents (cyclophosphamide, mechlorethamine, mephalin, chlorambucil, heamethylmelamine, thiotepa, busulfan, carmustine, lomustine, semustine), animetabolites (methotrexate, fluorouracil, floxuridine, cytarabine, 6-mercaptopurine, thioguanine, pentostatin), vinca alkaloids (vincristine, vinblastine, vindesine), epipodophyllotoxins (etoposide, etoposide orthoquinone, and teniposide), antibiotics (daunorubicin, doxorubicin, mitoxantrone, bisanthrene, actinomycin D, plicamycin, puromycin, and gramicidine D), paclitaxel, colchicine, cytochalasin B, emetine, maytansine, and amsacrine. Additional agents include aminglutethimide, cisplatin, carboplatin, mitomycin, altretamine, cyclophosphamide, lomustine (CCNU), carmustine (BCNU), irinotecan (CPT-11), alemtuzamab, altretamine, anastrozole, L-asparaginase, azacitidine, bevacizumab, bexarotene, bleomycin, bortezomib, busulfan, calusterone, capecitabine, celecoxib, cetuximab, cladribine, clofurabine, cytarabine, dacarbazine, denileukin diftitox, diethlstilbestrol, docetaxel, dromostanolone, epirubicin, erlotinib, estramustine, etoposide, ethinyl estradiol, exemestane, floxuridine, 5-flourouracil, fludarabine, flutamide, fulvestrant, gefitinib, gemcitabine, goserelin, hydroxyurea, ibritumomab, idarubicin, ifosfamide, imatinib, interferon alpha (2a, 2b), irinotecan, letrozole, leucovorin, leuprolide, levamisole, meclorethamine, megestrol, melphalin, mercaptopurine, methotrexate, methoxsalen, mitomycin C, mitotane, mitoxantrone, nandrolone, nofetumomab, oxaliplatin, paclitaxel, pamidronate, pemetrexed, pegademase, pegasparagase, pentostatin, pipobroman, plicamycin, polifeprosan, porfimer, procarbazine, quinacrine, rituximab, sargramostim, streptozocin, tamoxifen, temozolomide, teniposide, testolactone, thioguanine, thiotepa, topetecan, toremifene, tositumomab, trastuzumab, tretinoin, uracil mustard, valrubicin, vinorelbine, and zoledronate. Other suitable agents are those that are approved for human use, including those that will be approved, as chemotherapeutics or radiotherapeutics, and known in the art. Such agents can be referenced through any of a number of standard physicians' and oncologists' references (e.g., Goodman & Gilman's The Pharmacological Basis of Therapeutics, Ninth Edition, McGraw-Hill, N.Y., 1995) or through the National Cancer Institute website (fda.gov/cder/cancer/druglistfrarne.htm), both as updated from time to time.

Immunomodulatory drugs (IMiDs) such as thalidomide, lenalidomide, and pomalidomide stimulate both NK cells and T cells. As provided herein, IMiDs may be used with the iPSC-derived therapeutic immune cells for cancer treatments.

Other than an isolated population of iPSC-derived hematopoietic lineage cells and/or engagers included in the therapeutic compositions, the compositions suitable for administration to a subject/patient can further include one or more pharmaceutically acceptable carriers (additives) and/or diluents (e.g., pharmaceutically acceptable medium, for example, cell culture medium), or other pharmaceutically acceptable components. Pharmaceutically acceptable carriers and/or diluents are determined in part by the particular composition being administered, as well as by the particular method used to administer the therapeutic composition. Accordingly, there is a wide variety of suitable formulations of therapeutic compositions of the present invention (see, e.g., Remington's Pharmaceutical Sciences, 17^{th} ed. 1985, the disclosure of which is hereby incorporated by reference in its entirety).

In one embodiment, the therapeutic composition comprises the iPSC-derived T cells made by the methods and compositions disclosed herein. In one embodiment, the therapeutic composition comprises the pluripotent cell derived NK cells made by the methods and composition disclosed herein. In one embodiment, the therapeutic composition comprises the iPSC-derived CD34⁺ HE cells made by the methods and composition disclosed herein. In one embodiment, the therapeutic composition comprises the pluripotent cell derived HSCs made by the methods and composition disclosed herein. In one embodiment, the therapeutic composition comprises the pluripotent cell derived MDSC made by the methods and composition disclosed herein. A therapeutic composition comprising a population of iPSC-derived hematopoietic lineage cells, optionally in combination with an engager having a different tumor targeting specificity from the CAR of the iPSC-derived hematopoietic lineage cells, as disclosed herein can be administered separately by intravenous, intraperitoneal, enteral, or tracheal administration methods or in combination with other suitable compounds to affect the desired treatment goals.

These pharmaceutically acceptable carriers and/or diluents can be present in amounts sufficient to maintain a pH of the therapeutic composition of between about 3 and about 10. As such, the buffering agent can be as much as about 5% on a weight to weight basis of the total composition. Electrolytes such as, but not limited to, sodium chloride and potassium chloride can also be included in the therapeutic composition. In one aspect, the pH of the therapeutic composition is in the range from about 4 to about 10. Alternatively, the pH of the therapeutic composition is in the range from about 5 to about 9, from about 6 to about 9, or from about 6.5 to about 8. In another embodiment, the therapeutic composition includes a buffer having a pH in one of said pH ranges. In another embodiment, the therapeutic composition has a pH of about 7. Alternatively, the therapeutic composition has a pH in a range from about 6.8 to about 7.4. In still another embodiment, the therapeutic composition has a pH of about 7.4.

The invention also provides, in part, the use of a pharmaceutically acceptable cell culture medium in particular compositions and/or cultures of the present invention. Such compositions are suitable for administration to human subjects. Generally speaking, any medium that supports the maintenance, growth, and/or health of the iPSC-derived immune cells in accordance with embodiments of the invention are suitable for use as a pharmaceutical cell culture medium. In particular embodiments, the pharmaceutically acceptable cell culture medium is a serum free, and/or feeder-free medium. In various embodiments, the serum-free medium is animal-free, and can optionally be protein-free. Optionally, the medium can contain biopharmaceutically acceptable recombinant proteins. Animal-free medium refers to medium wherein the components are derived from non-animal sources. Recombinant proteins replace native animal proteins in animal-free medium and the nutrients are obtained from synthetic, plant or microbial sources. Protein-free medium, in contrast, is defined as substantially free of protein. One having ordinary skill in the art would appreciate that the above examples of media are illustrative and in no way limit the formulation of media suitable for use in the present invention and that there are many suitable media known and available to those in the art.

The iPSC-derived hematopoietic lineage cells can have at least 50%, 60%, 70%, 80%, 90%, 95%, 98%, or 99% T lineage cells, NK lineage cells, NKT lineage cells, proT cells, proNK cells, CD34⁺ HE cells, HSCs, B lineage cells, myeloid-derived suppressor cells (MDSCs), regulatory macrophages, regulatory dendritic cells, or mesenchymal stromal cells. In some embodiments, the isolated pluripotent stem cell derived hematopoietic lineage cells has about 95% to about 100% T lineage cells, NK lineage cells, proT cells, proNK cells, CD34⁺ HE cells, or myeloid-derived suppressor cells (MDSCs). In some embodiments, the present invention provides therapeutic compositions having purified iPSC derivative effector cells, such as a composition having an isolated population of about 95% T lineage cells, NK lineage cells, proT cells, proNK cells, CD34⁺ HE cells, or myeloid-derived suppressor cells (MDSCs) to treat a subject in need of the cell therapy.

In one embodiment, the combinational cell therapy, or composition used therefor, comprises a therapeutic protein or peptide and a population of effector cells derived from genomically engineered iPSCs comprising a genotype listed in Table 1, wherein the derived effector cells comprise a CAR and one or both of an engager and a CFR, as described herein. In some embodiments, the combinational cell therapy, or composition used therefor, comprises one of blinatumomab, catumaxomab, ertumaxomab, R06958688, AFM11, MT110/AMG 110, MT111/AMG211/MEDI-565, AMG330, MT112/BAY2010112, MOR209/ES414, MGD006/S80880, MGD007, and/or FBTA05, and a population of effector cells derived from genomically engineered iPSCs comprising a genotype listed in Table 1, wherein the derived effector cells comprise a CAR and one or both of an engager and a CFR, and optionally, an exogenous CD16 or a variant thereof, or other edits. In yet some other embodiments, the combinational cell therapy, or composition used therefor, comprises one of blinatumomab, catumaxomab, and ertumaxomab, and a population of effector cells derived from genomically engineered iPSCs comprising a genotype listed in Table 1, wherein the derived effector cells comprise a CAR and one or both of an engager and a CFR, exogenous CD16 or a variant thereof, and IL. In still some additional embodiments, the combinational cell therapy, or composition used therefor, comprises one of blinatumomab, catumaxomab, and ertumaxomab, and a population of effector cells derived from genomically engineered iPSCs comprising a genotype listed in Table 1, wherein the derived effector cells comprise a CAR and one or both of an engager and a CFR, exogenous CD16 or a variant thereof, CD38 knock out and one or more exogenous cytokines.

As a person of ordinary skill in the art would understand, both autologous and allogeneic hematopoietic lineage cells derived from iPSC based on the methods and compositions provided herein can be used in cell therapies as described above. For autologous transplantation, the isolated population of derived hematopoietic lineage cells are either complete or partial HLA-match with the patient. In another embodiment, the derived hematopoietic lineage cells are not HLA-matched to the subject, wherein the derived hematopoietic lineage cells are NK cells or T cell with HLA-I and/or HLA-II deficiency.

In some embodiments, the number of derived hematopoietic lineage cells in the therapeutic composition is at least 0.1×10⁵ cells, at least 1×10⁵ cells, at least 5×10⁵ cells, at least 1×10⁶ cells, at least 5×10⁶ cells, at least 1×10⁷ cells, at least 5×10⁷ cells, at least 1×10⁸ cells, at least 5×10⁸ cells, at least 1×10⁹ cells, or at least 5×10⁹ cells, per dose. In some embodiments, the number of derived hematopoietic lineage cells in the therapeutic composition is about 0.1×10⁵ cells to about 1×10⁶ cells, per dose; about 0.5×10⁶ cells to about 1×10⁷ cells, per dose; about 0.5×10⁷ cells to about 1×10⁸ cells, per dose; about 0.5×10⁸ cells to about 1×10⁹ cells, per dose; about 1×10⁹ cells to about 5×10⁹ cells, per dose; about 0.5×10⁹ cells to about 8×10⁹ cells, per dose; about 3×10⁹ cells to about 3×10¹⁰ cells, per dose, or any range in-between. Generally, 1×10⁸ cells/dose translates to 1.67×10⁶ cells/kg for a 60 kg patient.

In one embodiment, the number of derived hematopoietic lineage cells in the therapeutic composition is the number of immune cells in a partial or single cord of blood, or is at least 0.1×10⁵ cells/kg of bodyweight, at least 0.5×10⁵ cells/kg of bodyweight, at least 1×10⁵ cells/kg of bodyweight, at least 5×10⁵ cells/kg of bodyweight, at least 10×10⁵ cells/kg of bodyweight, at least 0.75×10⁶ cells/kg of bodyweight, at least 1.25×10⁶ cells/kg of bodyweight, at least 1.5×10⁶ cells/kg of bodyweight, at least 1.75×10⁶ cells/kg of bodyweight, at least 2×10⁶ cells/kg of bodyweight, at least 2.5×10⁶ cells/kg of bodyweight, at least 3×10⁶ cells/kg of bodyweight, at least 4×10⁶ cells/kg of bodyweight, at least 5×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, at least 30×10⁶ cells/kg of bodyweight, 1×10⁸ cells/kg of bodyweight, 5×10⁸ cells/kg of bodyweight, or 1×10⁹ cells/kg of bodyweight.

In one embodiment, a dose of derived hematopoietic lineage cells is delivered to a subject. In one illustrative embodiment, the effective amount of cells provided to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In another illustrative embodiment, the effective amount of cells provided to a subject is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, or about 10×10⁶ cells/kg, or more cells/kg, including all intervening doses of cells.

In another illustrative embodiment, the effective amount of cells provided to a subject is from about 2×10⁶ cells/kg to about 10×10⁶ cells/kg, about 3×10⁶ cells/kg to about 10×10⁶ cells/kg, about 4×10⁶ cells/kg to about 10×10⁶ cells/kg, about 5×10⁶ cells/kg to about 10×10⁶ cells/kg, 2×10⁶ cells/kg to about 6×10⁶ cells/kg, 2×10⁶ cells/kg to about 7×10⁶ cells/kg, 2×10⁶ cells/kg to about 8×10⁶ cells/kg, 3×10⁶ cells/kg to about 6×10⁶ cells/kg, 3×10⁶ cells/kg to about 7×10⁶ cells/kg, 3×10⁶ cells/kg to about 8×10⁶ cells/kg, 4×10⁶ cells/kg to about 6×10⁶ cells/kg, 4×10⁶ cells/kg to about 7×10⁶ cells/kg, 4×10⁶ cells/kg to about 8×10⁶ cells/kg, 5×10⁶ cells/kg to about 6×10⁶ cells/kg, 5×10⁶ cells/kg to about 7×10⁶ cells/kg, 5×10⁶ cells/kg to about 8×10⁶ cells/kg, or 6×10⁶ cells/kg to about 8×10⁶ cells/kg, including all intervening doses of cells.

In some embodiments, the therapeutic use of derived hematopoietic lineage cells is a single-dose treatment. In some embodiments, the therapeutic use of derived hematopoietic lineage cells is a multi-dose treatment. In some embodiments, the multi-dose treatment is one dose every day, every 3 days, every 7 days, every 10 days, every 15 days, every 20 days, every 25 days, every 30 days, every 35 days, every 40 days, every 45 days, or every 50 days, or any number of days in-between. In some embodiments, the multi-dose treatment comprises three, or four, or five, once weekly doses. In some embodiments of the multi-dose treatment comprising three, or four, or five, once weekly doses further comprise an observation period for determining whether additional single or multi doses are needed.

The compositions comprising a population of derived hematopoietic lineage cells and optionally an engager, of the invention can be sterile, and can be suitable and ready for administration (i.e., can be administered without any further processing) to human patients. A cell based composition that is ready for administration means that the composition does not require any further processing or manipulation prior to transplant or administration to a subject. In other embodiments, the invention provides an isolated population of derived hematopoietic lineage cells that are expanded and/or modulated prior to administration with one or more agents. For derived hematopoietic lineage cells that are genetically engineered to express a signaling complex and/or a CAR, the cells can be activated and expanded using methods as described, for example, in U.S. Pat. No. 6,352,694, the entire content of which is incorporated herein by reference.

In certain embodiments, the primary stimulatory signal and the co-stimulatory signal for the derived hematopoietic lineage cells can be provided by different protocols. For example, the agents providing each signal can be in solution or coupled to a surface. When coupled to a surface, the agents can be coupled to the same surface (i.e., in “cis” formation) or to separate surfaces (i.e., in “trans” formation). Alternatively, one agent can be coupled to a surface and the other agent in solution. In one embodiment, the agent providing the co-stimulatory signal can be bound to a cell surface and the agent providing the primary activation signal is in solution or coupled to a surface. In certain embodiments, both agents can be in solution. In another embodiment, the agents can be in soluble form, and then cross-linked to a surface, such as a cell expressing Fc receptors or an antibody or other binding agent which will bind to the agents such as disclosed in U.S. Pub. Nos. 2004/0101519 and 2006/0034810 for artificial antigen presenting cells (aAPCs) that are contemplated for use in activating and expanding T lymphocytes in embodiments of the present invention.

Some variation in dosage, frequency, and protocol will necessarily occur depending on the condition of the subject being treated. The person responsible for administration will, in any event, determine the appropriate dose, frequency and protocol for the individual subject.

### EXAMPLES

The following examples are offered by way of illustration and not by way of limitation.

### Example 1—Materials and Methods

To effectively select and test suicide systems under the control of various promoters in combination with different safe harbor loci integration strategies, a proprietary hiPSC platform of the applicant was used, which enables single cell passaging and high-throughput, 96-well plate-based flow cytometry sorting, to allow for the derivation of clonal hiPSCs with single or multiple genetic modulations.

hiPSC Maintenance in Small Molecule Culture: hiPSCs were routinely passaged as single cells once confluency of the culture reached 75%-90%. For single-cell dissociation, hiPSCs were washed once with PBS (Mediatech) and treated with Accutase (Millipore) for 3-5 min at 37° C. followed with pipetting to ensure single-cell dissociation. The single-cell suspension was then mixed in equal volume with conventional medium, centrifuged at 225×g for 4 min, resuspended in FMM, and plated on Matrigel-coated surface. Passages were typically 1:6-1:8, transferred tissue culture plates previously coated with Matrigel for 2-4 hr in 37° C. and fed every 2-3 days with FMM. Cell cultures were maintained in a humidified incubator set at 37° C. and 5% CO₂.

Human iPSC engineering with ZFN, CRISPR for targeted editing of modalities of interest: Using ROSA26 targeted insertion as an example, for ZFN mediated genome editing, 2 million iPSCs were transfected with a mixture of 2.5 μg ZFN-L (FTV893), 2.5 μg ZFN-R (FTV894) and 5 μg donor construct, for AAVS1 targeted insertion. For CRISPR mediated genome editing, 2 million iPSCs were transfected with a mixture of 5 μg ROSA26-gRNA/Cas9 (FTV922) and 5 μg donor construct, for ROSA26 targeted insertion. Transfection was done using Neon transfection system (Life Technologies) using parameters 1500V, 10 ms, 3 pulses. On day 2 or 3 after transfection, transfection efficiency was measured using flow cytometry if the plasmids contain artificial promoter-driver GFP and/or RFP expression cassette. On day 4 after transfection, puromycin was added to the medium at concentration of 0.1 μg/ml for the first 7 days and 0.2 μg/ml after 7 days to select the targeted cells. During the puromycin selection, the cells were passaged onto fresh matrigel-coated wells on day 10. On day 16 or later of puromycin selection, the surviving cells were analyzed by flow cytometry for GFP⁺ iPS cell percentage.

Bulk sort and clonal sort of genome-edited iPSCs: iPSCs with genomic targeted editing using ZFN or CRISPR-Cas9 were bulk sorted and clonal sorted for GFP⁺SSEA4⁺TRA181⁺ iPSCs after 20 days of puromycin selection. Single cell dissociated targeted iPSC pools were resuspended in chilled staining buffer containing Hanks' Balanced Salt Solution (MediaTech), 4% fetal bovine serum (Invitrogen), lx penicillin/streptomycin (Mediatech) and 10 mM Hepes (Mediatech); made fresh for optimal performance. Conjugated primary antibodies, including SSEA4-PE, TRA181-Alexa Fluor-647 (BD Biosciences), were added to the cell solution and incubated on ice for 15 minutes. All antibodies were used at 7 μL in 100 μL staining buffer per million cells. The solution was washed once in staining buffer, spun down at 225 g for 4 minutes and resuspended in staining buffer containing 10 μM Thiazovivn and maintained on ice for flow cytometry sorting. Flow cytometry sorting was performed on FACS Aria II (BD Biosciences). For bulk sort, GFP⁺SSEA4⁺TRA181V cells were gated and sorted into 15 ml canonical tubes filled with 7 ml FMM. For clonal sort, the sorted cells were directly ejected into 96-well plates using the 100 μM nozzle, at concentrations of 3 events per well. Each well was prefilled with 200 μL FMM supplemented with 5 μg/mL fibronectin and 1× penicillin/streptomycin (Mediatech) and previously coated overnight with 5× Matrigel. 5× Matrigel precoating includes adding one aliquot of Matrigel into 5 mL of DMEM/F12, then incubating overnight at 4° C. to allow for proper resuspension and finally adding to 96-well plates at 50 μL per well, followed by overnight incubation at 37° C. The 5× Matrigel is aspirated immediately before the addition of media to each well. Upon completion of the sort, 96-well plates were centrifuged for 1-2 min at 225 g prior to incubation. The plates were left undisturbed for seven days. On the seventh day, 150 μL of medium was removed from each well and replaced with 100 μL FMM. Wells were refed with an additional 100 μL FMM on day 10 post sort. Colony formation was detected as early as day 2 and most colonies were expanded between days 7-10 post sort. In the first passage, wells were washed with PBS and dissociated with 30 μL Accutase for approximately 10 min at 37° C. The need for extended Accutase treatment reflects the compactness of colonies that have sat idle in culture for prolonged duration. After cells are seen to be dissociating, 200 μL of FMM is added to each well and pipetted several times to break up the colony. The dissociated colony is transferred to another well of a 96-well plate previously coated with 5× Matrigel and then centrifuged for 2 min at 225 g prior to incubation. This 1:1 passage is conducted to spread out the early colony prior to expansion. Subsequent passages were done routinely with Accutase treatment for 3-5 min and expansion of 1:4-1:8 upon 75-90% confluency into larger wells previously coated with 1× Matrigel in FMM. Each clonal cell line was analyzed for GFP fluorescence level and TRA1-81 expression level. Clonal lines with near 100% GFP⁺ and TRA1-81V were selected for further PCR screening and analysis, and cryopreserved as a master cell bank. Flow cytometry analysis was performed on Guava EasyCyte 8 HT (Millipore) and analyzed using Flowjo (FlowJo, LLC).

### Example 2—T Cell Engagers Improve CAR-T Efficacy in Tumor Models

Although CAR-T cells have been shown to be effective and potent in treating several hematologic malignancies, engineered T cell therapies have had limited success in addressing solid tumors. Unlike liquid tumors where uniformly-expressed antigens are accessible and can be effectively targeted, tumor access and antigen heterogeneity are a significant barrier to the successful development of CAR-T cells in solid tumors.

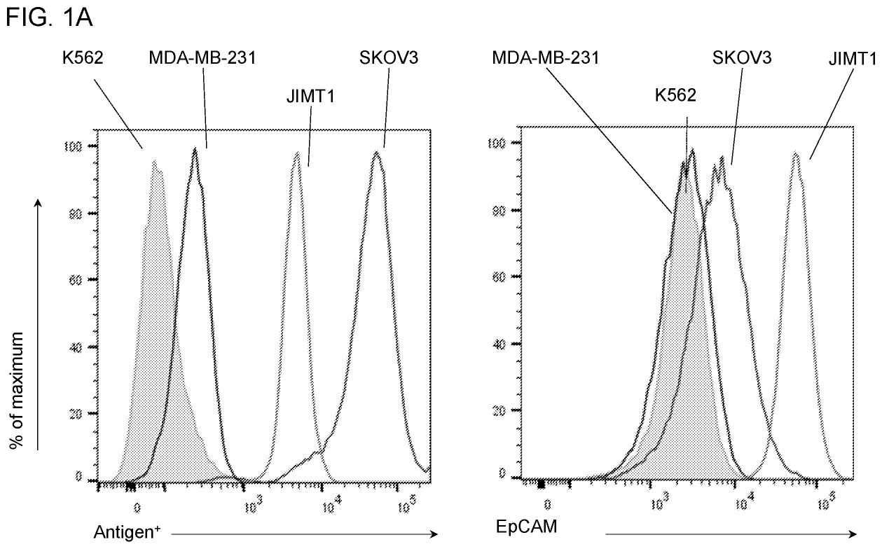

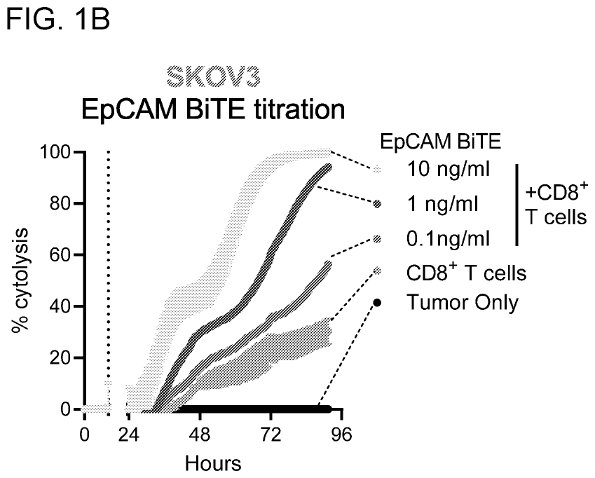

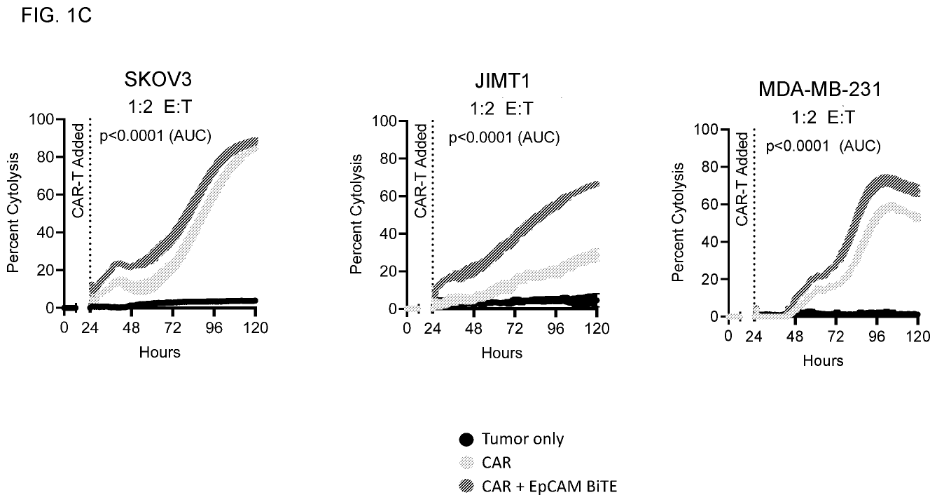

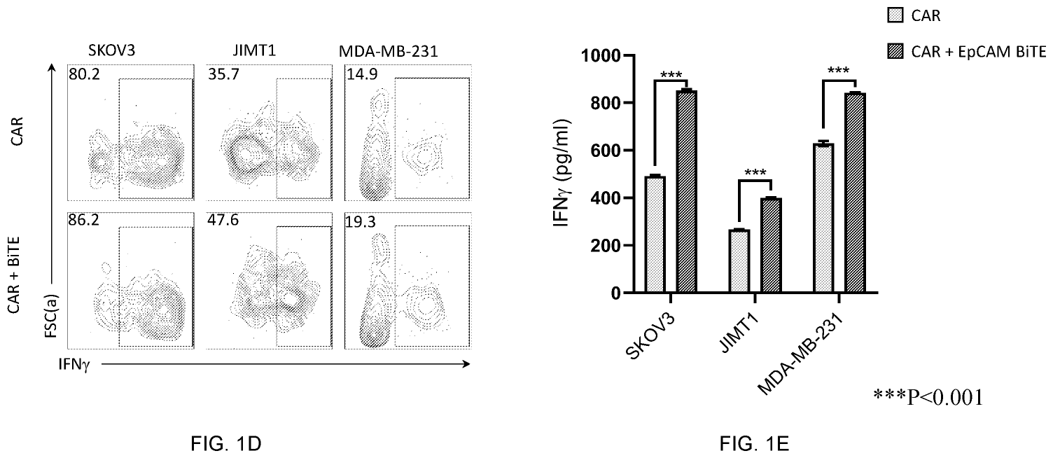

For the purpose of proof-of-concept, a combination of a bi-specific T cell engager (BiTE) targeting EpCAM with an antigen-specific CAR-T cell that targets a different antigen was investigated for their combined anti-tumor activity against heterogenous solid tumors. The expression level of a specific tumor antigen and the EpCAM antigen was assessed by flow cytometry in several tumor cells. As shown in FIG. 1A, SKOV3 is an Antigen^{High} tumor cell line, MDA-MB-231 is an Antigen^{Low} tumor cell line, whereas JIMT1 is medium in Antigen expression (Antigen^{Med}). SKOV3 cells were then co-cultured with CAR-T cells at an effector:target ratio of 10:1, and with EpCAM BiTE at the indicated concentrations ranging from 0 to 10 ng/ml for T cell cytolysis assessment. An engager dose-specific enhancement of CAR-T cytolytic activity was shown using the EpCAM-specific BiTE titration alongside with the Antigen-specific CAR-T cells (FIG. 1B). Further, SKOV3, JIMT1, and MDA-MB-231 tumor cells were co-cultured with the CAR-T cells at an E:T ratio of 1:2 with or without EpCAM BiTEs. The specific cytolysis of the CAR-T cell under different conditions was measured via xCelligence™ RTCA assay (FIG. 1C), with IFNγ production determined via intracellular cytokine staining (FIG. 1D) and IFNγ specific ELISA (FIG. 1E), all of which showed that T cell engagers improve CAR-T efficacy in 2D tumor models even at a low E:T ratio.

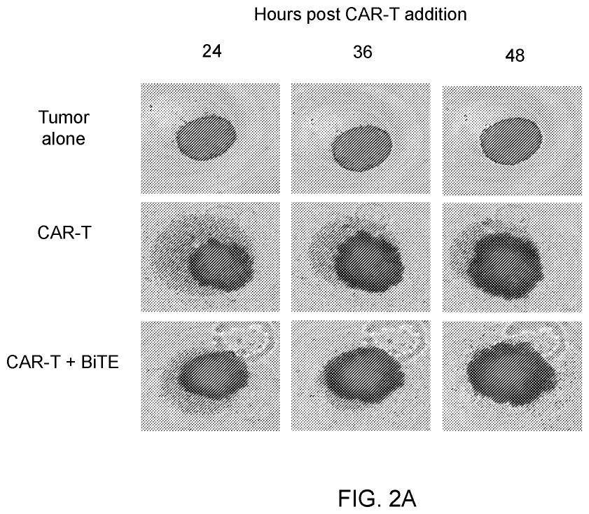

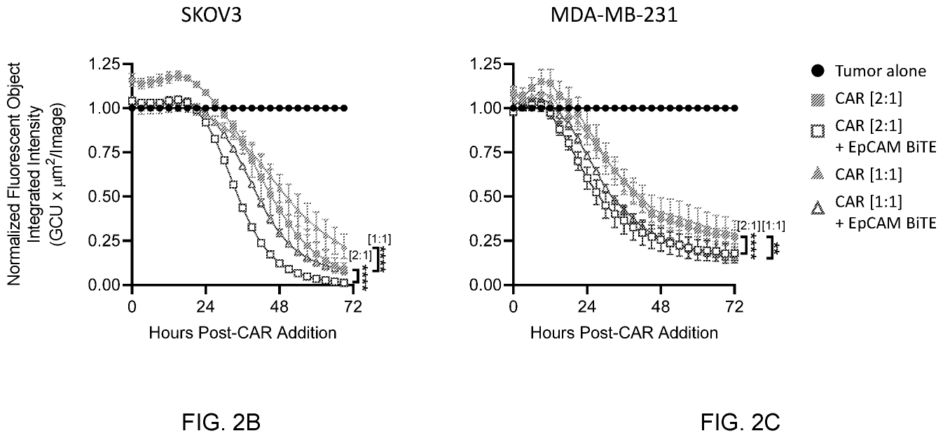

3D spheroids were established from SKOV3 or MDA-MB-231 tumor cells and co-cultured with the CAR-T cells at the indicated E:T ratios with and without EpCAM BiTEs. FIG. 2A shows representative images from SKOV3 tumor spheroids co-cultured with the CAR-T cells (2:1) with and without EpCAM BiTEs at the indicated time points, showing improved tumor penetration in the presence of combined CAR-T and BiTEs. As shown in FIG. 2B, SKOV3 spheroid clearance, determined via Incucyte assay and normalized to tumor alone, increased at a higher E:T ratio. Likewise, as shown in FIG. 2C, MDA-MB-231 spheroid clearance increased (although to a lesser extent) at a higher E:T ratio.

### Example 3—Multi-Antigen Targeting Controls Heterogenous Mixed Tumors

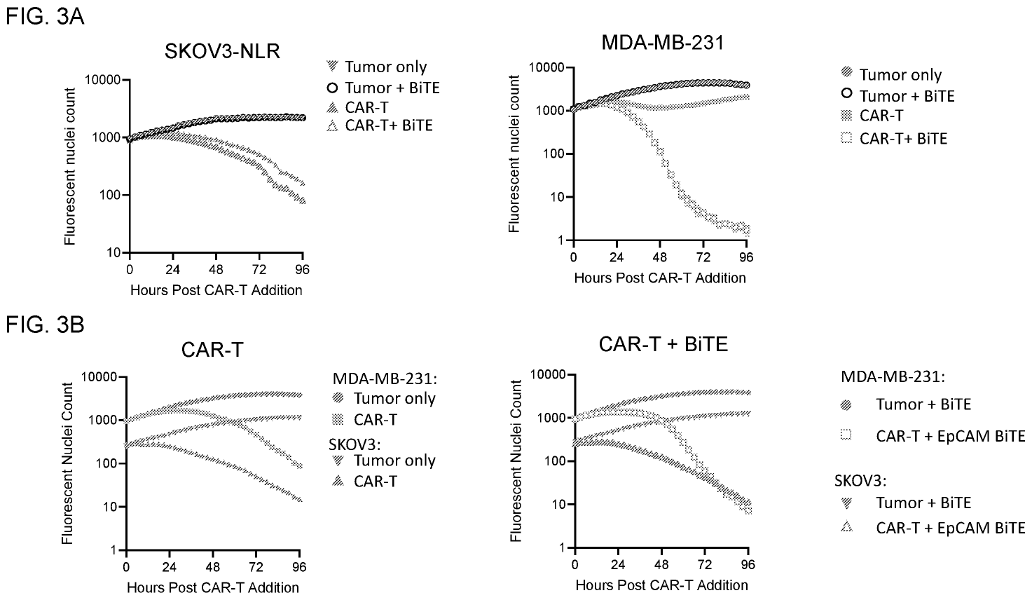

To demonstrate tumor growth control in a heterogenous mixed tumor environment, SKOV3-Nuc light red (NLR; Antigen^{High}) or MDA-MB-231-Nuc light green (NLG; Antigen^{Low}) were plated. Separately, a 3:1 (E:T) mixed culture of MDA-MB-231-NLG:SKOV3-NLR was plated. 24 hours after plating, the antigen-specific CAR-T cells, with and without the BiTEs, were co-cultured for an additional 96 hours at a 3:1 (E:T) ratio. Tumor cell cytolysis of the standard, or individual, cultures, as monitored via Incucyte™ assay, is shown in FIG. 3A, while cytoloysis of the mixed tumor cell cultures is shown in FIG. 3B. The CAR-T cells alone were unable to contain Antigen^{Low} tumors (FIG. 3A, right panel), whereas tumor growth was effectively controlled with the presence of both EpCAM-specific BiTEs and the CAR-T cells. As shown in FIG. 3B, the escape of Antigen^{Low} tumor cells in mixed heterogenous culture systems when using CAR-T cells alone was circumvented by the combination of the CAR-T cells and EpCAM-specific BiTEs.

Collectively, these data demonstrate that multi-antigen targeting mediated by BiTEs and CARs extends overall anti-tumor efficacy in preclinical models of heterogenous solid tumors.

### Example 4—B7H3 TriKE and Engineered iPSC-Derived Effector Cell for Enhanced Function and Specificity Against a Broad Range of Solid Tumors

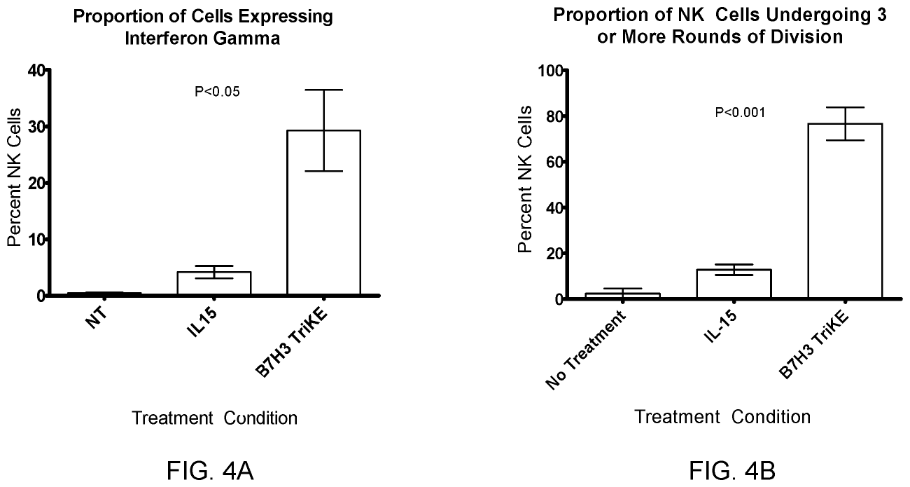

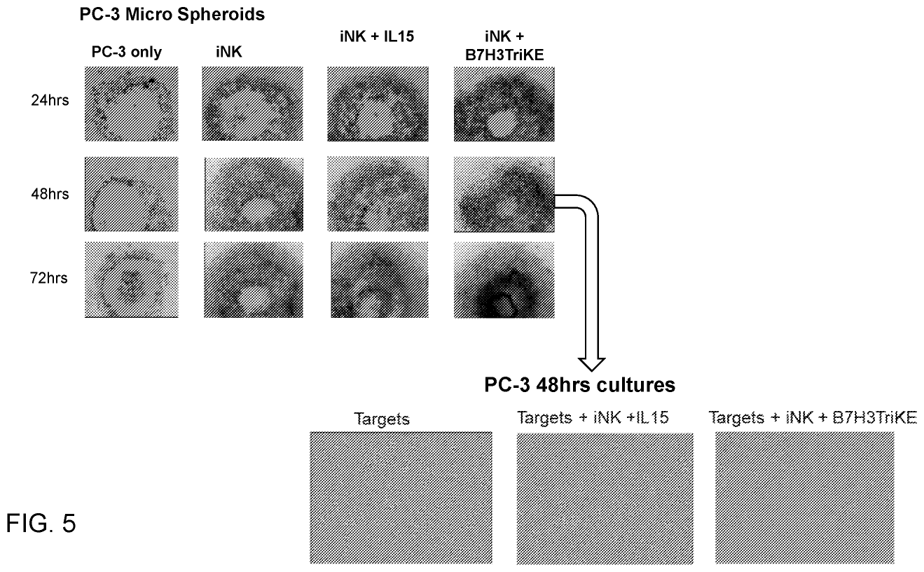

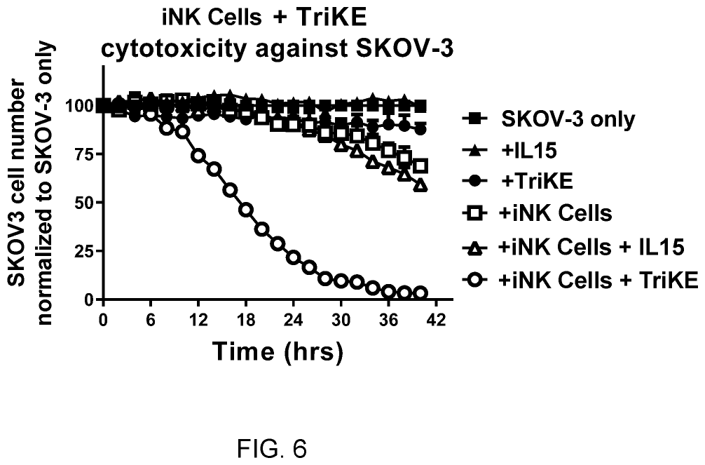

To further test the combinational use of an engager, a B7H3 TRiKE (tri-specific killer cell engager) was applied in multiple cell composition designs. B7H3 (CD276) belongs to the B7 family of immune checkpoint inhibitors, and has a wide range of expression on both solid and hematologic malignancies. B7H3 is an important mediator of tumor angiogenesis and metastasis, and a higher B7H3 expression in tumor generally correlates with poor prognosis. The B7H3 TriKE used here (B7H3-IL15-CD16) comprises an IL15 component, and its presence increased NK cell activation (FIG. 4A) and IL15 supplementation independent proliferation (FIG. 4B) compared to exogenous IL15 culturing condition. Using 3D spheroid tumor models of B7H3-expressing prostate cancer cells PC-3, it was shown in FIG. 5 that the engineered iPSC derived CAR-NK lineage cells target the prostate cancer cells more effectively in combination with the B7H3 TriKE. Further, as shown in FIG. 6, a combination of the engineered iPSC-derived CAR-NK lineage cells and B7H3 TriKE efficiently targeted SKOV-3 ovarian cancer cells in an Incucyte cytotoxicity assay. SKOV-3-NLR cells were plated one day before addition of effectors, which were added in a 2:1 effector-to-target (E:T) ratio in the presence anti-B7H3 TriKE or equimolar concentrations of IL-15.

### Example 5—Stepwise Engineering of iPSC and Validation of Modified Derivative Effector Cells

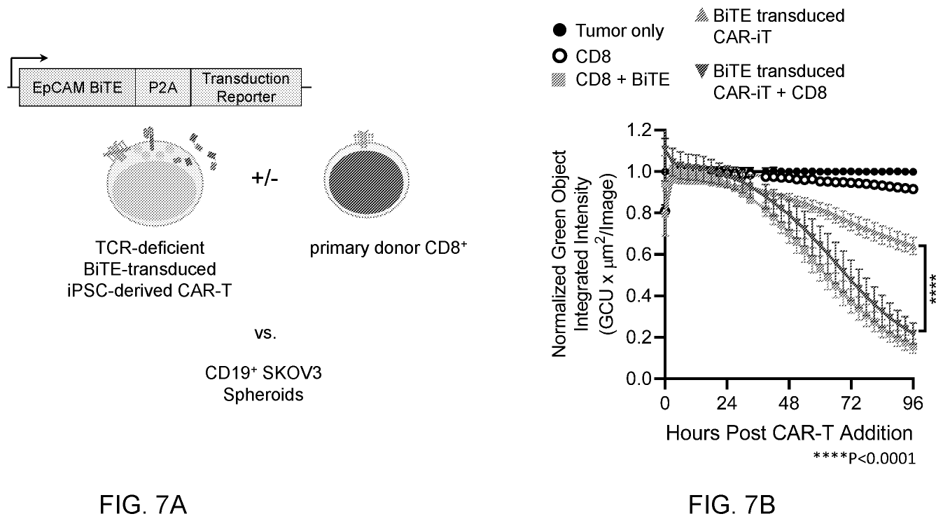

Next, to explore the feasibility of incorporating multi-antigen targeting and recruitment potential into CAR-iT cells by autonomous cell secretion of engager including BiTE or TRiKE through rational cell design, iPSC cells specific for CD19 were transduced with lentivirus carrying an EpCAM BiTE for proof-of-concept to determine effector cell differentiation and function profile (FIG. 7A). Reporter positive CAR-iT cells derived from said engineered iPSCs were co-cultured with CD19⁺ SKOV3 tumor spheroids along with primary unactivated CD8⁺ T cells. In separate control wells primary CD8⁺ T cells and BiTEs were co-cultured with CD19⁺ SKOV3 tumor spheroids and the spheroid clearance was measured via Incucyte assay. As shown in FIG. 7B, iPSC-derived CAR-T cells engineered to produce BiTEs activate and engage allogeneic CD8⁺ T cells to target tumor cells, indicating the capability of by-stander immune cell recruiting, infiltration and activation.

### Example 6—B7H3-CAR Configuration and B7H3-CAR Effector Cell Function Against Cancer Cells

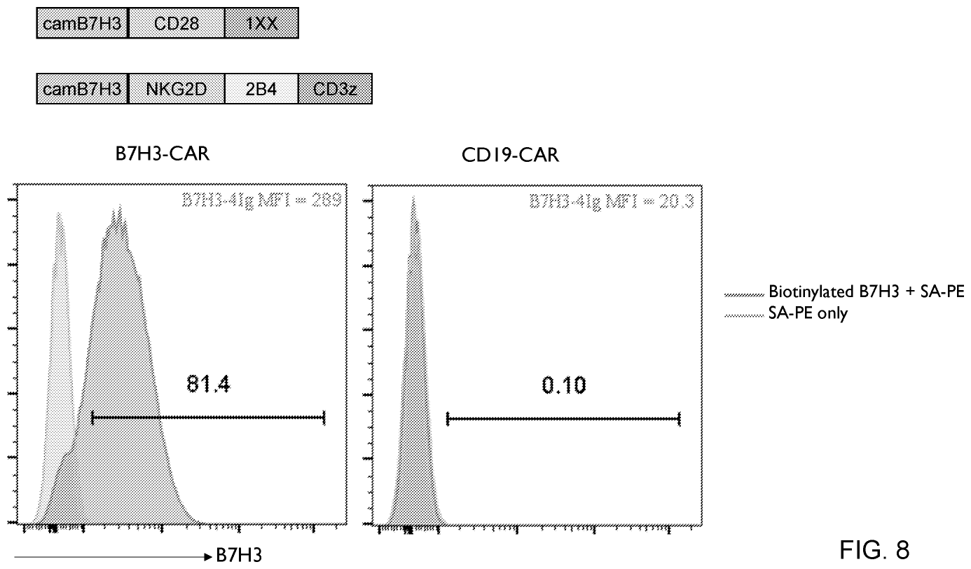

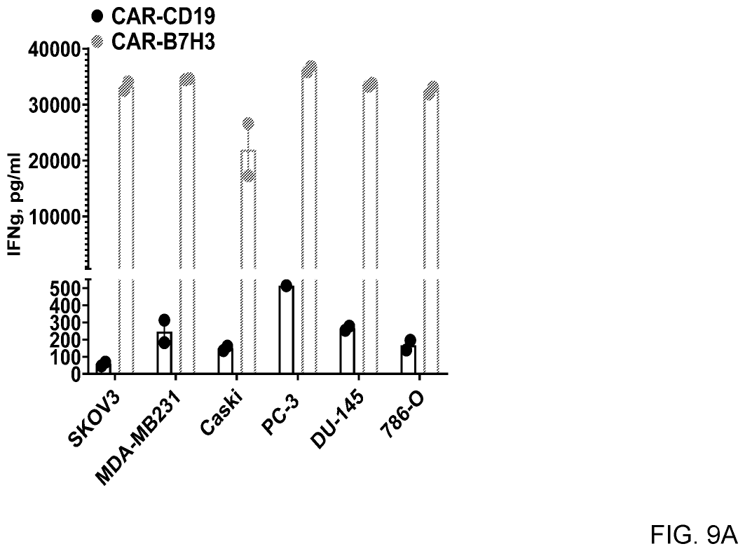

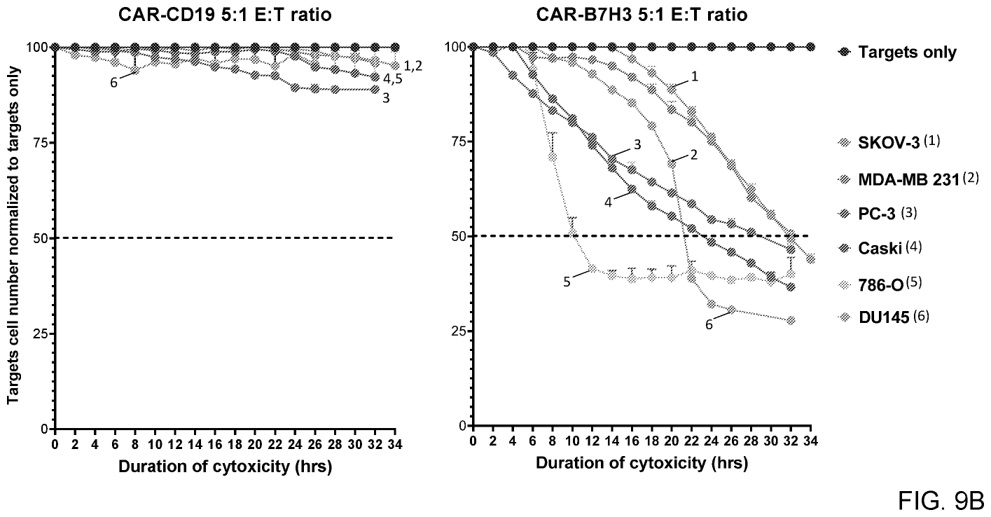

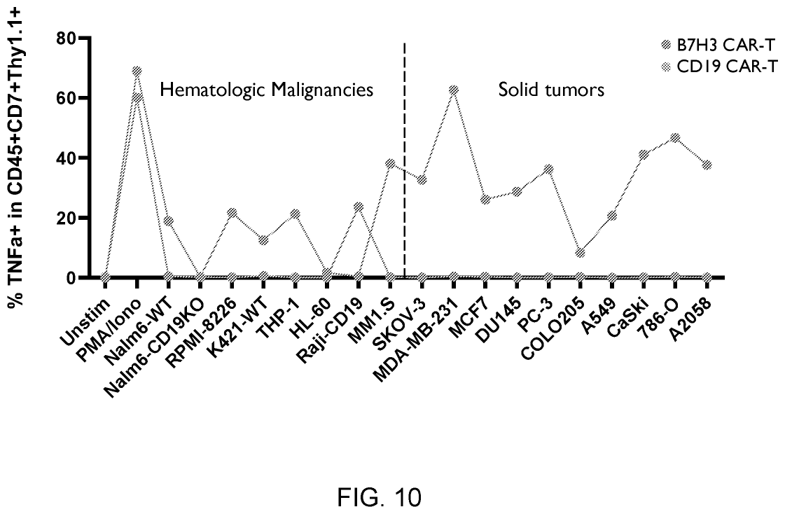

B7H3-CARs using a VHH domain (for example, SEQ ID NOs. 36-41 and variants thereof) of a single domain anti-B7H3 antibody for its binding region (camB7H3) were designed. The CD28-CD3ζ(1XX), NKG2D-2B4-CD3ζ, or 4-1BB-CD3ζ was used as exemplary costimulatory and signaling domain for the B7H3-CAR configuration, and the cell surface camB7H3-CAR expression is shown in transduced cells compared to control cells without B7H3-CAR transduction (FIG. 8). T cells transduced with B7H3-CAR showed heightened IFNγ expression indicating cell activation (FIG. 9A), and effective tumor cell recognition and elimination with durability across multiple solid tumor lines including ovarian cancer (SKOV3), triple negative breast cancer (MDA-MB 231), prostate cancer (PC-3, DU145), cervical cancer (Caski), and renal cell carcinoma (786-O) (FIG. 9B). In a separate assay, B7H3-CAR T cells and CD19-CAR T cells were incubated with a broader collection of tumor lines at an E:T ratio of 2:1 at 37° C., and after about 4 hours, CD45, CD7 and Thy1.1 cell surface staining were used to distinguish transduced CAR-T cells from residual tumor cells. The positive and negative B7H3 antigen recognition of these tumor lines by the B7H3-CAR expressing T cells are indicated through the % TNFα positive (TNFα⁺) CAR-T cells (FIG. 10).

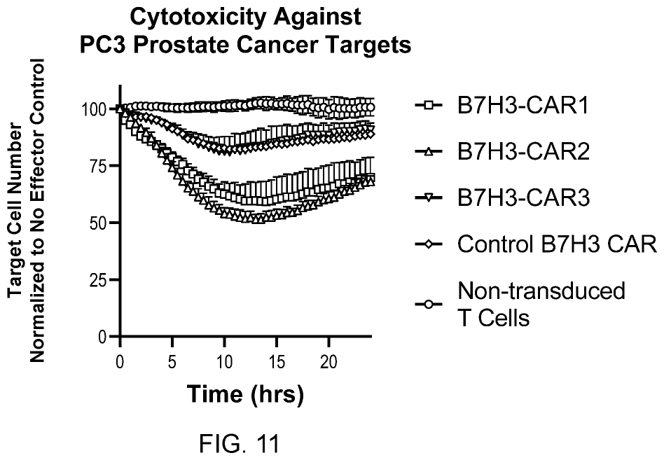

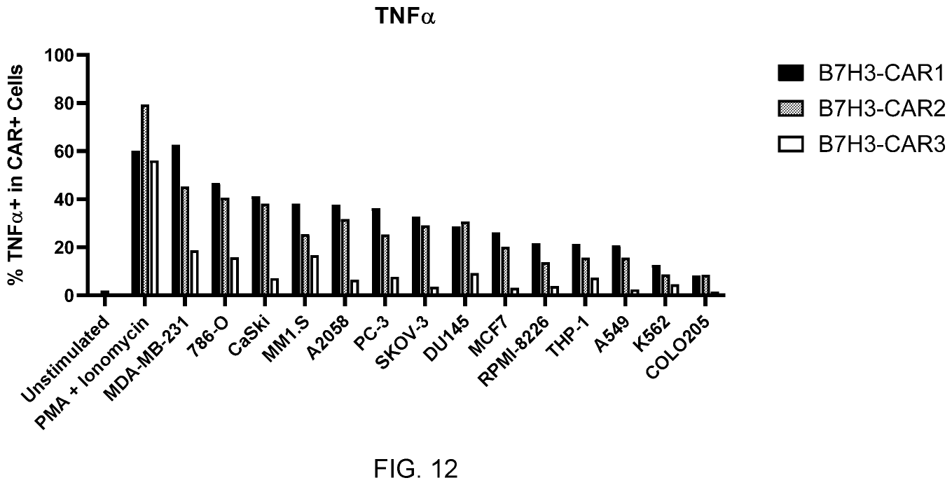

Different spacer (hinge between the ecto-binding domain and the transmembrane domain) designs (i.e., short-<80 a.a, medium-80-180 a.a, and long->180 a.a; CAR1-CAR3, respectively) were also screened to determine the optimal configuration for camB7H3-CAR targeting. All three candidate CAR designs each with a different spacer were used to generate CAR-T cells which were tested for cytotoxicity against PC3 prostate cancer cells. Non-transduced T cells were included as a negative control. As shown in FIG. 11, all CAR designs demonstrated cytotoxicity, however, B7H3-CAR1 and B7H3-CAR2 presented increased cytotoxicity compared to the B7H3-CAR3 design. Further, primary CAR-T cells expressing the three candidate B7H3-CAR designs were evaluated for intracellular production of TNFα following incubation with the indicated tumor cell lines. B7H3-CAR1 and B7H3-CAR2 continued to outperform the B7H3-CAR3 design, with B7H3-CAR1 showing the highest TNFα production in T effector cells (FIG. 12).

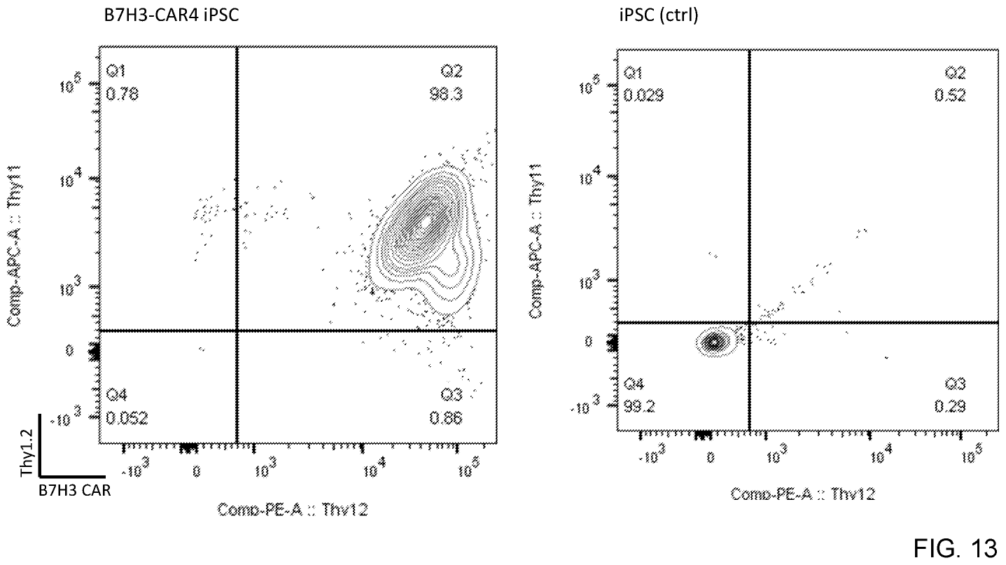

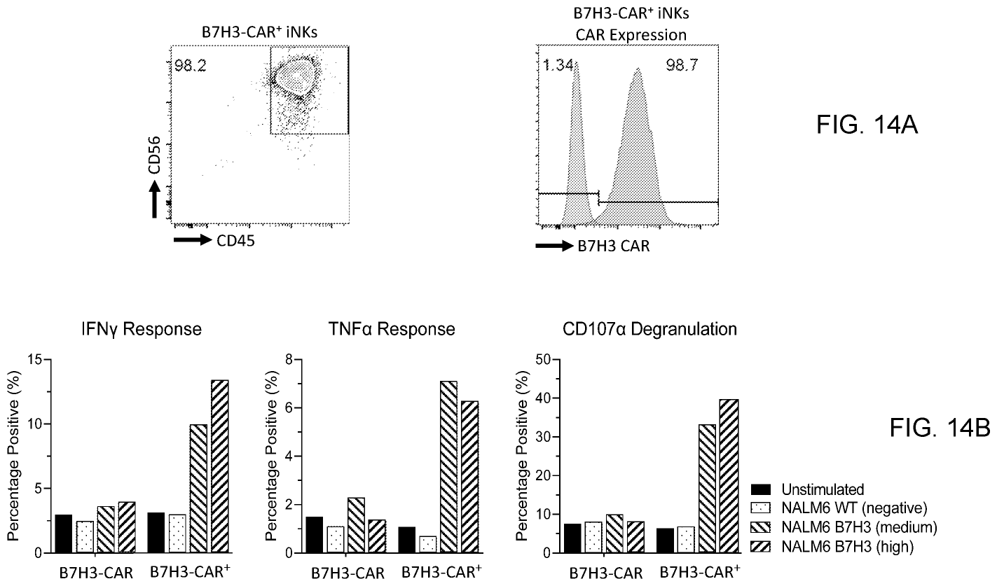

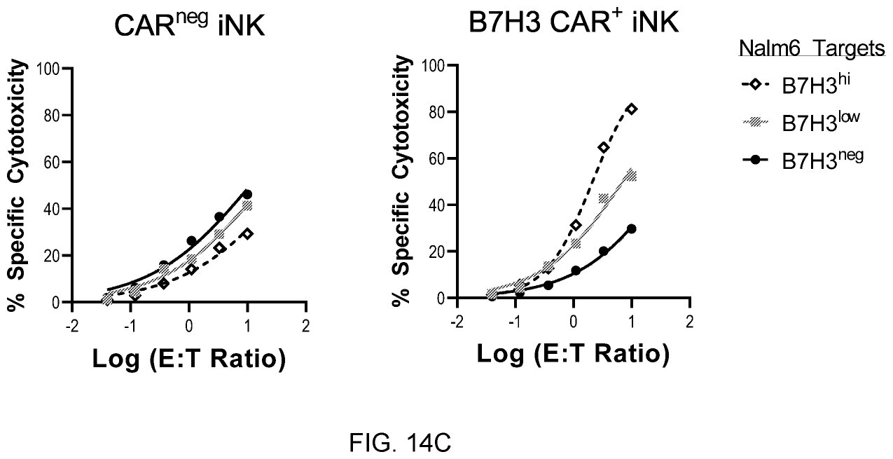

Further, camB7H3-CAR was transferred to iPSC-derived effector cells. For proof of concept, B7H3-CAR⁺ iNK cells were generated by first transducing a camB7H3-CAR construct into iPSC cell-line cells expressing hnCD16, an IL15 cytokine signaling complex, and having CD38 knocked out through prior targeted engineering. Anti-Thy1.1 mAb and biotinylated recombinant B7H3 protein were utilized in combination with Streptavidin-Phycoerythrin (SA-PE) to confirm transduction efficiency and detect surface camB7H3-CAR expression in the iPS cells (FIG. 13, left panel), compared to non-transduced controls (FIG. 13, right panel). The selected transduced clonal iPSC cells comprising the B7H3-CAR were then differentiated to generate iNK cells (FIG. 14A, left panel; gated at CD56 and CD45), with surface camB7H3-CAR expression demonstrated as compared to the non-transduced iNK cell control and the unstained iNK cell control (FIG. 14A, right panel).

To characterize the functionality of B7H3-CAR⁺ iNK as effector cells, the cells were incubated, at various E:T ratios, with Nalm6 cells that were either negative, low, or high B7H3 expressors for a cytotoxicity assay, with CAR-negative iNK cells as control. After co-culturing at 37° C. for 4 hrs, specific tumor cytotoxicity of the iNK cells was calculated using flow cytometry to determine the percentage of Caspase 3/7 positive tumor cells as a result of CAR iNK cell killing. The B7H3-CAR⁺ iNK cells showed increasing cytotoxicity with increasing levels of tumor cell surface B7H3 (FIG. 14C), demonstrating antigen-specific anti-tumor cytotoxicity in an antigen dose responsive manner.

To assess B7H3-dependent cytokine release and degranulation (CR/D) response, which is indicative of activated effector cells with the ability of targeted killing, highly pure and homogenous B7H3-CAR⁺ iNK cells (FIG. 14A) were cultured alone (unstimulated) or with NALM6 tumor cells overexpressing surface B7H3 protein at different levels (NALM6 WT-B7H3 negative, NALM6-B7H3 medium, and NALM6-B7H3 high) at a 2:1 E:T ratio in the presence of an anti-CD107ab antibody. B7H3-CAR⁺ iNK cells without B7H3-CAR transduction were used as control. Plates were incubated at around 37° C. for 4 hours, followed by surface staining with anti-CD56 and anti-hnCD16 to distinguish iNK cells from residual tumor cells. The percentage of IFNγ, TNFα and degranulating (CD107ab⁺) cells were quantified using intracellular cytokine staining, showing antigen-specific and antigen dose responsive effector cell activation in B7H3-CAR⁺ iNK cells (FIG. 14C).

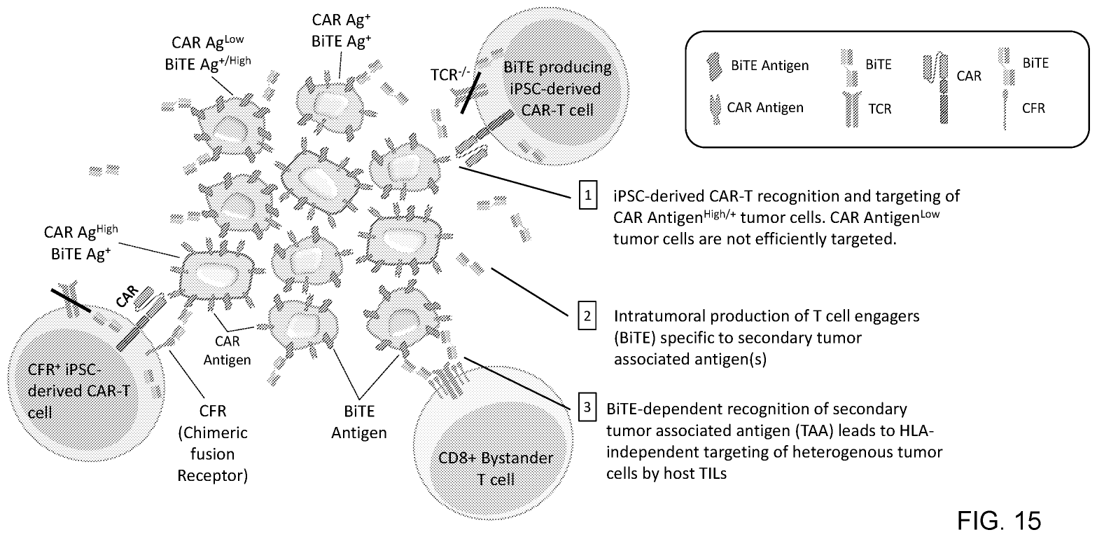

Without being limited by theory, FIG. 15 summarizes the rational design of effector cells including iPSC-derived CAR-T or NK cells to afford the effector cells enhanced capabilities in recognizing and targeting tumor cells heterogenous in expression levels of the primary tumor antigen targeted by a CAR, which primary tumor antigen includes B7H3. As demonstrated by data provided herein, the intratumoral production of an engager (BiTE or TRiKE) specific to a tumor associated antigen (TAA) that differs from that of the CAR as illustrated, enables engager-dependent recognition of a secondary tumor associated antigen, which could lead to HLA-independent targeting of heterogenous tumor cells by bystander T cells in the recipient immune system and also by effector cells through a surface triggering receptor for the engager. Such surface triggering receptor includes but is not limited to a CFR (chimeric fusion receptor) as described in this application. The signal transduction of CFR upon coupling with the engager, such as a BiTE or a TRiKE triggers additional killing capability of the CAR expressing effector cell, including but not limited to the B7H3-CAR effector cells depicted herein, against heterogenous tumor cells, thereby providing a novel approach for overcoming tumor CAR antigen evasion.

One skilled in the art would readily appreciate that the methods, compositions, and products described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A cell or a population thereof, wherein the cell comprises a polynucleotide encoding a CAR targeting a B7H3 tumor antigen, wherein the CAR comprises a binding domain comprising:
(i) an amino acid sequence that is of at least about 99%, 98%, 96%, 95%, 90%, 85%, or 80% identity to SEQ ID NO: 36, 37, 38, 39, 40, or 41;
(ii) an amino acid sequence represented by a variant of SEQ ID NO: 36, and wherein the variant has one or more mutations at positions comprising 1, 40, 46, 79, 87, 88, 89, 97, 98, and 117 of SEQ ID NO: 36;
(iii) an amino acid sequence represented by a variant of SEQ ID NO: 36, wherein the variant has one or more substitutions comprising Q1E, T40A, E46V, G79L, K87R, P88A, D89E, V97A, S98R, and Q117L according to SEQ ID NO: 36; or
(iv) an amino acid sequence represented by any of SEQ ID NOs: 36, 37, 38, 39, 40, and 41; and
wherein the cell is an eukaryotic cell, an animal cell, a human cell, an immune cell, an induced pluripotent cell (iPSC), or a derivative cell differentiated therefrom.

2. The cell or a population thereof of claim 1, wherein the cell further comprises one or more polynucleotides encoding an engager, and optionally a CFR (chimeric fusion receptor), wherein the engager has a tumor antigen targeting specificity that is not B7H3.

3. The cell or a population thereof of claim 2, wherein
(i) the engager comprises a first binding domain having a tumor targeting specificity to any one of ADGRE2, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CD5, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NY-ESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen;
(ii) the engager comprises a second binding domain having specificity that is different from the specificity of the first binding domain and is to an extracellular portion of CD3, CD28, CD5, CD16, CD64, CD32, CD33, CD89, NKG2C, NKG2D, or any functional variants thereof, or the second binding domain of the engager is specific to an ectodomain of the CFR; or
(iii) the engager comprises a cytokine or a variant thereof between the first and the second binding domains, wherein the cytokine comprises at least one of IL2, IL4, IL6, IL7, IL9, IL10, IL11, IL12, IL15, IL18, and IL21.

4. The cell or a population thereof of claim 2, wherein
(i) the CFR comprises an ectodomain fused to a transmembrane domain, which is operatively connected to an endodomain, and wherein the ectodomain, transmembrane domain and the endodomain do not comprise any endoplasmic reticulum (ER) retention signals or endocytosis signals;
(ii) the CFR comprises an ectodomain that comprises a full or partial length of an extracellular portion of a signaling protein comprising at least one of CD3ε, CD3γ, CD3δ, CD28, CD5, CD16, CD64, CD32, CD33, CD89, NKG2C, NKG2D, any functional variants, and a combination or a chimera thereof;
(iii) the CFR comprises an ectodomain that initiates signal transduction upon binding to a selected agonist;
(iv) the CFR comprises an endodomain that comprises a cytotoxicity domain comprising at least a full length or a portion of CD3ζ, 2B4, DAP10, DAP12, DNAM1, CD137 (4-1BB), IL21, IL7, IL12, IL15, NKp30, NKp44, NKp46, NKG2C, or NKG2D polypeptide; and optionally wherein the endodomain further comprises one or more of:
(a) a co-stimulatory domain comprising a full length or a portion of CD2, CD27, CD28, CD40L, 4-1BB, OX40, ICOS, PD-1, LAG-3, 2B4, BTLA, DAP10, DAP12, CTLA-4, or NKG2D polypeptide, or any combination thereof;
(b) a co-stimulatory domain comprising a full length or a portion of CD28, 4-1BB, CD27, CD40L, ICOS, CD2, or any combination thereof;
(c) a persistency signaling domain comprising a full length or a portion of an endodomain of a cytokine receptor comprising IL7R, IL15R, IL18R, IL12R, IL23R, or combinations thereof; and/or
(d) a full or a partial intracellular portion of a receptor tyrosine kinase (RTK), a tumor necrosis factor receptor (TNFR), an EGFR or a FAS receptor; or
(iv) the CFR is co-expressed with the engager or the CAR in separate constructs or in a bicistronic expression cassette.

5. The cell or a population thereof of any of claims 1-4, wherein the cell further comprises one or more of:
(i) CD38 knockout;
(ii) HLA-I deficiency and/or HLA-II deficiency;
(iii) introduction of HLA-G or non-cleavable HLA-G, or knockout of one or both of CD58 and CD54;
(iv) an exogenous CD16 or a variant thereof;
(v) a signaling complex comprising a partial or full peptide of a cell surface expressed exogenous cytokine and/or a receptor thereof;
(vi) at least one of the genotypes listed in Table 1;
(vii) disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or
(viii) introduction of at least one of HLA-E, 4-1BBL, CD3, CD4, CD8, CD16, CD47, CD113, CD131, CD137, CD80, PDL1, A_{2A}R, antigen-specific TCR, Fc receptor, an antibody or functional variant or fragment thereof, a checkpoint inhibitor, and surface triggering receptor for coupling with an agonist, in comparison to its counterpart primary cell.

6. The cell or a population thereof of any of claims 1-5, wherein the cell has therapeutic properties comprising one or more of:
(i) increased cytotoxicity;
(ii) improved persistency and/or survival;
(iii) enhanced ability in migrating, and/or activating or recruiting bystander immune cells, to tumor sites;
(iv) improved tumor penetration;
(v) enhanced ability to reduce tumor immunosuppression;
(vi) improved ability in rescuing tumor antigen escape;
(vii) controlled apoptosis;
(viii) enhanced or acquired ADCC; and
(ix) ability to avoid fratricide,
in comparison to its counterpart primary cell obtained from peripheral blood, umbilical cord blood, or any other donor tissues.

7. The cell or a population thereof of claim 6, wherein the exogenous CD16 or a variant thereof comprises at least one of:
(a) a high affinity non-cleavable CD16 (hnCD16);
(b) F176V and S197P in ectodomain domain of CD16;
(c) a full or partial ectodomain originated from CD64;
(d) a non-native (or non-CD16) transmembrane domain;
(e) a non-native (or non-CD16) intracellular domain;
(f) a non-native (or non-CD16) signaling domain;
(g) a non-native stimulatory domain; and
(h) transmembrane, signaling, and stimulatory domains that are not originated from CD16, and are originated from a same or different polypeptide.

8. The cell or a population thereof of claim 7, wherein the cell surface expressed exogenous cytokine or receptor thereof:
(a) comprises at least one of IL2, IL4, IL6, IL7, IL9, IL10, IL11, IL12, IL15, IL18, IL21, and its respective receptor(s); or
(b) comprises at least one of:
(i) co-expression of IL15 and IL15Rα by using a self-cleaving peptide;
(ii) a fusion protein of IL15 and IL15Rα;
(iii) an IL15/IL15Rα fusion protein with intracellular domain of IL15Rα truncated or eliminated;
(iv) a fusion protein of IL15 and membrane bound Sushi domain of IL15Rα;
(v) a fusion protein of IL15 and IL15Rβ;
(vi) a fusion protein of TL15 and common receptor γC, wherein the common receptor γC is native or modified; and
(vii) a homodimer of IL15Rβ,
wherein any one of (b)(i)-(vii) is optionally co-expressed with a CAR in separate constructs or in a bi-cistronic construct; or
(c) comprises at least one of:
(i) a fusion protein of IL7 and IL7Rα;
(ii) a fusion protein of IL7 and common receptor γC, wherein the common receptor γC is native or modified; and
(iii) a homodimer of IL7Rβ, wherein any one of (c)(i)-(iii) is optionally co-expressed with a CAR in separate constructs or in a bi-cistronic expression cassette;
and optionally,
(d) is transiently expressed.

9. The cell or a population thereof of claim 7, wherein the checkpoint inhibitor is an antagonist to one or more checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, and inhibitory KIR.

10. The cell or a population thereof of claim 1, wherein the cell comprises:
(i) one or more exogenous polynucleotides integrated in a safe harbor locus or a selected gene locus; or
(ii) more than two exogenous polynucleotides integrated in different safe harbor loci or two or more selected gene loci.

11. The cell or a population thereof of claim 10, wherein the safe harbor locus comprises at least one of AAVS1, CCR5, ROSA26, collagen, HTRP, H11, GAPDH, or RUNX1; and wherein the selected gene locus is one of B2M, TAP1, TAP2, Tapasin, NLRC5, CIITA, RFXANK, RFX5, RFXAP, TCR, NKG2A, NKG2D, CD38, CD25, CD69, CD44, CD58, CD54, CD56, CIS, CBL-B, SOCS2, PD1, CTLA4, LAG3, TIM3, or TIGIT; and/or wherein the integration of the exogenous polynucleotides knocks out expression of the gene in the locus.

12. The cell or a population thereof of claim 11, wherein the TCR locus is a constant region of TCR alpha and/or TCR beta (TRAC and/or TRBC).

13. The cell or a population thereof of claim 1, wherein the iPSC is a clonal iPSC, a single cell dissociated iPSC, an iPSC cell line cell, or an iPSC master cell bank (MCB) cell; or wherein the derivative cell comprises a derivative CD34⁺ cell, a derivative hematopoietic stem and progenitor cell, a derivative hematopoietic multipotent progenitor cell, a derivative T cell progenitor, a derivative NK cell progenitor, a derivative T lineage cell, a derivative NKT lineage cell, a derivative NK lineage cell, a derivative B lineage cell, or a derivative effector cell having one or more functional features that are not present in a counterpart primary T, NK, NKT, and/or B cell.

14. A composition comprising the cell or population thereof of any one of claims 1-13.

15. A composition for therapeutic use comprising the derivative cell of any one of claims 1-13, and one or more therapeutic agents.

16. The composition of claim 15, wherein the one or more therapeutic agents comprise a peptide, a cytokine, a checkpoint inhibitor, a mitogen, a growth factor, a small RNA, a dsRNA (double stranded RNA), mononuclear blood cells, feeder cells, feeder cell components or replacement factors thereof, a vector comprising one or more polynucleic acids of interest, an antibody, a chemotherapeutic agent or a radioactive moiety, or an immunomodulatory drug (IMiD).

17. The composition of claim 16, wherein:
(i) the checkpoint inhibitor comprises:
(a) one or more antagonists to checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/ILA-E, or inhibitory KIR;
(b) one or more of atezolizunab, avelumab, durvalumab, ipilimumab, IPH4102, IPH43, IPH33, lirimumab, monalizumab, nivolumab, pembrolizumab, and their derivatives or functional equivalents;
(c) at least one of atezolizumab, nivolumab, and pembrolizumab; or
(ii) the therapeutic agents comprise one or more of venetoclax, azacitidine, and pomalidomide.

18. The composition of claim 16, wherein the antibody comprises:
(a) anti-CD20, anti-HER2, anti-CD52, anti-EGFR, anti-CD123, anti-GD2, anti-PDL1, and/or anti-CD38 antibody;
(b) one or more of rituximab, veltuzumab, ofatumumab, ublituximab, ocaratuzumab, obinutuzumab, trastuzumab, pertuzumab, alemtuzumab, cetuximab, dinutuximab, avelumab, daratumumab, isatuximab, MOR202, 7G3, CSL362, elotuzumab, and their humanized or Fc modified variants or fragments and their functional equivalents and biosimilars; or
(c) daratumumab, and wherein the derivative hematopoietic cells comprise derivative NK cells or derivative T cells comprising a CD38 knockout, and optionally an expression of exogenous CD16 or a variant thereof.

19. Therapeutic use of the composition of any one of the claims 14-18 by introducing the composition to a subject suitable for adoptive cell therapy, wherein the subject has an autoimmune disorder; a hematological malignancy; a solid tumor; cancer, or a virus infection.

20. A composition comprising a cell or a population thereof, wherein the cell comprises one or more polynucleotides encoding a chimeric antigen receptor (CAR), an engager, and optionally a CFR (chimeric fusion receptor), wherein the CFR is optionally for engager coupling, and wherein the cell is an eukaryotic cell, an animal cell, a human cell, an immune cell, an induced pluripotent cell (iPSC), or a derivative cell differentiated therefrom.

21. The composition of claim 20, wherein: (i) the engager has a different tumor targeting specificity from the CAR; or (ii) the engager is co-expressed with the CAR or the CFR.

22. The composition of claim 20, wherein the CAR is:
(i) T cell specific or NK cell specific;
(ii) a bi-specific antigen binding CAR;
(iii) a switchable CAR;
(iv) a dimerized CAR;
(v) a split CAR;
(vi) a multi-chain CAR;
(vii) an inducible CAR;
(viii) co-expressed with another CAR;
(ix) co-expressed with a partial or full peptide of a cell surface expressed exogenous cytokine or a receptor thereof, optionally in separate constructs or in a bi-cistronic construct;
(x) co-expressed with a checkpoint inhibitor, optionally in separate constructs or in a bi-cistronic construct; and/or
(xi) specific to any one of ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CD5, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NY-ESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; and
wherein the CAR of any one of (i) to (xi) is optionally inserted at TRAC or TRBC locus, and/or is driven by an endogenous promoter of TCR, and/or the TCR is knocked out by the CAR insertion.

23. The composition of claim 20, wherein the CAR comprises:
(i) an antigen recognition region specific to B7H3;
(ii) a binding domain comprising an amino acid sequence that is of at least about 99%, 98%, 96%, 95%, 90%, 85%, or 80% identity to SEQ ID NO: 36, 37, 38, 39, 40, or 41;
(iii) a binding domain comprising an amino acid sequence represented by a variant of SEQ ID NO: 36, wherein the variant has one or more mutations at positions comprising 1, 40, 46, 79, 87, 88, 89, 97, 98, and 117 of SEQ ID NO: 36;
(iv) a binding domain comprising an amino acid sequence represented by a variant of SEQ ID NO: 36, wherein the variant has one or more substitutions comprising Q1E, T40A, E46V, G79L, K87R, P88A, D89E, V97A, S98R, and Q117L according to SEQ ID NO:36; or
(v) a binding domain comprising an amino acid sequence represented by any of SEQ ID NOs: 36, 37, 38, 39, 40, and 41.

24. The composition of claim 20 or 21, wherein:
(i) the engager comprises a first binding domain having a different tumor targeting specificity from the CAR, and wherein the first binding domain is specific to any one of ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CD5, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NY-ESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; or
(ii) the engager comprises a first binding domain having a different tumor targeting specificity from the CAR, and wherein the CAR comprises a binding domain specific to any one of B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, or ROR1; or
(iii) the engager comprises a second binding domain having specificity that is different from the specificity of the first binding domain and is to an extracellular portion of CD3, CD28, CD5, CD16, CD64, CD32, CD33, CD89, NKG2C, NKG2D, or any functional variants thereof; or
(iv) the engager comprises a cytokine or a variant thereof between the first and the second binding domains, wherein the cytokine comprises at least one of IL2, IL4, IL6, IL7, IL9, IL10, IL11, IL12, IL15, IL18, and IL21.

25. The composition of claim 20, wherein the cell further comprises one or more of:
(i) CD38 knockout;
(ii) HLA-I deficiency and/or HLA-II deficiency;
(iii) introduction of HLA-G or non-cleavable HLA-G, or knockout of one or both of CD58 and CD54;
(iv) an exogenous CD16 or a variant thereof;
(v) a chimeric fusion receptor (CFR);
(vi) an inactivation CAR;
(vii) a signaling complex comprising a partial or full peptide of a cell surface expressed exogenous cytokine and/or a receptor thereof;
(viii) at least one of the genotypes listed in Table 1;
(ix) disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or
(x) introduction of at least one of HLA-E, 4-1BBL, CD3, CD4, CD8, CD16, CD47, CD113, CD131, CD137, CD80, PDL1, A_{2A}R, antigen-specific TCR, Fc receptor, an antibody or functional variant or fragment thereof, a checkpoint inhibitor, and surface triggering receptor for coupling with an agonist, in comparison to its counterpart primary cell.

26. The composition of any one of claims 20-25, wherein the cell has therapeutic properties comprising one or more of:
(i) increased cytotoxicity;
(ii) improved persistency and/or survival;
(iii) enhanced ability in migrating, and/or activating or recruiting bystander immune cells, to tumor sites;
(iv) improved tumor penetration;
(v) enhanced ability to reduce tumor immunosuppression;
(vi) improved ability in rescuing tumor antigen escape;
(vii) controlled apoptosis;
(viii) enhanced or acquired ADCC; and
(ix) ability to avoid fratricide,
in comparison to its counterpart primary cell obtained from peripheral blood, umbilical cord blood, or any other donor tissues.

27. The composition of claim 25, wherein the exogenous CD16 or a variant thereof comprises at least one of:
(a) a high affinity non-cleavable CD16 (hnCD16);
(b) F176V and S197P in ectodomain domain of CD16;
(c) a full or partial ectodomain originated from CD64;
(d) a non-native (or non-CD16) transmembrane domain;
(e) a non-native (or non-CD16) intracellular domain;
(f) a non-native (or non-CD16) signaling domain;
(g) a non-native stimulatory domain; and
(h) transmembrane, signaling, and stimulatory domains that are not originated from CD16, and are originated from a same or different polypeptide.

28. The composition of any one of claims 20-25, wherein the CFR comprises an ectodomain fused to a transmembrane domain, which is operatively connected to an endodomain, and wherein the ectodomain, transmembrane domain and the endodomain do not comprise any endoplasmic reticulum (ER) retention signals or endocytosis signals.

29. The composition of claim 28, wherein:
(i) the ectodomain of the CFR comprises a full or partial length of an extracellular portion of a signaling protein comprising at least one of CD3ε, CD3γ, CD3δ, CD28, CD5, CD16, CD64, CD32, CD33, CD89, NKG2C, NKG2D, any functional variants, and a combination or a chimera thereof;
(ii) the ectodomain of the CFR initiates signal transduction upon binding to a selected agonist;
(iii) the endodomain of the CFR comprises a cytotoxicity domain comprising at least a full length or a portion of CD3ζ, 2B4, DAP10, DAP12, DNAM1, CD137 (4-1BB), IL21, IL7, IL12, IL15, NKp30, NKp44, NKp46, NKG2C, or NKG2D polypeptide; and optionally wherein the endodomain further comprises one or more of:
(a) a co-stimulatory domain comprising a full length or a portion of CD2, CD27, CD28, CD40L, 4-1BB, OX40, ICOS, PD-1, LAG-3, 2B4, BTLA, DAP10, DAP12, CTLA-4, or NKG2D polypeptide, or any combination thereof,
(b) a co-stimulatory domain comprising a full length or a portion of CD28, 4-1BB, CD27, CD40L, ICOS, CD2, or combinations thereof;
(c) a persistency signaling domain comprising a full length or a portion of an endodomain of a cytokine receptor comprising IL7R, IL15R, IL18R, IL12R, IL23R, or combinations thereof; and/or
(d) a full or a partial intracellular portion of a receptor tyrosine kinase (RTK), a tumor necrosis factor receptor (TNFR), an EGFR or a FAS receptor; or
(iv) the CFR is co-expressed with the engager in separate constructs or in a bicistronic expression cassette, and optionally the engager has binding specificity to the ectodomain of the CFR.

30. The composition of claim 29, wherein the selected agonist is (i) an antibody or a functional variant or fragment thereof; (ii) an agonistic ligand; or (iii) an engager; and
wherein the selected agonist:
(a) is encoded by the polynucleotide comprised in the cell of the composition or is comprised in the composition;
(b) comprises at least a first binding domain that is specific to at least one tumor antigen comprising B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, or ROR1; and optionally,
(c) comprises a second binding domain that is specific to a cell surface protein of:
(1) the cell of the composition; or
(2) a bystander effector cell;
wherein the cell surface protein comprises an extracellular portion of CD3, CD28, CD5, CD16, CD64, CD32, CD33, CD89, NKG2C, NKG2D, or any functional variants thereof.

31. The composition of claim 25, wherein the inactivation CAR:
(i) targets an upregulated surface protein in activated recipient immune cells; or
(ii) comprises at least one of a CD38-CAR, a CD25-CAR, a CD69-CAR, a CD44-CAR, a 4-1BB-CAR, an OX40-CAR, and a CD40L-CAR.

32. The composition of claim 25, wherein the cell surface expressed exogenous cytokine or receptor thereof:
(a) comprises at least one of IL2, IL4, IL6, IL7, IL9, IL10, IL11, IL12, IL15, IL18, IL21, and its respective receptor(s); or
(b) comprises at least one of:
(i) co-expression of IL15 and IL15Rα by using a self-cleaving peptide;
(ii) a fusion protein of IL15 and IL15Rα;
(iii) an IL15/IL15Rα fusion protein with intracellular domain of IL15Rα truncated or eliminated;
(iv) a fusion protein of IL15 and membrane bound Sushi domain of IL15Rα;
(v) a fusion protein of IL15 and IL15Rβ;
(vi) a fusion protein of TL15 and common receptor γC, wherein the common receptor γC is native or modified; and
(vii) a homodimer of IL15Rβ,
wherein any one of (b)(i)-(vii) is optionally co-expressed with a CAR in separate constructs or in a bi-cistronic construct; or
(c) comprises at least one of:
(i) a fusion protein of IL7 and IL7Rα;
(ii) a fusion protein of IL7 and common receptor γC, wherein the common receptor γC is native or modified; and
(iii) a homodimer of IL7Rβ, wherein any one of (c)(i)-(iii) is optionally co-expressed with a CAR in separate constructs or in a bi-cistronic expression cassette;
and optionally,
(d) is transiently expressed.

33. The composition of claim 25, wherein the checkpoint inhibitor is an antagonist to one or more checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, and inhibitory KIR.

34. The composition of claim 20, wherein the cell comprises:
(i) one or more exogenous polynucleotides integrated in a safe harbor locus or a selected gene locus; or
(ii) more than two exogenous polynucleotides integrated in different safe harbor loci or two or more selected gene loci.

35. The composition of claim 34, wherein the safe harbor locus comprises at least one of AAVS1, CCR5, ROSA26, collagen, HTRP, H11, GAPDH, or RUNX1; and wherein the selected gene locus is one of B2M, TAP1, TAP2, Tapasin, NLRC5, CIITA, RFXANK, RFX5, RFXAP, TCR, NKG2A, NKG2D, CD38, CD25, CD69, CD44, CD58, CD54, CD56, CIS, CBL-B, SOCS2, PD1, CTLA4, LAG3, TIM3, or TIGIT; and/or wherein the integration of the exogenous polynucleotides knocks out expression of the gene in the locus.

36. The composition of claim 35, wherein the TCR locus is a constant region of TCR alpha and/or TCR beta (TRAC and/or TRBC).

37. The composition of claim 20, wherein the derivative cell comprises a derivative CD34⁺ cell, a derivative hematopoietic stem and progenitor cell, a derivative hematopoietic multipotent progenitor cell, a derivative T cell progenitor, a derivative NK cell progenitor, a derivative T lineage cell, a derivative NKT lineage cell, a derivative NK lineage cell, a derivative B lineage cell, or a derivative effector cell having one or more functional features that are not present in a counterpart primary T, NK, NKT, and/or B cell.

38. The composition of claim 20, further comprising one or more therapeutic agents.

39. The composition of claim 38, wherein the one or more therapeutic agents comprise a peptide, a cytokine, a checkpoint inhibitor, an antibody or functional variant or fragment thereof, a mitogen, a growth factor, a small RNA, a dsRNA (double stranded RNA), mononuclear blood cells, feeder cells, feeder cell components or replacement factors thereof, a vector comprising one or more polynucleic acids of interest, a chemotherapeutic agent or a radioactive moiety, or an immunomodulatory drug (IMiD).

40. The composition of claim 39, wherein:
(a) the checkpoint inhibitor comprises:
(i) one or more antagonist checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, or inhibitory KIR;
(ii) one or more of atezolizumab, avelumab, durvalumab, ipilimumab, IPH4102, IPH43, IPH33, lirimumab, monalizumab, nivolumab, pembrolizumab, and their derivatives or functional equivalents; or
(iii) at least one of atezolizumab, nivolumab, and pembrolizumab; or
(b) the one or more therapeutic agents comprise one or more of venetoclax, azacitidine, and pomalidomide.

41. The composition of claim 39, wherein the antibody, or functional variant or fragment thereof comprises:
(a) anti-CD20, anti-CD22, anti-HER2, anti-CD52, anti-EGFR, anti-CD123, anti-GD2, anti-PDL1, and/or anti-CD38 antibody;
(b) one or more of rituximab, veltuzumab, ofatumumab, ublituximab, ocaratuzumab, obinutuzumab, ibritumomab, ocrelizumab, inotuzumab, moxetumomab, epratuzumab, trastuzumab, pertuzumab, alemtuzumab, cetuximab, dinutuximab, avelumab, daratumumab, isatuximab, MOR202, 7G3, CSL362, elotuzumab, and their humanized or Fc modified variants or fragments and their functional equivalents and biosimilars; or
(c) daratumumab, and wherein the derivative effector cell comprises a CD38 knockout, and optionally expresses CD16 or a variant thereof.

42. Therapeutic use of the composition of any one of claims 20-41 by introducing the composition to a subject suitable for adoptive cell therapy, wherein the subject has an autoimmune disorder, a hematological malignancy, a solid tumor, cancer, or a viral infection.

43. A method of manufacturing a derivative effector cell comprising a first polynucleotide encoding a chimeric antigen receptor (CAR) and one or more additional polynucleotides encoding one or both of an engager and a CFR, wherein the engager has a different tumor targeting specificity from the CAR, wherein the method comprises:
differentiating a genetically engineered iPSC, wherein the iPSC comprises the first and the one or more additional polynucleotides and optionally one or more genomic edits comprising:
(i) CD38 knockout;
(ii) HLA-I deficiency and/or HLA-II deficiency;
(iii) introduction of HLA-G or non-cleavable HLA-G, or knockout of one or both of CD58 and CD54;
(iv) an exogenous CD16 or a variant thereof;
(v) a chimeric fusion receptor (CFR);
(vi) an inactivation CAR;
(vii) a signaling complex comprising a partial or full peptide of a cell surface expressed exogenous cytokine and/or a receptor thereof;
(viii) at least one of the genotypes listed in Table 1;
(ix) disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; or
(x) introduction of at least one of HLA-E, 4-1BBL, CD3, CD4, CD8, CD16, CD47, CD113, CD131, CD137, CD80, PDL1, A_{2A}R, antigen-specific TCR, Fc receptor, an antibody or functional variant or fragment thereof, a checkpoint inhibitor, and surface triggering receptor for coupling with an agonist, in comparison to its counterpart primary cell.

44. The method of claim 43, wherein:
(i) the engager comprises a first binding domain having a different tumor targeting specificity from the CAR, and wherein the binding domain is specific to any one of ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CD5, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NY-ESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; or
(ii) the engager comprises a first binding domain having a different tumor targeting specificity from the CAR, and wherein the binding domain is specific to any one of B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, or ROR1; or
(iii) the engager comprises a second binding domain having specificity that is different from the specificity of the first binding domain and is to an extracellular portion of CD3, CD28, CD5, CD16, CD64, CD32, CD33, CD89, NKG2C, NKG2D, or any functional variants thereof, or
(iv) the engager comprises a cytokine or a variant thereof between the first and the second binding domains, wherein the cytokine comprises at least one of IL2, IL4, IL6, IL7, IL9, IL10, IL11, IL12, IL15, IL18, and IL21.

45. The method of claim 43, wherein the CAR is:
(i) T cell specific or NK cell specific;
(ii) a bi-specific antigen binding CAR;
(iii) a switchable CAR;
(iv) a dimerized CAR;
(v) a split CAR;
(vi) a multi-chain CAR;
(vii) an inducible CAR;
(viii) co-expressed with another CAR;
(ix) co-expressed with a partial or full peptide of a cell surface expressed exogenous cytokine and/or a receptor thereof, optionally in separate constructs or in a bi-cistronic construct;
(x) co-expressed with a checkpoint inhibitor, optionally in separate constructs or in a bi-cistronic construct; and/or
(xi) specific to any one of ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CD5, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NY-ESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; and optionally,
wherein the CAR of any one of (i) to (xi) is optionally inserted at a TCR locus, and/or is driven by an endogenous promoter of TCR, and/or the TCR is knocked out by the CAR insertion.

46. The method of claim 43, wherein the CAR comprises an antigen recognition region specific to B7H3.

47. The method of claim 43, wherein the exogenous CD16 or a variant thereof comprises at least one of:
(a) a high affinity non-cleavable CD16 (hnCD16);
(b) F176V and S197P in ectodomain domain of CD16;
(c) a full or partial ectodomain originated from CD64;
(d) a non-native (or non-CD16) transmembrane domain;
(e) a non-native (or non-CD16) intracellular domain;
(f) a non-native (or non-CD16) signaling domain;
(g) a non-native stimulatory domain; and
(h) transmembrane, signaling, and stimulatory domains that are not originated from CD16, and are originated from a same or different polypeptide.

48. The method of claim 43, wherein the CFR comprises an ectodomain fused to a transmembrane domain, which is operatively connected to an endodomain, and wherein the ectodomain, transmembrane domain and the endodomain do not comprise any endoplasmic reticulum (ER) retention signals or endocytosis signals.

49. The method of claim 48, wherein:
(i) the ectodomain of the CFR comprises a full or partial length of an extracellular portion of a signaling protein comprising at least one of CD3ε, CD3γ, CD3δ, CD28, CD5, CD16, CD64, CD32, CD33, CD89, NKG2C, NKG2D, any functional variants, and a combination or a chimera thereof;
(ii) the ectodomain of the CFR initiates signal transduction upon binding to a selected agonist; or
(iii) the endodomain of the CFR comprises a cytotoxicity domain comprising at least a full length or a portion of CD3ζ, 2B4, DAP10, DAP12, DNAM1, CD137 (4-1BB), IL21, IL7, IL12, IL15, NKp30, NKp44, NKp46, NKG2C, or NKG2D polypeptide; and optionally wherein the endodomain further comprises one or more of:
(a) a co-stimulatory domain comprising a full length or a portion of CD2, CD27, CD28, CD40L, 4-1BB, OX40, ICOS, PD-1, LAG-3, 2B4, BTLA, DAP10, DAP12, CTLA-4, or NKG2D polypeptide, or any combination thereof,
(b) a co-stimulatory domain comprising a full length or a portion of CD28, 4-1BB, CD27, CD40L, ICOS, CD2, or combinations thereof,
(c) a persistency signaling domain comprising a full length or a portion of an endodomain of a cytokine receptor comprising IL7R, IL15R, IL18R, IL12R, IL23R, or combinations thereof; and/or
(d) a full or a partial intracellular portion of a receptor tyrosine kinase (RTK), a tumor necrosis factor receptor (TNFR), an EGFR or a FAS receptor.

50. The method of claim 49, wherein the selected agonist is (i) an antibody or a functional variant or fragment thereof; (ii) an agonistic ligand; or (iii) an engager; and
wherein the selected agonist:
(a) is encoded by the polynucleotide comprised in the cell of the composition or is comprised in the composition;
(b) comprises at least a first binding domain that is specific to at least one tumor antigen comprising B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, or ROR1; and optionally,
(c) comprises a second binding domain that is specific to a cell surface protein of:
(1) the cell of the composition; or
(2) a bystander effector cell;
wherein the cell surface protein comprises an extracellular portion of CD3, CD28, CD5, CD16, CD64, CD32, CD33, CD89, NKG2C, NKG2D, or any functional variants thereof.

51. The method of claim 43, wherein the inactivation CAR:
(i) targets an upregulated surface protein in activated recipient immune cells; or
(ii) comprises at least one of a CD38-CAR, a CD25-CAR, a CD69-CAR, a CD44-CAR, a 4-1BB-CAR, an OX40-CAR, and a CD40L-CAR.

52. The method of claim 43, wherein the checkpoint inhibitor is an antagonist to one or more checkpoint molecules comprising PD-1, PDL-1, TIM-3, TIGIT, LAG-3, CTLA-4, 2B4, 4-1BB, 4-1BBL, A_{2A}R, BATE, BTLA, CD39, CD47, CD73, CD94, CD96, CD160, CD200, CD200R, CD274, CEACAM1, CSF-1R, Foxpl, GARP, HVEM, IDO, EDO, TDO, LAIR-1, MICA/B, NR4A2, MAFB, OCT-2, Rara (retinoic acid receptor alpha), TLR3, VISTA, NKG2A/HLA-E, and inhibitory KIR.

53. The method of claim of claim 43, wherein the iPSC comprises:
(i) one or more exogenous polynucleotides integrated in a safe harbor locus or a selected gene locus; or
(ii) more than two exogenous polynucleotides integrated in different safe harbor loci or two or more selected gene loci.

54. The method of claim 53, wherein the safe harbor locus comprises at least one of AAVS1, CCR5, ROSA26, collagen, HTRP, H11, GAPDH, or RUNX1; and wherein the selected gene locus is one of B2M, TAP1, TAP2, Tapasin, NLRC5, CIITA, RFXANK, RFX5, RFXAP, TCR, NKG2A, NKG2D, CD38, CD25, CD69, CD44, CD58, CD54, CD56, CIS, CBL-B, SOCS2, PD1, CTLA4, LAG3, TIM3, or TIGIT; and/or wherein the integration of the exogenous polynucleotides knocks out expression of the gene in the locus.

55. The method of claim 54, wherein the TCR locus is a constant region of TCR alpha and/or TCR beta (TRAC and/or TRBC).

56. The method of claim 43, wherein the derivative effector cell comprises a derivative CD34⁺ cell, a derivative hematopoietic stem and progenitor cell, a derivative hematopoietic multipotent progenitor cell, a derivative T cell progenitor, a derivative NK cell progenitor, a derivative T lineage cell, a derivative NKT lineage cell, a derivative NK lineage cell, a derivative B lineage cell; or a derivative effector cell having one or more functional features that are not present in a counterpart primary T, NK, NKT, and/or B cell.

57. The method of claim 43, wherein the cell surface expressed exogenous cytokine or receptor thereof:
(a) comprises at least one of IL2, IL4, IL6, IL7, IL9, IL10, IL11, IL12, IL15, IL18, IL21, and its respective receptor(s); or
(b) comprises at least one of:
(i) co-expression of IL15 and IL15Rα by using a self-cleaving peptide;
(ii) a fusion protein of IL15 and IL15Rα;
(iii) an IL15/IL15Rα fusion protein with intracellular domain of IL15Rα truncated or eliminated;
(iv) a fusion protein of IL15 and membrane bound Sushi domain of IL15Rα;
(v) a fusion protein of IL15 and IL15Rβ;
(vi) a fusion protein of TL15 and common receptor γC, wherein the common receptor γC is native or modified; and
(vii) a homodimer of IL15Rβ,
wherein any one of (b)(i)-(vii) is optionally co-expressed with the CAR in separate constructs or in a bi-cistronic construct; or
(c) comprises at least one of:
(i) a fusion protein of IL7 and IL7Rα;
(ii) a fusion protein of IL7 and common receptor γC, wherein the common receptor γC is native or modified; and
(iii) a homodimer of IL7Rβ, wherein any one of (c)(i)-(iii) is optionally co-expressed with the CAR in separate constructs or in a bi-cistronic construct;
and optionally,
(d) is transiently expressed.

58. The method of any one of claims 43-57, further comprising genomically engineering a clonal iPSC to knock in polynucleotides encoding the CAR and one or both of the engager and the CFR; and optionally:
(i) to knock out CD38,
(ii) to knock out B2M and/or CIITA,
(iii) to knock out one or both of CD58 and CD54, and/or
(iv) to introduce HLA-G or non-cleavable HLA-G, a high affinity non-cleavable CD16 or a variant thereof, and/or a partial or full peptide of a cell surface expressed exogenous cytokine and/or a receptor thereof.

59. The method of claim 58, wherein the genomic engineering comprises targeted editing.

60. The method of claim 59, wherein the targeted editing comprises deletion, insertion, or in/del, and wherein the targeted editing is carried out by CRISPR, ZFN, TALEN, homing nuclease, homology recombination, or any other functional variation of these methods.

61. A method of improving tumor cell control and clearance comprising administering to a subject in need thereof the composition of any one of claims 20-41, optionally wherein the tumor is a solid tumor or the tumor is heterogenous.

62. The method of claim 61, wherein the cells of the composition express an antibody or functional variant or fragment thereof, or an engager.

63. The method of claim 61, wherein the composition comprises an engager.

64. The method of any one of claims 61-63, wherein:
(i) the engager comprises a first binding domain having a different tumor targeting specificity from the CAR, and wherein the binding domain is specific to any one of ADGRE2, B7H3, carbonic anhydrase IX (CAIX), CCR1, CCR4, carcinoembryonic antigen (CEA), CD3, CD5, CD7, CD8, CD10, CD20, CD22, CD30, CD33, CD34, CD38, CD41, CD44, CD44V6, CD49f, CD56, CD70, CD74, CD99, CD123, CD133, CD138, CD5, CLEC12A, an antigen of a cytomegalovirus (CMV) infected cell, epithelial glycoprotein-2 (EGP-2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-α, Ganglioside G2 (GD2), Ganglioside G3 (GD3), human Epidermal Growth Factor Receptor 2 (HER2), human telomerase reverse transcriptase (hTERT), ICAM-1, Integrin B7, Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1-CAM), LILRB2, melanoma antigen family A 1 (MAGE-A1), MICA/B, MR1, Mucin 1 (Muc-1), Mucin 16 (Muc-16), Mesothelin (MSLN), NKCSI, NKG2D ligands, c-Met, NY-ESO-1, oncofetal antigen (h5T4), PDL1, PRAME, prostate stem cell antigen (PSCA), PRAME prostate-specific membrane antigen (PSMA), tumor-associated glycoprotein 72 (TAG-72), TIM-3, TRBC1, TRBC2, vascular endothelial growth factor R2 (VEGF-R2), Wilms tumor protein (WT-1), and a pathogen antigen; or
(ii) the engager comprises a first binding domain having a different tumor targeting specificity from the CAR, and wherein the binding domain is specific to any one of B7H3, CD10, CD19, CD20, CD22, CD24, CD30, CD33, CD34, CD38, CD44, CD79a, CD79b, CD123, CD138, CD179b, CEA, CLEC12A, CS-1, DLL3, EGFR, EGFRvIII, EpCAM, FLT-3, FOLR1, FOLR3, GD2, gpA33, HER2, HM1.24, LGR5, MSLN, MCSP, MICA/B, PSMA, PAMA, P-cadherin, or ROR1; or
(iii) the engager comprises a second binding domain having specificity that is different from the specificity of the first binding domain and is to an extracellular portion of CD3, CD28, CD5, CD16, CD64, CD32, CD33, CD89, NKG2C, NKG2D, or any functional variants thereof, or
(iv) the engager comprises a cytokine or a variant thereof between the first and the second binding domains, wherein the cytokine comprises at least one of IL2, IL4, IL6, IL7, IL9, IL10, IL11, IL12, IL15, IL18, and IL21.

65. The method of claim 61, wherein the cells of the composition are iPSC-derived effector cells further comprising one or more of:
(i) a CD38 knockout;
(ii) an exogenous CD16 or a variant thereof;
(iii) HLA-I and/or HLA-II deficiency;
(iv) introduction of HLA-G or non-cleavable HLA-G, or knockout of one or both of CD58 and CD54;
(v) a CFR;
(vi) a signaling complex comprising a partial or full peptide of a cell surface expressed exogenous cytokine or a receptor thereof;
(vii) an inactivation CAR;
(viii) disruption of at least one of B2M, CIITA, TAP1, TAP2, Tapasin, NLRC5, RFXANK, RFX5, RFXAP, TCR, NKG2A, NKG2D, CD25, CD69, CD44, CD56, CIS, CBL-B, SOCS2, PD1, CTLA4, LAG3, TIM3, and TIGIT; and/or
(ix) introduction of at least one of HLA-E, 4-1BBL, CD3, CD4, CD8, CD16, CD47, CD113, CD131, CD137, CD80, PDL1, A_{2A}R, antigen-specific TCR, Fc receptor, an antibody or functional variant or fragment thereof, a checkpoint inhibitor, and surface triggering receptor for coupling with an agonist.

66. The method of claim 61, wherein administration of the cells of the composition results in one or more of:
(i) increased cytotoxicity;
(ii) improved persistency and/or survival;
(iii) enhanced ability in migrating, and/or activating or recruiting bystander immune cells, to tumor sites;
(iv) improved tumor penetration;
(v) enhanced ability to reduce tumor immunosuppression;
(vi) improved ability in rescuing tumor antigen escape;
(vii) controlled apoptosis;
(viii) enhanced or acquired ADCC; and
(ix) ability to avoid fratricide,
in comparison to administration of their counterpart primary cells.

67. A chimeric antigen receptor (CAR) comprising a binding domain that comprises:
(i) an amino acid sequence that is of at least about 99%, 98%, 96%, 95%, 90%, 85%, or 80% identity to SEQ ID NO: 36, 37, 38, 39, 40, or 41;
(ii) an amino acid sequence represented by a variant of SEQ ID NO: 36, and wherein the variant has one or more mutations at positions comprising 1, 40, 46, 79, 87, 88, 89, 97, 98, and 117 of SEQ ID NO: 36;
(iii) an amino acid sequence represented by a variant of SEQ ID NO: 36, wherein the variant has one or more substitutions comprising Q1E, T40A, E46V, G79L, K87R, P88A, D89E, V97A, S98R, and Q117L according to SEQ ID NO:36; or
(iv) an amino acid sequence represented by any of SEQ ID NOs: 36, 37, 38, 39, 40, and 41.

68. The CAR of claim 67, further comprising a hinge peptide that comprises no more than 80 amino acids, or comprises between 80 to 180 amino acids, or comprises no more than 229 amino acids.

69. The CAR of claim 67, wherein the CAR has at least one of the following characteristics:
(i) being T cell specific;
(ii) being NK cell specific;
(iii) binding to tumor cell surface B7H3;
(iv) reducing tumor cell surface shedding of B7H3 antigen; or
(v) increasing tumor cell surface B7H3 density.

70. The CAR of claim 67, wherein when the CAR is expressed in an effector cell, said effector cell has one or more of the following characteristics:
(i) enhancing effector cell activation and killing function compared to a corresponding effector cell lacking the chimeric receptor; and
(ii) capable of in vivo tumor progression control, tumor cell burden reduction, tumor clearance, and/or improving rate of survival of a subject carrying the tumor compared to a corresponding cell lacking the chimeric receptor.

71. A method of manufacturing the derivative cell comprising a polynucleotide encoding the CAR of any one of claims 67-69, wherein the method comprises differentiating an iPSC to obtain the derivative cells, wherein the polynucleotide encoding the CAR is introduced into the iPSC before differentiation or is introduced to the derivative cells after iPSC differentiation.
